(19) United States
(12) Patent Application Publication
(10) Pub. No.: US 2025/0161049 A1
(43) Pub. Date: May 22, 2025

(54) VALVE LEAFLET TREATMENT TECHNIQUES

(71) Applicant: Edwards Lifesciences Innovation (Israel) Ltd., Caesarea (IL)

(72) Inventors: Carmel Peleg, Pardes Hana-Karkur (IL); Gidon Pesach, Kfar Vitkin (IL); Eran Hoffer, Yahud (IL); Yaron Herman, Givat Ada (IL); Ido Halabi, Givatayim (IL); Bar Eytan-Vaisman, Gedera (IL)

(21) Appl. No.: 18/943,745

(22) Filed: Nov. 11, 2024

Related U.S. Application Data
(63) Continuation of application No. PCT/IB2023/054824, filed on May 10, 2023.
(60) Provisional application No. 63/369,927, filed on Jul. 29, 2022.
(60) Provisional application No. 63/341,376, filed on May 12, 2022.

(51) Int. Cl. A61F 2/24

(52) U.S. Cl. CPC A61F 2/2457; A61F 2/2463; A61F 2/2466; A61F 2220/0016

(57) ABSTRACT

An implant (150) comprises a patch (210), a patch anchor (240), a downstream assembly (300) comprising a winch (320) coupled to a winch anchor (310), and a tether (160) tethering the winch to the patch. A delivery tool (400) has a distal portion that is transluminally advanceable to a heart while the implant is mounted on the delivery tool. The delivery tool has a shaft, a clasp (430), comprising an upstream support and a downstream support, the clasp configured to grasp a portion of a leaflet of a valve of the heart between the upstream support and the downstream support of the clasp, and a driver, configured to anchor the patch to the portion of the leaflet using the patch anchor while the portion of the leaflet remains grasped by the clasp. Other embodiments are also described.

### CROSS-REFERENCE TO RELATED APPLICATIONS

This application is a continuation of International Patent Application No. PCT/IB2023/054824, filed May 10, 2023, which claims the benefit of U.S. Patent Application No. 63/341,376, filed May 12, 2022, and the benefit of U.S. Patent Application No. 63/369,927, filed Jul. 29, 2022, the entire disclosures all of which are incorporated by reference for all purposes. This application is related to International Patent Application No. PCT/IB2021/060436, filed Nov. 11, 2021, the entire disclosure which is incorporated by reference for all purposes.

### BACKGROUND

Ischemic heart disease can cause mitral regurgitation by the combination of ischemic dysfunction of the papillary muscles, and the dilatation of the left ventricle that is present in ischemic heart disease, with the subsequent displacement of the papillary muscles and the dilatation of the mitral valve annulus.

Dilation of the annulus of the mitral valve prevents the valve leaflets from fully coapting when the valve is closed. Mitral regurgitation of blood from the left ventricle into the left atrium results in increased total stroke volume and decreased cardiac output, and ultimate weakening of the left ventricle secondary to a volume overload and a pressure overload of the left atrium.

Chronic or acute left ventricular dilatation can lead to papillary muscle displacement with increased leaflet tethering due to tension on chordae tendineae, as well as annular dilatation.

### SUMMARY

This summary is meant to provide some examples and is not intended to be limiting of the scope of the invention in any way. For example, any feature included in an example of this summary is not required by the claims, unless the claims explicitly recite the features. Also, the features described can be combined in a variety of ways. Various features and steps as described elsewhere in this disclosure can be included in the examples summarized here.

In some implementations, systems and/or apparatuses are provided comprising leaflet patches (e.g., leaflet-augmentation patches), repair chords, and/or delivery tools for implantation thereof. The systems/apparatuses can comprise subvalvular apparatus and/or components. In some implementations, the systems/apparatuses are provided for facilitating leaflet augmentation.

In some implementations, the systems, apparatuses, and methods described herein can be used for providing artificial chordae tendineae and/or leaflet augmentation for the left side of the heart. In some implementations, the systems, apparatuses, and methods described herein can be used for providing artificial chordae tendineae and/or leaflet augmentation for the right side of the heart. In some implementations, the systems, apparatuses, and methods described herein can be used to adjust a length between two portions of the heart wall.

The systems, apparatuses, devices, components, etc. above can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The method(s) herein and any methods of using the systems, assemblies, apparatuses, devices, etc. herein can be performed on a living animal or on a simulation, such as on a cadaver, cadaver heart, simulator (e.g., with the body parts, heart, tissue, etc. being simulated), etc.

There is therefore provided, in accordance with some implementations, a system for use with a valve disposed between an atrium and a ventricle of a real or simulated heart of a real or simulated subject, the system including an implant, and/or a delivery tool.

The implant may include a patch, a patch anchor, a downstream assembly, and/or a tether.

The patch may include a flexible sheet. The downstream assembly may include a ventricular anchor. The tether may tether the downstream assembly to the patch.

The delivery tool may have a distal portion that is transluminally advanceable to the heart while the implant is mounted on the delivery tool.

The delivery tool may include a shaft, a clasp, and/or a driver.

The shaft may define a longitudinal axis of the delivery tool.

The clasp may include an upstream support and a downstream support, the clasp being transitionable between:
an open state in which the upstream support and the downstream support are positioned away from each other, and in which the clasp is configured to receive a portion of a leaflet of the valve between the upstream support and the downstream support, and/or
a grasping state, the clasp being configured to grasp the portion of the leaflet received between the upstream support and the downstream support by being transitioned from the open state toward the grasping state while the portion of the leaflet remains disposed between the upstream support and the downstream support.

The driver may be configured to anchor the patch to the portion of the leaflet using the patch anchor while the portion of the leaflet remains grasped by the clasp.

For some implementations, the clasp is transitionable toward the open state subsequently to anchoring of the patch to the leaflet in order to release, from the clasp, the portion of the leaflet with the patch anchored thereto.

For some implementations, in the grasping state, the upstream support and the downstream support are closer to each other than in the open state.

For some implementations:
the ventricular anchor is a first ventricular anchor,
the downstream assembly further includes a second ventricular anchor, and/or
the tether tethers the patch to both the first ventricular anchor and the second ventricular anchor.

For some implementations, the patch includes a first part of the sheet, and a second part of the sheet is shaped to extend away from the patch in a manner that defines the tether.

For some implementations, the clasp includes a grasping indicator, flexibly coupled to the upstream support in a manner in which, upon grasping of the portion of the leaflet between the upstream support and the downstream support, the portion of the leaflet moves the grasping indicator with respect to the upstream support in a manner that is detectable fluoroscopically.

For some implementations, the patch anchor is coupled to the patch in a manner that facilitates the anchoring of the patch to the portion of the leaflet by:
allowing the driver to temporarily move the patch anchor away from the patch while the patch anchor remains coupled to the patch, and/or
biasing the patch anchor to return toward the patch.

For some implementations, the delivery tool is configured such that a steerable part of the shaft, distal from the clasp, is steerable via operation of an extracorporeal proximal portion of the delivery tool.

For some implementations, the implant is mounted or mountable on the delivery tool such that the tether extends from the downstream assembly, alongside the shaft, past the clasp, and to the patch.

For some implementations, the clasp, in both the open state and the grasping state, is disposed entirely laterally from the shaft.

For some implementations, the ventricular anchor includes a helical tissue-engaging element.

For some implementations, the tether extends from the downstream assembly to the patch, and back to the downstream assembly.

For some implementations:
the ventricular anchor is a first ventricular anchor,
the downstream assembly further includes a second ventricular anchor, and/or
the tether extends from the first ventricular anchor to the patch, and back to the second ventricular anchor.

For some implementations:
the implant includes an upstream assembly including the patch and the patch anchor, and/or
the tether is slidably coupled to the upstream assembly.

For some implementations, the upstream assembly defines an eyelet, and the tether is slidably coupled to the upstream assembly by being threaded through the eyelet.

For some implementations:
the downstream assembly includes a winch coupled to the ventricular anchor, and/or the tether is arranged in a pulley arrangement in which:
a first end of the tether is operatively coupled to the winch,
a bight of the tether is slidably coupled to the upstream assembly, and/or
a second end of the tether is fixed to the downstream assembly.

For some implementations, the winch has a housing, fixedly attached to the ventricular anchor, and the second end of the tether is fixed to the housing.

For some implementations, the patch has a lip region, and the tether is attached to the patch via two lateral lines that diverge away from the tether and from each other, and that are attached to opposing lateral sites in the lip region.

For some implementations, the attachment of the tether to the patch via the two lateral lines is such that tension applied to the tether flexes the patch medially, the patch being configured to elastically flex medially.

For some implementations, the system further includes a medial line connecting the tether to a medial site in the lip region in a manner that limits an extent to which tension applied to the tether flexes the patch medially.

For some implementations:
the downstream assembly further includes a winch, the ventricular anchor being a winch anchor that is coupled to the winch, and/or
the tether tethers the winch to the patch.

For some implementations:
the winch includes a housing and a spool disposed therein, the spool operatively coupled to the tether such that actuation of the winch tensions the tether,
the tether extends, from the spool and out of an aperture of the housing, the aperture having a rim, and/or
the downstream assembly further includes a spring, coupled to the housing in a manner that urges the tether away from contact with the rim.

For some implementations, the spring is a volute spring.

For some implementations, the spring is a cantilever spring.

For some implementations, the spring is a wave spring.

For some implementations, the spring is coupled to the housing in a manner that urges the tether away from contact with a side of the rim that is furthest away from the winch anchor.

For some implementations, the downstream assembly includes a helix that defines:
the spring, and/or
a gripping region adapted to grip the tether.

For some implementations, the spring defines a helix having a series of turns.

For some implementations, the helix extends circumferentially around an exterior of the winch housing.

For some implementations, the spring is adapted to grip the tether in between the turns of the helix.

For some implementations, the delivery tool further includes a driveshaft subassembly, the driveshaft subassembly including one or more driveshafts, extending through the shaft, and operatively coupled to the downstream assembly in a manner that configures the driveshaft subassembly:
to anchor the winch anchor to ventricular tissue of the heart by applying an anchoring force to the winch anchor, and/or
to actuate the winch independently of applying the anchoring force.

For some implementations:
the delivery tool is configured to actuate the winch by applying torque to the winch via the driveshaft subassembly, and/or
the downstream assembly includes a slip clutch that operatively couples the driveshaft subassembly to the winch in a manner that limits a magnitude of torque that the delivery tool may apply to the winch.

For some implementations:
the driveshaft subassembly includes a reference-force tube that extends through the shaft and is engaged with the downstream assembly, and/or
the one or more driveshafts extend through the reference-force tube to the downstream assembly, and/or
the driveshaft subassembly is configured to actuate the winch by applying torque to the winch while the reference-force tube provides a reference force to the downstream assembly.

For some implementations, the downstream assembly and the delivery tool are configured to facilitate the delivery tool rotating the winch anchor with respect to the shaft without actuating the winch.

For some implementations, the driver is configured to anchor the patch to the portion of the leaflet by driving the patch anchor through the portion of the leaflet grasped by the clasp.

For some implementations, the patch anchor is a toggle that is biased to automatically widen upon deployment.

For some implementations, the toggle has a cellular structure that is biased to automatically widen by foreshortening.

For some implementations, the delivery tool is configured to anchor the downstream assembly to ventricular tissue of the ventricle by anchoring the ventricular anchor to the ventricular tissue.

For some implementations, the ventricular anchor includes a tissue-engaging element, and the delivery tool is configured to anchor the downstream assembly to the ventricular tissue by driving the tissue-engaging element into the ventricular tissue.

For some implementations, the implant is mounted or mountable on the delivery tool such that the ventricular anchor is disposed at a distal end of the shaft.

For some implementations, the delivery tool further includes a driveshaft subassembly, the driveshaft subassembly including one or more driveshafts extending through the shaft and operatively coupled to the downstream assembly in a manner that configures the driveshaft subassembly to anchor the ventricular anchor to the ventricular tissue by applying an anchoring force to the ventricular anchor.

For some implementations, the delivery tool includes a capsule coupled to a distal end of the shaft, the distal portion of the delivery tool being transluminally advanceable to the heart while the downstream assembly is housed within the capsule.

For some implementations, the capsule includes a shroud formed from a resilient polymer.

For some implementations, the capsule further includes a housing having multiple fingers that are flexible, distributed circumferentially to approximate a tubular shape, and embedded within the shroud.

For some implementations:
the downstream assembly further includes a winch, the ventricular anchor being a winch anchor that is coupled to the winch, and/or
the delivery tool further includes a driveshaft subassembly, the driveshaft subassembly including one or more driveshafts, extending through the shaft, and operatively coupled to the downstream assembly in a manner that configures the driveshaft subassembly to screw the tissue-engaging element into the ventricular tissue by applying torque to the winch anchor without rotating the winch with respect to the ventricular tissue.

For some implementations, the distal portion of the delivery tool is coupled to the implant in a manner that configures the driveshaft subassembly to screw the tissue-engaging element into the ventricular tissue by applying the torque to the winch anchor without rotating the winch with respect to the shaft.

For some implementations:
the delivery tool includes a capsule coupled to a distal end of the shaft, the distal portion of the delivery tool being transluminally advanceable to the heart while the downstream assembly is housed within the capsule, and/or
the capsule and the winch are shaped to inhibit rotation of the winch with respect to the shaft while the driveshaft subassembly screws the tissue-engaging element into the ventricular tissue.

For some implementations:
the capsule defines a track with which the winch is engaged while housed by the capsule, and/or
the capsule and the downstream assembly configure the driveshaft subassembly to screw the tissue-engaging element into the ventricular tissue in a manner in which the downstream assembly advances distally out of the capsule, with the winch sliding linearly along the track.

For some implementations:
the track is a lateral opening in the capsule,
the winch defines an aperture through which the tether extends out of the winch to the patch, and/or
protrusion of the aperture into the lateral opening configures the driveshaft subassembly to screw the tissue-engaging element into the ventricular tissue in a manner in which the downstream assembly advances distally out of the capsule, with the aperture of the winch sliding linearly along the lateral opening.

For some implementations, the capsule includes:
a housing that houses the winch and that defines the lateral opening; and/or
a resilient shroud that shrouds the housing.

For some implementations:
the shroud shrouds a distal region of the lateral opening, leaving a proximal region of the lateral opening exposed to define a window of the capsule, and/or
the distal portion of the delivery tool is transluminally advanceable to the heart while the downstream assembly is housed within the capsule with the aperture exposed at the window.

For some implementations, the shroud defines a slit that extends distally from the window, aligned with the lateral opening.

For some implementations, protrusion of the aperture into the lateral opening configures the driveshaft subassembly to screw the tissue-engaging element into the ventricular tissue in a manner in which the downstream assembly advances distally out of the capsule, with the aperture of the winch transiently separating the shroud at the slit as the aperture slides linearly along the lateral opening.

For some implementations, the implant includes an upstream assembly including the patch anchor coupled to the patch.

For some implementations, the upstream assembly further includes a cord via which the patch anchor is coupled to the patch.

For some implementations, the patch anchor is a toggle anchor.

For some implementations, the toggle anchor is a helical coil that defines a lumen therethrough.

For some implementations, the driver is configured to drive the anchor through the leaflet while the driver extends through the lumen.

For some implementations, the system further includes a retrieval line that extends away from the toggle anchor, the retrieval line being threaded through turns of the coil in a manner in which tensioning the retrieval line stiffens the anchor by compressing the turns against each other.

For some implementations, the helical coil extends helically around and along a toggle axis, and the system further includes a retrieval line that extends along the toggle axis and away from the toggle anchor in a manner in which tensioning the retrieval line stiffens the anchor by compressing the turns against each other.

For some implementations, the retrieval line is fixed to a first end of the toggle anchor and extends along the toggle axis to a second end of the toggle anchor and, from the second end of the toggle anchor, away from the toggle anchor.

For some implementations, the cord is connected to a midportion of the coil.

For some implementations, the cord is connected to the midportion by looping around a turn of the coil.

For some implementations:
the toggle anchor has a tip, a heel, and an eyelet partway between the tip and the heel, and/or
the heel is flared in a manner that:
facilitates passage of the heel through the leaflet in a first direction, and/or inhibits passage of the heel through the leaflet in a second direction that is opposite to the first direction.

For some implementations, the heel defines wings adapted to transiently flex medially toward each other during passage of the heel through the leaflet in the first direction.

For some implementations, the heel defines wings adapted to flex laterally away from each other upon the heel being pushed against the leaflet in the second direction.

For some implementations:
the toggle anchor has a tip, a heel, and a lateral eyelet partway between the tip and the heel, and/or
the cord is connected to the toggle anchor via the lateral eyelet in a manner in which tensioning the cord extends the heel away from the lateral eyelet.

For some implementations:
the toggle anchor has a sharp point, and/or
the cord is connected to the toggle anchor via the lateral eyelet in a manner in which tensioning the cord concurrently (i) extends the heel away from the lateral eyelet and (ii) retracts the point toward the lateral eyelet.

For some implementations, the system further includes a retrieval line, threaded through the toggle anchor in a manner in which tensioning the retrieval line retracts the heel toward the lateral eyelet.

For some implementations, the toggle anchor further includes a spring, configured to bias the heel to extend away from the lateral eyelet.

For some implementations, the toggle anchor has a sharp point, and the spring is configured to bias the point to retract toward the lateral eyelet.

For some implementations:
the toggle anchor has a tip, a heel, and a lateral eyelet partway between the tip and the heel,
a first segment of the toggle anchor defines the tip,
a second segment of the toggle anchor is slidably coupled to the first segment, and/or the system further includes a longitudinal member, extending through the lateral eyelet, and connected to the toggle anchor in a manner in which pulling of the longitudinal member slides the second segment axially with respect to the first segment.

For some implementations, the second segment of the toggle anchor defines the heel.

For some implementations, the driver is configured to push the toggle anchor tip-first through the portion of the leaflet, the driver having a drive head, and a rod extending proximally from the drive head, the drive head being connected to the heel via complimentary geometry in a manner that (i) preferentially allows deflection rather than lateral translation of the toggle anchor with respect to the driver, and (ii) allows the heel to disconnect from the driver upon the toggle anchor reaching a predetermined angle with respect to the driver.

For some implementations:
the drive head has a first distally-facing face, and defines a shoulder that defines a second distally-facing face proximal from the first face,
at the heel, the toggle anchor defines a lateral opening through which the shoulder protrudes, such that:
the driver is configured to push the toggle anchor tip-first through the portion of the leaflet by (i) the second distally-facing face pushing distally on the toggle anchor at the lateral opening, and (ii) the first distally-facing face pushing distally on the toggle anchor substantially opposite the lateral opening, and/or
the toggle anchor is allowed to disconnect from the driver by deflecting about a point on the driver proximal from the second distally-facing face such that the lateral opening moves laterally away from the shoulder.

For some implementations:
the drive head defines a knob, and a neck that connects the knob to the rod,
at the heel, the toggle anchor defines appendages that extend proximally beyond the knob and, proximally from the knob, medially toward each other and toward the neck, such that:
the appendages inhibit proximal retraction and lateral translation of the driver from the toggle anchor, and/or
deflection of the toggle anchor with respect to the driver urges the knob between the appendages such that the appendages deflect laterally away from each other and from the neck.

For some implementations:
the drive head defines a socket that has a rim,
at the heel, the toggle anchor defines a knob,
the knob is disposed in the socket in a manner that inhibits lateral translation of the toggle anchor from the driver, and/or
deflection of the toggle anchor with respect to the driver presses the rim against the toggle anchor in a manner that levers the knob distally out of the socket.

For some implementations, coupling of the tether to the upstream assembly is such that pulling on the tether pulls on the cord in a manner that draws the patch anchor toward the patch.

For some implementations, the upstream assembly includes a one-way mechanism through which the cord extends, the one-way mechanism being:
mounted on the patch,
configured to facilitate passage of the cord through the one-way mechanism in a first direction that draws the patch anchor toward the patch, and/or
configured to inhibit passage of the cord through the one-way mechanism in a second direction that is opposite to the first direction.

For some implementations, the upstream assembly is configured such that pulling on the tether pulls the cord through the one-way mechanism in the first direction.

For some implementations, the delivery tool is configured to pull on the tether such that the tether pulls the cord through the one-way mechanism in the first direction.

For some implementations, the delivery tool is configured to pull on the tether by moving the downstream assembly away from the upstream assembly subsequently to anchoring the patch to the portion of the leaflet.

For some implementations, the patch anchor has a sharpened tip, and is configured to be driven by the driver through the leaflet with the sharpened tip penetrating the leaflet.

For some implementations, the delivery tool further includes a hollow needle, and the patch anchor is configured to be driven by the driver through the leaflet while disposed within the hollow needle.

For some implementations, the delivery tool further includes a hollow needle configured to pierce the leaflet, and the driver is configured to drive the patch anchor out of the hollow needle while the hollow needle extends through the leaflet.

For some implementations, the patch anchor includes a toggle that defines an eyelet partway along the toggle, the cord being attached to the patch anchor at the eyelet.

For some implementations, the eyelet extends transversely entirely through the toggle.

For some implementations, the toggle is substantially tubular, having a lateral wall that defines a lumen.

For some implementations, the upstream assembly further includes a spring configured to tension the cord.

For some implementations, the spring is a compression spring.

For some implementations, the spring lies substantially flat with respect to the patch.

For some implementations, the spring is configured to facilitate the driver driving the patch anchor through the leaflet by transiently straining in response to tension applied to the cord by the driver pushing the patch anchor away from the patch and through the leaflet.

For some implementations, the spring is coupled to the sheet in a manner in which the patch transiently linearly contracts as the spring transiently strains.

For some implementations, the spring is coupled to the sheet in a manner in which the spring slides across the sheet as the spring transiently strains.

For some implementations:
the patch has a lip and a root,
the driver is configured to anchor the root of the patch to the leaflet such that the lip of the patch extends toward an opposing leaflet of the valve, and/or
the patch includes at least one frame that defines:
a lip brace at the lip of the patch, and/or
a root brace at the root of the patch.

For some implementations, the spring is configured such that the transient straining consists substantially of transient compression of the spring between the lip brace and the root brace.

For some implementations, the at least one frame defines a patch-anchor support coupled to the root brace, the cord extending from the spring, through the patch-anchor support, to the patch anchor.

For some implementations, the tether is connected to the lip brace.

For some implementations, the spring is attached to the root brace.

For some implementations, the spring extends from the root brace to the lip brace.

For some implementations, the spring extends from the root brace to the lip brace along a midline of the patch.

For some implementations, the spring does not extend to the lip brace.

For some implementations, the clasp defines slot, and the driver is configured to anchor the patch to the leaflet by driving the patch anchor through the leaflet and the slot.

For some implementations, the clasp defines a resilient tooth configured to facilitate the patch anchor being driven by the driver through the slot, and to inhibit the patch anchor from being withdrawn, in a reverse direction, through the slot.

For some implementations, the tooth is configured to be transiently pushed aside by the patch anchor being driven by the driver through the slot.

For some implementations, the delivery tool is configured to orient the driver with respect to the slot, such that, as the driver drives the patch anchor through the slot, the patch anchor rubs along a rim of the slot.

For some implementations, the slot is defined by the downstream support of the clasp.

For some implementations, the clasp defines a slot guard, configured to obstruct tissue of the heart from entering the slot.

For some implementations, the patch is coupled to the patch anchor via a cord, and the slot guard:
is resilient,
has a resting position in which it covers an entrance to the slot, thereby obstructing tissue of the heart from entering the slot, and/or
is transiently deflectable away from the slot by the cord, thereby facilitating exiting of the cord from the slot.

For some implementations, a free end of the slot guard is tucked underneath the downstream support.

For some implementations, the delivery tool further includes a capsule at a distal end of the shaft, the capsule configured to house the downstream assembly.

For some implementations, the capsule includes a shroud formed from a resilient polymer.

For some implementations, the capsule further includes a housing having multiple fingers that are flexible, distributed circumferentially to approximate a tubular shape, and embedded within the shroud.

For some implementations, the capsule is shaped to define a lateral window therein.

For some implementations, the capsule is shaped to define a narrow slit that extends between the lateral window and an open distal end of the capsule.

For some implementations, the delivery tool has an extracorporeal proximal portion that includes a clasp controller operatively coupled to the clasp such that operation of the clasp controller transitions the clasp between the open state and the grasping state.

For some implementations, the clasp controller is operatively coupled to the upstream support of the clasp such that operation of the clasp controller transitions the clasp between the open state and the grasping state via movement of the upstream support with respect to the shaft.

For some implementations:
the delivery tool further includes a pair of clasp-control wires via which the clasp controller is operatively coupled to the clasp, and/or the extracorporeal portion includes a lever:
via which the clasp controller is operatively coupled to both wires of the pair, and/or
adapted to pivot in a manner that balances the wires of the pair with respect to each other.

For some implementations, the lever has a fulcrum at which the clasp controller is pivotably attached to the lever, and each wire of the pair is coupled to the lever at respective opposite sides of the fulcrum.

For some implementations, the extracorporeal proximal portion further includes a driver controller operatively coupled to the driver such that operation of the driver controller induces the driver to anchor the patch anchor to the leaflet.

For some implementations:
the patch anchor is a first patch anchor,
the implant further includes a second patch anchor,
the driver is a first driver, configured to anchor the patch to the portion of the leaflet by pushing the first patch anchor through the portion of the leaflet,
the delivery tool further includes a second driver, configured to anchor the patch to the portion of the leaflet by pushing the second patch anchor through the portion of the leaflet, and/or
the extracorporeal portion includes a lever:

via which the driver controller is operatively coupled to the first and second drivers, and/or adapted to pivot in a manner that balances the first driver with the second driver.

For some implementations, the lever has a fulcrum at which the driver controller is pivotably attached to the lever, and the first and second drivers are coupled to the lever at respective opposite sides of the fulcrum.

For some implementations:
within the distal portion of the delivery tool the shaft has a proximal part and a distal part, and/or
the extracorporeal proximal portion of the delivery tool further includes a shaft extender, operatively coupled to the shaft such that operation of the shaft extender reversibly extends the distal part of the shaft distally from the proximal part of the shaft.

For some implementations, the clasp is coupled to the shaft such that extension of the distal part of the shaft distally from the proximal part of the shaft deflects the downstream support with respect to the shaft.

For some implementations:
the delivery tool includes a frame that defines the downstream support, and/or a first part of the frame is attached to the proximal part of the shaft, and a second part of the frame is attached to the distal part of the shaft, such that adjusting a degree of extension of the distal part of the shaft from the proximal part of the shaft deflects the downstream support with respect to the shaft.

For some implementations, the distal part of the shaft includes a steerable part, and the attachment of the first part of the frame and the second part of the frame to the proximal part of the shaft and the second part of the shaft, respectively, is such that extension of the distal part of the shaft distally from the proximal part of the shaft beyond a threshold extent causes the frame to pull the distal part of the shaft to deflect.

For some implementations, the clasp is coupled to the shaft such that extension of the distal part of the shaft distally from the proximal part of the shaft deflects both the downstream support and the upstream support with respect to the shaft.

For some implementations, the clasp is coupled to the shaft such that extension of the distal part of the shaft distally from the proximal part of the shaft deflects both the downstream support and the upstream support with respect to the shaft without changing a disposition between the downstream support and the upstream support.

For some implementations, the clasp is coupled to the shaft such that extension of the distal part of the shaft distally from the proximal part of the shaft deflects both the downstream support and the upstream support with respect to the shaft while the clasp remains in the grasping state.

For some implementations:
the extracorporeal proximal portion further includes an anchor controller, and/or
the delivery tool further includes a driveshaft subassembly that includes one or more driveshafts extending through the shaft, the driveshaft subassembly configured such that, in at least one state of the delivery tool, the driveshaft subassembly operatively couples the anchor controller to the ventricular anchor such that operation of the anchor controller applies an anchoring force to the ventricular anchor.

For some implementations:
the downstream assembly further includes a winch, the ventricular anchor being a winch anchor that is coupled to the winch,
the tether tethers the winch to the patch, and/or
the extracorporeal proximal portion further includes a winch controller, the driveshaft subassembly configured such that, in at least one state of the delivery tool,
the driveshaft subassembly operatively couples the winch controller to the winch such that operation of the winch controller actuates the winch.

For some implementations:
operation of the winch controller actuates the winch by applying torque to the winch via the driveshaft subassembly, and/or
the downstream assembly includes a slip clutch that operatively couples the driveshaft subassembly to the winch in a manner that limits a magnitude of torque that the driveshaft assembly may apply to the winch.

For some implementations, the driveshaft subassembly includes:
a winch-control driveshaft via which the winch controller is operatively coupled to the winch, and/or
an anchor-control driveshaft disposed through the winch-control driveshaft, and via which the anchor controller is operatively coupled to the anchor.

For some implementations:
the anchor-control driveshaft operatively couples the anchor controller to the anchor via engagement of the anchor by a distal-end portion of the anchor-control driveshaft,
the delivery tool further includes a release spring that is biased to pull the anchor-control driveshaft proximally away from the anchor, the engagement of the anchor by the distal-end portion of the anchor-control driveshaft resisting the pulling of the anchor-control driveshaft by the release spring, and/or
the driveshaft subassembly further includes, at the distal-end portion of the anchor-control driveshaft, a lock-rod that maintains the engagement of the anchor by the distal-end portion of the anchor-control driveshaft, such that retraction of the lock-rod from the distal-end portion of the anchor-control driveshaft triggers the release spring to pull the anchor-control driveshaft proximally away from the anchor.

For some implementations:
the release spring is a first release spring, and/or
the delivery tool further includes a second release spring that is biased to pull the winch-control driveshaft proximally away from the winch, the engagement of the anchor by the distal-end portion of the anchor-control driveshaft resisting the pulling of the winch-control driveshaft by the second release spring, such that the retraction of the lock-rod from the distal-end portion of the anchor-control driveshaft also triggers the second release spring to pull the winch-control driveshaft proximally away from the winch.

For some implementations, the bias of the first release spring also maintains the winch-control driveshaft in engagement with the winch by exerting a distally-directed force on the winch-control driveshaft.

For some implementations, the system is configured such that the triggering of the first release spring and the second release spring by the retraction of the lock-rod from the distal-end portion of the anchor-control driveshaft separates the downstream assembly from the delivery tool.

For some implementations:
the downstream assembly further includes a winch, the ventricular anchor being a winch anchor that is coupled to the winch,
the driveshaft subassembly includes a downstream-assembly-control driveshaft, the system has an anchoring state in which the anchor controller is operatively coupled to the winch anchor via the downstream-assembly-control driveshaft such that operation of the anchor controller applies the anchoring force to the winch anchor, and/or
the system has a winching state in which the downstream-assembly-control driveshaft is:

operatively uncoupled from the winch anchor such that operation of the anchor controller does not apply the anchoring force to the winch anchor, and/or operatively coupled to the winch such that rotation of the downstream-assembly-control driveshaft actuates the winch.

For some implementations, the downstream assembly includes an axle that is axially movable within the downstream assembly such that:
positioning the axle in a first axial position within the downstream assembly places the system in the anchoring state, and/or
positioning the axle in a second axial position within the downstream assembly places the system in the winching state.

For some implementations:
the winch includes a spool disposed therein, the spool operatively coupled to the tether such that rotation of the spool tensions the tether,
the axle defines a protruding rim therearound,
the downstream assembly includes a spring-loaded detent that is biased to protrude into a recess defined by a surface of the spool, thereby maintaining the spool in a locked state in which the spool cannot rotate, and/or
transitioning the axle to the second axle position automatically unlocks the winch by the rim pushing the detent out of the recess, thereby allowing rotation of the spool.

For some implementations:
the downstream-assembly-control driveshaft defines an oblique slot, the axle defines a transverse pin,
in an engaged state of the downstream-assembly-control driveshaft in which the downstream-assembly-control driveshaft is locked to the axle:

the transverse pin is disposed transversely within the slot of the downstream-assembly-control driveshaft, and/or
a reference-force tube of the driveshaft subassembly is disposed over the oblique slot and the axle in a manner that cooperates with the transverse pin within the slot to prevent proximal movement of the downstream-assembly-control driveshaft away from the axle by obstructing lateral movement of the downstream-assembly-control driveshaft with respect to the axle, and/or
retracting the reference-force tube from over the oblique slot and the axle allows the downstream-assembly-control driveshaft to move proximally away from the axle by allowing the slot to slide obliquely off the pin.

For some implementations, the first axial position is distal to the second axial position.

For some implementations, the system further has a neutral state in which the downstream-assembly-control driveshaft is coupled to the downstream assembly but is operatively uncoupled from both the winch anchor and the winch.

For some implementations:
in the anchoring state, the downstream-assembly-control driveshaft is disposed in a first axial position with respect to the downstream assembly,
in the winching state, the downstream-assembly-control driveshaft is disposed in a second, different, axial position with respect to the downstream assembly, and/or
the delivery tool is transitionable between the anchoring state and the winching state via axial movement of the downstream-assembly-control driveshaft with respect to the downstream assembly.

For some implementations:
the first axial position is distal to the second axial position, and/or
the delivery tool is transitionable from the anchoring state to the winching state via proximal movement of the downstream-assembly-control driveshaft with respect to the downstream assembly.

For some implementations, the delivery tool further includes a mount, configured to support the patch mounted thereon, and configured to carry the patch toward the clasp while the clasp is in the grasping state.

For some implementations, the mount is configured to carry the patch toward the upstream support of the clasp by moving, with the patch mounted thereon, distally toward the clasp while the clasp is in the grasping state.

For some implementations, the mount is configured to carry the patch toward the upstream support of the clasp by moving, with the patch mounted thereon, distally and laterally toward the clasp while the clasp is in the grasping state.

For some implementations, the delivery tool includes a beam that provides a mechanical linkage between the shaft and the mount, the mechanical linkage linking distalward movement of the mount with lateral movement of the mount.

For some implementations:
the mount has a retracted position, the distal portion of the delivery tool being transluminally advanceable to the heart while the mount is in the retracted position with the patch mounted on the mount,
the mount has a primed position in which the mount is disposed closer to the clasp than in the retracted position, and/or
the driver is configured to anchor the patch to the leaflet by, while the mount is in the primed position with the patch mounted on the mount, driving the patch anchor through the leaflet.

For some implementations:
the mount defines a channel therein,
the distal portion of the delivery tool is transluminally advanceable to the heart while the mount is in the retracted position with the patch mounted on the mount and the patch anchor disposed within the channel, and/or
the driver is configured to anchor the patch to the portion of the leaflet by driving the patch anchor out of the channel and through the portion of the leaflet.

For some implementations:

the delivery tool includes a needle disposed within the channel,
the distal portion of the delivery tool is transluminally advanceable to the heart while the mount is in the retracted position with the patch mounted on the mount and the patch anchor disposed within the needle within the channel, and/or
the delivery tool is configured to facilitate the driver driving the patch anchor through the portion of the leaflet by advancing the needle out of the channel.

For some implementations, the delivery tool further includes a spring that biases the needle to retract into the channel.

For some implementations, the delivery tool further includes a mount-control rod, operatively coupled to the mount in a manner that configures the mount-control rod to transition the mount between the retracted position and the primed position.

For some implementations, the mount-control rod is operatively coupled to the mount by being coupled to the needle.

For some implementations, the operative coupling of the mount-control rod to the mount is such that:
from the retracted position, pushing of the needle by the mount-control rod pushes, via the spring, the mount toward the primed position, and/or
while the mount is in the primed position, pushing of the needle by the mount-control rod strains the spring and advances the needle out of the channel.

For some implementations, the delivery tool includes a spring configured to bias the mount toward assuming the primed position.

For some implementations, the spring is a spring-loaded beam that provides a mechanical linkage between the shaft and the mount, and that biases the mount toward assuming the primed position by biasing the mount to move distalward and laterally.

For some implementations, the driver includes a rod and a drive head, the drive head being coupled to the mount such that tension on the rod constrains the mount in the retracted position.

For some implementations:
the patch anchor and the drive head are disposed within a channel defined in the mount,
relieving the tension on the rod allows the spring to move the mount into the primed position, and/or
while the mount is in the primed position, pushing on the rod moves the drive head through the channel and deploys the patch anchor out of the channel.

For some implementations, the clasp is transitionable between the open state and the grasping state while the mount remains in the retracted position.

For some implementations, the delivery tool has an extracorporeal proximal portion that includes a mount controller operatively coupled to the mount such that operation of the mount controller moves the mount between the retracted position and the primed position.

For some implementations, the delivery tool further includes a mount-control rod via which the mount controller is operatively coupled to the mount.

For some implementations:
the mount-control rod is a first mount-control rod,
the delivery tool further includes a second mount-control rod via which the mount controller is operatively coupled to the mount, and/or
the extracorporeal portion includes a lever:

via which the mount controller is operatively coupled to the first and second mount-control rods, and/or adapted to pivot in a manner that balances the first mount-control rod with the second mount-control rod.

For some implementations, the lever has a fulcrum at which the mount controller is pivotably attached to the lever, and the first and second mount-control rods are coupled to the lever at respective opposite sides of the fulcrum.

For some implementations, the extracorporeal proximal portion further includes a driver controller operatively coupled to the driver such that operation of the driver controller induces the driver to drive the patch anchor through the leaflet.

For some implementations, the mount-control rod is tubular, and the driver extends from the driver controller, through the mount-control rod.

For some implementations, the extracorporeal proximal portion of the delivery tool further includes a clasp controller operatively coupled to the clasp such that operation of the clasp controller transitions the clasp between the open state and the grasping state.

For some implementations, the delivery tool further includes a clasp-control wire via which the clasp controller is operatively coupled to the mount.

For some implementations:
the clasp-control wire is a first clasp-control wire,
the delivery tool further includes a second clasp-control wire via which the clasp controller is operatively coupled to the mount, and/or
the extracorporeal portion includes a lever:
via which the clasp controller is operatively coupled to the first and second clasp-control wires, and/or
adapted to pivot in a manner that balances the first clasp-control wire with the second clasp-control wire.

For some implementations, the lever has a fulcrum at which the clasp controller is pivotably attached to the lever, and the first and second clasp-control wires are coupled to the lever at respective opposite sides of the fulcrum.

For some implementations, the mount controller is configured to, while the clasp is in the grasping state, move the mount between the retracted position and the primed position by sliding the mount over and along the clasp-control wire toward the clasp.

For some implementations, the clasp controller is configured to, while the mount is in the retracted position, transition the clasp from the grasping state to the open state by retracting the clasp-control wire through the mount.

For some implementations, the delivery tool further includes one or more wraps, the distal portion of the delivery tool being transluminally advanceable to the heart while the mount is in the retracted position with the patch held against the mount by the one or more wraps wrapped around the patch and the mount.

For some implementations, the one or more wraps are one or more kirigami wraps.

For some implementations, the delivery tool further includes a release mechanism, adapted to release the patch from against the mount by applying tension to the one or more kirigami wraps.

For some implementations, the delivery tool further includes a release mechanism, adapted to release the patch from against the mount by releasing tension in the one or more kirigami wraps.

For some implementations, the distal portion of the delivery tool is transluminally advanceable to the heart while the mount is in the retracted position with the patch held against the mount by the one or more wraps wrapped around the patch, the mount, and the shaft.

For some implementations, the delivery tool further includes one or more spring-loaded brackets configured to hold the wraps taut.

For some implementations, the delivery tool further includes a rod that cooperates with the spring-loaded brackets to hold the wraps taut, and that is retractable to release the one or more wraps.

For some implementations, in the retracted position, the mount curves in an arc partway around the shaft.

For some implementations, the mount has a convex outer surface, and the patch is mounted on the mount in a manner in which the patch lies in a curve against the convex outer surface of the mount.

For some implementations, the mount is shaped to house the patch anchor while the patch is mounted on the mount.

For some implementations, the patch anchor is coupled to the patch, and the system is configured such that housing of the patch anchor by the mount secures the patch to the mount.

For some implementations, the patch is coupled to the patch anchor via a cord, and is secured to a surface of the mount by the patch anchor being disposed in a channel defined in the surface of the mount, the channel being shaped to:
facilitate sliding of the patch anchor along the channel, and/or
obstruct the patch anchor from exiting the channel laterally.

For some implementations, the driver is configured to anchor the patch to the leaflet by, while the mount is in the primed position with the patch mounted on the mount, driving the patch anchor along the channel, out of an end of the channel, and through the leaflet.

For some implementations, the cord extends from the patch anchor, laterally out of the channel to the patch.

For some implementations:
within the distal portion of the delivery tool the shaft is telescopic,
the delivery tool has a delivery state in which:
the shaft is telescopically extended,
the clasp faces distally, and/or
the distal portion of the delivery tool is transluminally advanceable to the heart.

For some implementations, in the delivery state, the downstream support is deflected distally compared to in the open state.

For some implementations, in the delivery state, the downstream support is disposed adjacent to, and substantially parallel with, the shaft.

For some implementations, in the delivery state, the clasp is closed.

For some implementations, the delivery tool has a contracted state in which:
the shaft is telescopically contracted, and/or
the clasp faces proximally.

For some implementations, the distal portion of the delivery tool is configured to be advanced downstream through the valve while in the contracted state.

For some implementations, in the contracted state, the clasp is closed.

For some implementations, in the contracted state, the downstream support is deflected proximally compared to in the open state.

For some implementations, in the contracted state, the clasp extends further laterally from the shaft than in the open state.

For some implementations, the delivery tool has an extracorporeal proximal portion that includes a shaft extender, operatively coupled to the shaft such that operation of the shaft extender reversibly extends a distal part of the shaft distally from a proximal part of the shaft.

For some implementations, the clasp is coupled to the shaft such that extension of the distal part of the shaft distally from the proximal part of the shaft deflects the downstream support with respect to the shaft.

For some implementations, the patch is substantially trapezoid.

For some implementations:
the patch has a lip and a root,
the driver is configured to anchor the root of the patch to the leaflet such that the lip of the patch extends toward an opposing leaflet of the valve, and/or
the lip is longer than the root.

For some implementations, the delivery tool includes a retrieval line, releasably coupled to the anchor such that tensioning the retrieval line de-anchors the patch anchor from the leaflet.

For some implementations, the delivery tool has an extracorporeal portion, and the retrieval line extends:
from a first end portion of the retrieval line at the extracorporeal portion, transluminally through the delivery tool to the anchor,
at the anchor, the retrieval line loops through the anchor,
from the anchor, transluminally back through the delivery tool to the extracorporeal portion where the retrieval line defines a second end portion.

For some implementations, both the first end portion and the second end portion are coupled to a bobbin that is mounted on the extracorporeal portion.

For some implementations, each of the first end portion and the second end portion extends, from the bobbin, proximally along the extracorporeal portion, towards a bearing, and, at the bearing, turns back on itself to extend distally through the delivery tool to the anchor such that sliding the bobbin distally along the extracorporeal portion tensions the retrieval line.

For some implementations:
the first end portion is wrapped circumferentially around the bobbin,
the bobbin defines a trough that provides accessibility to the first end portion, and/or
the retrieval line is releasable from the anchor by cutting the first end portion via the trough, and subsequently dismounting the bobbin from the extracorporeal portion and moving the bobbin away from the extracorporeal portion.

For some implementations, the bobbin defines a lateral slit, and the bobbin is dismountable from the extracorporeal portion by moving the bobbin laterally off the extracorporeal portion via the lateral slit.

For some implementations, the trough is a trough of a series of troughs that are distributed circumferentially around the bobbin.

For some implementations:
the patch anchor includes a tubular toggle, and includes a retrieval feature including a notch at a heel of the toggle and a retrieval eyelet, and/or
the retrieval line:
extends, colinearly with the toggle, into a lumen of the toggle at the heel of the toggle, exits a lateral wall of the toggle via the retrieval eyelet, and/or loops back to itself via the notch to connect to itself.

For some implementations, the retrieval line is releasably coupled to the anchor such that tensioning the retrieval line facilitates de-anchoring of the patch anchor from the leaflet by reorienting the patch anchor.

There is further provided, in accordance with some implementations, an apparatus for use with a valve disposed between an atrium and a ventricle of a real or simulated heart of a real or simulated subject, the valve having at least a first leaflet and a second leaflet.

The apparatus may include an implant that includes a leaflet-augmentation patch, and/or a patch anchor.

The leaflet-augmentation patch may include:
a flexible sheet, and/or
a frame that supports the flexible sheet.

The patch anchor may be coupled to the patch in a manner that facilitates anchoring of the patch to the first leaflet by:
allowing the patch anchor to be temporarily moved away from the patch while the patch anchor remains coupled to the patch, and/or
biasing the patch anchor to return toward the patch.

For some implementations, the patch anchor has a sharpened tip, and is configured to be driven through the first leaflet with the sharpened tip penetrating the first leaflet.

For some implementations, the patch anchor is configured to be driven through the first leaflet while disposed within a hollow needle.

For some implementations, the patch anchor includes a tubular toggle and includes a retrieval feature including a notch at a heel of the toggle, and a retrieval eyelet, the apparatus further including a retrieval line that:
extends, colinearly with the toggle, into a lumen of the toggle at the heel of the toggle,
exits a lateral wall of the toggle via the retrieval eyelet, and/or
loops back to itself via the notch to connect to itself.

For some implementations, the implant includes:
an upstream assembly including the patch and the patch anchor,
a downstream assembly including a ventricular anchor, and/or
a tether, tethering the patch to the ventricular anchor.

For some implementations:
the ventricular anchor is a first ventricular anchor,
the downstream assembly further includes a second ventricular anchor, and/or
the tether tethers the patch to both the first ventricular anchor and the second ventricular anchor.

For some implementations, the tether extends from the downstream assembly to the patch, and back to the downstream assembly.

For some implementations:
the ventricular anchor is a first ventricular anchor,
the downstream assembly further includes a second ventricular anchor, and/or
the tether extends from the first ventricular anchor to the patch, and back to the second ventricular anchor.

For some implementations, the tether is slidably coupled to the upstream assembly.

For some implementations, the upstream assembly defines an eyelet, and the tether is slidably coupled to the upstream assembly by being threaded through the eyelet.

For some implementations, the downstream assembly includes a winch coupled to the ventricular anchor, and/or the tether is arranged in a pulley arrangement in which:
a first end of the tether is operatively coupled to the winch,
a bight of the tether is slidably coupled to the upstream assembly, and/or
a second end of the tether is fixed to the downstream assembly.

For some implementations, the winch has a housing, fixedly attached to the ventricular anchor, and the second end of the tether is fixed to the housing.

For some implementations, the patch anchor is a toggle that is biased to automatically widen upon deployment.

For some implementations, the toggle has a cellular structure that is biased to automatically widen by foreshortening.

For some implementations, the apparatus further includes a delivery tool, configured to deliver the implant to the heart, and to anchor the patch to the first leaflet by:
anchoring the patch anchor to the first leaflet by temporarily moving the patch anchor away from the patch while the patch anchor remains coupled to the patch, and/or
subsequently, releasing the patch anchor such that the implant responsively returns the patch anchor toward the patch.

For some implementations, the delivery tool is configured to move the patch anchor away from the patch by driving the patch anchor through the first leaflet.

For some implementations, the delivery tool is configured to deliver the implant to the heart with the patch mounted laterally on the delivery tool.

For some implementations:
the implant includes an upstream assembly that includes the patch and the patch anchor, and/or
the implant further includes:
a downstream assembly including a winch coupled to a winch anchor that is configured to anchor the downstream assembly to tissue of the ventricle; and/or a tether, tethering the winch to the patch.

For some implementations, the patch includes a first part of the sheet, and a second part of the sheet is shaped to extend away from the patch in a manner that defines the tether.

For some implementations, the implant further includes a cord via which the patch anchor is coupled to the patch.

For some implementations, the patch includes a spring, and the patch anchor is coupled to the spring in the manner that biases the patch anchor to return toward the patch.

For some implementations, the frame defines the spring.

For some implementations, the spring is a compression spring.

For some implementations, the spring lies substantially flat with respect to the patch.

For some implementations, the implant further includes a cord via which the patch anchor is coupled to the spring.

For some implementations, the spring is configured to facilitate driving of the patch anchor through the first leaflet by transiently straining in response to tension applied to the cord by pushing the patch anchor away from the patch and through the first leaflet.

For some implementations, the spring is coupled to the sheet in a manner in which the patch transiently linearly contracts as the spring transiently strains.

For some implementations, the spring is coupled to the sheet in a manner in which the spring slides across the sheet as the spring transiently strains.

For some implementations:
the patch has a lip and a root,
the cord couples the patch anchor to the patch in a manner in which anchoring the patch anchor to the first leaflet positions the patch such that the lip of the patch extends toward the second leaflet, and/or
the frame defines:
a lip brace at the lip of the patch, and/or
a root brace at the root of the patch.

For some implementations, the spring is configured such that the transient straining consists substantially of transient compression of the spring between the lip brace and the root brace.

For some implementations, the patch defines, along a midline of the patch, a root-to-lip axis between the lip and the root, and the spring is configured such that the transient straining consists substantially of deflection of the spring with respect to the root-to-lip axis.

For some implementations, the spring is configured such that the transient straining consists substantially of deflection of the spring toward the root-to-lip axis.

For some implementations, the spring is a first spring, and the frame further includes a second spring, the first spring and the second spring configured such that the transient straining consists substantially of deflection of the first spring and the second spring toward each other.

For some implementations, the cord extends back and forth between the first spring and the second spring.

For some implementations, the frame defines a patch-anchor support coupled to the root brace, the cord extending from the spring, through the patch-anchor support, to the patch anchor.

For some implementations, the spring is attached to the root brace.

For some implementations, the spring is configured such that the transient straining consists substantially of transient deflection of the spring with respect to the root brace.

For some implementations, the spring does not extend to the lip brace.

For some implementations, the spring extends from the root brace to the lip brace.

For some implementations, the spring extends from the root brace to the lip brace along a midline of the patch.

For some implementations:
the spring is a first spring, extending from the root brace to the lip brace along a first lateral edge of the patch, and/or
the frame defines a second spring, extending from the root brace to the lip brace along a second lateral edge of the patch.

For some implementations, the patch anchor includes a toggle that defines an eyelet substantially midway along the toggle, the cord being attached to the patch anchor at the eyelet.

For some implementations, the apparatus further includes a retrieval line, extending from an end of the toggle, and configured to de-anchor the patch anchor from the first leaflet upon tensioning of the retrieval line.

For some implementations, the eyelet extends transversely entirely through the toggle.

For some implementations, the toggle is substantially tubular, having a lateral wall that defines a lumen.

For some implementations, the lateral wall defines two lateral holes adjacent to each other, the eyelet being defined by a part of the lateral wall disposed between the two lateral holes.

There is further provided, in accordance with some implementations, a system for use with a valve disposed between an atrium and a ventricle of a real or simulated heart of a real or simulated subject, the system including an implant, and/or a delivery tool. The implant may include a tether, and/or an assembly. The assembly may include a winch, and/or a winch anchor. The winch may include a housing and a spool disposed therein, the tether extending from the winch, and the spool operatively coupled to the tether such that actuation of the winch tensions the tether. The winch anchor may be coupled to the winch.

The delivery tool may have a distal portion transluminally advanceable to the heart while coupled to the implant. The delivery tool may include a driveshaft subassembly that includes a reference-force tube and/or a driveshaft. The reference-force tube may be coupled to the housing. The driveshaft may extend through the reference-force tube.

In some implementations, the system:
has an anchoring state in which the driveshaft is:
operatively coupled to the winch anchor such that rotation of the driveshaft applies an anchoring force to the winch anchor, and/or
operatively uncoupled from the winch such that rotation of the driveshaft does not actuate the winch, and/or
has a winching state in which the driveshaft is:

operatively uncoupled from the winch anchor such that rotation of the driveshaft does not apply the anchoring force to the winch anchor, and/or operatively coupled to the winch such that rotation of the driveshaft actuates the winch.

For some implementations, the system further has a neutral state in which the driveshaft is coupled to the implant but is operatively uncoupled from both the winch anchor and the winch.

For some implementations, the assembly includes an axle that is axially movable within the assembly such that:
positioning the axle in a first axial position within the assembly places the system in the anchoring state, and/or
positioning the axle in a second axial position within the assembly places the system in the winching state.

For some implementations:
the winch includes a spool disposed therein, the spool operatively coupled to the tether such that rotation of the spool tensions the tether,
the axle defines a protruding rim therearound,
the assembly includes a spring-loaded detent that is biased to protrude into a recess defined by a surface of the spool, thereby maintaining the spool in a locked state in which the spool cannot rotate, and/or
transitioning the axle to the second axle position automatically unlocks the winch by the rim pushing the detent out of the recess, thereby allowing rotation of the spool.

For some implementations:
the driveshaft defines an oblique slot,
the axle defines a transverse pin,
in an engaged state of the driveshaft in which the driveshaft is locked to the axle:
the transverse pin is disposed transversely within the slot of the downstream-assembly-control driveshaft, and/or
the reference-force tube is disposed over the oblique slot and the axle in a manner that cooperates with the transverse pin within the slot to prevent proximal movement of the driveshaft away from the axle by obstructing lateral movement of the driveshaft with respect to the axle, and/or
retracting the reference-force tube from over the oblique slot and the axle allows the driveshaft to move proximally away from the axle by allowing the slot to slide obliquely off the pin.

For some implementations, the first axial position is distal to the second axial position.

For some implementations:
in the anchoring state, the driveshaft is disposed in a first axial position with respect to the winch,
in the winching state, the driveshaft is disposed in a second, different, axial position with respect to the winch, and/or
the delivery tool is transitionable between the anchoring state and the winching state via axial movement of the driveshaft with respect to the winch.

For some implementations:
the first axial position is distal to the second axial position, and/or
the delivery tool is transitionable from the anchoring state to the winching state via proximal movement of the driveshaft with respect to the winch.

There is further provided, in accordance with some implementations, a system, for use with a real or simulated tissue of a real or simulated subject, the system including a toggle anchor and/or a delivery tool.

The toggle anchor may have a tip and a heel, and define an anchor axis therebetween.

The delivery tool may define a channel in which the toggle anchor is disposed, and/or include a driver configured to push the toggle anchor, tip-first, distally out of and away from the channel, the driver having a drive head, and a rod extending proximally from the drive head, the drive head being connected to the heel via complimentary geometry in a manner that (i) preferentially allows deflection rather than lateral translation of the toggle anchor with respect to the driver, and (ii) allows the heel to disconnect from the driver upon the anchor reaching a predetermined angle with respect to the driver.

For some implementations:
the drive head defines a knob, and a neck that connects the knob to the rod, and/or at the heel, the toggle anchor defines appendages that extend proximally beyond the knob and, proximally from the knob, medially toward each other and toward the neck, such that:
the appendages inhibit proximal retraction and lateral translation of the driver from the toggle anchor, and/or
deflection of the toggle anchor with respect to the driver urges the knob between the appendages such that the appendages deflect laterally away from each other and from the neck.

For some implementations:
the drive head defines a socket that has a rim,
at the heel, the toggle anchor defines a knob,
the knob is disposed in the socket in a manner that inhibits lateral translation of the toggle anchor from the driver, and/or
deflection of the toggle anchor with respect to the driver presses the rim against the toggle anchor in a manner that levers the knob distally out of the socket.

For some implementations, the delivery tool is transluminally advanceable to the tissue.

For some implementations, the tip of the anchor has a sharp point.

For some implementations:
the drive head has a first distally-facing face, and defines a shoulder that defines a second distally-facing face proximal from the first face, and/or
at the heel, the toggle anchor defines a lateral opening through which the shoulder protrudes, such that:
the driver is configured to push the toggle anchor tip-first through the tissue by (i) the second distally-facing face pushing distally on the toggle anchor at the lateral opening, and (ii) the first distally-facing face pushing distally on the toggle anchor substantially opposite the lateral opening, and/or
the toggle anchor is allowed to disconnect from the drive head by deflecting about a point on the driver proximal from the second distally-facing face such that the lateral opening moves laterally away from the shoulder.

For some implementations, the driver further includes a stabilizer, configured such that pushing, by the driver, of the tip of the toggle anchor against the tissue moves the stabilizer into a stabilizing position with respect to the toggle anchor via axial sliding of the stabilizer relative to the toggle anchor, the stabilizer, in the stabilizing position, inhibiting deflection of the toggle anchor with respect to the driver.

For some implementations, the system further includes a cord attached to the toggle anchor.

For some implementations, the system includes an implant including the toggle anchor, the cord, and another component, the cord connecting the other component to the toggle anchor such that the toggle anchor is configured to anchor the other component to the tissue.

There is further provided, in accordance with some implementations, a system, including an implant, and/or a delivery tool. The implant may include a toggle anchor having a body, a tip, and a heel, the toggle anchor defining an anchor axis between the tip and the heel. The delivery tool may be configured to transluminally advance the implant to a real or simulated tissue of a real or simulated subject while the implant is coupled to a distal portion of the tool, and/or include a driver that includes a drive head and a rod extending proximally from the drive head, the driver configured to push the toggle anchor tip-first through the tissue.

The system may include an extendable member, and may be configured such that, upon the driver pushing the tip of the toggle anchor against the tissue, the extendable member responsively slides axially with respect to the body.

For some implementations, at least the tip of the toggle anchor is hollow.

For some implementations, the extendable member is a component of the delivery tool.

For some implementations, the extendable member is a component of the toggle anchor.

For some implementations, the extendable member is a post.

For some implementations:
the system defines a sharp point, configured to pierce the tissue in a manner that facilitates the driver pushing the toggle anchor tip-first through the tissue,
the system has a resting state in which the sharp point is functionally obscured, and/or
the system is configured such that, upon the driver pushing the tip of the toggle anchor against the tissue, the extendable member automatically functionally exposes
the sharp point by sliding axially with respect to the body.

For some implementations:
the sharp point is defined by the tip of the toggle anchor,
in the resting state, the extendable member functionally obscures the sharp point, and/or
the system is configured such that, upon the driver pushing the tip of the toggle anchor against the tissue, the extendable member automatically functionally exposes
the sharp point by sliding proximally away from the sharp point.

For some implementations, the extendable member is a component of the toggle anchor.

For some implementations, the system is configured such that, upon the driver pushing the tip of the toggle anchor against the tissue, the extendable member slides proximally away from the sharp point by sliding into an interior of the toggle anchor.

For some implementations, the system is configured such that, upon the driver pushing the tip of the toggle anchor against the tissue, the extendable member slides proximally away from the sharp point by sliding over an exterior of the toggle anchor.

For some implementations:
the sharp point is defined by the extendable member, in the resting state, the toggle anchor functionally obscures the sharp point, and/or
the system is configured such that, upon the driver pushing the tip of the toggle anchor against the tissue, the extendable member automatically slides distally beyond
the tip, thereby functionally exposing the sharp point.

For some implementations, the extendable member is a component of the delivery tool, and includes a needle that defines the sharp point.

For some implementations, in the resting state, the sharp point is functionally obscured by the toggle anchor.

For some implementations, in the resting state, the sharp point is functionally obscured by being disposed within the body of the toggle anchor.

There is further provided, in accordance with some implementations, a system, including an implant and/or a delivery tool.

The implant may include a toggle anchor having a body, a tip, and a heel, the toggle anchor defining an anchor axis between the tip and the heel.

The delivery tool may be configured to transluminally advance the implant to a real or simulated tissue of a real or simulated subject while the implant is coupled to a distal portion of the tool.

The delivery tool may include a driver and/or a stabilizer. The driver may include a drive head and a rod extending proximally from the drive head, the driver configured to push the toggle anchor tip-first through the tissue. The stabilizer may be configured such that pushing, by the driver, of the tip of the toggle anchor against the tissue moves the stabilizer into a stabilizing position with respect to the toggle anchor via axial sliding of the stabilizer relative to the toggle anchor, the stabilizer, in the stabilizing position, inhibiting deflection of the toggle anchor with respect to the driver.

For some implementations, the delivery tool includes a spring that biases the stabilizer away from the stabilizing position.

For some implementations, the delivery tool is configured such that the axial sliding of the stabilizer relative to the toggle anchor is accompanied by movement of the drive head proximally toward the rod.

For some implementations, the drive head is coupled to the rod via a compression spring that compresses upon the driver pushing the tip of the toggle anchor against the tissue, the compression of the spring facilitating the axial sliding of the stabilizer relative to the toggle anchor.

For some implementations, the compression spring is configured to facilitate disengagement of the toggle anchor from the driver upon cessation of the pushing by the driver.

For some implementations, the stabilizer includes a post configured such that pushing, by the driver, of the tip of the toggle anchor against the tissue moves the stabilizer into the stabilizing position via sliding of the post distally into the toggle anchor.

For some implementations, at least the heel of the toggle anchor is tubular, and the post is configured such that pushing, by the driver, of the tip of the toggle anchor against the tissue moves the stabilizer into the stabilizing position via axial sliding of the post into a tubular lumen defined by the toggle anchor.

For some implementations, the stabilizer is disposed inside the driver.

For some implementations, the drive head is coupled to the rod via a compression spring that extends over at least part of the post.

For some implementations, the delivery tool is configured such that the sliding of the post distally into the toggle anchor is accompanied by movement of the drive head proximally toward the rod.

For some implementations, the stabilizer includes a receptacle configured such that pushing, by the driver, of the tip of the toggle anchor against the tissue moves the stabilizer into the stabilizing position via sliding of the heel proximally into the receptacle.

For some implementations, the heel is dimensioned to fit snugly within the receptacle.

For some implementations, the receptacle is tubular.

For some implementations, the receptacle is a cup.

For some implementations, the drive head is coupled to the rod via a compression spring that extends through at least part of the receptacle.

For some implementations, the delivery tool is configured such that the axial sliding of the heel into the receptacle is accompanied by sliding of the drive head proximally into the receptacle.

For some implementations, the delivery tool is configured such that the axial sliding of the heel into the receptacle is accompanied by movement of the drive head proximally toward the rod.

There is further provided, in accordance with some implementations, apparatus for use with a real or simulated tissue, the apparatus including an implant that includes a toggle anchor and/or a longitudinal member.

The toggle anchor may have a tip, a heel, and an anchor axis between the tip and the heel, and define a lateral eyelet partway between the tip and the heel.

The toggle anchor may include:
a first segment, defining the tip, and/or
a second segment, slidably coupled to the first segment, and defining the heel.

The longitudinal member may extend through the lateral eyelet, and be connected to the toggle anchor in a manner in which pulling of the longitudinal member slides the second segment axially with respect to the first segment.

For some implementations, the longitudinal member is connected to the toggle anchor in a manner in which pulling of the longitudinal member slides the heel toward the lateral eyelet.

For some implementations, the longitudinal member is connected to the toggle anchor in a manner in which pulling of the longitudinal member slides the heel away from the lateral eyelet.

For some implementations, the longitudinal member is connected to the toggle anchor in a manner in which pulling of the longitudinal member extends the heel away from the first segment such that the lateral eyelet becomes disposed substantially midway between the tip and the heel of the toggle anchor.

For some implementations:
the longitudinal member is attached to an attachment point of the toggle anchor, and/or
prior to the longitudinal member being pulled, the longitudinal member defines a path that includes at least one turn between the lateral eyelet and the attachment point.

For some implementations, the longitudinal member is connected to the toggle anchor in a manner in which the sliding of the second segment axially with respect to the first segment is accompanied by sliding of the longitudinal member out of the lateral eyelet.

For some implementations, at least part of the second segment is coaxial with at least part of the first segment.

For some implementations, the second segment is telescopically coupled to the first segment, and the longitudinal member is connected to the toggle anchor in a manner in which pulling of the longitudinal member slides the second segment telescopically with respect to the first segment.

For some implementations, the second segment is coupled to the first segment such that the second segment is axially slidable within the first segment.

For some implementations, the toggle anchor includes a spring that biases the second segment toward a predetermined axial position with respect to the first segment.

For some implementations, the longitudinal member is connected to the toggle anchor in a manner in which pulling of the longitudinal member slides the second segment axially away from the predetermined axial position with respect to the first segment.

For some implementations, the longitudinal member is connected to the toggle anchor in a manner in which pulling of the longitudinal member strains the spring.

For some implementations, the longitudinal member is connected to the toggle anchor in a manner in which pulling of the longitudinal member changes an axial length of the toggle anchor by sliding the second segment with respect to the first segment.

For some implementations, the longitudinal member is connected to the toggle anchor in a manner in which pulling of the longitudinal member increases the axial length of the toggle anchor by sliding the second segment with respect to the first segment.

For some implementations, the longitudinal member is connected to the toggle anchor in a manner in which pulling of the longitudinal member reduces the axial length of the toggle anchor by sliding the second segment with respect to the first segment.

For some implementations, the second segment defines a sharp point at an opposite end of the second segment from the heel, and the apparatus is configured such that pulling of the longitudinal member slides the second segment with respect to the first segment in a manner that draws the sharp point into the first segment.

For some implementations, the apparatus is configured such that pulling of the longitudinal member slides the second segment with respect to the first segment in a manner that draws the sharp point into the first segment and extends the heel away from the first segment.

For some implementations, the implant further includes a frame, and the longitudinal member is a cord that connects the toggle anchor to the frame.

For some implementations, the frame includes a spring that pulls on the cord.

For some implementations, the longitudinal member is a retrieval line, configured to pull the toggle anchor out of the tissue.

For some implementations, the lateral eyelet is disposed at an end of the first segment that is closest to the heel.

There is further provided, in accordance with some implementations, apparatus for use with a real or simulated tissue, the apparatus including an implant that includes a toggle anchor, a cord, and/or a retrieval line.

The toggle anchor may have a tip, a heel, and/or a lateral eyelet. The lateral eyelet may be partway between the tip and the heel.

The cord may be connected to the toggle anchor via the lateral eyelet.

The retrieval line may be threaded through the toggle anchor in a manner in which tensioning the retrieval line retracts the heel toward the lateral eyelet.

For some implementations, the toggle anchor is a helical coil that defines a lumen therethrough.

For some implementations, the retrieval line is threaded through turns of the helical coil in a manner in which tensioning the retrieval line stiffens the anchor by compressing the turns against each other.

For some implementations, the toggle anchor further includes a spring, configured to bias the heel to extend away from the lateral eyelet.

For some implementations:
the toggle anchor has a sharp point, and/or
the spring is configured to bias the point toward the lateral eyelet.

For some implementations:
the toggle anchor includes:
a body that defines a lumen and a retrieval eyelet, the retrieval eyelet opening into the lumen; and/or
a stock, at least part of which is disposed within the lumen, and/or
the cord extends through the retrieval eyelet to the stock such that tensioning the retrieval line retracts the heel by sliding the stock with respect to the body.

For some implementations, the cord extends through the retrieval eyelet and a transverse channel in the stock, and is attached to a side of the body opposite the retrieval eyelet.

For some implementations, the stock is shaped to define the heel.

For some implementations, the body defines the lateral eyelet.

There is further provided, in accordance with some implementations, apparatus for use with a real or simulated tissue, the apparatus including an implant that includes a toggle anchor and/or a cord.

In some implementations, the toggle anchor has a tip, a heel, and/or a lateral eyelet partway between the tip and the heel.

The cord may be connected to the toggle anchor via the lateral eyelet in a manner in which tensioning the cord extends the heel away from the lateral eyelet.

For some implementations:
the toggle anchor has a sharp point, and/or
the cord is connected to the toggle anchor via the lateral eyelet in a manner in which tensioning the cord concurrently (i) extends the heel away from the lateral eyelet and (ii) retracts the point toward the lateral eyelet.

For some implementations:
the toggle anchor includes:
a body that defines a lumen and the lateral eyelet, the lateral eyelet opening into the lumen; and/or
a stock, at least part of which is disposed within the lumen, and/or the cord extends through the lateral eyelet to the stock such that tensioning the cord concurrently extends the heel and retracts the point by sliding the stock with respect to the body.

For some implementations, the stock is shaped to define the heel and the point.

For some implementations, the cord extends through the lateral eyelet and a transverse channel in the stock, and is attached to a side of the body opposite the lateral eyelet.

There is further provided, in accordance with some implementations, a system for use with a real or simulated heart of a real or simulated subject, the system including an implant including a tether, and/or an assembly.

The assembly may include a winch, a winch anchor, and/or a spring. The winch anchor may be coupled to the winch.

The winch may include a housing and a spool disposed therein.

The spool may be operatively coupled to the tether such that actuation of the winch tensions the tether.

The tether may extend from the spool and out of an aperture of the housing. The aperture may have a rim.

The spring may be coupled to the housing in a manner that urges the tether away from contact with the rim.

For some implementations, the spring is a volute spring.

For some implementations, the spring is a cantilever spring.

For some implementations, the spring is a wave spring.

For some implementations, the spring is coupled to the housing in a manner that urges the tether away from contact with a side of the rim that is furthest away from the winch anchor.

For some implementations, the assembly is a first assembly of the implant, and the implant further includes a second assembly including an anchor, the first assembly and the second assembly being connected via the tether.

For some implementations:
the heart has an atrium, a ventricle, and a valve therebetween,
the system further includes a delivery tool, having a distal portion transluminally advanceable to the heart while coupled to the implant, and adapted to:
anchor the anchor into a leaflet of the valve, and/or anchor the winch anchor into tissue of the ventricle, such that the tether extends from
the anchor at the leaflet, to the winch anchor within the ventricle.

For some implementations, the first assembly includes a helix that is shaped to define:
the spring; and/or
a gripping region adapted to grip the tether between turns of the helix.

For some implementations, the spring defines a helix having a series of turns that extend circumferentially around the housing, and during ventricular systole of the heart, a pitch between turns of a first portion of the helix is reduced.

For some implementations, the spring is adapted to grip the tether in between turns of a second portion of the helix.

For some implementations, the spring defines a helix having a series of turns.

For some implementations, the helix extends circumferentially around an exterior of the winch housing.

For some implementations, the spring is adapted to grip the tether in between the turns of the helix.

There is further provided, in accordance with some implementations, a method of connecting a tether to a component of an implant, the method including:
forming a bight in the tether by looping an end portion of the tether around a part of the component, and/or
closing the bight into a loop by burrowing the end portion coaxially through a stretch of the tether, such that the stretch squeezes on the end portion therewithin.

For some implementations:
the bight is a first bight,
the loop is a first loop, burrowing the end portion coaxially through the stretch includes burrowing a first part of the end portion coaxially through a stretch, and/or
the method further includes:

forming a second bight in the end portion, and/or closing the second bight into a second loop by burrowing a second part of the end portion coaxially through the stretch, such that the first part and the second part extend alongside each other within the stretch.

For some implementations:
the bight is a first bight,
the loop is a first loop,
the stretch is a first stretch, and/or
the method further includes:

forming a second bight in the end portion, and/or closing the second bight into a second loop by burrowing the end portion coaxially through a second stretch of the tether, such that the second stretch squeezes on the end portion therewithin.

For some implementations:
the end portion is a first end portion of the tether,
a second end portion of the tether extends, from the stretch, away from the first end portion, an end of the second end portion being coupled to a downstream assembly, and/or
looping the end portion of the tether around the part of the component includes looping the first end portion of the tether around a part of a leaflet patch of an upstream assembly.

For some implementations, the stretch is a braid, and burrowing the end portion coaxially through the stretch includes burrowing the end portion coaxially through the stretch such that strands of the braid are pushed apart.

For some implementations, the stretch includes strands of a weave, and burrowing the end portion coaxially through the stretch includes burrowing the end portion coaxially through the stretch such that the strands of the weave are pushed apart.

For some implementations, the method further includes, subsequently to burrowing the end portion coaxially through the stretch, trimming an end part of the end portion that extends, from out of the stretch to an end of the tether.

There is further provided, in accordance with some implementations, an apparatus for use with a real or simulated tissue, the apparatus including an implant that includes a toggle anchor, a retrieval adapter, and/or a retrieval line.

The toggle anchor may have a heel defining a retrieval eyelet.

The retrieval adapter may have a first loop at a first end and a second loop at a second end, the first loop extending through the retrieval eyelet.

The retrieval line may be looped through the second loop in a manner in which pulling on the retrieval line reorients the toggle anchor for retrieval.

For some implementations:
the toggle anchor is a first toggle anchor,
the retrieval adapter is a first retrieval adapter, the apparatus further includes a second toggle anchor, and a second retrieval adapter, and/or
the retrieval line is looped through both the second loop of the first retrieval adapter, and through a second loop of the second retrieval adapter, such that pulling on the retrieval line reorients both the first toggle anchor and the second toggle anchor for retrieval.

There is further provided, in accordance with some implementations, a system for use with a real or simulated subject, the system including a toggle anchor, a cord, and/or a retrieval line.

The toggle anchor may be in the form of a helical coil. The coil may have a longitudinal axis that extends from a first end portion of the coil to a second end portion of the coil.

The cord may be connected to the coil at a site between the first end portion and the second end portion, and may extend, from the site, orthogonally away from the longitudinal axis.

The retrieval line may extend from the first end portion to the second end portion, and away from the toggle anchor, the retrieval line being fixed to the toggle anchor in a manner in which tensioning the retrieval line stiffens the anchor by compressing turns of the coil against each other.

For some implementations, the retrieval line is fixed to the end portion of the toggle anchor.

For some implementations, the system further includes a driver, adapted to drive the toggle anchor from a first side of a cardiovascular tissue of the subject, through the tissue to an opposite side of the tissue, such that, at the opposite side:
the longitudinal axis of the helical coil lies parallel with the tissue, and/or
the coil is in a non-compressed state in which turns of the coil can move with respect to each other.

For some implementations, the driver is adapted to deliver the toggle anchor through the tissue while the driver extends through a lumen defined by the coil.

There is further provided, in accordance with some implementations, apparatus for use with a real or simulated subject, the apparatus including a medical tool that includes an extracorporeal part, a shaft, and/or a pair of wires.

The extracorporeal part may be at a proximal end of the tool.

The shaft may extend distally from the extracorporeal part, and may be configured to be transluminally advanced into the subject.

The pair of wires may extend, from the extracorporeal part, along the shaft, to a distal part of the tool.

The extracorporeal part may include a controller, and/or a lever.

The lever may operatively couple the controller to the distal part via the pair of wires by:
the controller being pivotably attached to the lever at a fulcrum of the lever, and/or each wire of the pair being coupled to the lever at a respective opposite side of the fulcrum,
such that actuation of the controller manipulates the distal part of the tool while the lever balances the wires with respect to each other by pivoting dynamically.

For some implementations, the controller is slidable axially along the extracorporeal part such that, while the lever continues to balance the wires with respect to each other, sliding the controller in a first axial direction moves the distal part of the tool in the first axial direction.

There is further provided, in accordance with some implementations, apparatus for use with a real or simulated subject, the apparatus including an implant, and/or a delivery tool.

The delivery tool may include a kirigami wrap and/or a release mechanism.

The kirigami wrap may be adapted to hold the implant. The delivery tool may be configured to transluminally advance the implant into the subject while the implant is held by the kirigami wrap.

The release mechanism may be operatively coupled to the kirigami wrap in a manner in which actuating the release mechanism releases the hold of the kirigami wrap on the implant.

There is further provided, in accordance with some implementations, a system for use with a real or simulated heart of a real or simulated subject, the system including an implant including a tether, and/or an assembly.

The assembly may include a winch, a winch anchor, and/or a shock absorber. The winch anchor may be coupled to the winch.

The winch may include a housing and a spool disposed therein.

The spool may be operatively coupled to the tether such that actuation of the winch tensions the tether.

The tether may extend from the spool and out of an aperture of the housing.

The shock absorber may be coupled to the housing in a manner that mitigates forces acting on the winch anchor.

The present invention will be more fully understood from the following detailed description of examples thereof, taken together with the drawings, in which:

### BRIEF DESCRIPTION OF THE DRAWINGS

FIGS. 1A-B, 2A-R, 3A-B, 4A-C, 5A-B, and 6A-C are schematic illustrations of an example system(s) and example techniques for using the system(s) to treat a heart of a subject, in accordance with some implementations;

FIGS. 7A-C are schematic illustrations of an example grasping indicator, in accordance with some implementations;

FIGS. 8A-B and 9A-B are schematic illustrations of example steerable versions of a delivery tool, in accordance with some implementations;

FIGS. 10A-B are schematic illustrations of an example patch anchor, in accordance with some implementations;

FIGS. 11A-B are schematic illustrations of an example patch anchor, in accordance with some implementations;

FIG. 12 is a schematic illustration of at least part of an example implant, in accordance with some implementations;

FIG. 13 is a schematic illustration of at least part of an example implant, in accordance with some implementations;

FIGS. 14, and 15A-B are schematic illustrations of an example implant and a technique for use therewith, in accordance with some implementations;

FIGS. 16, 17, 18A-B, and 19A-D are schematic illustrations of example implants in which one or more ventricular anchors are connected to an upstream assembly via multiple stretches of a tether, in accordance with some implementations;

FIGS. 20A-E are schematic illustrations of a distal region of an example delivery tool for an implant, in accordance with some implementations;

FIGS. 21A-E are schematic illustrations of some components of an example driveshaft subassembly, and a downstream assembly of an implant, in accordance with some implementations;

FIGS. 22A-C, 23A-D, 24A-C, 25A-E, 26A-E, 27A-D, 28A-B, 29A-D, 30A-B, 31A-D, 32A-B, 33A-D, 34A-B, and 35A-C are schematic illustrations of various patch anchors and/or anchor drivers, in accordance with some implementations;

FIGS. 36A-C are schematic illustrations of a patch anchor that is in the form of a helical coil, in accordance with some implementations;

FIGS. 37A-C are schematic illustrations of a technique for coupling a tether to a component of an implant, in accordance with some implementations;

FIGS. 38A-E and 39 are schematic illustrations of a technique for coupling, via a tether, two components of a system thereto, in accordance with some implementations;

FIGS. 40A-C, 41A-C, and 42 are schematic illustrations of various systems and techniques for preventing a patch anchor from being inadvertently withdrawn back through tissue of the heart, in accordance with some implementations;

FIGS. 43A-B are schematic illustrations of a kirigami wrap for holding a component of an implant against a delivery tool, in accordance with some implementations;

FIGS. 44A-B are schematic illustrations of a shock-absorbing spring for a ventricular anchor, in accordance with some implementations;

FIGS. 45 and 46 are schematic illustrations of a torque limiter for preventing over-tensioning of a tether, in accordance with some implementations;

FIGS. 47A-B are schematic illustrations of an exemplary downstream assembly, in accordance with some implementations;

FIGS. 48A-D are schematic illustrations of an exemplary coupling of a driveshaft subassembly to a downstream assembly, in accordance with some implementations; and

FIGS. 49, 50A-B, 51A-B, 52A-B, and 53A-B are schematic illustrations of a proximal portion of a delivery tool, in accordance with some implementations.

### DETAILED DESCRIPTION

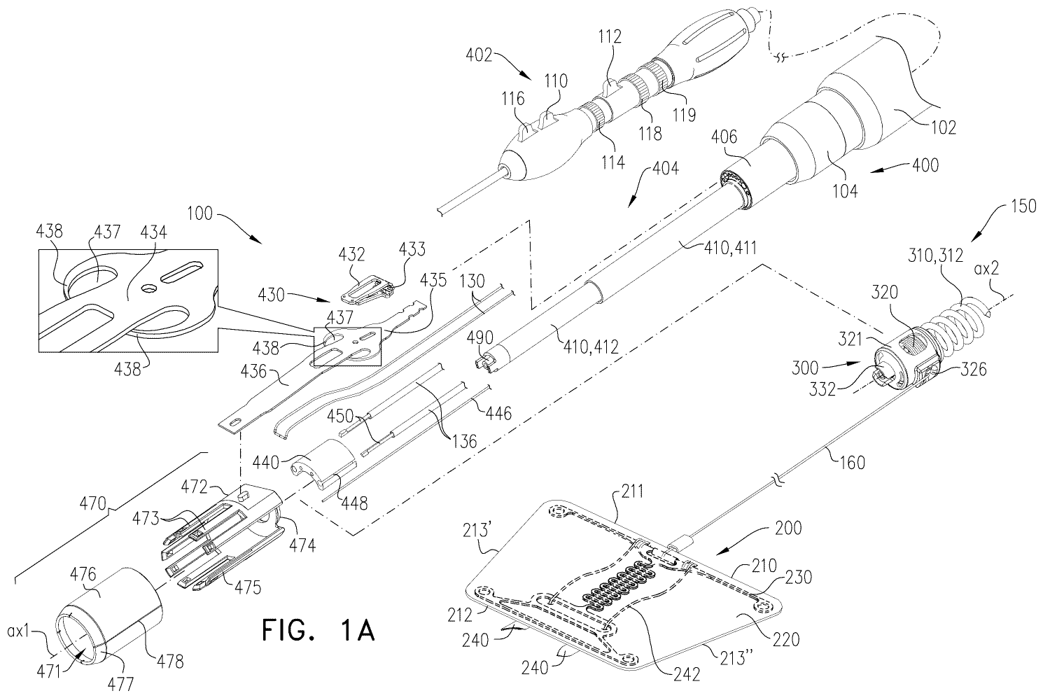

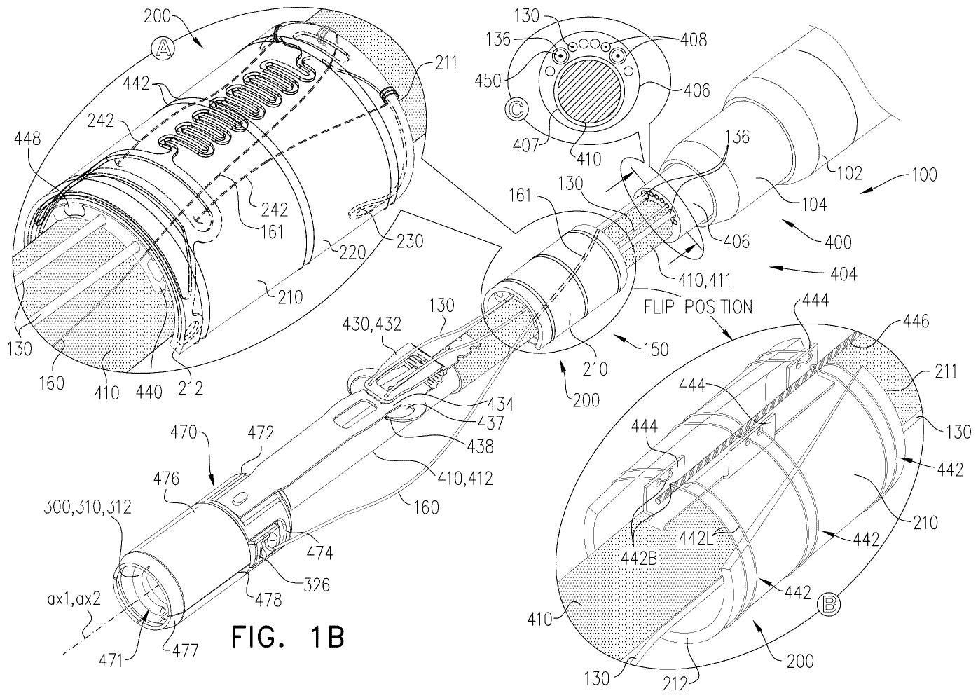

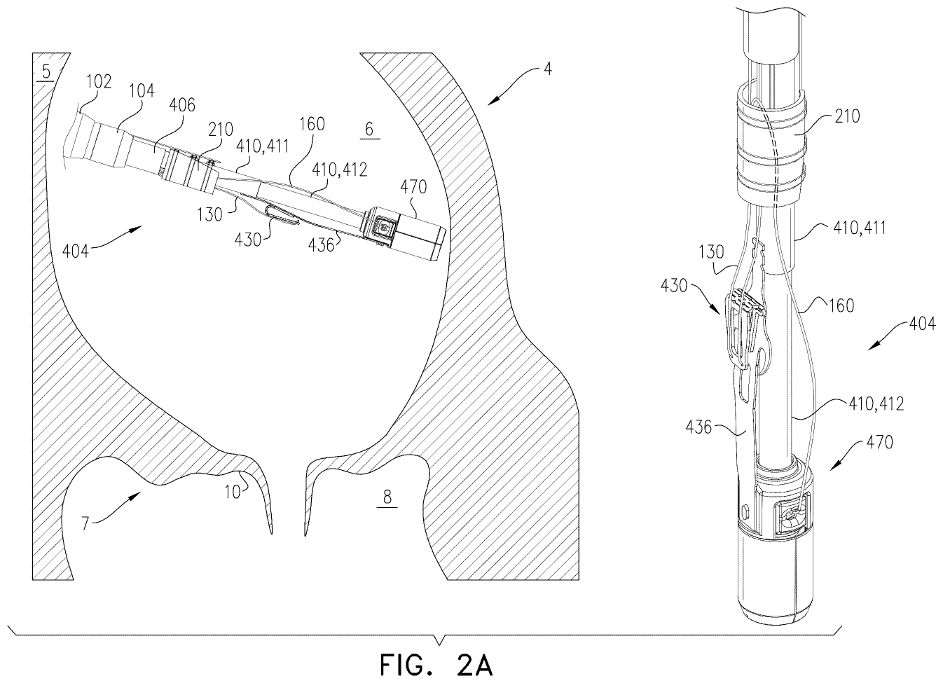

Reference is made to FIGS. 1A-B, 2A-R, 3A-B, 4A-C, 5A-B, and 6A-C, which are schematic illustrations of an example system 100, and example techniques for using the system to treat a heart 4 of a subject, in accordance with some implementations. In particular, system 100 is used to reduce (e.g., eliminate) regurgitation through an atrioventricular valve 7 of the heart, caused by suboptimal coaptation of the leaflets of the valve. System 100 comprises an implant 150, and a delivery tool 400 for delivering and implanting the implant.

FIG. 1A shows system 100 with an exploded view of delivery tool 400, and implant 150 separate from the delivery tool. FIG. 1B shows system 100 assembled, with implant 150 loaded onto delivery tool 400. Implant 150 comprises a patch 210 (e.g., a leaflet-augmentation patch), and can further comprise at least one patch anchor 240. In the example shown, implant 150 comprises two patch anchors 240. Patch 210 and patch anchor 240 can be considered to be components of an upstream assembly 200 of implant 150. Implant 150 further comprises a downstream assembly 300, and a tether 160 that tethers the downstream assembly to upstream assembly 200.

In some implementations, tether 160 comprises a suture. In some implementations, tether 160 comprises a flexible and/or superelastic material, e.g., ePTFE, nitinol, PTFE, polyester, stainless steel, or cobalt chrome. In some implementations, tether 160 is coated with polytetrafluoroethylene (PTFE).

Inset frame A of FIG. 1B uses transparency to illustrate frame 230 within patch 210, whereas inset frame B, for the sake of clarity, does not. Inset B is a view of the opposite side of the apparatus than that shown in inset A. Inset frame C of FIG. 1B shows a cross-section through tool 400. It is to be noted that, for the sake of simplicity, in this cross-section shaft 410 (described hereinbelow) is shown as solid, even though it is tubular, and even though other components such as a driveshaft subassembly 490 (e.g., driveshafts of the driveshaft subassembly) can extend through it.

Patch 210 comprises a flexible sheet 220 and can further comprise at least one frame 230 to which the sheet is attached. Sheet 220 can comprise a polymer such as polyethylene, expanded polytetrafluoroethylene, or polyethylene terephthalate. Sheet 220 can have the structure of a fabric or a film. Frame 230 can provide patch 210 with mechanical properties that would not be provided by sheet 220 alone. Such properties are described in more detail hereinbelow. Although the present disclosure generally refers to sheet 220 in the singular, patch 210 can comprise more than one sheet, arranged in layers, e.g., with frame 230 disposed between the sheets. (This can alternatively be described as sheet 220 comprising multiple layers, e.g., being a multi-layer sheet.) In some implementations, the composition and/or structure of sheet 220, techniques for manufacturing the sheet, and/or techniques for incorporating frame 230 within patch 210 can be as described, mutatis mutandis, in U.S. Patent Application No. 63/341,354 to Vaid et al., filed May 12, 2022, and/or International Patent Application Publication No. WO2023219968 A1 to Vaid et al., filed May 8, 2023 each of which is incorporated herein by reference in its entirety.

Implant 150 (e.g., upstream assembly 200 thereof) can be provided with at least one patch anchor 240 coupled to patch 210, e.g., as shown. However, in some implementations (e.g., for some variants of implant 150 and/or of system 100) anchoring of the patch can include coupling a patch anchor to the patch (e.g., driving the patch anchor through the patch) in situ.

Downstream assembly 300 comprises an anchor 310 for anchoring to ventricular tissue (e.g., a ventricular anchor). Downstream assembly 300 can also comprise a winch 320 coupled to anchor 310, e.g., with the winch disposed within, or forming part of, the head of the anchor. Thus, for applications in which downstream assembly 300 comprises winch 320, anchor 310 can be considered to be a winch anchor. Winch anchor 310 has a tissue-engaging element 312 (e.g., one or more of a screw, helix, dart, pin, hook, staple, barb, arm, sharpened portion, etc.), which can be configured to be driven into tissue. In some implementations, tissue-engaging element 312 is configured to be driven into the tissue along an anchor axis ax2 of the winch anchor. In the example shown, tissue-engaging element 312 is a helical tissue-engaging element, configured to be screwed into tissue along axis ax2. However, it is to be noted that winch anchor 310 can comprise a different type of tissue-engaging element such as, but not limited to, a dart, pin, hook, staple, barb, arm, etc.

Winch 320 comprises a spool 322 (e.g., see FIGS. 3A-4C) that, as shown, can be mounted such that it and/or its axis of rotation is colinear with anchor axis ax2. However, other spool orientations are possible.

Tether 160 is coupled to patch 210, and extends therefrom to winch 320, thereby tethering the winch to the patch. As shown, tether 160 can enter winch 320 via a lateral aperture 326 in a housing 321 of the winch, and/or can reach spool 322 in an orientation that is substantially orthogonal to anchor axis ax2. Although housing 321 is referred to as the housing of winch 320, in some implementations it can be considered to be the housing of downstream assembly 300.

Tether 160 is operatively coupled to winch 320, such that actuation of the winch can adjust an effective length of the tether, e.g., a length of the tether between the winch and patch 210.

Delivery tool 400 has a distal portion 404 that is transluminally (e.g., transfemorally) advanceable to the heart, and can have an extracorporeal proximal portion 402 that can comprise handles and/or controls via which the operator (e.g., the physician) can control (e.g., steer, actuate, etc.) components at the distal portion of the tool, e.g., in order to deliver and implant implant 150. Delivery tool 400 comprises a shaft 410, a clasp 430, and at least one driver 450.

Delivery tool 400 can comprise an overtube 406 defining a primary lumen 407 through which shaft 410 extends. Overtube 406 can also define one or more auxiliary lumens 408 that provide communication to distal portion 404, e.g., for one or more other components of delivery tool 400 to extend therethrough.

Shaft 410 defines a longitudinal axis ax1 of delivery tool 400. In some implementations, and as shown, shaft 410 (and lumen 407 through which it extends) is eccentric with respect to overtube 406, and therefore even if longitudinal axis ax1 is central with respect to shaft 410, it may not be central with respect to delivery tool 400 as a whole. In the example shown, auxiliary lumens 408 are disposed generally on one side of primary lumen 407, e.g., they are distributed circumferentially around less than 220 degrees (e.g., less than 200 degrees, such as less than 180 degrees) around the primary lumen. This arrangement can advantageously facilitate efficient inclusion of clasp 430 within the overall diameter of delivery tool 400. For example, and as shown, clasp 430 can be disposed on the same side of shaft 410 as auxiliary lumens 408 are disposed, but distally from the distal ends of the auxiliary lumens.

Shaft 410 (e.g., a distal end thereof) is advanceable into ventricle 8 that is downstream of valve 7 (e.g., as described in more detail hereinbelow). As shown in FIG. 1B, shaft 410 (e.g., the distal end thereof) is coupled to downstream assembly 300 of implant 150. As described in more detail hereinbelow, this coupling configures delivery tool 400 to (i) anchor winch anchor 310 to ventricular tissue of the heart by applying an anchoring force to the winch anchor, and (ii) for applications in which downstream assembly 300 comprises winch 320, to actuate the winch. That is, delivery tool 400 (e.g., one or more components thereof) can remain coupled to downstream assembly 300 throughout the anchoring of winch anchor 310 and actuation of winch 320. Furthermore, and as described in more detail hereinbelow, delivery tool 400 can be enabled to actuate winch 320 independently of applying the anchoring force to winch anchor 310.

Clasp 430 comprises a downstream support 434 and, in some implementations, can also comprise an upstream support 432. Clasp 430 is transitionable between (i) an open state, and (ii) a grasping state (e.g., a closed state). In the open state, clasp 430 is configured to receive a portion of a leaflet (e.g., leaflet 10) of valve 7. For example, in the open state, upstream support 432 and downstream support 434 can be positioned away from each other, to receive the portion of the leaflet between the upstream support and the downstream support. Clasp 430 is configured to grasp the portion of the leaflet (e.g., between upstream support 432 and downstream support 434) by being transitioned from the open state toward the grasping state while the portion of the leaflet is disposed within the clasp (e.g., between the upstream support and the downstream support). In the grasping state, upstream support 432 and downstream support 434 can be closer to each other than in the open state. In some implementations, in the grasping state, in the absence of an obstruction (e.g., the portion of the leaflet) upstream support 432 and downstream support 434 are in contact with each other, e.g., press against each other.

Driver 450 is configured to anchor patch 210 to the leaflet (e.g., to the portion of the leaflet grasped between upstream support 432 and downstream support 434) using patch anchor 240, e.g., by driving the patch anchor through the leaflet. FIGS. 1A-5B (as well as other figures) show a configuration in which (i) patch anchor 240 is a toggle anchor with a tip 250 that has a sharp point or edge (e.g., that is sharpened to a point/edge), and (ii) driver 450 is configured to drive the patch anchor through the leaflet by pushing a heel 252 of the patch anchor distally while the patch anchor is substantially colinear with the driver and/or with the vector of pushing, e.g., without the patch anchor being disposed within a needle. However, the scope of the disclosure includes examples (e.g., modifications of system 100) in which a needle is employed, e.g., patch anchor 240 can be disposed within, and advanced out of, a needle that punctures the leaflet, e.g., tip 250 may not have a sharp point/edge.

In some implementations, tool 400 comprises a capsule 470 at a distal end of shaft 410. Capsule 470 is configured to house downstream assembly 300 of implant 150 during delivery and implantation of the implant. In some implementations, capsule 470 is dimensioned to conceal tissue-engaging element 312 in order to reduce a likelihood of inadvertently engaging and/or injuring tissue with the tissue-engaging element during transluminal advancement of distal portion 404 of tool 400.

Capsule 470 has an open distal end 471 via which downstream assembly 300 is deployable. For applications in which winch 320 (e.g., housing 321 thereof) has lateral aperture 326 through which tether 160 passes, capsule 470 can define a lateral window 474 in order to allow the tether to reach the winch, e.g., by the capsule housing downstream assembly 300 in an orientation in which lateral window 474 aligns with aperture 326.

Capsule 470 can be a unitary element or, as shown, can comprise a housing 472 and a shroud 476. Shroud 476 can cover a distal part of housing 472 and can even extend distally beyond the housing to form a rim 477. Shroud 476 can be formed from a material that is softer and/or more flexible than that of housing 472 (e.g., the shroud can comprise a polymer or a silicone), so as to reduce a potential for injuring tissue. For applications in which shroud 476 extends distally beyond the housing to form rim 477, the rim can therefore serve as an atraumatic tip, which can be particularly advantageous for placement of capsule 470 against ventricular tissue during driving of tissue-engaging element 312 of anchor 310 into the ventricular tissue.

In some implementations, housing 472 itself can also be configured to contribute to the atraumatic nature of capsule 470. For example, and as shown, the distal part of housing 472 can be defined by a plurality of fingers 473 distributed (e.g., parallel with each other) circumferentially to approximate a tubular shape, but with gaps therebetween. For such implementations, fingers 473 can be embedded within shroud 476. By being formed in this manner, the distal part of housing 472 can be more flexible than if it were substantially tubular (e.g., without gaps between fingers). Thus, upon distal capsule 470 being pressed against ventricular tissue, housing 472, and therefore the capsule, can responsively flex, e.g., rather than injuring the tissue.

In some implementations, capsule 470 defines an elongate lateral opening that extends proximally from a distal opening of the housing, e.g., is open to the distal opening of the housing. For example, for applications in which capsule 470 comprises housing 472 and shroud 476, the housing can define an elongate lateral opening 475 that extends proximally from a distal opening of the housing, e.g., is open to the distal opening of the housing. For such implementations, and as shown, shroud 476 can substantially cover a distal region of elongate lateral opening 475, such that window 474 is defined by a proximal region of the elongate lateral opening, e.g., proximally from the shroud. For some such implementations, shroud 476 defines a narrow slit 478 that extends between a distal opening of the shroud (which can serve as open distal end 471 of capsule 470) and window 474. Slit 478 can be in alignment with elongate lateral opening 475.

Elongate lateral opening 475, fingers 473, and/or slit 478 can be substantially parallel with axis ax1.

Narrow slit 478 is configured to facilitate tether 160 passing therethrough during deployment of downstream assembly 300 from capsule 470, but also is hypothesized to reduce a likelihood of a deleterious interaction with tissue during advancement of the capsule to the ventricle, such as inadvertent capture of a chorda tendinea within elongate lateral opening 475, compared with an otherwise similar capsule that has only elongate lateral opening 475.

Narrow slit 478 is narrower than elongate lateral opening 475 and can be less than 1 mm wide. In some implementations, narrow slit 478 is closed at rest, e.g., its sides are in contact with each other, and are configured to transiently part as tether 160 passes therebetween during deployment of downstream assembly 300 from capsule 470.

Implant 150 is loaded on delivery tool 400 with upstream assembly 200 disposed proximally from downstream assembly 300. As shown, upstream assembly 200 can be secured laterally from shaft 410. In some implementations, and as shown, upstream assembly 200 is mounted on a mount 440 that can be disposed laterally from shaft 410. For such implementations, this mounting of upstream assembly 200 can be such that patch 210 lies against a surface of mount 440. For some such implementations, mount 440 can have a convex outer surface (e.g., the mount can be substantially arc-shaped, curving partway around shaft 410), and patch 210 can lie in a curve against the convex outer surface of the mount, e.g., as shown. In some implementations, patch 210 is held against mount 440 in this manner by one or more wraps 442 wrapped around the patch and the mount. For some such implementations, and as shown, wraps 442 also wrap around shaft 410, thereby holding patch 210 to the shaft. As described in more detail hereinbelow, mount 440 is configured to carry patch 210 toward clasp 430.

In some implementations, and as shown (e.g., in inset B of FIG. 1B), each wrap 442 comprises a flexible loop (e.g., a closed loop) that itself is looped around patch 210 so as to form (i) a pair of longitudinal portions 442L that extend around the patch, and (ii) two bights 442B, each bight connecting an end of one of the longitudinal portions of the pair to an end of the other longitudinal portion of the pair. For each wrap 442, one of the bights is secured to a bracket 444, and a rod 446 extends through the other one of the bights, thereby holding the wrap looped around the patch. Brackets 444 can be spring-loaded, thereby holding wraps 442 taut. In some implementations, and as shown, the orientation in which wraps 442 and brackets 444 are arranged alternates, so that rod 446 is held in place. To release wraps 442 (e.g., to release patch 210 from being held against mount 440 by the wraps), rod 446 is retracted. This is described, in the context of implantation of implant 150, with reference to FIG. 2I. However, it is to be noted that other types of wraps can be used.

As shown, tether 160 can be attached (e.g., fixedly attached) to a lip region of patch 210, e.g., at or proximate a lip 211 of the patch. As described in more detail hereinbelow, lip 211 is the edge of the patch that, after implantation, is disposed furthest from the root of the leaflet to which the patch is secured. Furthermore, lip 211 can also be the edge of the patch furthest from patch anchor 240. Patch 210 can also be considered to have a root region, e.g., at or proximate a root edge 212 of patch. Root edge 212 is the edge of the patch that is opposite lip 211, and that, as described in more detail hereinbelow, after implantation, is disposed closest to the root of the leaflet to which the patch is secured. Patch anchor 240 can be disposed at a root region of patch 210, e.g., at or proximate root edge 212.

Patch 210 can also have lateral edges, e.g., two lateral edges 213′ and 213″, on opposite sides of the patch. The lateral edges of patch 210 can extend between lip 211 and root edge 212.

In some implementations, and as shown, patch 210 is wider (e.g., a distance between lateral edges 213′ and 213″ is greater) toward (e.g., at) lip 211 than toward (e.g., at) root edge 212. For example, patch 210 can approximate a trapezoid (e.g., an isosceles trapezoid) in shape, with lip 211 being the longer base of the trapezoid, and root 212 being the shorter base of the trapezoid.

Implant 150 can be loaded on delivery tool 400 with patch 210 oriented with lip 211 proximal from root 212, e.g., as shown. In this orientation, for applications in which tether 160 is attached to the lip region of patch 210, the tether therefore can extend past root edge 212 and alongside the patch on its route to the lip region. For example, and as shown, a portion 161 of tether 160 can extend alongside the patch, on the side of the patch that faces shaft 410 (e.g., on the concave side of the patch). For applications in which delivery tool 400 comprises mount 440, portion 161 of tether 160 can be disposed (e.g., sandwiched) between the patch and the mount, e.g., as shown in FIG. 1B.

In some implementations, alternatively or in addition to patch 210 being held against the surface of mount 440 (e.g., by wraps 442), the patch can be secured to tool 400 (e.g., to the mount) by patch anchors 240. For example, mount 440 can be shaped to house (or can comprise one or more components that are configured to engage) patch anchors 240. In the example shown, mount 440 is shaped to define channels (e.g., grooves) 448 shaped to receive patch anchors 240 (e.g., one channel per patch anchor). As shown, channels 448 can be defined in the lateral/convex surface of mount 440, e.g., the surface against which patch 210 is typically disposed. Channels 448 can be shaped to allow patch anchors 240 to slide along the channel but to obstruct the patch anchors from exiting the channel laterally. For example, each channel 448 can be narrower at the surface of the mount than deeper into the mount. For example, and as shown, each channel 448 can have a cross-sectional shape of a major circular segment, with its chord being open at the surface of the mount, e.g., as shown. (It is to be understood that noncircular equivalents can also be used, mutatis mutandis.) Thus, for applications in which implant 150 is provided with patch anchors 240 coupled to patch 210, disposition of patch anchors 240 within channels 448 secures the patch to mount 440. The relevance of this is discussed hereinbelow with reference to FIGS. 2I-K.

In some implementations in which implant 150 is provided with patch anchors 240 coupled to patch 210, this coupling is provided by cords 242, e.g., each patch anchor is coupled to the patch by a respective cord. For example, each patch anchor 240 can define an eyelet 244 through at least one lateral wall (e.g., through just one lateral wall, or through the entire anchor), via which cord 242 is attached to the patch anchor. Eyelet 244 may be approximately midway along anchor 240, and/or may be a pair of eyelets. For some such implementations in which patch anchors 240 are disposed in channels 448, and in which the channels are shaped to inhibit the patch anchors from exiting the channel laterally, each cord 242 extends away from its patch anchor (e.g., substantially orthogonally from the anchor axis of the patch anchor) to patch 210 by exiting the channel laterally, thereby securing patch 210 to mount 440. This is visible, for example, in FIG. 2I.

In some implementations, and as shown, patch anchor 240 can be provided with a retrieval feature 241 (e.g., a retrieval eyelet) to which a retrieval line can be releasably attached. Retrieval feature 241 can be disposed at the heel 252 of the patch anchor or another location. Examples of such a retrieval lines and retrieval features are described in more detail hereinbelow.

In some implementations, delivery tool 400 is configured such that mount 440 is movable between a retracted position and a primed position. FIG. 1B shows mount 440 in its retracted position, which is typically also the position of the mount during transluminal advancement of distal portion 404 of tool 400, e.g., as shown in FIG. 2A. In the primed position (FIG. 2J), mount 440 is closer to clasp 430 than in the retracted position and can be in contact with the clasp (e.g., with upstream support 432 thereof). As described in more detail hereinbelow, drivers 450 can be configured to drive patch anchors 240 through the leaflet while the mount is in the primed position, e.g., as shown in FIG. 2K.

For applications in which wraps 442 are used, the wraps can hold patch 210 against mount 440 while the mount is in its retracted position. In some implementations, wraps 442 are released prior to mount 440 moving into its primed position, e.g., as shown in FIG. 2I.

In some implementations, and as shown in FIG. 2B, in the retracted position of mount 440 channels 448 are substantially parallel with axis ax1. In some implementations, and as shown in FIG. 2J, in the primed position of mount 440 channels 448 are oblique with respect to axis ax1, e.g., due to deflection of the mount during the transition toward its primed position.

In some implementations in which patch anchor 240 is disposed in channel 448, driver 450 is configured to anchor the patch to the leaflet (described hereinbelow) by driving the patch anchor out of a distal end of the channel. For some such implementations, driver 450 enters the channel via a proximal end of the channel. In some implementations, delivery tool 400 is provided with a distal end (e.g., a driver head) of driver 450 already disposed within channel 448. In some implementations, driver 450 merely abuts patch anchor 240, whereas for other implementations the driver head is configured to engage and/or grip the anchor (e.g., the driver head and/or the anchor comprise features that facilitate engagement and/or gripping of the anchor by the driver head).

As described hereinabove, delivery tool 400 comprises at least one driver 450. In the example shown, tool 400 comprises one driver 450 per patch anchor 240, e.g., two drivers.

Extracorporeal proximal portion 402 can comprise one or more controllers. The representation of these controllers in FIG. 1A is purely schematic, and each of these controllers can be, or can comprise, a knob, wheel, lever, slider, or other control element or interface via which the operator (e.g., the physician) can operate tool 400 to deliver and implant implant 150, e.g., using the techniques described herein.

In some implementations, proximal portion 402 comprises a clasp controller 110, which is operatively coupled to clasp 430 (e.g., to upstream support 432 thereof) such that operation of the clasp controller transitions the clasp between its open and grasping (e.g., closed) states. This operative coupling can be provided by a wire 130 that is attached to upstream support 432. In the example shown, two wires (e.g., parallel with each other) are used—although, as shown, these could be formed from a single length of wire that loops through upstream support 432 and turns back on itself. Operating clasp controller 110 to pulling on wire 130 transitions clasp 430 between its open and grasping states by moving (e.g., deflecting) upstream support 432 with respect to downstream support 434, and typically also with respect to shaft 410. For example, clasp 430 can be biased (e.g., spring-loaded) toward being in its grasping state, the clasp can be opened by pulling (e.g., tensioning) wire 130, and the clasp can be closed simply by releasing the tension on the wire, e.g., allowing the biasing (e.g., spring-loading) of the clasp to responsively transition the clasp toward its grasping state.

In some implementations, proximal portion 402 comprises a driver controller 112, which is operatively coupled to drivers 450 such that operation of the driver controller induces the driver to drive patch anchor 240 through the leaflet to which upstream assembly 200 is to be anchored. In the example shown, operating driver controller 112 pushes drivers 450 distally such that each driver pushes the heel of a respective patch anchor 240 distally.

Proximal portion 402 can comprise a mount controller 116, operatively coupled to mount 440 such that operation of the mount controller moves the mount between its retracted position and its primed position. This operative coupling can be provided by one or more mount-control rods 136, a distal end of which can be fixed to mount 440. In some implementations, mount-control rods 136 extend through dedicated auxiliary lumens 408. In some implementations, each mount-control rod 136 can be tubular, and can share an auxiliary lumen with another control component of tool 400, e.g., with the other control component extending through the tubular mount-control rod. For example, and as shown, drivers 450 can extend through mount-control rods 136. Alternatively, mount-control rods 136 can be non-hollow, e.g., can run substantially parallel with drivers 450.

In some implementations, within distal portion 404 shaft 410 has a proximal part 411 and a distal part 412, which are axially slidable with respect to each other, e.g., in a telescopic arrangement, as shown. For some such implementations, proximal portion 402 comprises a shaft controller 114 (e.g., a shaft extender), operatively coupled to shaft 410 such that operation of the shaft controller reversibly extends the distal part of the shaft distally from the proximal part of the shaft. It is to be noted that distal part 412 can extend proximally at least as far as proximal part 411, but is nonetheless referred to as the “distal” part because it extends further distally than the proximal part.

It is to be noted that the functions of the various controllers of proximal portion 402 can be separated into single-function controllers or combined into multi-function controllers.

Proximal part 411 can be tubular, e.g., to house distal part 412. Distal part 412 can be tubular, e.g., to house one or more driveshafts that control downstream assembly 300, e.g., as described hereinbelow.

In some implementations, clasp 430 (e.g., downstream support 434 thereof) is coupled to shaft 410 such that extension of distal part 412 distally from proximal part 411 moves (e.g., deflects) downstream support 434 with respect to the shaft. For example, delivery tool 400 can comprise one or more frame elements 436 (e.g., arms, extensions, strips, ribbons, wedges, sheets, etc.) that are coupled to shaft 410, and that cooperate with the shaft to define a mechanical linkage that moves (e.g., deflects) downstream support 434 with respect to the shaft. In the example shown, a single frame element 436, preconfigured to bend or articulate in a particular manner (e.g., by the use of flexure joints), provides this function. One end of the frame element is coupled to proximal part 411 of the shaft, and the other end of the frame element is coupled to distal part 412 of the shaft. It is to be noted that a similar effect can be achieved by using multiple frame elements articulatably (e.g., hingedly) coupled to each other.

In some implementations, and as shown, a unitary piece of stock material defines upstream support 432, downstream support 434, and a flexure joint 433 that articulatably couples the upstream support to the downstream support. For such implementations, and as further shown, downstream support 434 is fixed to a region 435 of frame element 436. However, it is to be understood that for other implementations downstream support 434 can simply be defined by region 435, e.g., a unitary piece of stock material can define frame element 436 and downstream support 434. For such other implementations, upstream support 432 can be formed from a separate piece of material, and articulatably coupled to downstream support 434.

In some implementations, in the absence of tension on wire 130, axial movement of distal part 412 with respect to proximal part 411 moves (e.g., deflects) both downstream support 434 and upstream support 432 with respect to shaft 410. For example, clasp 430 can be biased (e.g., spring-loaded) toward being in its grasping state, and can remain in that state (e.g., a disposition between upstream support 432 and downstream support 434 can remain constant) as the downstream support moves (e.g., deflects) with respect to shaft 410. FIGS. 1B and 2A show a state in which distal part 412 is extended sufficiently such that downstream support 434 and, due to a lack of tension on wire 130, upstream support 432 point distally, e.g., with clasp 430 in its grasping state.

In some implementations, and as shown, clasp 430 defines one or more slots 437 via which driver 450 is configured to drive patch anchors 240 (e.g., one slot per patch anchor). In some implementations, it is downstream support 434 (whether as part of a unitary piece of stock material that also defines upstream support 432, or whether defined by part of frame element 436) that defines slots 437. That is, downstream support 434 provides an opposing force during driving of patch anchors 240 through the leaflet, and the patch anchors are positioned to pass through the downstream support at slots 437, e.g., as described in more detail with reference to FIGS. 2K-L. As also described in more detail hereinbelow, this results in cord 242 being threaded through slot 437. In order to allow cord 242 to exit slot 437 laterally, but to obstruct tissue (e.g., a chorda tendineae) from entering and becoming trapped in the slot, clasp 430 (e.g., downstream support 434 thereof) can define or comprise at least one slot guard 438, e.g., a respective slot guard for each slot. In some implementations, and as shown, slot guard 438 is resilient, has a resting position in which it obstructs (e.g., completely covers) an entrance to the slot, and is transiently deflectable away from the slot by the cord exiting the slot, thereby facilitating exiting of the cord from the slot, e.g., as described with reference to FIGS. 2L, and 6A-C.

It is to be noted that the scope of the present disclosure includes variants of system 100 in which (i) delivery tool 400 comprises one or more needles, (ii) the tip of patch anchor 240 may not have a sharp point, and (iii) rather than the patch anchor being driven through the leaflet directly, the needle penetrates the leaflet, and the patch anchor is subsequently advanced out of the needle.

Reference is now made to FIGS. 2A-R, which are schematic illustrations showing at least some steps in a technique for treating valve 7 of a heart of a subject, in accordance with some implementations. Although the technique is shown for use with system 100, In some implementations variants of system 100 (e.g., comprising variants of implant 150 and/or of tool 400), and/or other systems can be used instead. In each of FIGS. 2A-D and 2F-N, the left-side frame illustrates the position and/or interaction of system 100 with the heart, while the right-side image emphasizes the state of the system itself.

With implant 150 loaded on distal portion 404 of delivery tool 400, the distal portion is transluminally advanced to heart 4 of the subject, e.g., to an atrium 6 upstream of valve 7. For example, and as shown, distal portion 404 can be transluminally (e.g., via the inferior or superior vena cava) and transseptally advanced into the left atrium of the heart (FIG. 2A).

Transluminal advancement of tool 400 can be facilitated by one or more catheters 102, 104, one or more of which can be steerable (e.g., actively steerable, e.g., using pull-wires or other components known in the art). In some implementations, catheter 102 and/or catheter 104 can be advanced to the atrium, and tool 400 can be subsequently advanced through the catheter(s). In some implementations, at least catheter 104 is advanced with tool 400 (with implant 150 mounted thereon) disposed within the catheter. For some such implementations, capsule 470 (with downstream assembly 300 disposed therein) can be disposed outside of the distal end of catheter 104 during such advancement of the catheter and the tool.

In the example shown, tool 400 is transluminally advanced while in a delivery state (FIG. 2A). In the delivery state, patch 210 can be held against mount 440 and/or shaft 410 by wraps 442, e.g., to facilitate smooth advancement, and/or to protect the patch. In some implementations, and as shown, in the delivery state, clasp 430 is in a low-profile state. For some such implementations, and as shown, in the low-profile state clasp 430 is closed (e.g., is in its grasping state) but is deflected distally (e.g., the clasp faces distally, with both upstream support 432 and downstream support 434 deflected distally), e.g., such that downstream support 434 is disposed adjacent to and substantially parallel with shaft 410 (e.g., distal part 412 thereof). As described hereinabove, this is achieved by extending distal part 412 of shaft 410 from proximal part 411 (e.g., telescopically extending the shaft), thereby straightening frame element 436. For other such implementations, in the low-profile state clasp 430 is over-opened, such that upstream support 432 is substantially collinear with downstream support 434 (e.g., by extending distal part 412 while also tensioning wires 130).

A low-profile state is advantageous for transluminal advancement, but in some instances may be disadvantageous for maneuvering within the heart due to the relatively long configuration of distal portion 404 resulting from extending distal part 412. Thus, in some implementations, once distal portion 404 has passed through (e.g., passed entirely through) interatrial septum 5 and/or is disposed within (e.g., disposed entirely within) atrium 6, distal portion 404 is transitioned into a contracted state, e.g., by withdrawing distal part 412 of shaft 410 into proximal part 411 (e.g., telescopically contracting the shaft), such as by operating shaft controller 114 (FIG. 2B). Clasp 430 is also typically closed in the contracted state of distal portion 404, but deflected proximally (e.g., the clasp faces proximally). Thus, the widest part of distal portion 404 in the contracted state (at the level of clasp 430) is wider than the widest part in the low-profile configuration, but the shorter overall length of the distal portion advantageously facilitates a smaller “turning circle” as the distal portion is steered within the heart.

Distal portion 404 (e.g., in its contracted state) is then turned toward valve 7 (FIG. 2C), and clasp 430 is transitioned into its open state in which upstream support 432 and downstream support 434 are positioned away from each other, and in which the clasp is configured to receive a portion of a leaflet of the valve between the upstream support and the downstream support (FIG. 2D). Although FIGS. 2C-D show distal portion 404 being turned toward valve 7 prior to clasp 430 being opened, it is to be understood that these steps may be performed in the opposite order, or concurrently.

In some implementations, and as shown, the transitioning of clasp 430 into its open state is performed in a single step by partially extending distal part 412 (thereby deflecting downstream support 434) while maintaining tension on wire 130 (thereby retaining upstream support 432 substantially stationary). However, it is to be understood that the scope of the disclosure includes performing the transition in discrete steps of: (i) while the clasp remains closed, deflecting the entire clasp (e.g., in a downstream direction) so that the clasp (or at least downstream support 434 thereof) is substantially orthogonal to shaft 410, and therefore protrudes maximally laterally, such as by operating shaft controller 114; and (ii) subsequently, transitioning clasp into its open state by deflecting upstream support 432 (e.g., by tensioning wire 130) while downstream support 434 remains stationary, such as by operating clasp controller 110.

The deflection of clasp 430 in the transition between FIG. 2C and FIG. 2D shows the partial extension of distal part 412 being performed by retracting proximal part 411 proximally, e.g., in a manner that maintains capsule 470 substantially stationary relative to the anatomy and retracts patch 210 proximally/upstream relative to the anatomy. However, it is to be understood that the extension could also be achieved in a manner that maintains patch 210 substantially stationary relative to the anatomy and advances capsule 470 distally toward/into the ventricle.

While clasp 430 remains in its open state, distal portion 404 is advanced distally through valve 7 into ventricle 8 (FIGS. 2E-F). The passage of the widest part of distal portion 404 (e.g., frame element 436 and/or downstream support 434) through valve 7 may be identifiable using imaging techniques (e.g., fluoroscopy and/or ultrasound), e.g., due to their obstruction of leaflet 10 from moving toward shaft 410 and/or the opposing leaflet during ventricular systole (FIG. 2E). Furthermore, the subsequent restoration of movement of leaflet 10 toward shaft 410 and/or the opposing leaflet during ventricular systole may also be identifiable using such imaging techniques, and may indicate that downstream support 434 has passed beyond leaflet 10 sufficiently to facilitate subsequent capture of the leaflet (FIG. 2F).

Distal portion 404 is then manipulated to move clasp 430 to receive a portion of leaflet 10 (FIG. 2G). For example, and as shown, distal portion 404 can be moved proximally until leaflet 10 rests upon downstream support 434 and/or resists pulling of the distal portion into the atrium. While the portion of leaflet 10 remains between upstream support 432 and downstream support 434, clasp 430 is closed (e.g., is transitioned toward its grasping state), thereby grasping the portion of the leaflet (FIG. 2H). As described hereinabove, this can be achieved by pushing wires 130 distally, and/or allowing them to be pulled distally by upstream support 432.

For applications in which tool 400 comprises wraps 442, the wraps can be released at this stage, if not earlier. The inset of FIG. 2H shows wraps 442 holding patch 210 securely against mount 440 (not visible). As described hereinabove, and as shown in FIG. 2H, in some implementations wraps 442 can extend around shaft 410. For such implementations, wraps 442 should therefore be released so as to facilitate subsequent steps in which patch 210, carried by mount 440, is moved away from shaft 410. FIG. 2I shows wraps 442 having been released by retraction of rod 446, and patch 210 responsively unwrapping from around shaft 410. Brackets 444 are shown as having changed shape in response to the release of tension on wraps 442.

In some implementations, such unwrapping of patch 210 can be passive, e.g., following its release, merely in response to movement of blood. In some implementations, e.g., for applications in which patch 210 comprises a frame 230, the frame can comprise a spring or otherwise be biased to open up the patch.

While the portion of leaflet 10 remains grasped by clasp 430, mount 440 is advanced toward clasp 430, e.g., upstream support 432 thereof (FIG. 2J). This new position of mount 440 can be considered to be a “primed” position, whereas the previous position of the mount can be considered to be a “retracted” position. As shown, the advancement of mount 440 into its primed position can be performed by operating mount controller 116 to advance mount-control rods 136 to push the mount over and along wires 130 (FIG. 2J).

It is to be noted that, as described hereinabove, despite the unwrapping of patch 210 from around shaft 410 and/or mount 440, for applications in which the patch is coupled to the mount via anchors 240, the patch can remain coupled to the mount at this stage, e.g., as shown in the inset of FIG. 2I. Therefore, as mount 440 is advanced toward clasp 430, the mount carries patch 210 toward the clasp (FIG. 2J).

It is to be noted that the movement of mount 440, and thereby of patch 210, toward clasp 430 can include distal and/or downstream movement. It is also to be noted that this movement can also include lateral movement, e.g., movement away from shaft 410, toward the opening end of clasp 430. As shown in FIG. 2H, for applications in which wires 130 are used to operate clasp 430, the articulation of upstream support 432 during closure of the clasp can pull the wires laterally. Thus, for some such implementations in which, additionally, mount 440 is slid along wires 130, the wires serve as rails that guide the mount not just distally, but obliquely (e.g., distolaterally) to the clasp.

The steps shown in FIGS. 2K-L are also shown, from a different perspective, in FIGS. 6A-C. However, for the sake of clarity, leaflet 10 is not shown in FIGS. 6A-C.

While (i) the portion of leaflet 10 remains grasped by clasp 430, and (ii) mount 440 is disposed at the clasp (e.g., in its primed position), drivers 450 are used to drive patch anchors 240 through the leaflet (e.g., through the grasped portion of the leaflet), such as by operating driver controller 112, thereby anchoring patch 210 to the leaflet (FIG. 2K and FIG. 6A). For example, and as shown, patch anchors 240 can be driven out of the end of channels 448, and through leaflet 10 and slot 437. It is hypothesized that lateral portion of the movement of mount 440 and patch 210 prior to anchoring advantageously positions patch anchors 240 (and typically the patch) away from the lip of leaflet 10, e.g., toward the root of the leaflet, and that this advantageously enhances the positioning of the patch and/or the reliability of its anchoring.

Clasp 430 is then reopened, and distal portion 404 is moved away from patch 210 and the leaflet 10 to which it is anchored (FIG. 2L and FIGS. 6B-C). It is to be noted that this can be facilitated by (i) patch 210 becoming automatically released from mount 440 upon patch anchors 240 exiting the mount (FIG. 6A), and (ii) cords 242, which became disposed through slots 437 during anchoring, exiting the slots laterally, optionally facilitated by transient deflection of slot guards 438 (e.g., away from each other) as the cords slip through as clasp 430 is moved away (FIG. 6B). Once clear of cords 242, slot guards 438 can then return to their original resting state, e.g., deflecting toward each other (FIG. 6C).

In some implementations, and as shown, for each slot guard 438, a free end of the slot guard can be tucked underneath another portion of downstream support 434, such that slots 437 are in effect completely closed. This may greatly obstruct inadvertent introduction of tissue (e.g., chordae tendinea) into slot 437, e.g., because pushing of the slot guard in that direction does not result in opening of the slot. This tucked configuration is particularly visible in FIGS. 6A-C and in the inset of FIG. 1A.

Patch 210 can be anchored to leaflet 10 in a manner in which the patch (e.g., lip 211 thereof) overhangs the lip of the leaflet, e.g., extends further into ventricle 8 than does the leaflet. Thus, patch 210 can serve as an extension of leaflet 10, advantageously facilitating coaptation with the opposing leaflet following implantation.

In some implementations, subsequently to anchoring patch 210 to leaflet 10, downstream assembly 300 is anchored to tissue of ventricle 8. In some implementations, shaft 410 is extended in order to reach the ventricular tissue, e.g., by operating shaft controller 114 (FIG. 2M). As shown, this can return clasp 430 to its low-profile state. In some implementations, tether 160 is released (e.g., unspooled) from winch 320 at generally the same time, in order to accommodate the increased distance between upstream assembly 200 (e.g., patch 210 thereof) and downstream assembly 300 (e.g., the winch thereof). It can also be advantageous to avoid releasing a large excess of tether 160 relative to the distance of downstream assembly 300 from upstream assembly 200 in order to maintain control of the behavior of the tether in the flow of blood, e.g., to reduce a likelihood of the tether becoming tangled or caught on tool 400. In some implementations, coordination between advancement of downstream assembly 300 and unspooling of tether 160 can be achieved using systems, apparatuses, and/or techniques described in U.S. Provisional Patent Application No. 63/336,489 to Pesach et al., filed Apr. 29, 2022, and/or International Patent PCT/IB2023/054169 to Pesach et al., filed Apr. 24, 2023, each of which is incorporated by reference herein in its entirety.

FIG. 3A is a schematic illustration showing an exploded view of (i) some components of driveshaft subassembly 490 at a distal end of distal portion 404 of delivery tool 400, and (ii) downstream assembly 300. FIG. 3B is an inverted version of FIG. 3A, showing an inverted view of each component. FIGS. 4A-C show the components that are shown in FIGS. 3A-B, but assembled (e.g., in the manner shown in FIG. 1B) and in cross-section.

Once a site in the ventricle has been selected, downstream assembly 300 is anchored to the site by anchoring winch anchor 310 to the tissue, e.g., by driving tissue-engaging element 312 into the tissue (FIG. 2N). This can be achieved by operating an anchor controller 118 at proximal portion 402. Anchor controller 118 is operatively coupled to winch anchor 310 such that operation of the anchor controller applies an anchoring force to the winch anchor (FIG. 4A). For example, delivery tool 400 can comprise an anchor-control driveshaft 480 that operatively couples anchor controller 118 to winch anchor 310. In some implementations, and as shown, the anchoring force is (or includes) torque, which is applied via engagement between anchor-control driveshaft 480 and winch anchor 310. Winch anchor 310 can comprise a driver interface 316 that is a component of, is defined by, or is fixedly coupled, to a head 314 of the winch anchor. Driveshaft 480 engages driver interface 316 and applies torque to the driver interface via this engagement. For example, and as shown, driveshaft 480 can comprise or define, at a distal end of the driveshaft, a drive head 483 that comprises one or more (e.g., two) spurs 484 that are held in engagement with driver interface 316 by a lock-rod 486. That is, lock-rod 486 locks the engagement between drive head 483 and driver interface 316.

In some implementations, and as shown by the transition from FIG. 2M to FIG. 2N, capsule 470 (e.g., rim 477 of shroud 476 of the capsule) is placed (e.g., pressed) against the ventricular tissue prior to winch anchor 310 being advanced out of the capsule, such that at no time is tissue-engaging element 312 exposed from the capsule, thereby advantageously reducing a likelihood of ensnaring and/or injuring tissue. It is to be noted that, although winch anchor 310 is shown being anchored in the vicinity of the apex of the heart, it can be anchored at a different position, such as to the interventricular septum or another part of the ventricular wall.

Driving winch anchor 310 into the tissue advances the entire of downstream assembly 300 distally through capsule 470 toward the surface of the tissue. For applications in which capsule 470 (e.g., housing 472 thereof) defines elongate lateral opening 475 (described with reference to FIGS. 1A-B), as downstream assembly moves distally through the capsule, aperture 326, which typically protrudes from housing 472, protrudes into lateral opening 475 and moves axially along the lateral opening, rotationally restricted by the lateral opening. That is, lateral opening 475 can serve as a linear track along which winch 320 (e.g., housing 321 thereof) can slide as winch anchor 310 is driven into the tissue. Such a configuration allows capsule 470 to provide a reference force to winch housing 321 continuously throughout the anchoring of anchor 310, such that the winch housing does not rotate along with the anchor. (Such rotation of the housing might otherwise deleteriously wrap tether 160 about tool 400, e.g., shaft 410 thereof.) For applications in which capsule 470 comprises shroud 476, as downstream assembly moves distally through the capsule, tether 160 enters slit 478 from window 474, and extends from aperture 326 out of progressively more downstream parts of slit 478. Depending on the extent to which aperture 326 protrudes from housing 472, the aperture can transiently separate shroud 476 at slit 478 as it moves along the lateral opening 475 and the slit. FIG. 2N shows anchor 310 fully anchored, with aperture 326 separating shroud 476 at a part of slit 478 close to the open distal end of the capsule, and tether 160 extending therefrom.

Once winch anchor 310 has been anchored, the effective length of tether 160 (e.g., the length of the tether between winch 320 and patch 210) can be adjusted in order to achieve optimal hemodynamics, e.g., minimal regurgitation between the leaflets of valve 7. Although patch 210 itself is a leaflet-augmenting patch that itself may improve coaptation between the leaflets by providing an extended and/or surrogate coaptation surface, it is hypothesized that tether 160-especially when of an optimal length—can further improve coaptation, e.g., by directing and/or limiting movement of the patch, and the leaflet to which it is anchored, during the heart cycle. This length adjustment can be achieved by operating a winch controller 119 at proximal portion 402.

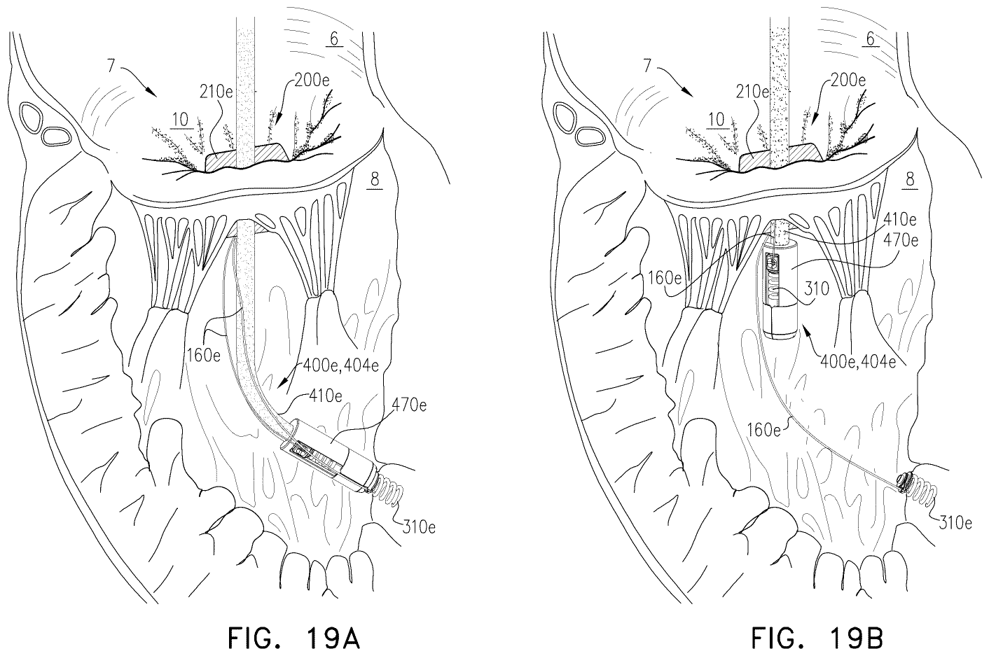

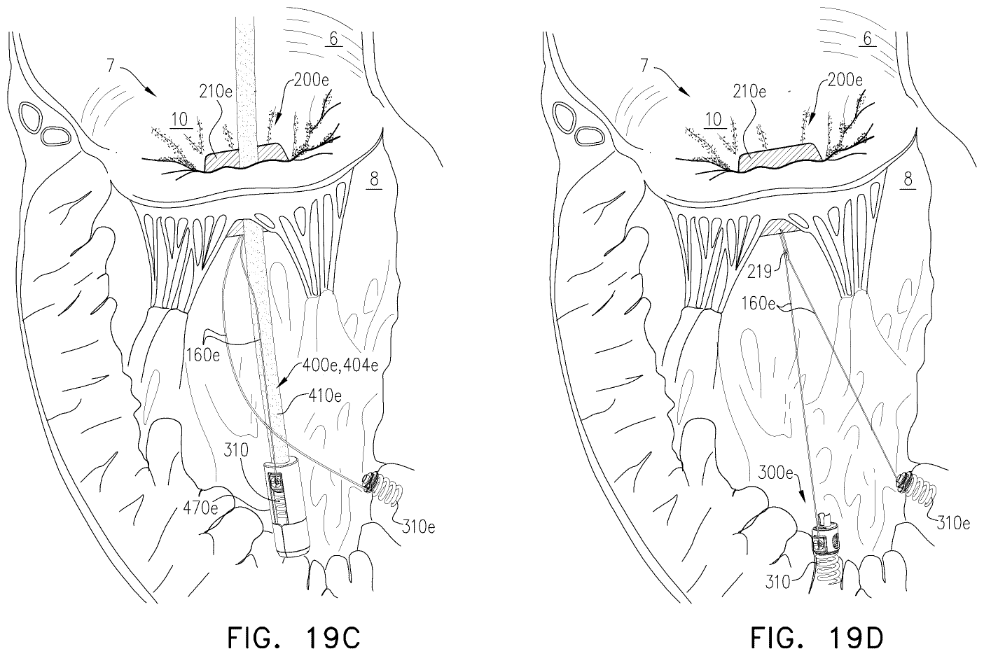

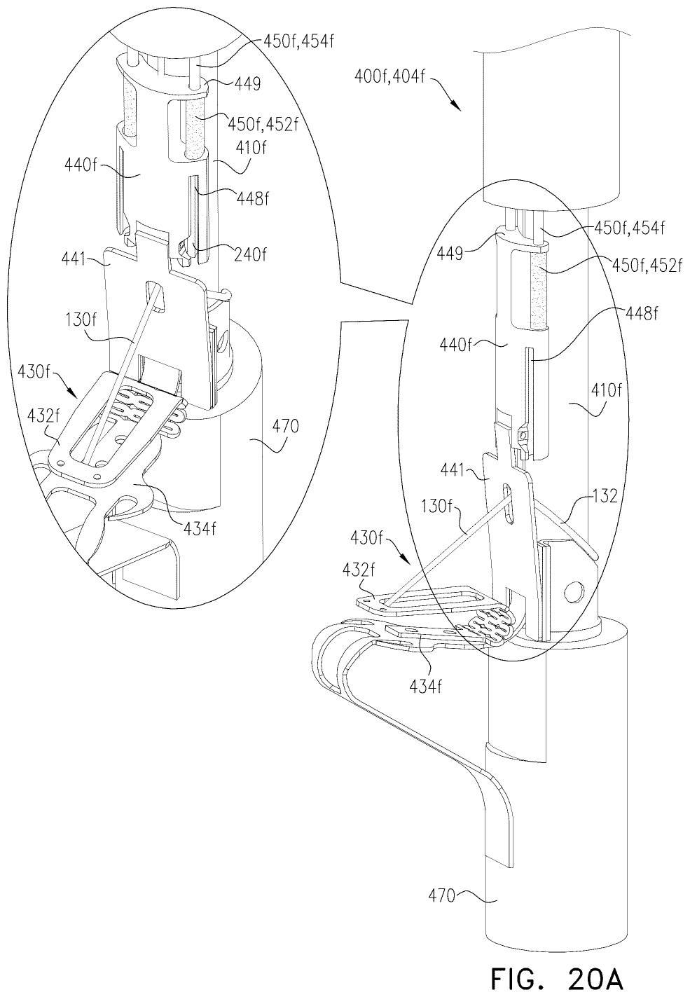

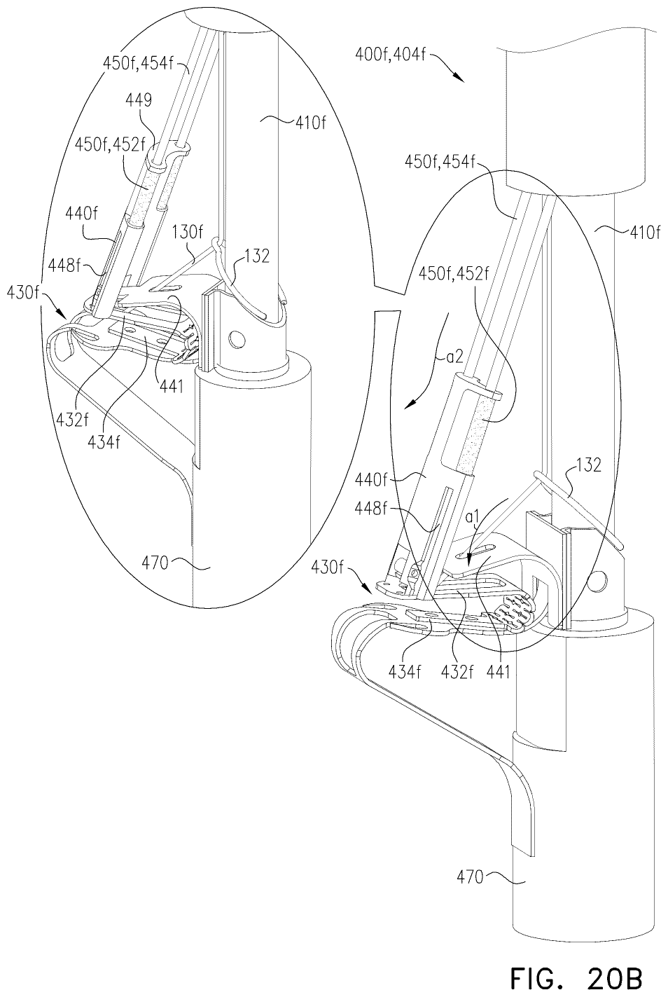

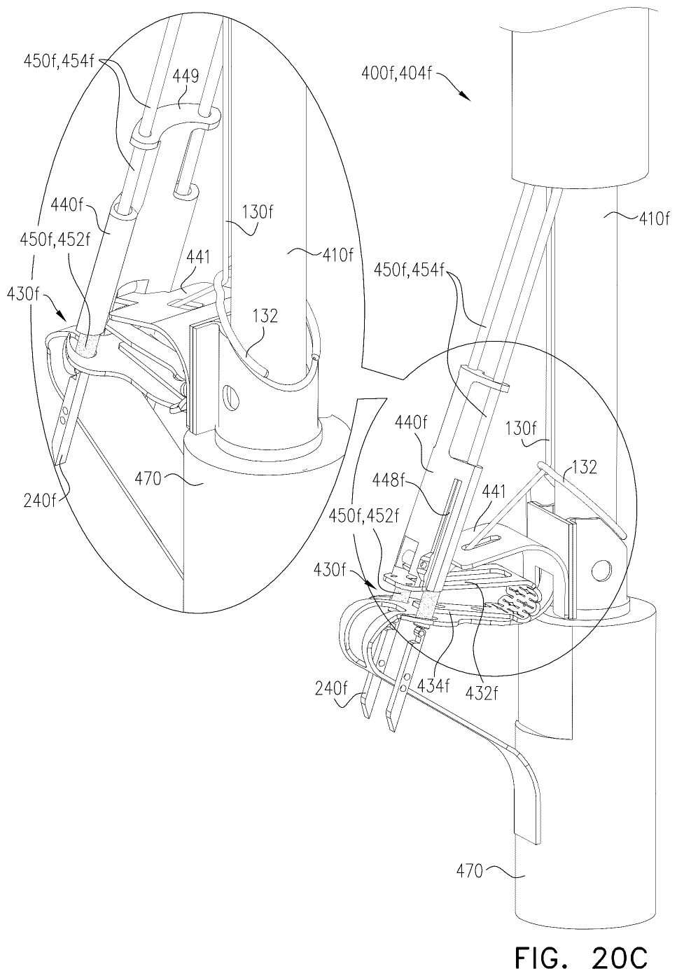

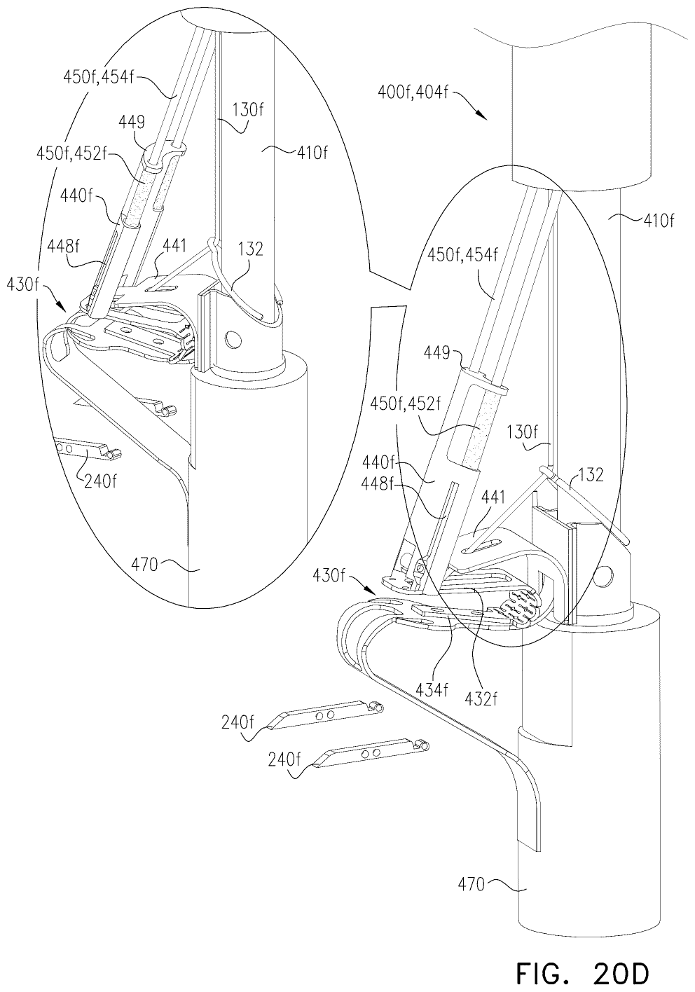

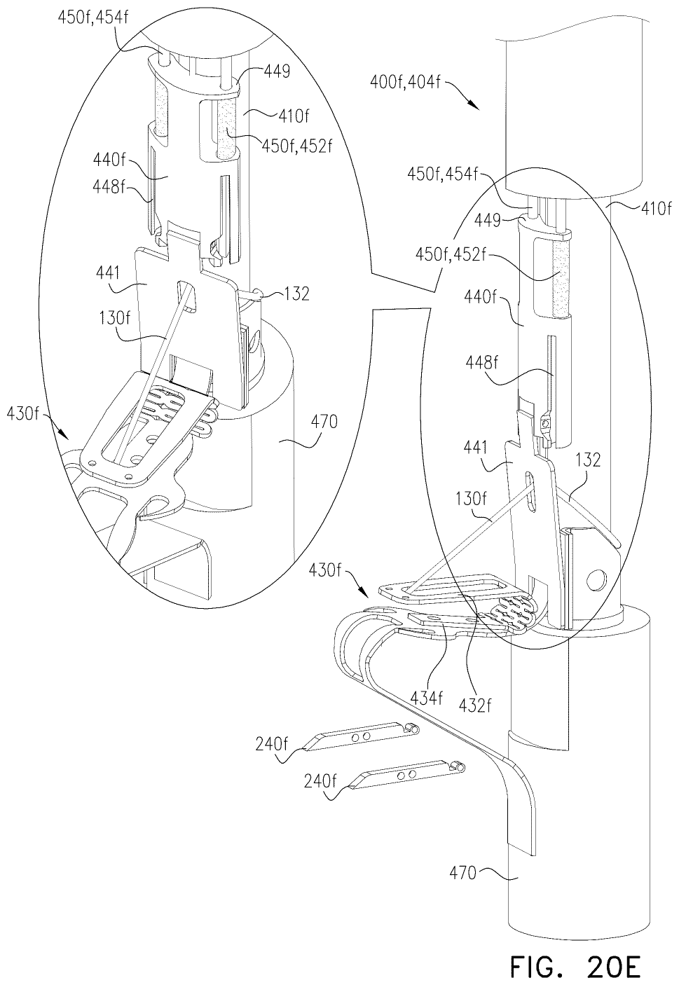

In some implementations, prior to the length adjustment, most of tool 400 is withdrawn out of ventricle 8, e.g., out of the heart, and/or out of the body of the subject entirely (FIG. 20). Such withdrawal primarily consists of withdrawing shaft 410, to which mount 440, clasp 430, and capsule 470 remain attached. This leaves behind driveshaft subassembly 490 of tool 400 extending through valve 7 to downstream assembly 300, to which it remains coupled. It is hypothesized that this advantageously facilitates accurate hemodynamic monitoring during adjustment of the effective length of tether 160 (described hereinbelow), due to driveshaft subassembly 490 typically being slimmer and/or more flexible than shaft 410, or tool 400 as a whole, thereby being less likely to create significant hemodynamic artifacts by (i) pressing on downstream assembly 300 and the ventricular tissue to which it is anchored, (ii) obstructing leaflet movement and coaptation, and/or (iii) directly obstructing or causing turbulence in blood flow. In this state, the leaflets of valve 7 can coapt around driveshaft subassembly 490, e.g., as shown in FIG. 20.

Nonetheless, to further facilitate accurate hemodynamic monitoring during adjustment of the effective length of tether 160, driveshaft subassembly 490 can be moved laterally toward a commissure (e.g., pivoting on downstream assembly 300), further reducing any interference it may have on the behavior of the leaflets (FIG. 2P).

Despite the advantages, described hereinabove, of withdrawing shaft 410 prior to adjustment of the effective length of tether 160, in some implementations, the length adjustment is performed while shaft 410 and capsule 470 remain in place.

Driveshaft subassembly 490 comprises at least one driveshaft, and can further comprise a reference-force tube 492, e.g., as described hereinbelow.

Winch controller 119 is operatively coupled to winch 320 such that operation of the winch controller actuates the winch (FIG. 4A). For example, delivery tool 400 (e.g., driveshaft subassembly 490 thereof) can comprise a winch-control driveshaft 482 that operatively couples winch controller 119 to winch 320. In some implementations, and as shown, actuation of winch 320 is achieved by applying torque to the winch, e.g., via engagement between winch-control driveshaft 482 and winch 320. Winch 320 can comprise a driveshaft interface 324 that is defined by, is a component of, or is fixedly coupled to, spool 322 of the winch. Driveshaft 482 engages driveshaft interface 324 and applies torque to the driveshaft interface via this engagement. For example, and as shown, driveshaft 482 can comprise or define, at a distal end of the driveshaft, a drive head 485 that comprises one or more (e.g., two) projections 488 that extend into recesses defined by interface 324, e.g., the torque can be applied to spool 322 via these projections. This engagement can be maintained indirectly via the locking, by lock-rod 486, between drive head 483 and driver interface 316, e.g., due to preloading between driveshafts 480 and 482. For example, irrespective of advancement of tool 400 through the vasculature, and throughout steps of the implantation procedure, driveshaft 480 can be kept under a limited amount of tension while driveshaft 482 can be kept under a limited amount of axial compression.

A reference force tube 492 is also engaged with downstream assembly 300. As shown, this engagement can be with housing 321. For example, reference force tube 492 (e.g., a distal end thereof) and housing 321 can comprise or define complimentary couplings (e.g., mating surfaces) 494 and 331, respectively. Downstream assembly 300 (e.g., housing 321 thereof) can therefore be considered to comprise or define a reference-force-tube interface 332 that comprises one or more couplings (e.g., mating surfaces) 331, reference force tube 492 engaging the reference-force-tube interface. The engagement between reference force tube 492 and reference-force-tube interface 332 can rotationally lock the reference force tube to the reference-force-tube interface, allowing the reference force tube to provide a reference force during rotation of winch 320. Inter alia, this rotational locking and reference force facilitate rotation of spool 322 without rotation of housing 321 (or revolution of aperture 326 about axis ax2), which is hypothesized to be advantageous due to the presence of tether 160 extending between downstream assembly 300 and upstream assembly 200. For example, were housing 321 to revolve during rotation of spool 322, tether 160 might become wrapped around driveshaft subassembly 490.

In some implementations, the engagement between reference force tube 492 and housing 321 can be maintained indirectly via the locking, by lock-rod 486, between drive head 483 and driver interface 316, e.g., due to preloading between driveshaft 480 and reference force tube 492. For example, irrespective of advancement of tool 400 through the vasculature, and throughout steps of the implantation procedure, driveshaft 480 can be kept under a limited amount of tension while reference force tube 492 can be kept under a limited amount of axial compression.

FIG. 2Q shows tether 160 after its length has been adjusted. This is represented by tether 160 appearing slack in FIG. 2P and taut in FIG. 2Q, although it is to be understood that this is schematic and is not intended to be limiting with respect to the adjustment of the length of the tether.

Once an optimal effective length of tether 160 has been achieved, driveshaft subassembly 490 is disengaged from downstream assembly 300, and thereby from implant 150 as a whole (FIGS. 2R, and FIGS. 4B-C). For example, and as shown, lock-rod 486 can be withdrawn at least sufficiently to allow disengagement of driveshaft 480 from interface 316 via medial deflection of spurs 484 (FIG. 4B). Once disengaged, driveshaft 480, and driveshaft subassembly 490 as a whole, can be withdrawn from downstream assembly 300 (FIG. 4C). Upon this withdrawal of driveshaft subassembly 490 from downstream assembly 300, and more specifically upon withdrawal of winch-control driveshaft 482 (e.g., projections 488 thereof) from winch 320 of the downstream assembly, one or more spring-loaded detents 328 can responsively move to engage housing 321 in a manner that locks spool 322 from rotating with respect to the housing (e.g., locks winch 320) such that tension on tether 160 cannot unspool the tether, thereby fixing the effective length of the tether. In some implementations, winch 320 comprises a number of detents 328 that is equal to the number of projections 488, each of the projections retaining a respective one of the detents from engaging housing 321 until the projection is withdrawn.

In some implementations, and as shown, this movement of detents 328 is a medial movement. In some implementations, housing 321 defines a set of ridges 330 between which detents 328 can become disposed (and/or recesses into which the detents can become disposed) upon withdrawal of driveshaft 482.

In some implementations, housing 321 comprises at least two subcomponents that are secured to each other during manufacture, such as a first subcomponent (e.g., an annular or circumferential subcomponent) 321a and a second subcomponent (e.g., a lid subcomponent) 321b. For some such implementations, and as shown, ridges 330 are defined by second subcomponent 321b. For some such implementations, reference-force-tube interface 332 is defined by second subcomponent 321b.

In some implementations, within downstream assembly 300, tissue-engaging element 312, head 314, and driver interface 316 are rotationally fixed with respect to each other and are collectively rotatably coupled to housing 321 and to spool 322.

In some implementations, within downstream assembly 300, detents 328 (e.g., a frame defining the detents) are, even while unlocked by the presence of driveshaft 482, rotationally fixed with respect to spool 322, e.g., (i) via one or more tongues 329 defined by the frame that defines the detents being disposed in one or more recesses 323 defined in spool 322 (or vice versa), and/or (ii) due to detents 328 being disposed in slots 325 defined by the spool.

In some implementations, within downstream assembly 300, spool 322 is rotatably coupled to housing 321, except when detents 328, which are rotationally fixed with respect to the spool, lock to the housing.

Slots 325 can also provide space for detents 328 to deflect between their unlocked and locked states.

In some implementations, and as shown, downstream assembly 300 can comprise a hub 317 or axle on which spool 322 and detents 328 (e.g., a frame defining the detents) are mounted, providing at least some of the rotatable couplings described hereinabove.

In some implementations, housing 321 is mounted to be rotatable (e.g., freely rotatable) with respect to anchor 310. It is hypothesized that this can advantageously allow the housing to naturally find a rotational orientation in which lateral aperture 326 is optimally positioned, e.g., in response to movement and tension of tether 160.

FIGS. 5A-B are schematic illustrations of upstream assembly 200, in accordance with some implementations. As described hereinabove, patch 210 can comprise a flexible sheet 220 and can also comprise at least one frame 230 supporting the sheet. As further described hereinabove, upstream assembly 200 can be provided with at least one patch anchor 240 coupled to patch 210.

Frame 230 supports sheet 220, thereby providing patch 210 with a shape.

However, frame 230 can be flexible, such that patch 210 can be responsive to conditions in the heart, e.g., to the shape of one or both leaflets, in order to facilitate optimal coaptation. In some implementations, frame 230 (and thereby patch 210) is configured to be more flexible on one axis than on another axis. For example, frame 230 can provide greater flexibility along a root-to-lip axis ax3 of patch 210 (e.g., with lip 211 moving with respect to root 212), than along a mediolateral axis ax4 of the patch, transverse to the root-to-lip axis (e.g., with one lateral edge 213 moving with respect to the other lateral edge). This higher mediolateral rigidity can facilitate patch 210 opening upon release from wraps 442, and/or can advantageously inhibit the patch from folding in on itself after implantation.

Frame 230 can comprise a root brace 232, which can comprise a beam that extends along root 212, e.g., from one lateral edge 213 to the other. Frame 230 can also define a lip brace 231, which can comprise a beam that extends substantially along lip 211, e.g., from one lateral edge 213 to the other. Lip brace 231 and root brace 232 can provide patch 210 with a degree of mediolateral rigidity, e.g., as described in the preceding paragraph.

Frame 230 can comprise a spring 234, which can run between lip brace 231 and root brace 232. In the example shown, spring 234 runs substantially along a root-to-lip midline of patch 210 (e.g., along axis ax3, if axis ax3 is a central root-to-lip axis).

Spring 234 can provide the root-to-lip flexibility described hereinabove. However, spring 234 can also provide patch-anchor tightening functionality, e.g., as described hereinbelow.

FIG. 5A shows face-on and isometric views of patch 210 in a resting state thereof, e.g., prior to loading onto delivery tool 400. In some implementations, other than the curvature of patch 210 around mount 440 and shaft 410 while loaded on delivery tool 400, FIG. 5A can also represent the state of the patch during the initial stages of delivery.

In some implementations, upstream assembly 200 can have patch-anchor tightening functionality. That is, the upstream assembly itself (e.g., patch 210, such as frame 230) can be configured to tighten patch anchor 240-such as by drawing it toward to the patch (e.g., automatically). For example, upstream assembly 200 can bias patch anchor 240 toward patch 210, but can allow the patch anchor to be temporarily moved away from the patch during anchoring. In some implementations, and as shown, this biasing is via upstream assembly 200 applying tension to cords 242. For some such implementations, this is achieved as follows:

As described hereinabove, cords 242 couple patch anchors 240 to patch 210. For example, and as shown, each patch anchor can be coupled to the patch by a respective cord. However, rather than each cord 242 being secured to patch 210 (e.g., frame 230 thereof) at a point 214 that the cord reaches the patch, the cord passes through the patch at point 214, and extends along the patch (e.g., in a root-to-lip direction, such as substantially parallel with axis ax3) to lip brace 231, to which it is secured. Thus, cord 242 is slidably coupled to patch 210 at point 214, which can be at or proximate to root 212, and is fixedly attached to the patch at lip brace 231, which can be at or proximate to lip 211.

In some implementations, and as shown, cords 242 extend along the patch within sheet 220, such as between layers thereof, thereby advantageously allowing the surface of the patch that is presented to the opposing leaflet to be smooth and cord-free. For such implementations, cords 242 can be slidable within sheet 220.

In FIG. 5A, cords 242 are shown as relatively slack. As patch anchors 240 are driven through to the far (e.g., downstream) side of leaflet 10 by drivers 450 (e.g., as described hereinabove with reference to FIGS. 2J-K), the patch anchors are temporarily moved (e.g., pushed) away from patch 210, which remains on the near (e.g., upstream) side of the leaflet. This tensions cords 242, which thus pull lip brace 231 toward root brace 232, placing spring 234 under stress (FIG. 5B). This is represented in FIGS. 5A-B by linear compression of spring 234 and patch 210, temporarily reducing a root-to-lip length L1 of the patch (FIG. 5A) to a reduced length L2 (FIG. 5B), e.g., linearly contracting the patch. In some implementations, placing spring 234 under stress slides the spring across the sheet. Upon the release of patch anchors 240 from drivers 450, spring 234 returns toward its original (e.g., resting) state, drawing (e.g., pulling) the patch anchors back toward patch 210, and thereby securing the patch to the leaflet by sandwiching the leaflet between the patch anchors and the patch, e.g., as illustrated by the transition from FIGS. 2K and 5B to FIGS. 2L and 5A. It is hypothesized that providing the upstream assembly with self-tightening patch anchors such as these (e.g., by including the patch with a spring that is biased to draws the patch anchors tight) can advantageously obviate a need for a discrete tightening or locking step to be performed, and/or for a discrete locking component to be included.

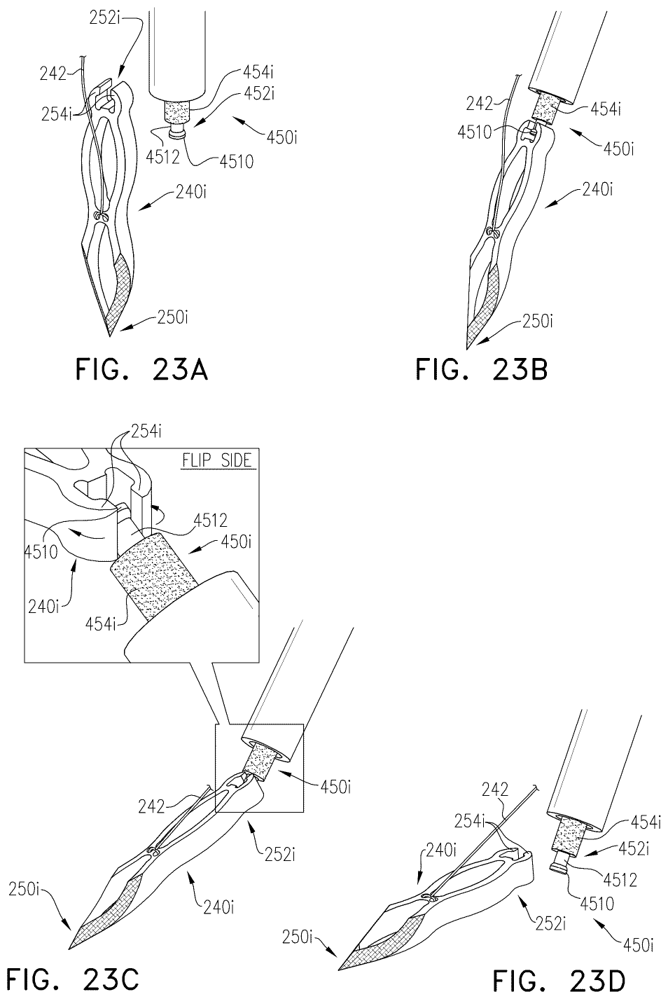

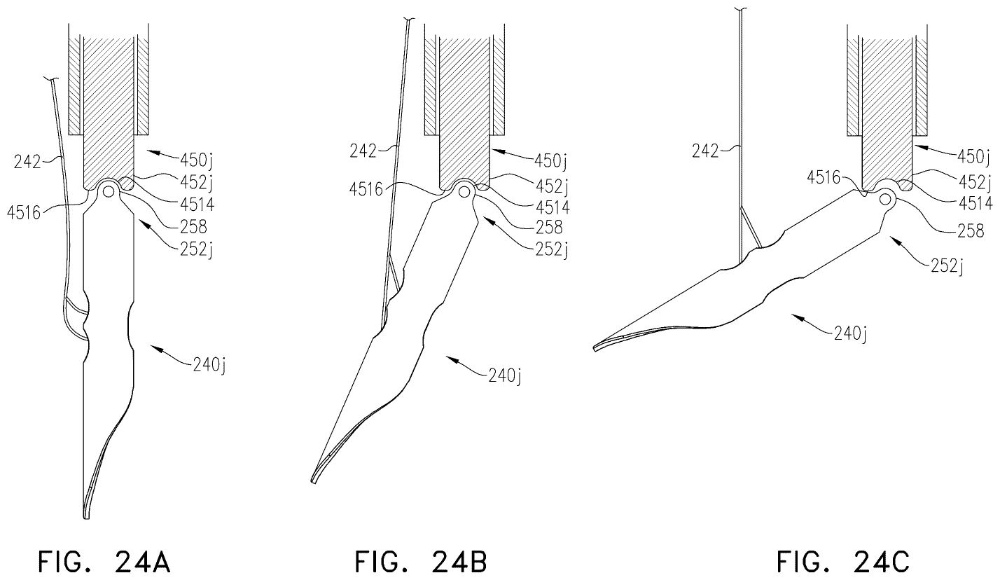

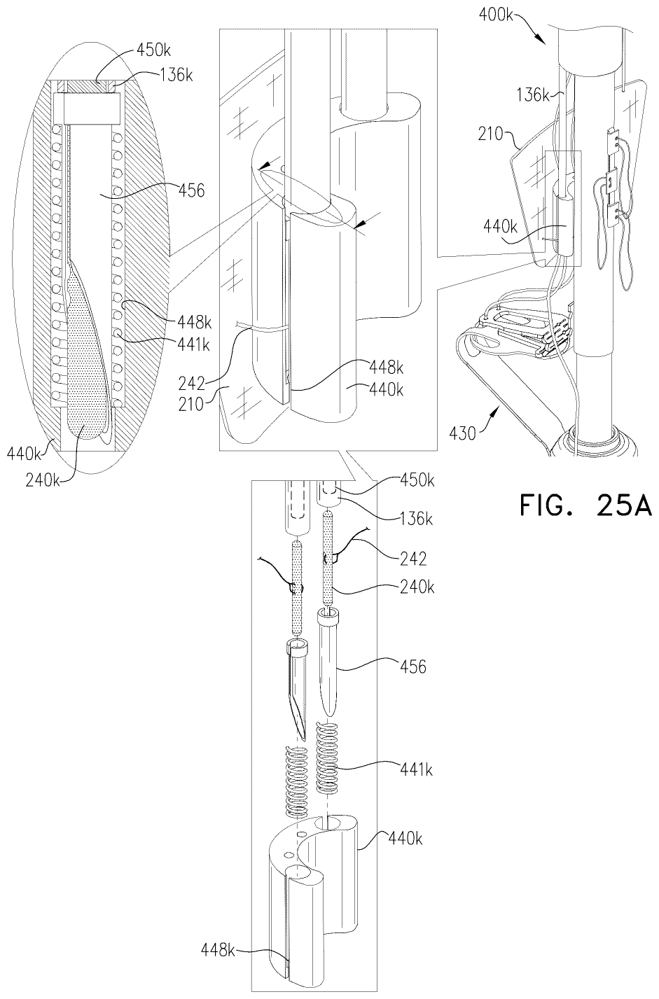

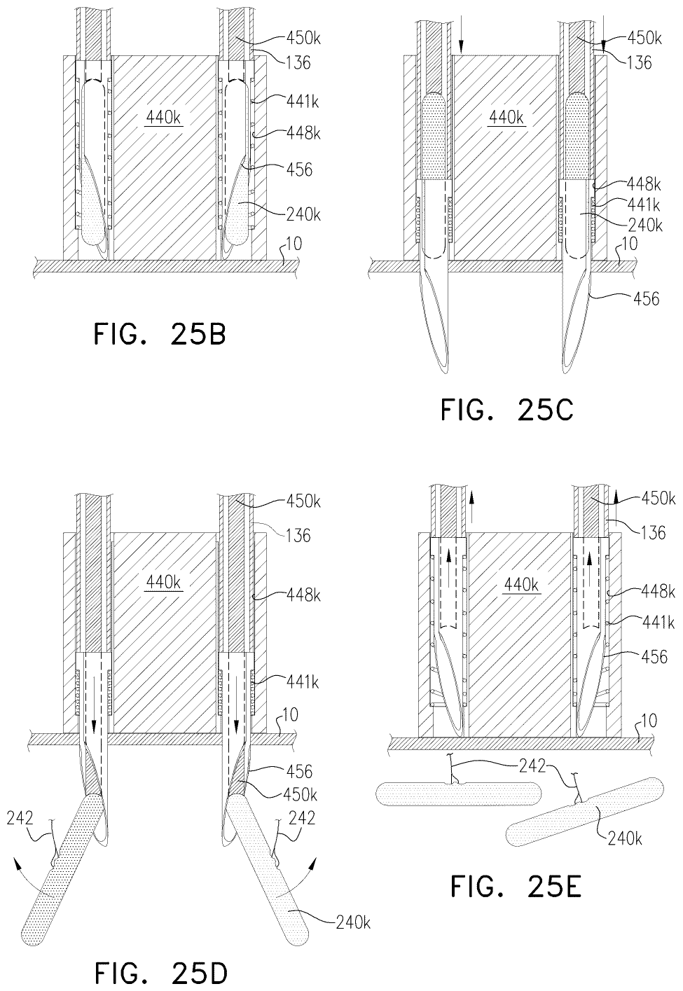

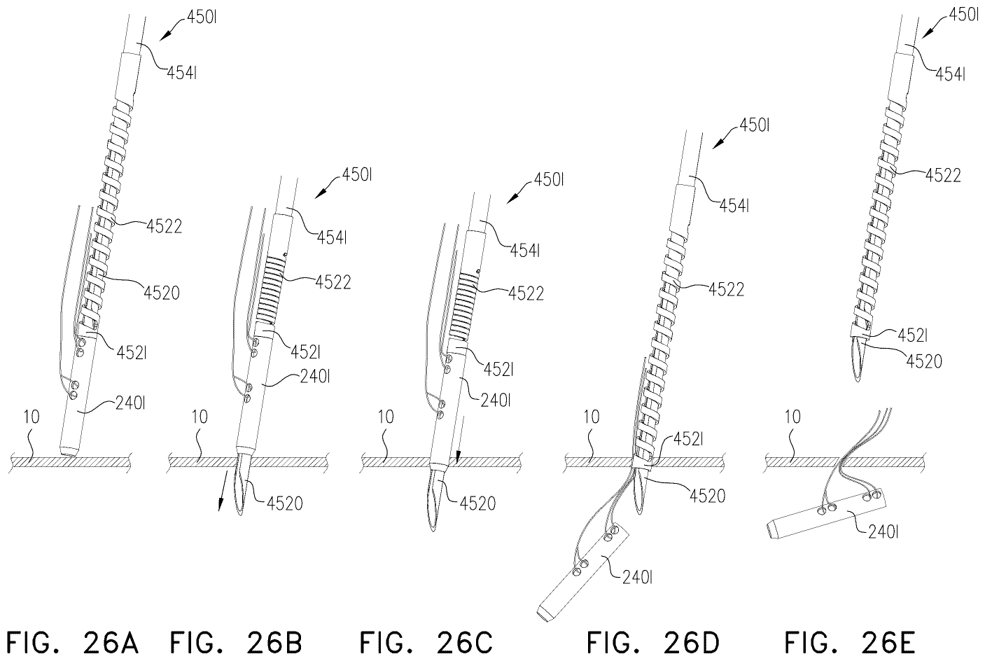

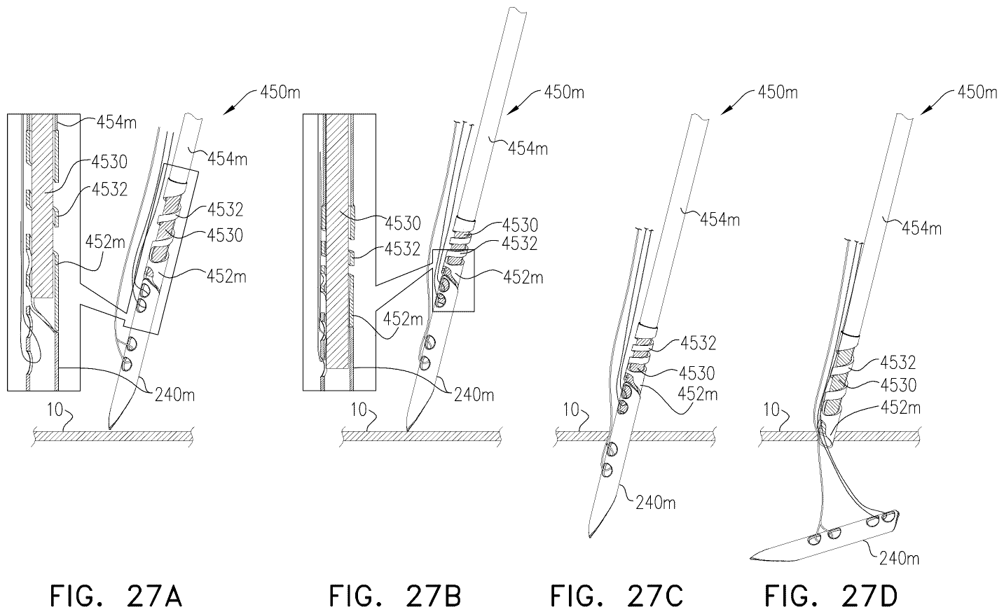

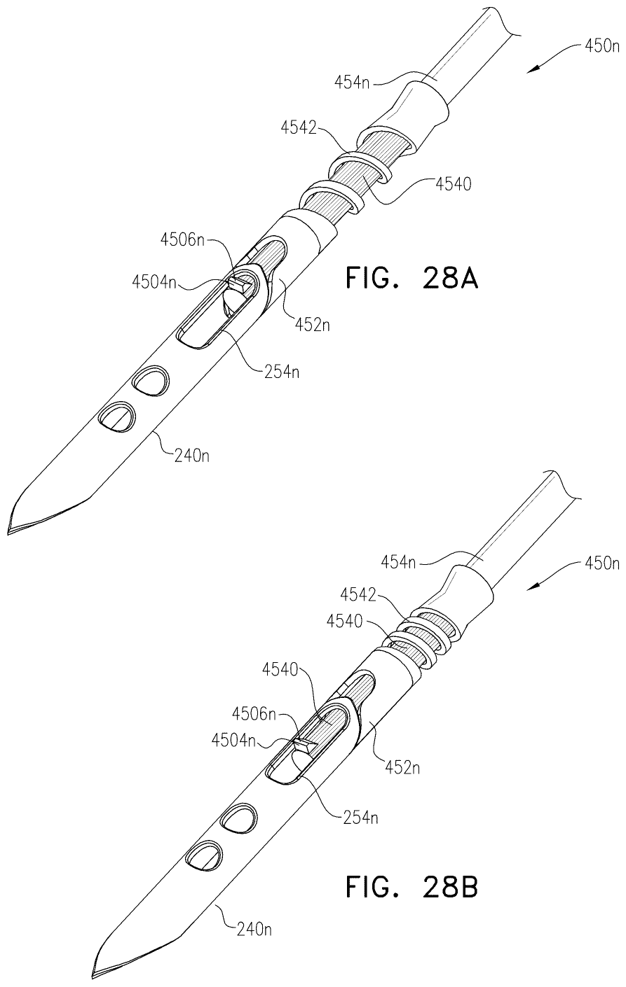

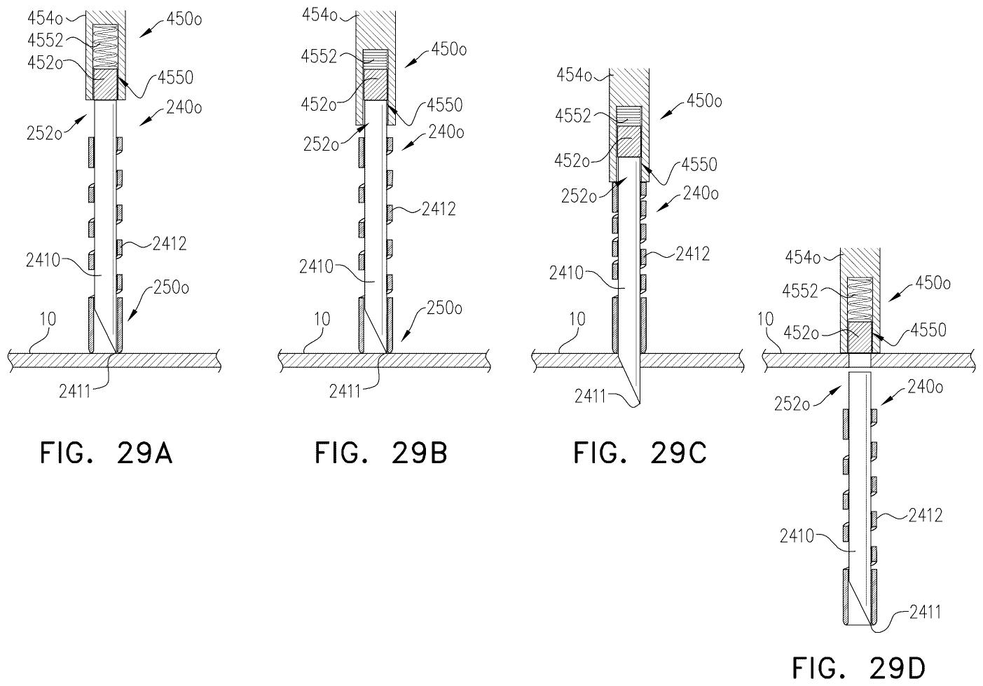

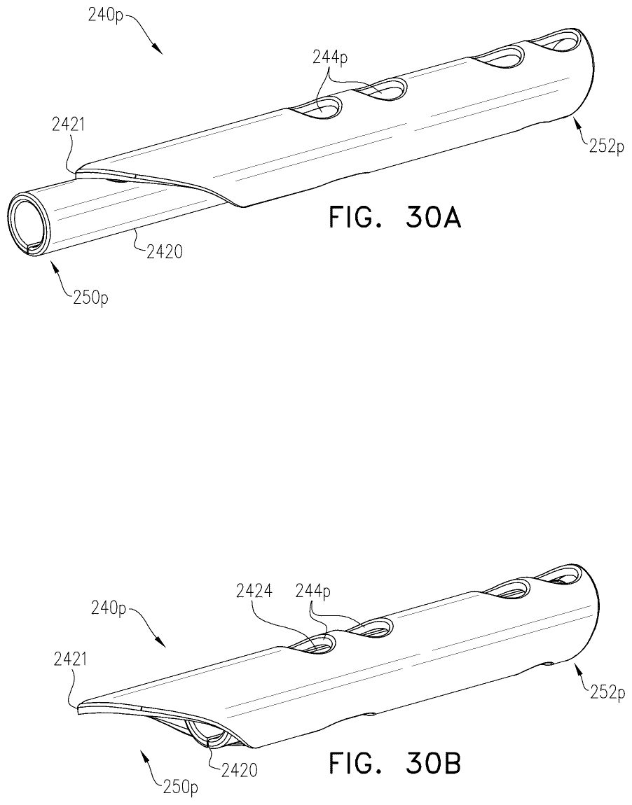

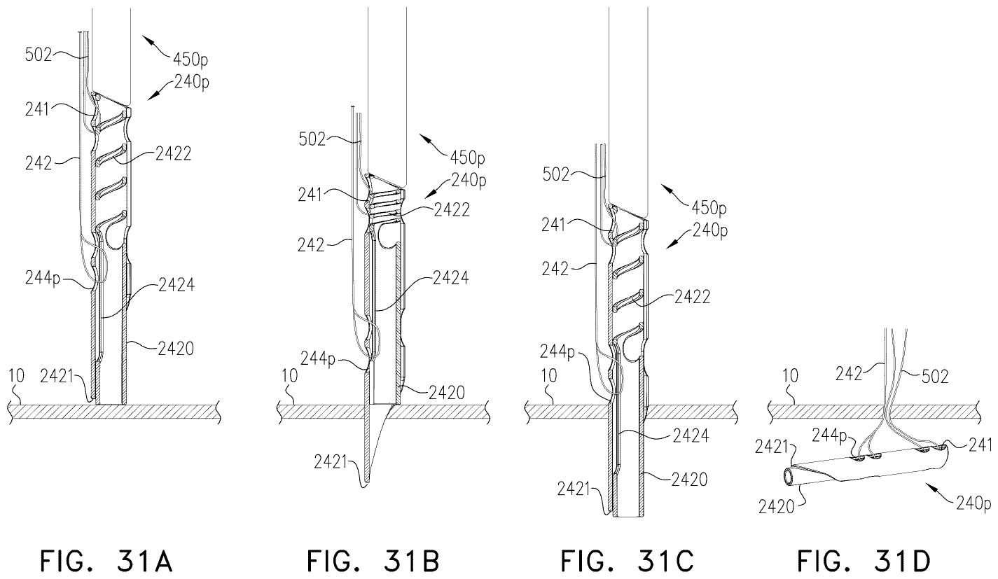

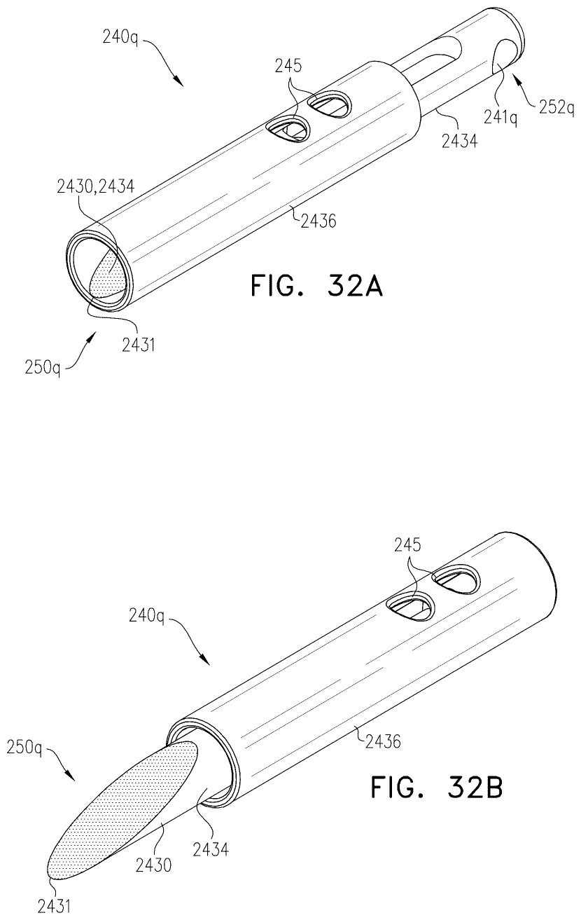

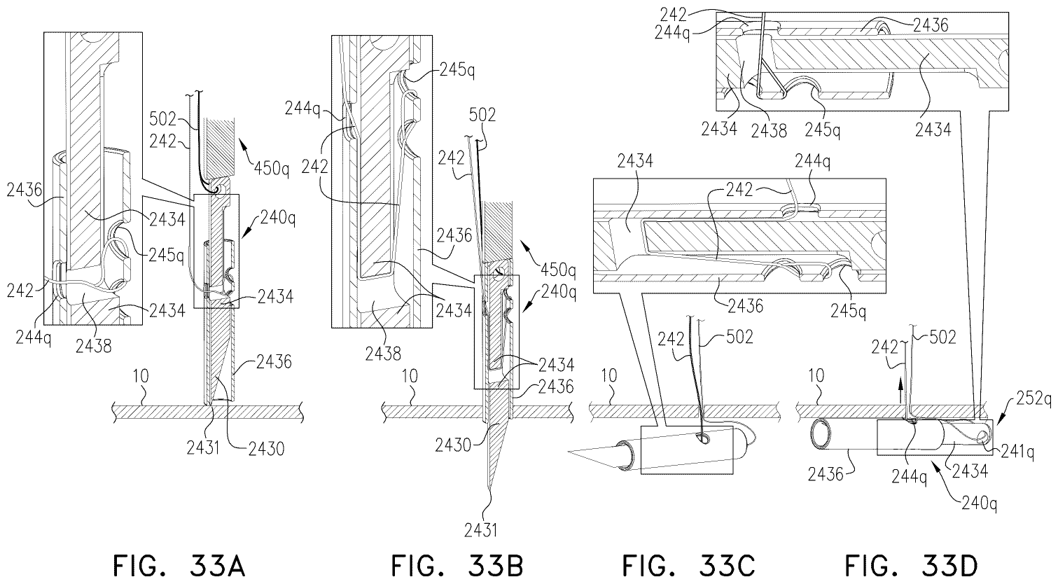

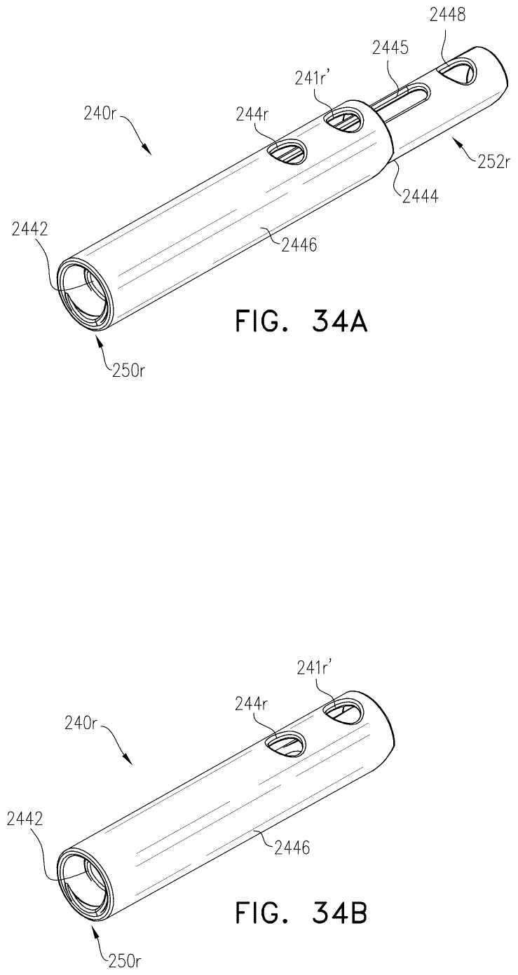

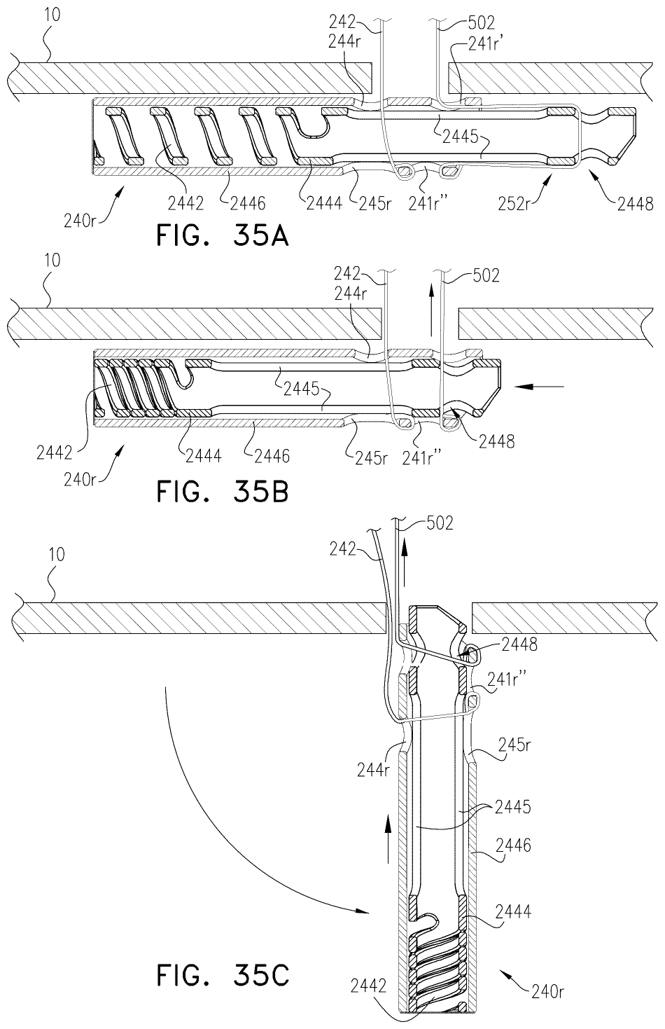

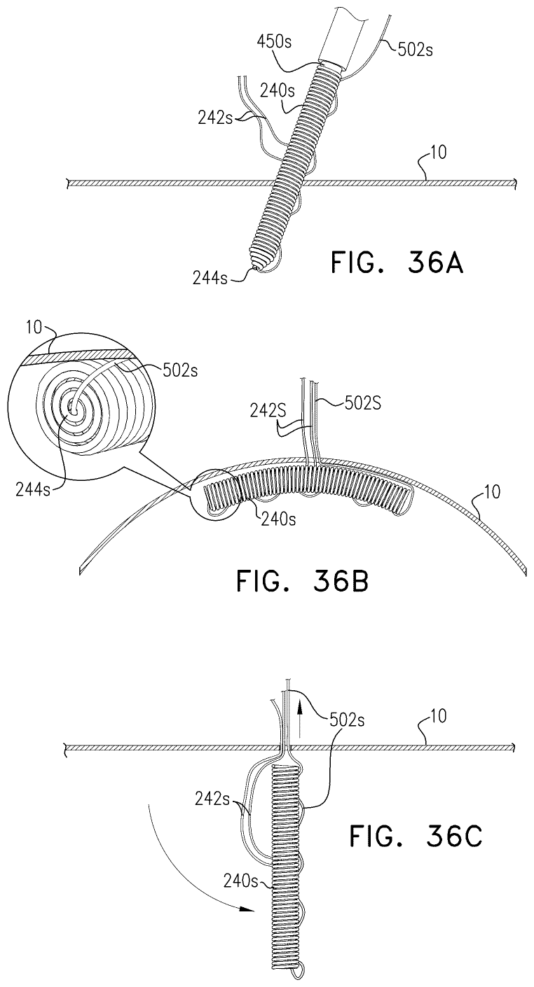

It is to be noted that the scope of the present disclosure includes the use of other patch-anchor tightening mechanism (e.g., one or more spring, elastic region, tensioner, winch, screw, etc.) that may or may not contract the patch linearly, or that may or may not contract the patch at all. Some non-limiting examples of other patch-anchor tightening mechanisms are described in International Patent Application No. PCT/IB2021/060436 to Tennenbaum et al., filed Nov. 11, 2021, entitled “Valve leaflet treatment systems and methods,” and which published as WO 2022/101817 (e.g., with reference to FIGS. 36A-K thereof), which is incorporated herein by reference.

In some implementations, and as shown, frame 230 defines at least one patch-anchor support 236, which can be positioned and shaped to partially or completely surround point 214. In some implementations, and as shown, spring 234 can be coupled to root brace 232 via patch-anchor support 236. Patch-anchor support 236 can advantageously provide one or more of the following benefits: (1) to serve to reinforce the patch at point 214, e.g., to protect against cord 242 cutting through sheet 220; (2) to serve as a bearing surface over which cord 242 slides as it moves through point 214; and/or (3) to provide opposition (e.g., an opposing force) against which patch anchors 240 can press the leaflet, thereby improving sandwiching of the leaflet between the patch anchors and patch 210, e.g., compared to a similar patch in which the patch anchors press the leaflet against part of the patch in which sheet 220 is not supported by frame 230.

As described hereinabove, the tether that tethers the upstream assembly to the downstream assembly can be attached to a lip region of the patch. For applications in which the patch comprises a frame that defines a lip brace, the tether can be attached to the lip brace. As shown in FIGS. 5A-B, for patch 210 tether 160 is attached to lip brace 231 (e.g., to an eyelet defined by the lip brace), at the middle of lip 211. It is to be noted that, although tether 160 and cords 242 are both attached to lip brace 231, they act at least partly independently, e.g., with spring 234 tensioning cords 242 irrespective of whether tether 160 pulls on the lip brace. Furthermore, lip brace 231 can be configured to distribute at least some of the force exerted on patch 210 by tether 160 (e.g., during ventricular systole) across the width of the patch, e.g., across the width of lip 211. Nonetheless, the finite length of cords 242 can limit the degree to which such force is experienced by patch 210 itself (e.g., by sheet 220), e.g., preventing the patch from being stretched deleteriously. That is, once cord 242 pulls its patch anchor tight against the leaflet, firmly sandwiching the leaflet against contact face 221 of patch 210, the cord can inhibit further distancing of lip brace 231 from root brace 232.

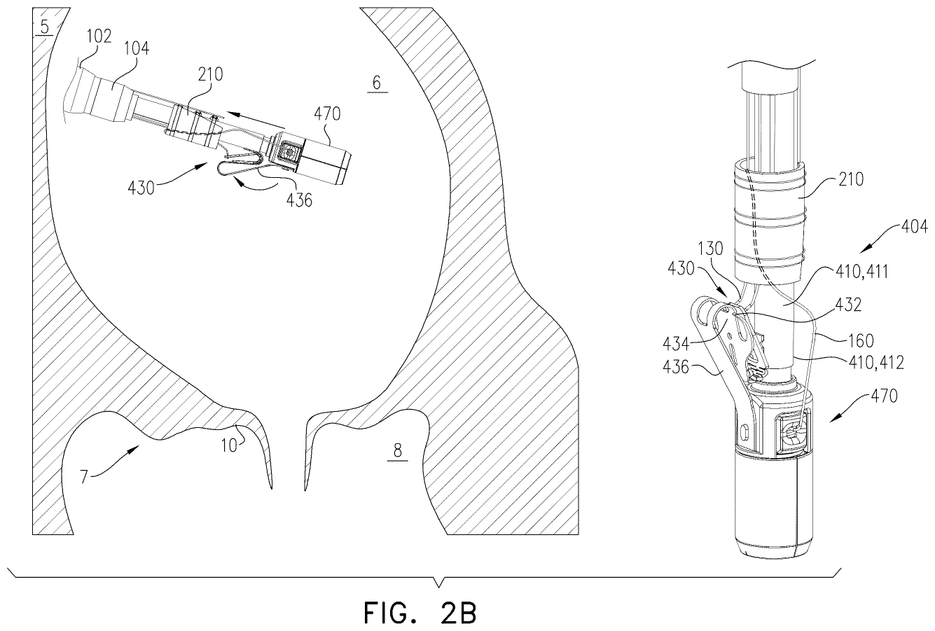

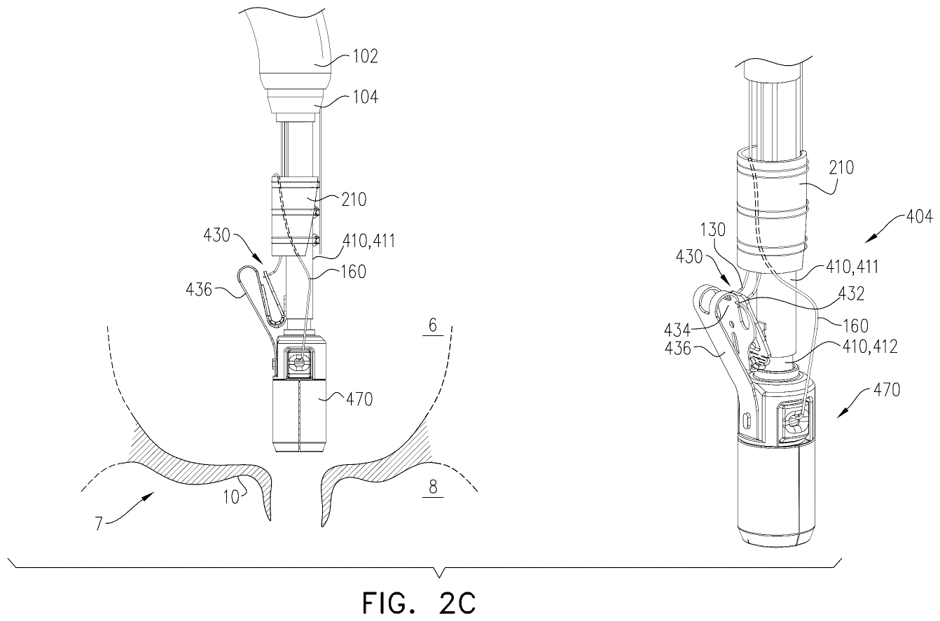

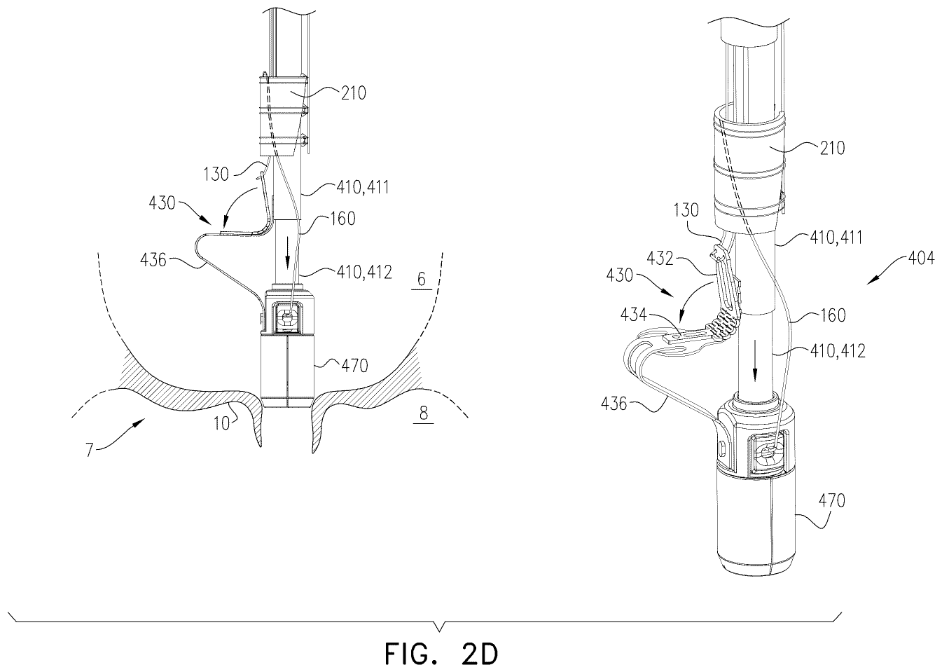

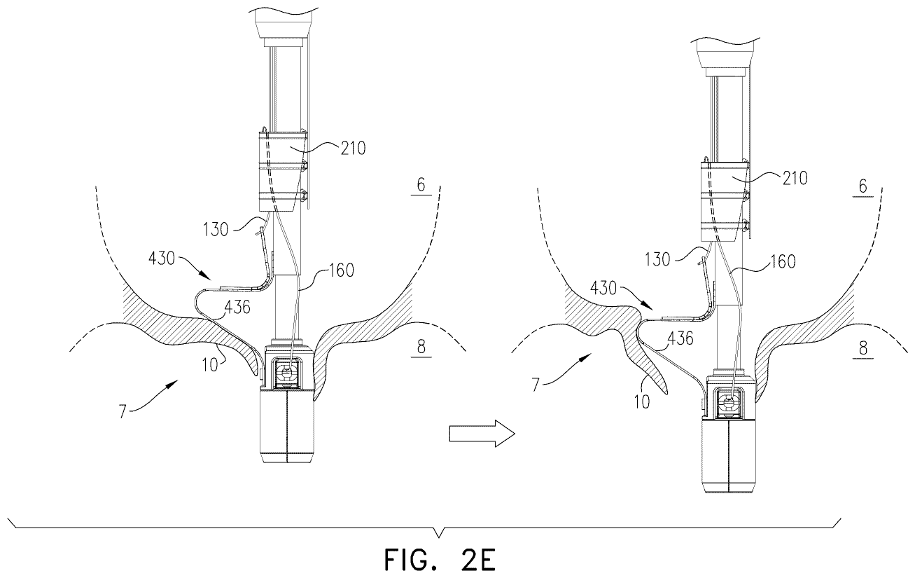

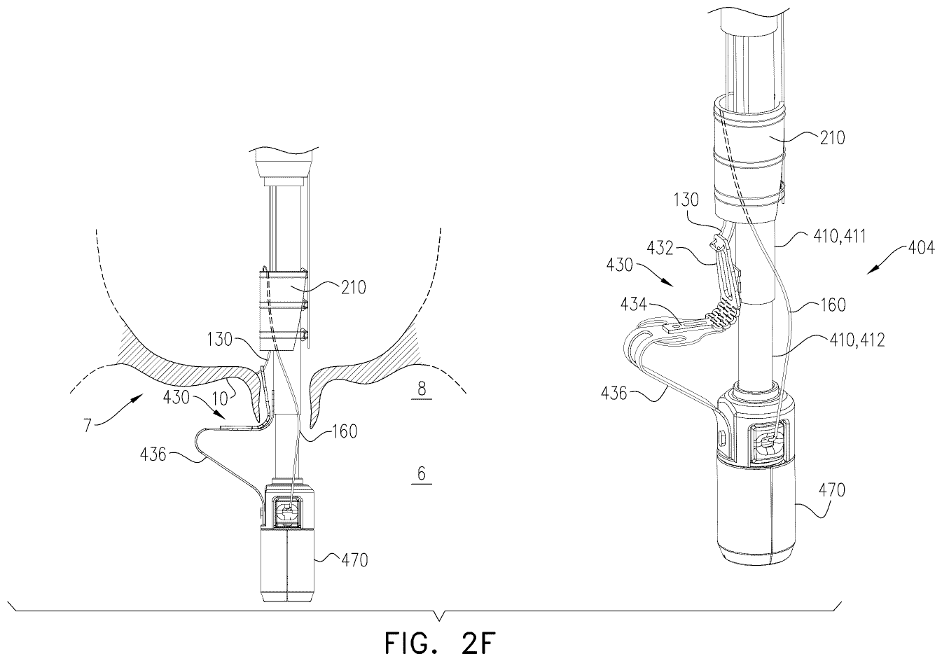

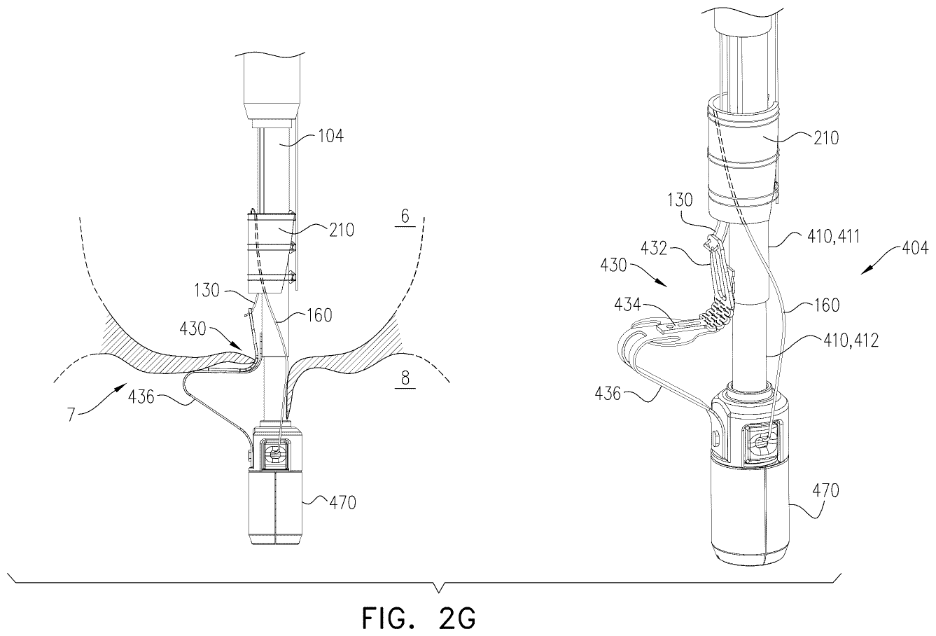

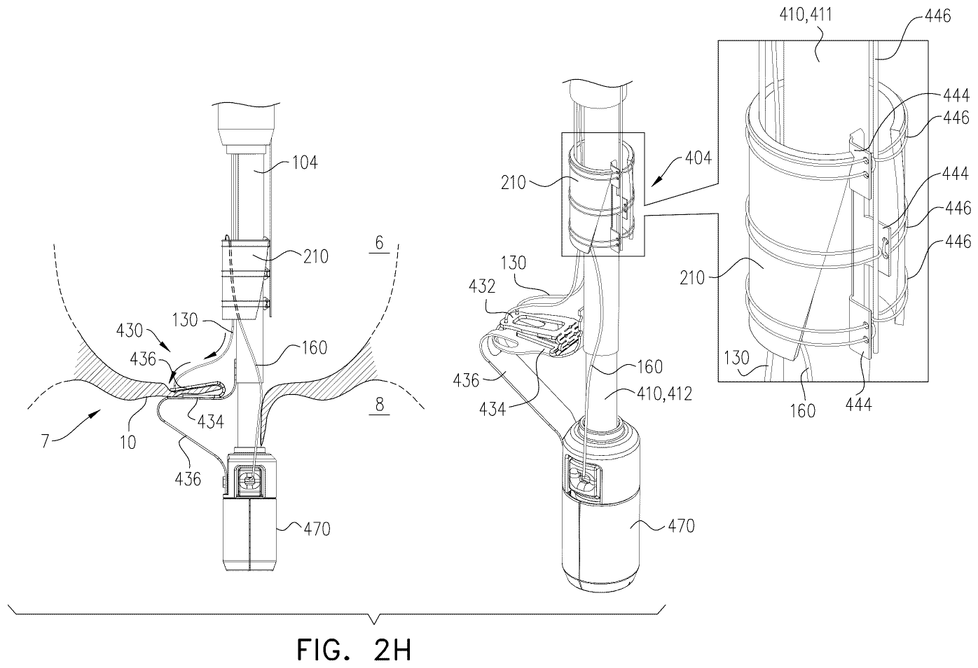

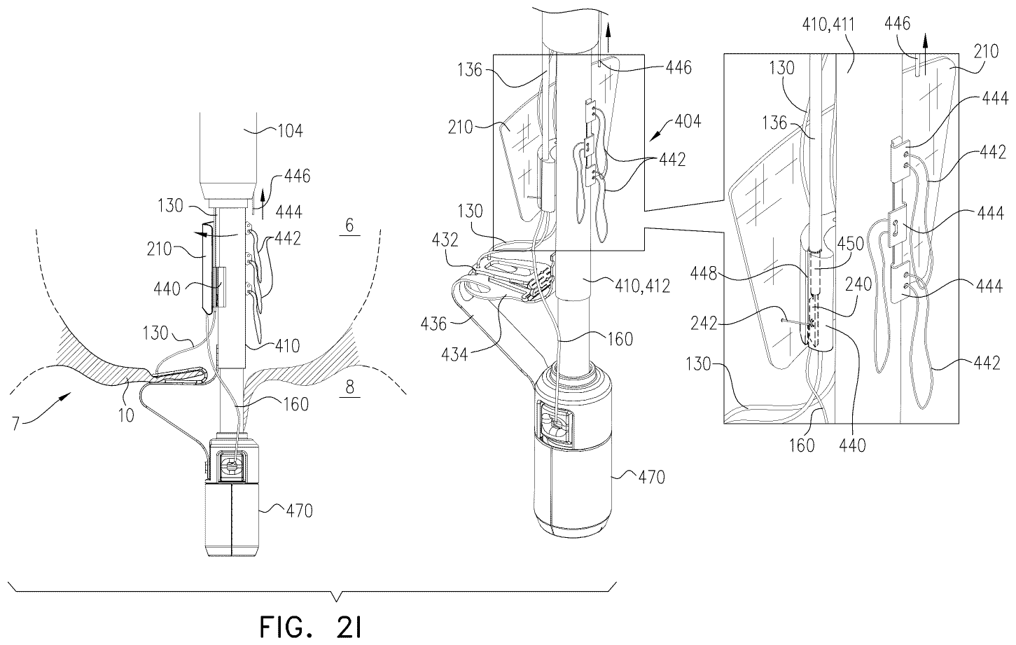

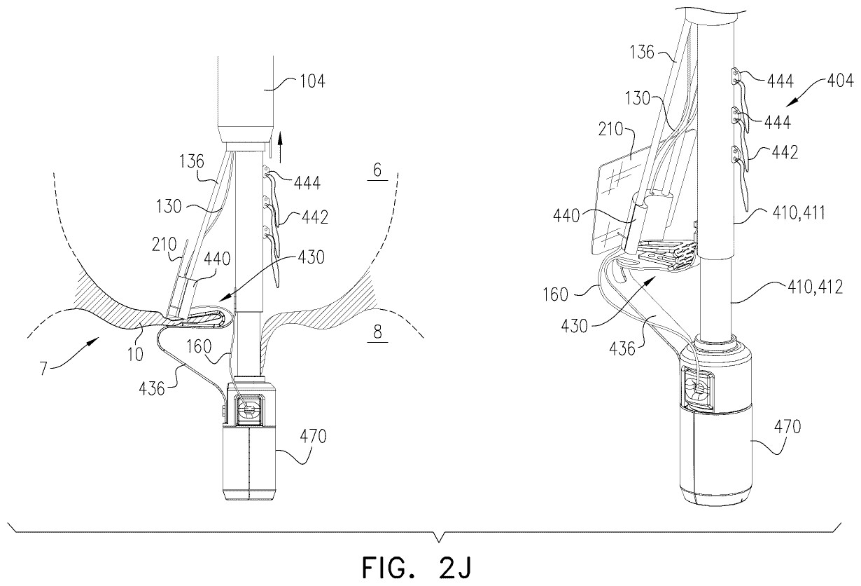

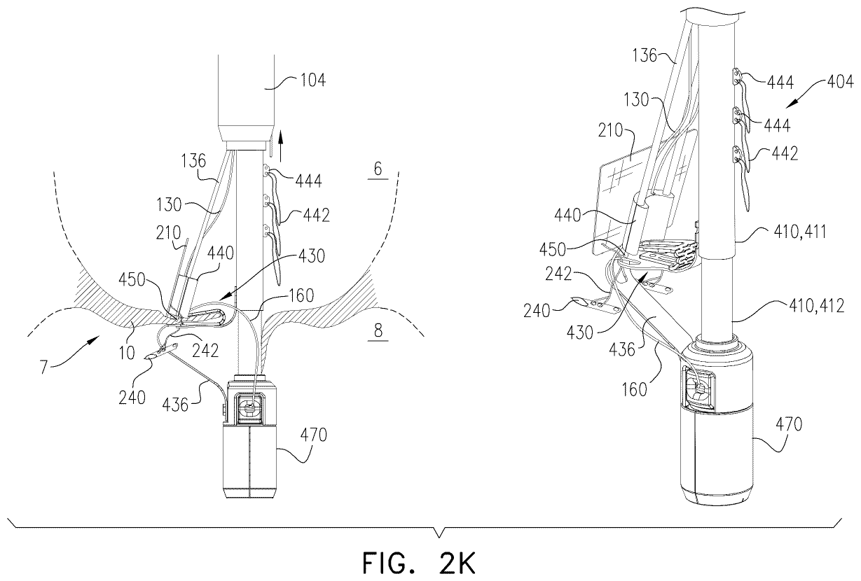

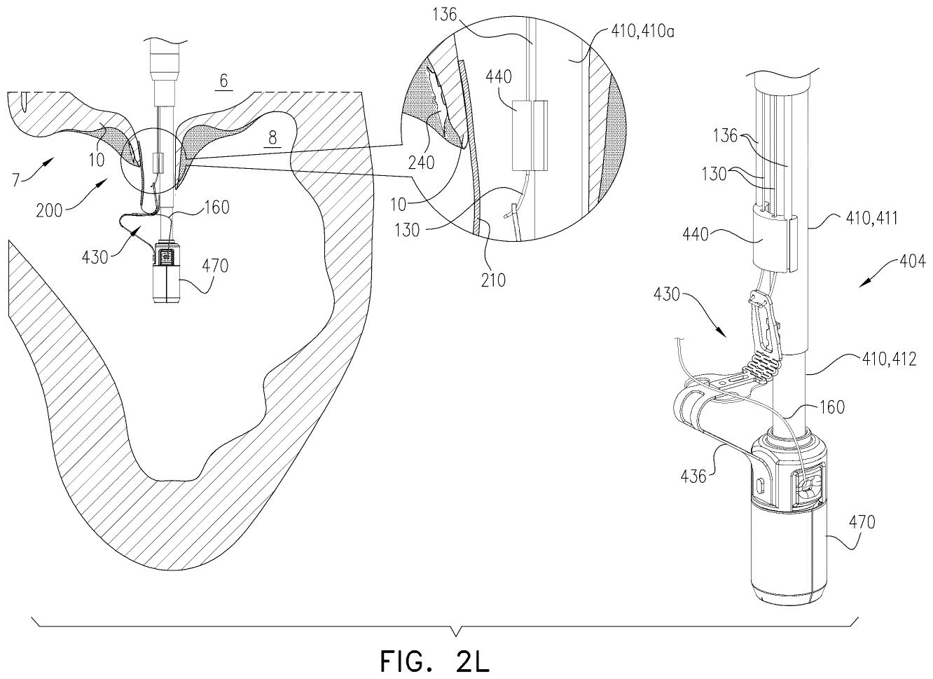

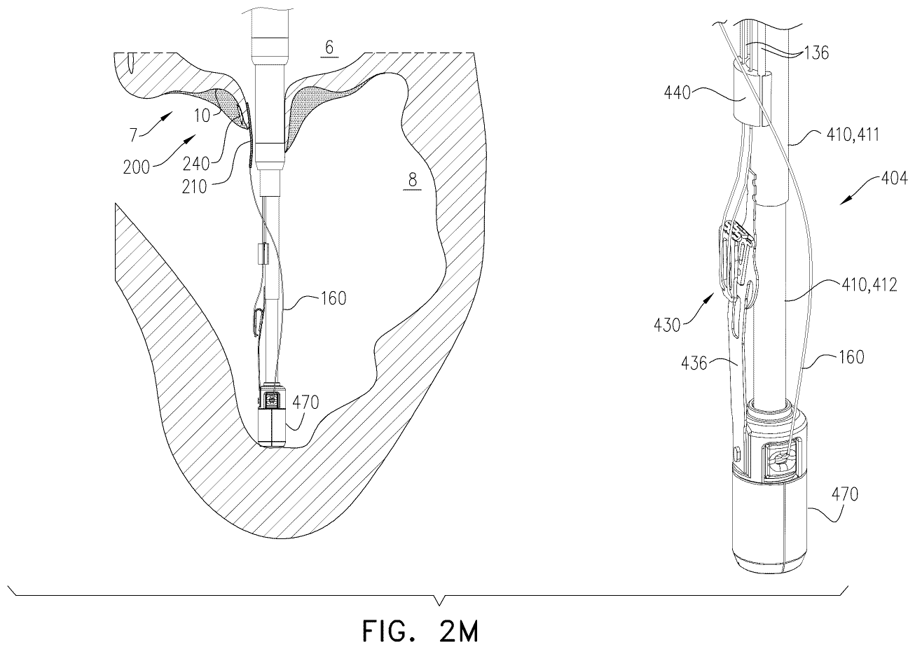

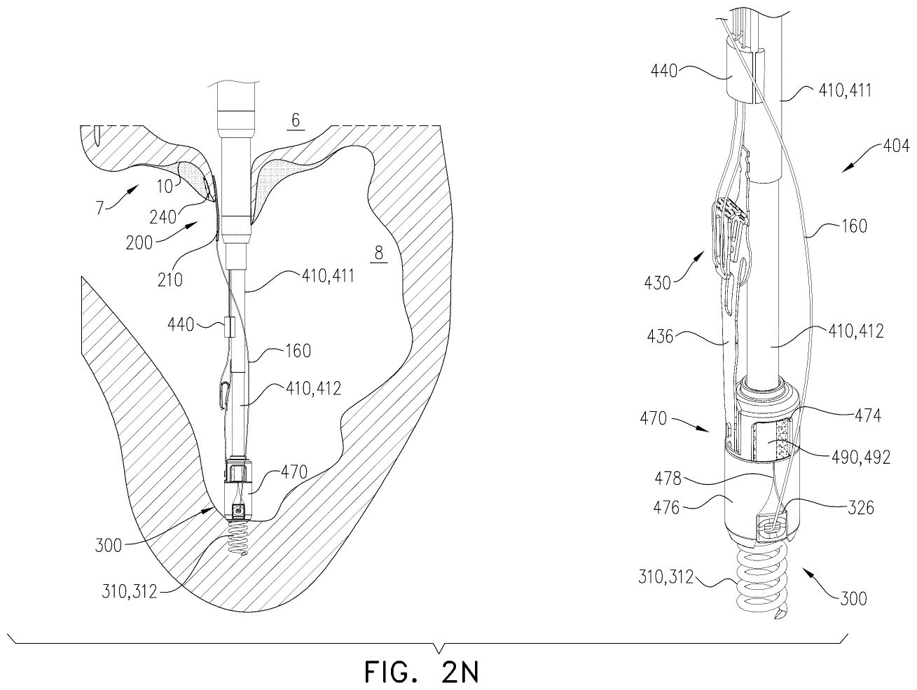

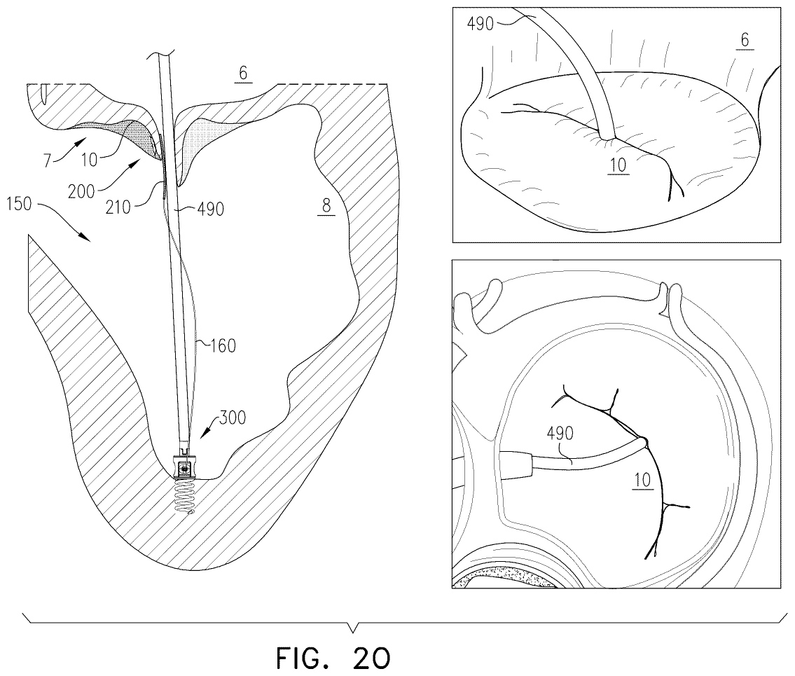

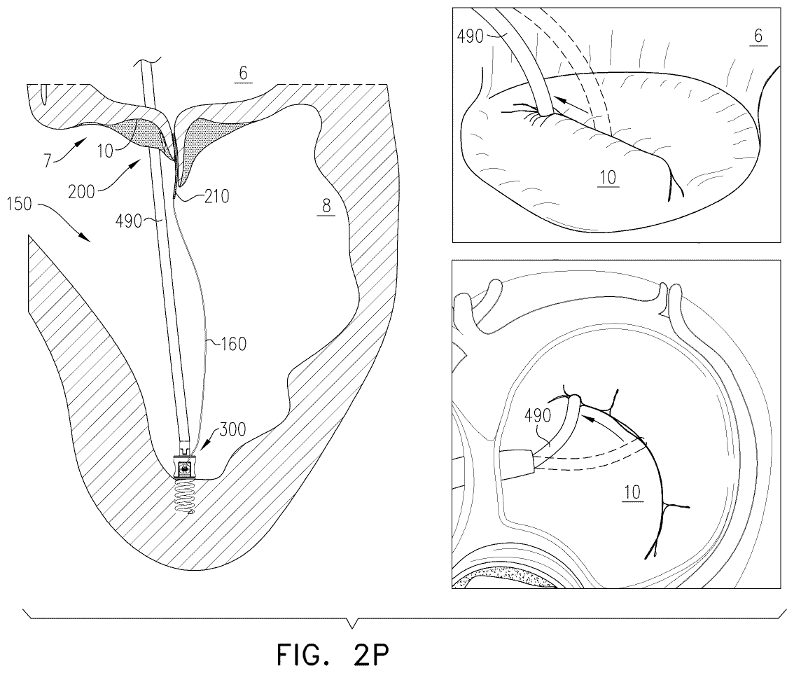

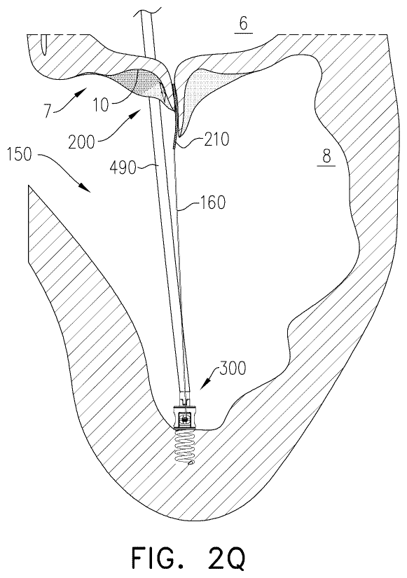

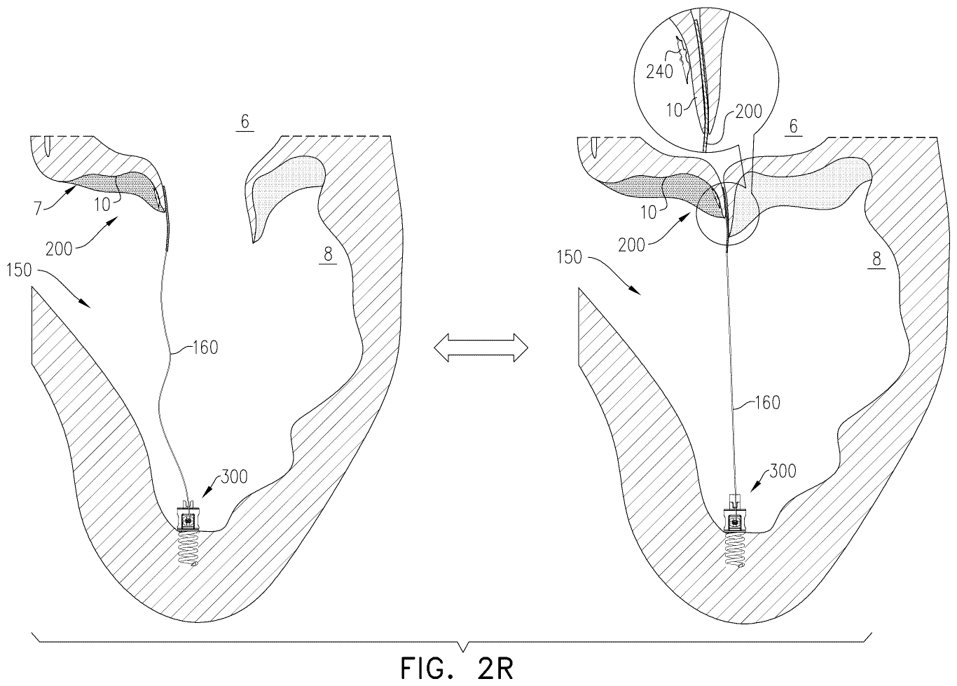

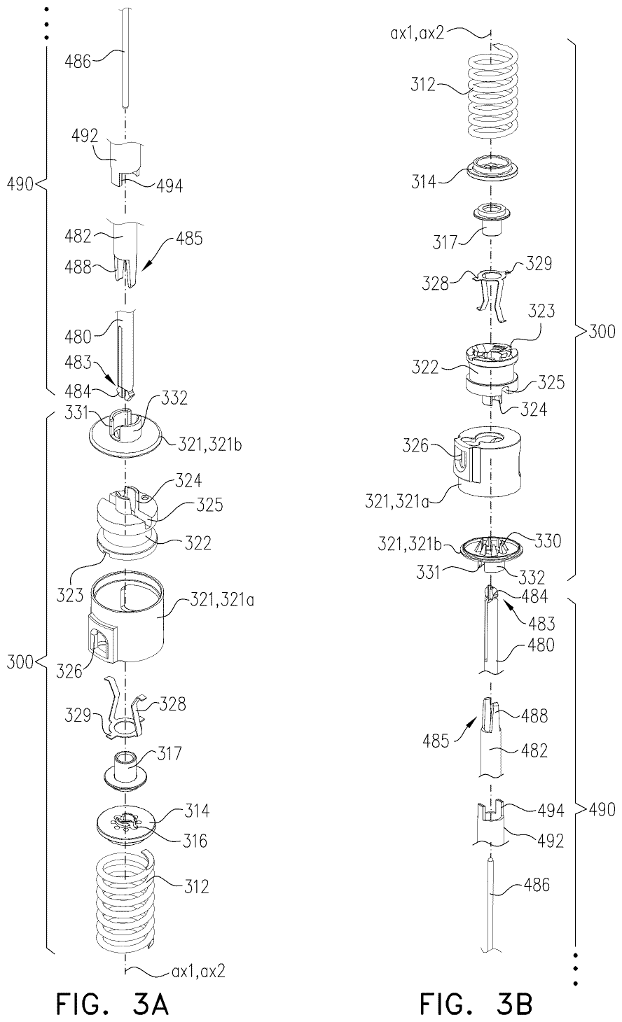

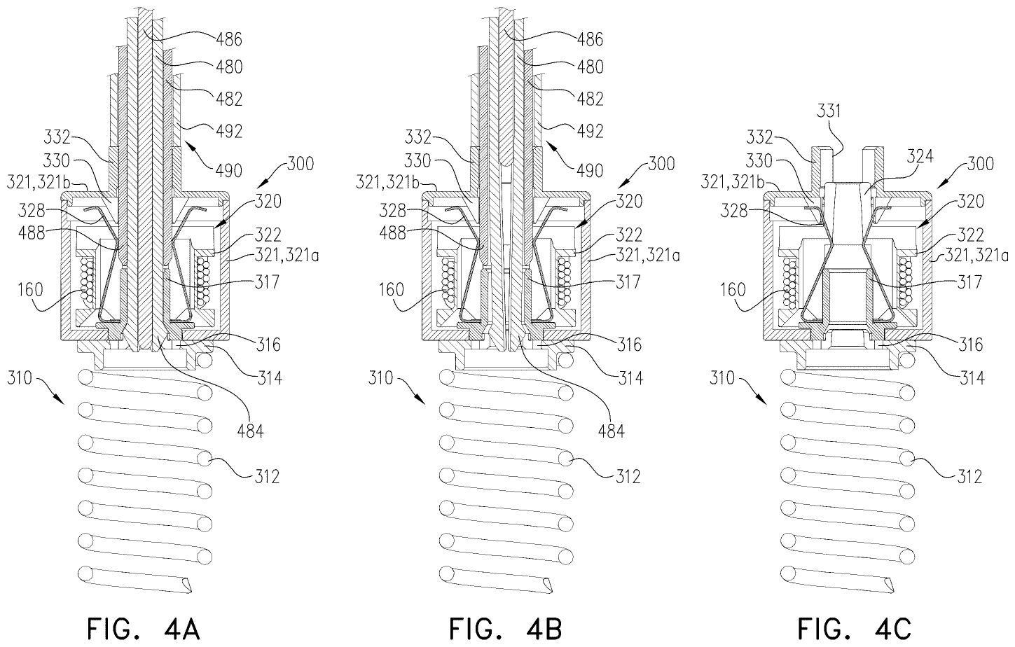

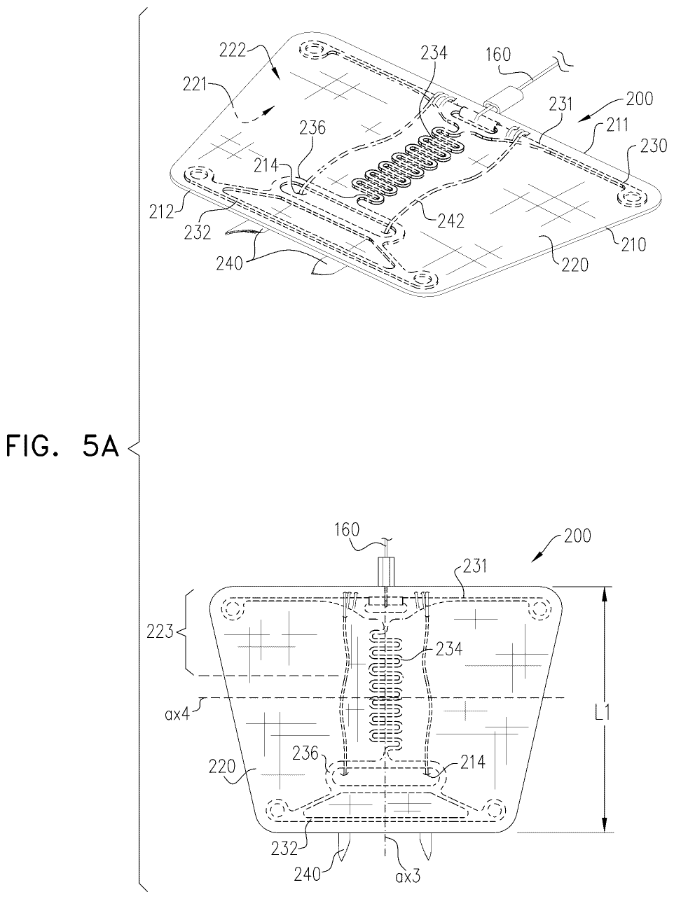

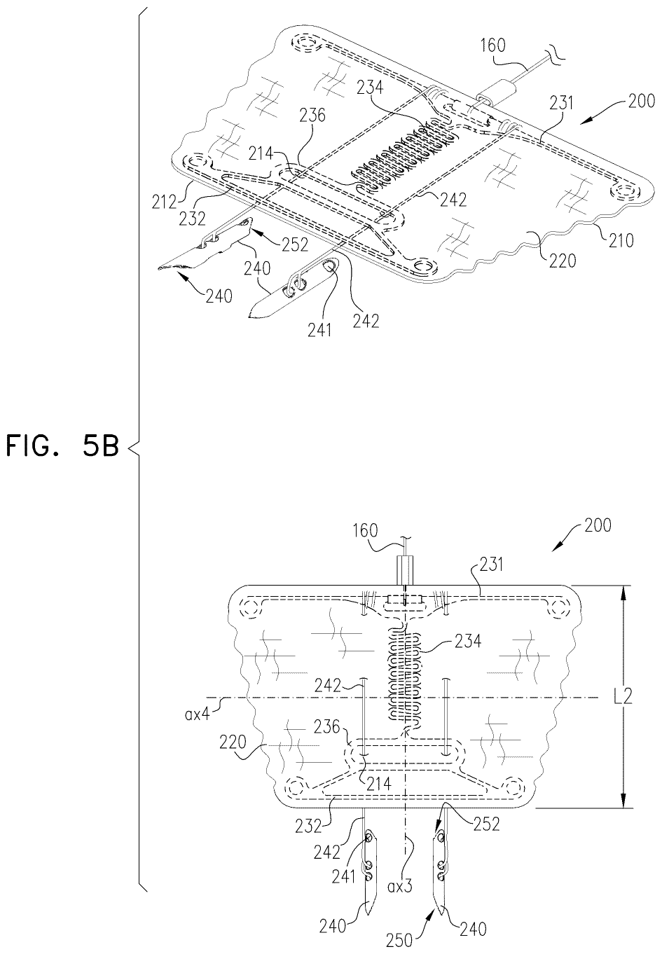

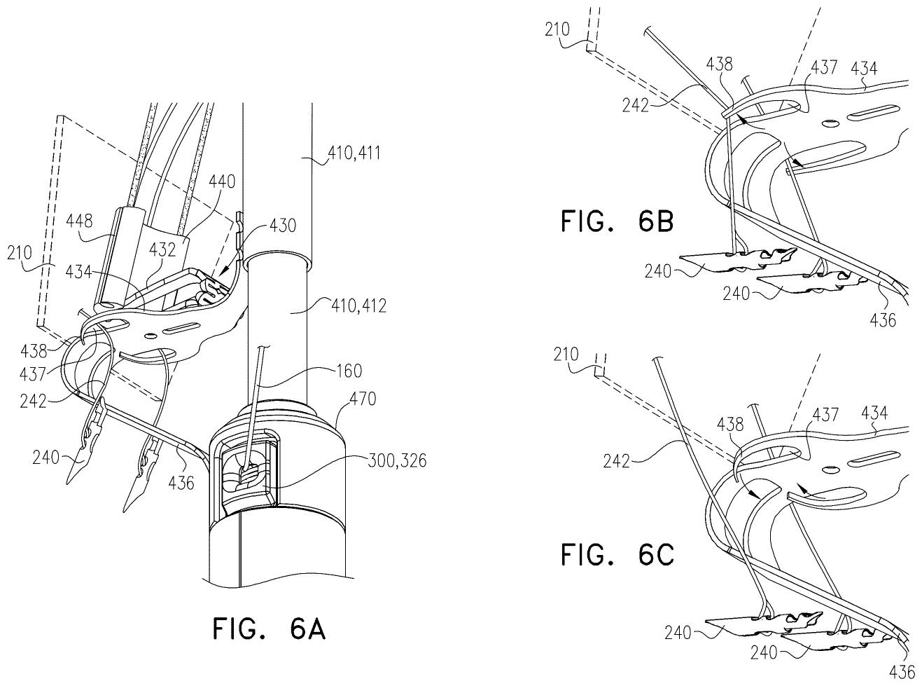

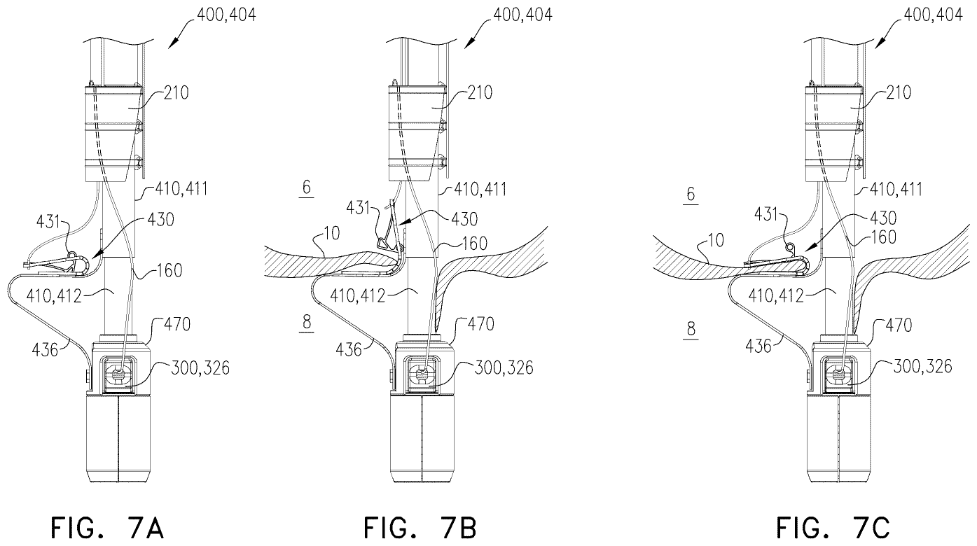

Reference is now made to FIGS. 7A-C, which are schematic illustrations of a grasping indicator 431, in accordance with some implementations. Indicator 431 can be a component of clasp 430 or can be coupled to the clasp. Indicator 431 can be radiopaque and/or echogenic, and is configured to change its position and/or orientation (e.g., with respect to upstream support 432 and/or downstream support 434) responsively to successful grasping of leaflet 10 by clasp 430. This configuration may be due to the shape and/or size of indicator 431, and/or due to the nature of its coupling to upstream support 432 and/or downstream support 434. The change of position and/or orientation of indicator 431 is detectable (e.g., visually) using imaging (e.g., fluoroscopy or ultrasound), allowing the operator (e.g., the physician) to verify successful grasping of leaflet 10 prior to driving patch anchors 240.

In the example shown, indicator 431 comprises a radiopaque material, and is flexibly coupled to upstream support 432. In its resting state, indicator 431 protrudes only minimally, or does not protrude, from clasp 430 (FIG. 7A). When clasp 430 is opened, indicator 431 can remain stationary with respect to upstream support 432, e.g., can remain in its resting state (FIG. 7B). However, when clasp 430 is reclosed to grasp leaflet 10, the presence of the leaflet between upstream support 432 and downstream support 434 pushes (e.g., deflects) indicator 431 to protrude from clasp 430, e.g., to protrude through an opening in the upstream support. This protrusion is detectable (e.g., visually) using imaging (e.g., fluoroscopy), indicating successful grasping of leaflet 10 prior to driving patch anchors 240. To achieve this effect, in the resting state, part of indicator 431 can protrude from upstream support 432 toward downstream support 434 (e.g., can protrude into the gap between these supports) such that, when leaflet 10 is clasped, the leaflet displaces the part of the indicator, and thereby the entire indicator. It is to be noted that the part of indicator 431 that protrudes into the gap between the supports can be disposed deeper into clasp 430 (e.g., closer to the point of articulation between the supports) than are slots 437 (e.g., the points through which patch anchors will pass), thereby ensuring that the indication is only provided if leaflet 10 is clasped sufficiently deep within clasp 430 that patch anchors will be driven through leaflet tissue sufficiently far from the lip of the leaflet.

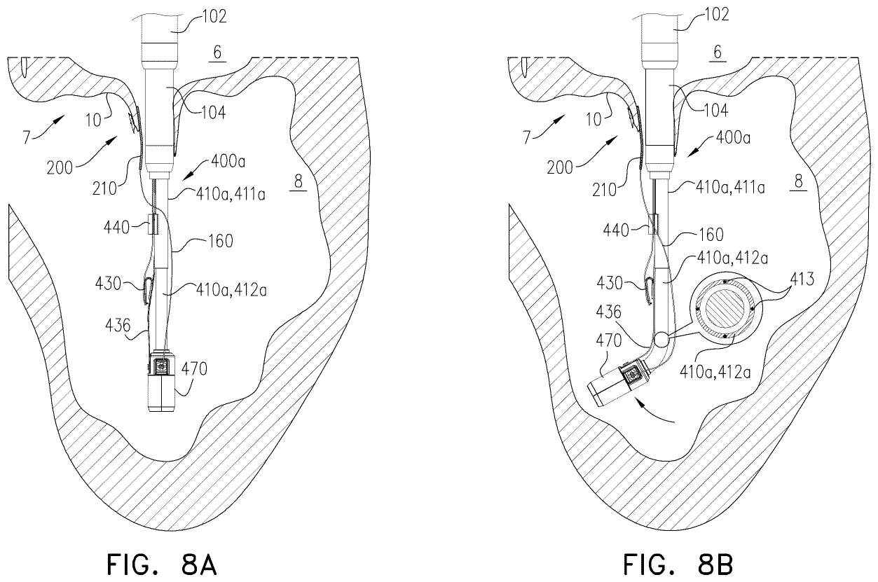

Reference is now made to FIGS. 8A-B and 9A-B, which are schematic illustrations of steerable variants of delivery tool 400, in accordance with some implementations. With reference to these figures, and throughout the present application, the suffixation of a letter (e.g., a, b, etc.) to a reference numeral indicates that the element assigned the reference numeral with the suffix is a variant of another element described herein that has the same name and that has the same reference numeral without the suffix or with a different suffix. In each case, the variant can be as described for (e.g., can have a similar structure and/or function as) the other element except as noted. For example, delivery tool 400a and delivery tool 400b, described with reference to FIGS. 8A-B and 9A-B, respectively, are variants of delivery tool 400, and can be as described for delivery tool 400 except as noted. Furthermore, in each case, the variant can be substituted, mutatis mutandis, with the other element, or with other variants of the element described herein.

As described hereinabove, advancement of tool 400, with implant 150 mounted thereon, through the vasculature can be facilitated by one or more catheters 102, 104, one or more of which can be steerable (e.g., actively steerable, e.g., using pull-wires or other components known in the art). To facilitate this, tool 400 is largely flexible along its length, e.g., such that it can passively follow the path of, and/or can be passively bent by, the catheters. An exception to this can be parts of shaft 410 at distal portion 404, e.g., the telescopic arrangement of proximal part 411 and distal part 412. However, in some implementations, these too can be flexible. Furthermore, and as shown in FIGS. 8A-B and 9A-B, in some implementations, at the distal portion of the delivery tool, the shaft itself can have a steerable part that is actively steerable independently of catheters 102 and 104, e.g., via operation of extracorporeal proximal portion 402 (e.g., operation of a controller thereof).

FIGS. 8A-B show a delivery tool 400a, which is steerable using one or more pull-wires 413, e.g., disposed within the wall of the shaft of the delivery tool. In the example shown, four pull-wires 413 are disposed within the wall of shaft 410a, e.g., extending through distal part 412a of the shaft. However, fewer (e.g., one, two, or three) or more pull-wires can be used. Pull-wires 413 operatively couple extracorporeal proximal portion 402 (e.g., a controller thereof) to the steerable part of shaft 410a, such that the extracorporeal proximal portion can be used to bend the shaft (e.g., part 412a thereof) in a similar manner to the way that pull-wires are used to bend steerable catheters. FIG. 8A shows the distal portion of tool 400a having been advanced into ventricle 8 but not actively steered. In this state (e.g., a resting state), the steerable part of the shaft can be substantially straight. However, in some implementations, the steerable part of the shaft (e.g., distal part 412a of shaft 410a) can be biased to assume a curved shape, e.g., in a resting state thereof.

FIG. 8B shows the distal portion of tool 400a being actively bent via tensioning of at least one of pull-wires 413. In the example shown, it is distal part 412a of shaft 410a that bends responsively to tensioning of the pull-wire, e.g., that is configured to be sufficiently flexible to bend. The resulting deflection of the distal portion of tool 400a can advantageously allow the operator greater control over the positioning of downstream assembly 300, and/or greater choice regarding the site at which the downstream assembly 300 will be anchored.

FIGS. 9A-B show a delivery tool 400b, which is steerable via extension and contraction of its shaft 410b. As described hereinabove for delivery tool 400, frame element 436 can extend from proximal part 411b of shaft 410b to distal part 412b of the shaft, thereby enabling deflection of at least part of clasp 430 via extension and contraction (e.g., telescopically). In delivery tool 400b, sufficient extension of distal part 412b of shaft 410b from proximal part 411b of the shaft causes the shaft (e.g., distal part 412b thereof) to bend, thereby deflecting capsule 470 and downstream assembly 300 therewithin. As shown, this can be facilitated by configuring frame element 436 (e.g., its length between its attachment points to proximal part 411b and distal part 412b) such that, beyond a threshold degree of extension of distal part 412b from proximal part 411b, further extension of the distal part from the proximal part causes the frame element to pull the distal part of the shaft to deflect to the side on which the frame element is disposed, e.g., by tensioning the frame element.

FIG. 9A shows the distal portion of tool 400b having been advanced into ventricle 8 but not actively steered. In this state (e.g., a resting state), the distal portion of the tool can be substantially straight. However, in some implementations, the distal portion of the tool (e.g., distal part 412b of shaft 410b) can be biased to assume a curved shape, e.g., in a resting state thereof.

FIG. 9B shows the distal portion of tool 400b being actively bent via extension of distal part 412b from proximal part 411b. In the example shown, this is achieved by retracting proximal part 411b proximally, e.g., toward atrium 6. However, it is to be understood that this can similarly be achieved by advancing distal part 412b distally (e.g., deeper into ventricle 8)—or a combination of moving both proximal part 411b proximally and distal part 412b distally.

In the example shown, it is distal part 412b of shaft 410b that responsively bends, e.g., that is configured to be sufficiently flexible to bend. The resulting deflection of the distal portion of tool 400b can advantageously allow the operator greater control over the positioning of downstream assembly 300, and/or greater choice regarding the site at which the downstream assembly 300 will be anchored.

In some implementations, delivery tool 400b differs from tool 400 only in that, at its distal portion, shaft 410b (e.g., a distal part 412b thereof) is sufficiently flexible to bend as described, and/or in that shaft 400b and frame element 436 are dimensioned appropriately for this behavior.

It is to be noted that, for both tool 400a and tool 400b, the part of the shaft that is actively bendable can be disposed distally from mount 440 (e.g., distally from the axial location at which patch 210 was mounted) and proximally from capsule 470. In some implementations, and as shown, the part of the shaft that is actively bendable is disposed distally from clasp 430.

Reference is made to FIGS. 10A-B, which are schematic illustrations of a patch anchor 240a, in accordance with some implementations. Patch anchor 240a can be considered to be a variant of patch anchor 240 and, in any of the implants or systems described herein, can be used in place of patch anchor 240 mutatis mutandis.

Similarly to patch anchor 240, patch anchor 240a comprises a toggle, e.g., is a toggle anchor. However, patch anchor 240a is also biased to automatically widen upon deployment. FIG. 10A shows patch anchor 240a constrained within a channel 448a, and FIG. 10B shows the patch anchor having been deployed out of the channel (e.g., by a driver 450a) and having automatically widened in response to becoming unconstrained by the channel. Patch anchor 240a can therefore be formed (e.g., cut) from an elastic, superelastic, and/or shape memory material such as nitinol or cobalt-chrome.

In some implementations, and as shown, patch anchor 240a has a cellular structure (e.g., similar to that of a stent) and widens by foreshortening. For some such implementations, the cellular structure defines exactly two expanding cells, e.g., as shown. Patch anchor 240a can define an eyelet 244a through the toggle, via which cord 242 is attached to the patch anchor. Eyelet 244a can be disposed between the two cells, e.g., as shown.

In some implementations, channel 448a can be a channel defined by a mount such as mount 440. For some such implementations, a tip 250a of patch anchor 240a has a sharpened point (e.g., is sharpened to a point), e.g., as shown.

In some implementations, channel 448 can be defined by a needle that can itself be configured to pierce leaflet 10. For some such implementations, patch anchor 240a has a blunt tip.

The widening of patch anchor 240a can distribute force over a larger surface area of the leaflet, and thus over more collagen fibers of the leaflet. This can therefore increase the anchoring force/reliability compared with narrower and/or non-widening patch anchors. Furthermore, for applications in which the widening occurs via foreshortening, due to the resulting smaller length of patch anchor 240a, its ends may be less likely to deleteriously poke the leaflet to which it is anchored. Even In some implementations in which the widening does not occur via foreshortening, the increased width can allow patch anchor 240a to be manufactured shorter than a similar non-widening toggle anchor-thereby similarly reducing poking of the leaflet.

In some implementations, patch anchor 240a includes a retrieval feature 241a via which a retrieval line 502 is connected to the patch anchor. In some implementations, and as shown, retrieval feature 241a is situated toward (e.g., at) the proximal end of the patch anchor (e.g., of the toggle)—herein referred to as the “heel” of the patch anchor, which is at the opposite end to the tip of the patch anchor. In the example shown, retrieval feature 241a is, or includes a post around which retrieval line 502 is looped.

During anchoring of patch anchor 240a (e.g., during implantation of a patch to which it belongs), retrieval line 502 extends proximally from retrieval feature 241a. Should it be determined, during the course of implantation, that the patch anchor should be retrieved, pulling on retrieval line 502 facilitates retrieval (e.g., de-anchoring) of the patch anchor. Because retrieval feature 241a is disposed at the heel of the patch anchor, pulling on retrieval line 502 can (i) reorient the patch anchor axially (e.g., in alignment with the vector along which it was advanced, such as in alignment with a hole in the leaflet through which it was passed), and (ii) withdraw the patch anchor proximally. System 100 (e.g., delivery tool 400 thereof) can be modified to accommodate retrieval line 502, and to facilitate use thereof.

Reference is made to FIGS. 11A-B, which are schematic illustrations of a patch anchor 240b, in accordance with some implementations. Patch anchor 240b is a toggle anchor, and can have features of other patch anchors described herein. For example, patch anchor 240b can define at least one eyelet 244b via which cord 242 is attached to the patch anchor. Similarly, patch anchor 240b can have a sharpened tip. Patch anchor 240b has a retrieval feature 241b via which retrieval line 502 is connected to the patch anchor. Retrieval feature 241b includes a retrieval eyelet 246 and a notch 247, both cut in the circumferential wall of the toggle, which can be substantively tubular. Notch 247 is disposed at the heel of patch anchor 240b, and is defined by one or more parts of the circumferential wall of the toggle being removed. Retrieval eyelet 246 is disposed close to notch 247, and can be disposed on the same side as the notch. In the example shown, retrieval eyelet 246 is disposed just distally from notch 247 in the same circumferential position as the notch.

Retrieval line 502 is connected to patch anchor 240b by extending axially, at the heel of the patch anchor, colinearly into the lumen of the toggle (which can be substantially tubular), exiting the lateral wall of the toggle via retrieval eyelet 246, and looping back via notch 247 to connect with itself at reference numeral 504, which may represent a knot.

For applications in which 504 represents a knot, it is to be noted that the arrangement shown in FIGS. 11A-B results in knot 504 being situated within the width of patch anchor 240b, e.g., is tucked inside the patch anchor. This can advantageously reduce a likelihood of the knot obstructing retrieval of the toggle anchor through leaflet 10.

It is to be noted that the arrangement shown in FIGS. 11A-B results in retrieval line 502 exiting from patch anchor 240b colinearly with the patch anchor. That is, at the heel of the patch anchor, retrieval line 502 is aligned with the long axis of the patch anchor. Furthermore, retrieval line 502 extends from the very heel of patch anchor 240b, e.g., the exit point of the retrieval line from the patch anchor is the part of the patch anchor furthest from the tip of the anchor. Thus, if retrieval line 502 is pulled, it will naturally align patch anchor 240b with the vector of the pulling, thereby facilitating retrieval of the patch anchor through the hole in the leaflet through which the patch anchor was previously introduced (and through which the retrieval line extends), e.g., without the heel of the patch anchor catching on the edge of the hole in the leaflet.

Reference is made to FIG. 12, which is a schematic illustration of at least part of an implant 150a, in accordance with some implementations. Similarly, to 150, implant 150a comprises an upstream assembly 200a that comprises a patch 210a that can comprise a frame 230a. Also similarly to implant 150, implant 150a comprises a tether 160a that tethers upstream assembly 200a to a downstream assembly (not shown). The downstream assembly of implant 150a can be as described for other downstream assemblies herein, mutatis mutandis. For implant 150a, a single sheet 220a is shaped (e.g., cut and/or folded) to serve both as the sheet of patch 210a (analogous to sheet 220) and as tether 160a. That is, sheet 220a is shaped into a patch portion (e.g., a patch-shaped portion) and a tether portion (e.g., a tether-shaped portion). In the example shown, the patch portion is trapezoidal, and the tether portion is ribbon-shaped. In the example shown, the patch portion has tabs that are folded to cover parts of the frame. In the example shown, the tether portion is cut to approximately three times the width as tether 160a, and lateral parts of the tether portion are folded over a medial part of the tether portion to form the tether (e.g., on opposite sides of the medial part, thereby forming an s-shaped cross-section) such that the tether is approximately three times as thick as sheet 220a. However, other formations of the tether portion are possible, including the tether portion simply being cut into the tether shape.

Reference is made to FIG. 13, which is a schematic illustration of at least part of an implant 150b, in accordance with some implementations. Similarly to 150, implant 150b comprises an upstream assembly 200b that comprises a patch 210b that comprises a flexible sheet 220b and can comprise a frame (not shown). Also similarly to implant 150, implant 150b comprises a tether 16ob that tethers upstream assembly 200b to a downstream assembly (not shown). The downstream assembly of implant 150b can be as described for other downstream assemblies herein, mutatis mutandis. Implant 150 is shown with tether 160 attached at a single medial position on lip 211. In implant 150b, tether 16ob is attached at two lateral positions on lip 211b of patch 210b, e.g., via two lateral lines 162 that diverge laterally away from each other and from an upstream end of tether 160b. In some implementations, and as shown, these lateral attachments are in addition to the medial attachment, which can be via a medial line 164. This arrangement can be alternatively viewed as tether 16ob branching into two or three branches that are attached to lip 211b. In some implementations, lines 162 and 164 are fixedly attached to tether 160b and to each other, e.g., at a node 166.

FIG. 13 shows implant 150b in the absence of the anatomy, but behaving as though anchors 240 are attached to leaflet 10 and tension on tether 16ob is oscillating between low (left image) and high (right image), e.g., as may occur during the cardiac cycle, with the left frame representing the implant during ventricular diastole, and the right frame representing the implant during ventricular systole. As tension on tether 160b increases (from left image to right image), the lateral attachments urge lip 211b, and in some implementations most or all of patch 210b, to curve (e.g., flex) medially (e.g., about root-to-tip axis ax3) to become convex on the upstream surface of the patch. Thus, the surface of patch 210b presented to the opposing leaflet is convex, and the lateral edges of the patch are held away from the opposing leaflet. This can advantageously enhance coaptation and/or reduce rubbing of the lateral edges of the patch against the opposing leaflet. For applications in which patch 210b is resilient (e.g., comprises a resilient frame such as one those described herein), the curving (e.g., flexing) of the patch can also serve as a force-dampener or shock-absorber, advantageously softening the impact experienced by the patch (and the point at which it is anchored to leaflet 10) as the end of tether 16ob is reached during each ventricular systole.

For applications in which medial attachment (e.g., medial line 164) is also included, the medial attachment can serve to limit the extent by which patch 210b curves in response to tension on tether 160b. In the example shown, the length of medial line 164 is such that some slack remains even when no slack remains in lateral lines 162 (center image). This allows tension in lateral lines 162 to curve the patch as the slack in medial line 164 is taken up. However, once no slack remains in medial line 164, additional tension on tether 16ob does not result in additional curving of the patch. This limiting of the extent by which patch 210b curves can advantageously prevent the patch from curving to such an extent that its lateral edges are held away from coapting with the opposing leaflet.

Reference is now made to FIGS. 14, and 15A-B, which are schematic illustrations of an implant 150c and a technique for use therewith, in accordance with some implementations. FIGS. 15A-B show implant 150c being implanted using tool 400, but it is to be understood that implant 150c can optionally be implanted using another delivery tool such as, but not limited to, the variants of delivery tool 400 described herein.

Described hereinabove (e.g., with reference to FIGS. 5A-B) is a patch-anchor tightening functionality of upstream assembly 200 of implant 150. In contrast, implant 150c comprises an upstream assembly 200c whose patch anchors become tightened, but not by the upstream assembly (or the implant) itself. In the example shown, the patch anchors of upstream assembly 200c are patch anchors 240, described hereinabove. However, other patch anchors, including but not limited to other toggle anchors, can be used instead, mutatis mutandis. For example, whereas the patch-anchor tightening functionality of upstream assembly 200 can be provided by the upstream assembly (e.g., a spring thereof) biasing the patch anchor toward the patch (e.g., by pulling on a cord via which the patch anchor is connected to the patch), upstream assembly 200c does not bias the anchor toward its patch 210c (e.g., does not comprise such a spring). Instead, the anchor tightening is achieved by pulling via the delivery tool.

FIG. 15a shows anchors 240 having been driven through leaflet 10, and clasp 430 having been opened to release the leaflet. FIG. 15a can therefore be considered analogous to FIG. 2L. However, in contrast to FIG. 2L, in FIG. 15a anchors 240 remain untightened, e.g., they have not been drawn toward patch 210c and/or sandwiched leaflet 10 against the patch. As shown, patch 210c is typically slack at this point, e.g., because it does not comprise a spring that spreads the patch open.

FIG. 15b shows at least capsule 470 of tool 400, and optionally the entirety of distal portion 404 of the tool, being moved away from patch 210c (e.g., from upstream assembly 200c)—such as by being advanced downstream and/or further into ventricle 8. FIG. 15b can therefore be considered to be analogous to FIG. 2M. However, in contrast to FIG. 2M, as capsule 470 (with downstream assembly 300 therewithin) moves away from patch 210c, tether 160 is allowed to become tight (e.g., the tether is not let out) such that the tether pulls on patch 210c. Patch 210c, and its connection to tether 160, is such that this pulling pulls on cords 242c in a manner that tightens patch anchors 240, e.g., by drawing the patch anchors toward the patch. In the example shown, this is achieved by tether 160 being connected to a lip brace 231c of patch 210c (e.g., similarly to as described for implant 150), and cords 242c also being connected to the lip brace (e.g., also similarly to as described for implant 150). The pulling of cords 242c by the pulling of tether 160 pulls the cords through sites 214c at which the cords pass into and/or through the patch. Patch 210c comprises (e.g., at each site 214c) a one-way mechanism 216 (such as a ratchet, zip tie like mechanism, etc.) that allows cord 242c to pass therethrough only in the direction that tightens patch anchor 240, e.g., in the direction that pulls the patch anchor toward the patch. Thus, once patch anchor 240 is tightened it remain tightened, even when tether 160 is not under tension, and even when the portion of cords 242c on the opposite side of mechanism 216 from patch anchor 240 (e.g., the portion of the cord that is within and/or alongside patch 210c) is not under tension. Patch 210c can comprise patch-anchor support 236 (described hereinabove)—and, for such implementations, each mechanism 216 can be mounted on the patch-anchor support.

Because the tightening of patch anchors 240 of upstream assembly 200c does not require patch 210c to maintain a force on cords 242c, e.g., by being held in a spread-out state (e.g., the patch does not comprise a spring), the characteristics of the patch can be more tailorable to the leaflet-augmentation functionality of the patch, e.g., without trade-offs required for maintaining a force on the cords.

In some implementations, and as shown, patch 210c can comprise no frame components between root brace 232c and lip brace 231c. Furthermore, in some implementations patch 210 can comprise no frame components between root brace 232c and the lip of the patch, e.g., the patch may not comprise a root brace. This lack of frame components may advantageously enhance the flexibility of the patch.

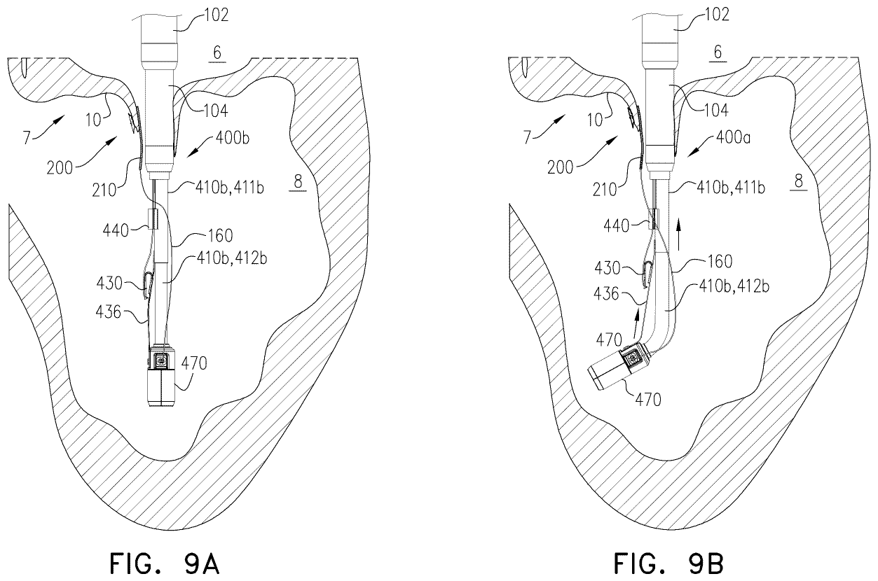

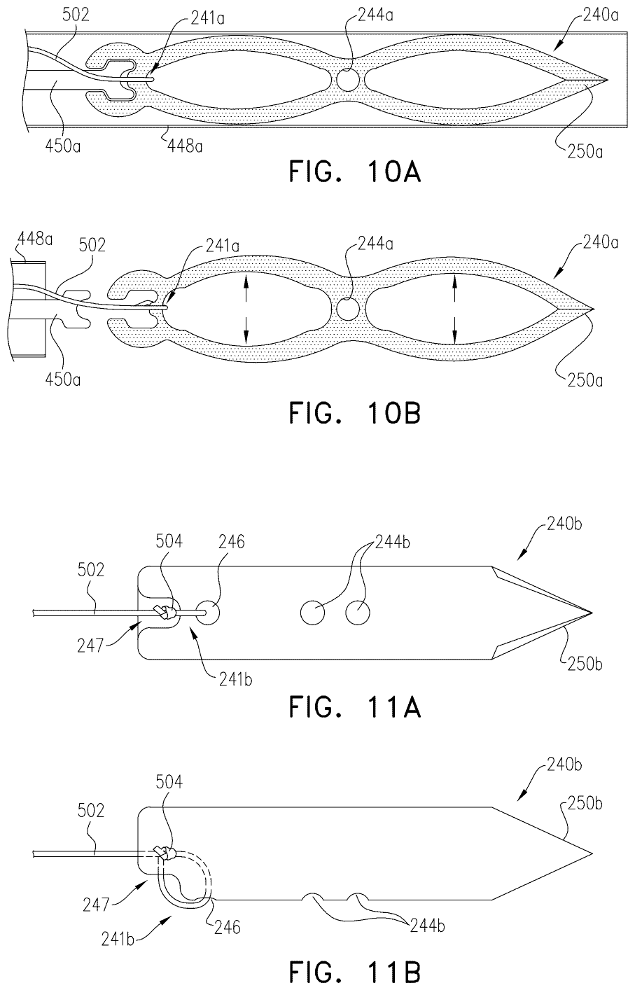

Reference is made to FIGS. 16, 17, 18A-B, and 19A-D, which are schematic illustrations of implants that are variants of implant 150 in which one or more ventricular anchors are connected to an upstream assembly via multiple (e.g., two) stretches of a tether, in accordance with some implementations.

FIG. 16 shows an implant 150d whose tether 160d extends, not just from downstream assembly 300d of the implant (e.g., winch 320 thereof) to upstream assembly 200d of the implant (e.g., patch 210d thereof), but also back to the downstream assembly (e.g., to housing 321d or anchor 310 thereof) to which the tether (e.g., the end of the tether) is fixed. This arrangement advantageously provides additional strength to the tethering due to the connection between the upstream and downstream assemblies being connected via two stretches of the tether.

Tether 16od can be connected to upstream assembly 200d (e.g., to patch 210d) slidably, e.g., by the tether (e.g., a bight of the tether) being threaded through an eyelet 219 of the upstream assembly. In the example shown, eyelet 219 is defined or provided by a ring that is flexibly connected to patch 210d. However, it is to be understood that the eyelet can be provided in a different manner, such as by being defined by the frame of patch 210d. For applications in which tether 160d is slidably connected to upstream assembly 200d, the arrangement of tether 160d (which can be considered a pulley arrangement) provides mechanical advantage for the shortening of the tether by winch 320. Thus, the torque necessary to be applied to winch 320 in order to shorten tether 16od can be advantageously reduced (e.g., approximately halved) compared to a similar implant that does not have such a pulley arrangement, e.g., implant 150. Furthermore, for each rotation of winch 320, the reduction of the effective length of tether 160d is less (e.g., approximately half) compared to a similar implant that does not have such a pulley arrangement. This can advantageously provide the operator with a greater degree of control (e.g., finer granularity) over the shortening tether 160d.

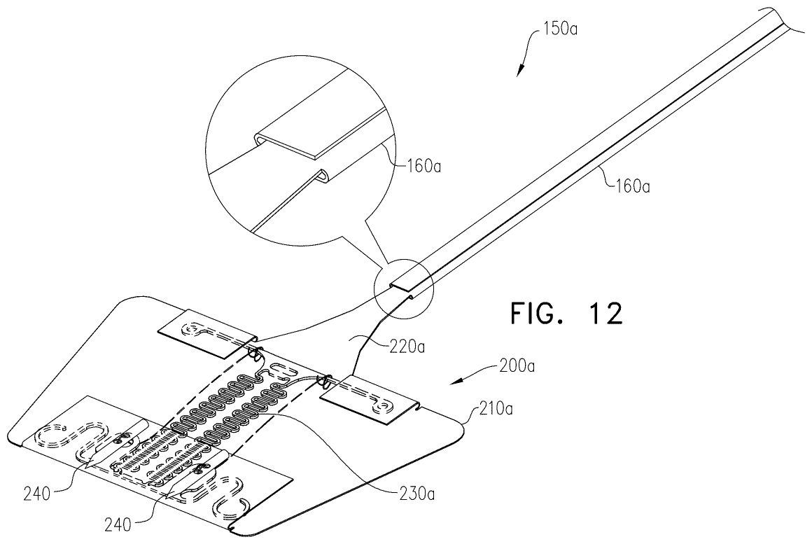

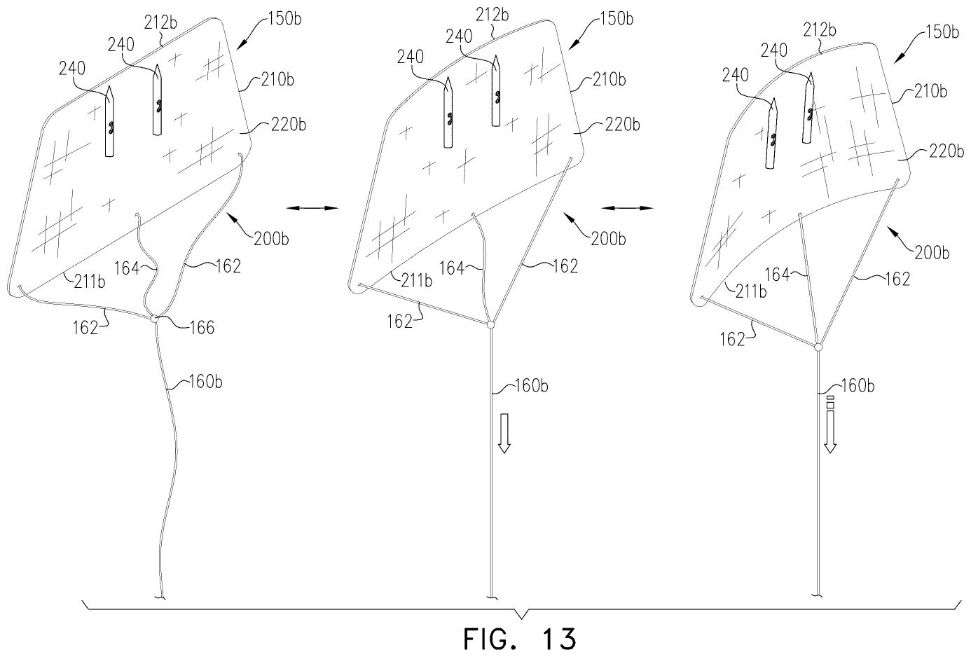

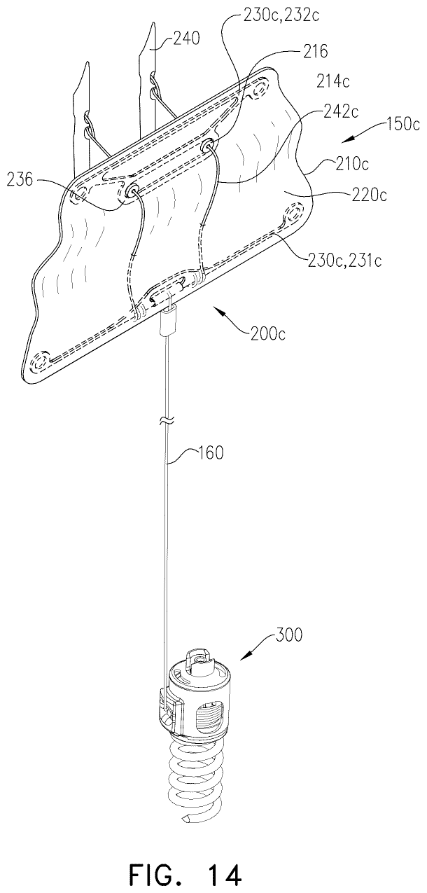

FIG. 17 shows an implant 150e, following its implantation. Like other downstream assemblies described herein, downstream assembly 300e of implant 150e comprises ventricular anchor 310. However, implant 150e further comprises a supplementary ventricular anchor 310e-which may or may not be considered to be a component of downstream assembly 300e. Similarly to the tether of implant 150d, tether 160e of implant 150e can be slidably connected to upstream assembly 200e, e.g., in a pulley arrangement. However, tether 160e (e.g., the end of tether) is fixed to supplementary ventricular anchor 310e. Thus, while implant 150e has advantages described for implant 150d, supplementary ventricular anchor 310e can further provide additional ventricular anchoring strength. Because the operator is able to select anchoring sites for each of the ventricular anchors (e.g., independently of each other), implant 150e can advantageously allow additional flexibility and/or control over the behavior of patch 210e, e.g., due to the vectors of the lengths of tether 160e between the anchors and the patch.

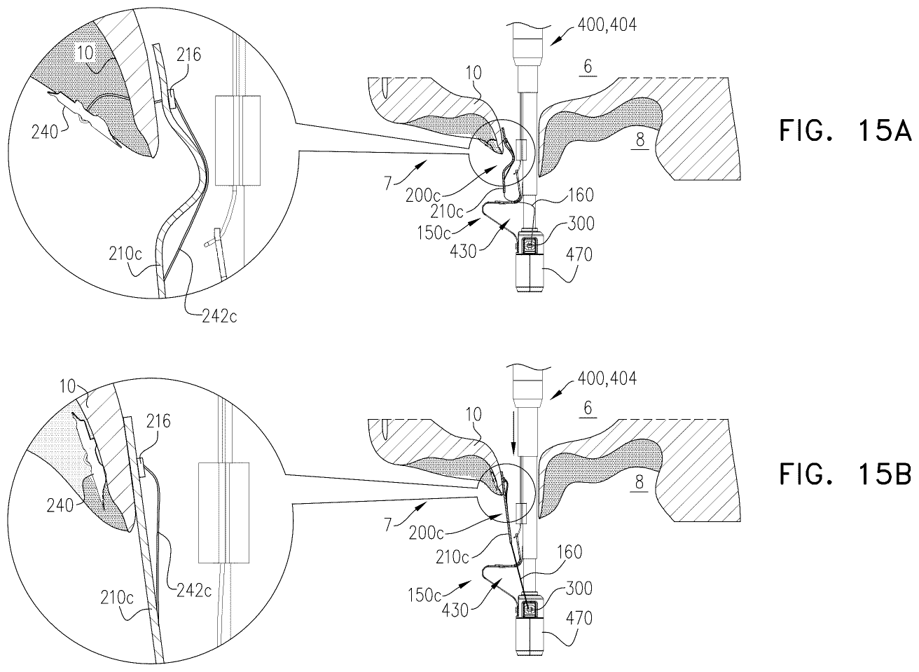

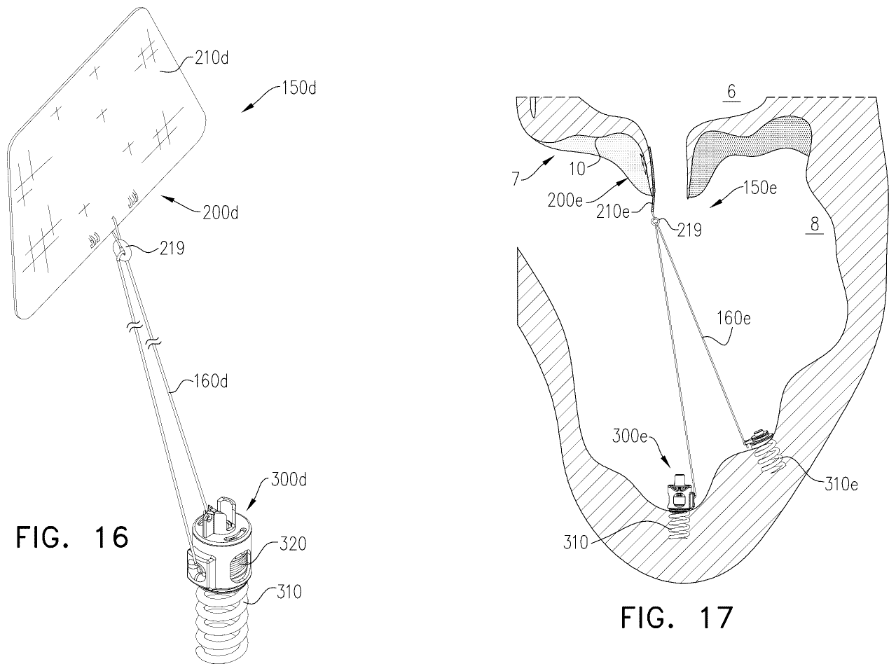

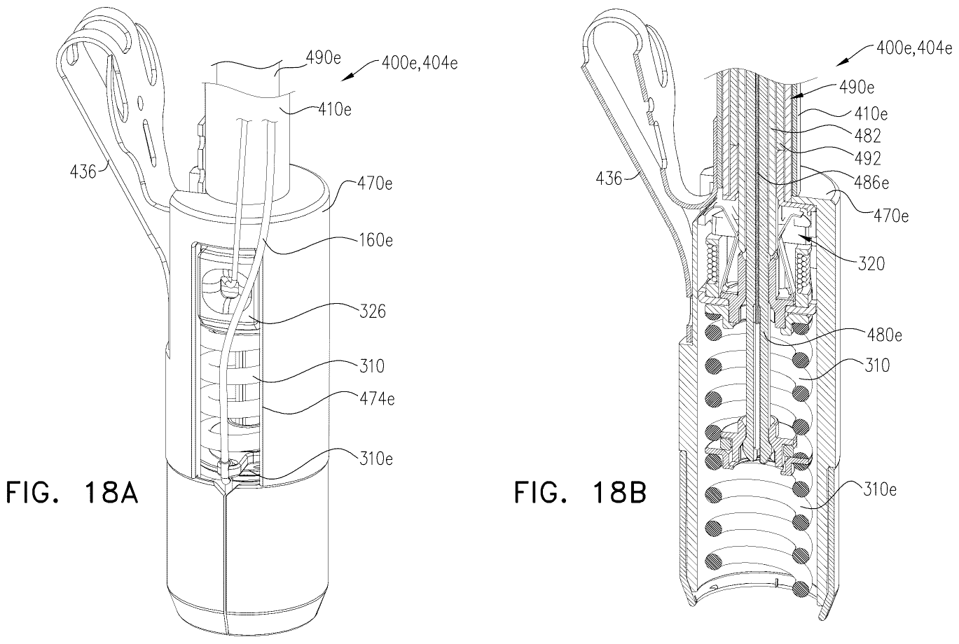

FIGS. 18A-B are schematic perspective and cross-sectional views, respectively, of a distal portion 404e of a delivery tool 400e, for use with implant 150e, in accordance with some implementations. Tool 400e can be as described for tool 400, except for the adaptations via which it accommodates implant 150e and the implantation thereof. As elsewhere herein, the suffix “e” has been added to certain reference numerals in order to indicate that certain components of tool 400e are variants of corresponding components of tool 400.

In some implementations, delivery tool 400e can comprise a capsule 470e at a distal end of the shaft 410e of the delivery tool. Capsule 470e can be configured to house anchor 310e and anchor 310 with winch 320 coupled thereto (e.g., downstream assembly 300e) during delivery and implantation of the implant. Capsule 470e can have an open distal end via which the anchors are deployable. For applications in which winch 320 (e.g., its housing) has lateral aperture 326 through which tether 160e passes, capsule 470e can define a lateral window 474e in order to allow the tether to reach the winch, e.g., as described hereinabove for capsule 470, mutatis mutandis.

Shaft 410e is tubular such that other components such as a driveshaft subassembly 490e (e.g., driveshafts of the driveshaft subassembly) can extend through it. Driveshaft subassembly 490e comprises at least one driveshaft, such as winch-control driveshaft 482 (described hereinabove) and an anchor-control driveshaft 480e. Anchor-control driveshaft 480e can be as described for anchor-control driveshaft 480 except that it is axially movable from engagement with anchor 310e (shown in FIG. 18B) to engagement with anchor 310, in order to anchor the two anchors sequentially. Engagement with, and disengagement from, each of the anchors can be facilitated by a lock-rod 486e.

Driveshaft subassembly 490e can further comprise a reference-force tube 492 through which the one or more driveshafts extend, and which is configured to provide a reference force to the downstream assembly while the driveshaft subassembly is applying torque to the winch.

FIGS. 19A-D show at least some steps in a technique for implanting implant 150e, in accordance with some implementations. For the sake of simplicity and clarity, various components of tool 400e, such as its clasp and its mount, have been omitted from FIGS. 19A-D. However, tool 400e can comprise such components, which can be similar or identical to those described for tool 400. For example, tool 400e can comprise clasp 430 and/or mount 440. Moreover, the components of tool 400e that interact with (e.g., implant) upstream assembly 200e can be as described for those of tool 400. Furthermore, the proximal portion of tool 400e can be as described for proximal portion 402 of tool 400, mutatis mutandis.

Anchor 310e is anchored to ventricular tissue, e.g., at a first site within ventricle 8 (FIG. 19A). This is achieved by driveshaft 482e applying an anchoring force to anchor 310e, e.g., applying torque to screw the tissue-engaging element of the anchor into the tissue. Driveshaft 482e is then disengaged from anchor 310e, and is retracted to engage anchor 310. At this point, capsule 470 can be retracted away from the ventricular tissue in preparation for its re-advancement to a second site in the ventricle (FIG. 19B). In this state, tether 160e extends from anchor 310e (anchored in ventricle 8), to upstream assembly 200e, and back to anchor 310 (within capsule 470e). Capsule 470e is then advanced to a second site within the ventricle, where anchor 310 will be anchored to ventricular tissue (FIG. 19C). Once anchor 310 has been anchored (again, by application of force by driveshaft 482e), and the length of tether 160 has been adjusted (e.g., by actuating winch 320), tool 400e can be removed, leaving implant 150e in place (FIG. 19D).

The adjustment of implant 150e (e.g., the length of tether 160e thereof) can be similar to as described for implant 150, mutatis mutandis. For example, following anchoring of anchor 310, shaft 410e can be withdrawn, leaving driveshaft assembly 490e, exposed, extending to winch 320. In this state, winch-control driveshaft 482 can be used to apply torque to the winch until a desired behavior of implant 150 (e.g., of patch 210e thereof) has been achieved. At that point, driveshaft subassembly 490e can be disengaged and withdrawn.

Reference is now made to FIGS. 20A-E, which are schematic illustrations of a distal portion 404f of a delivery tool 400f for implant 150 (or variants thereof), in accordance with some implementations. Delivery tool 400f can be generally as described for delivery tool 400, mutatis mutandis, except for the structure and function of its clasp (or at least the upstream support thereof), its mount, and its drivers for the patch anchors of the implant.

Tool 400f comprises a shaft 410f, a clasp 430f mounted on the shaft, and a mount 440f disposed alongside the shaft. At least an upstream support 432f of the clasp can be controllable by clasp controller (at a proximal region of tool 400f; not shown) that is operatively coupled to clasp 430f. This operative coupling can be provided by a wire 130f that is coupled to upstream support 432f. In the example show, a single wire 130f is used, but multiple wires 130f can be used. Pulling on wire 130f (e.g., by operating the clasp controller) transitions clasp 430f between its open and grasping states by moving (e.g., deflecting) upstream support 432f with respect to a downstream support 434f of clasp 430f, and typically also with respect to shaft 410f. Wire 130f can be attached to upstream support 432f. The vector along which wire 130f slides can be controlled by a bearing 132 (e.g., a loop) around which the wire slides. Similarly, to clasp 430 and wire 130 of tool 400, clasp 430f can be biased (e.g., spring-loaded) to close (e.g., to move toward its grasping state), such that releasing tension on wire 130 allows the clasp to responsively close.

Unlike in tool 400, wire 130f may not serve as a guide for mount 440f (e.g., as a rail along/over which the mount is advanced). Rather, in tool 400f, such guidance is provided by a mechanical linkage between mount 440f and shaft 410f. For the sake of simplicity and clarity, other than patch anchors 240f, FIGS. 20A-E do not show the implant itself. Similarly, FIGS. 20A-E do not show the anatomy (e.g., leaflet 10). Concurrent reference to FIGS. 2G-K and 6A-C may be useful for context and comparison.

The mechanical linkage includes a beam 441 (which can resemble a panel) that is pivotably attached at one end to shaft 410f and pivotably attached at the other end to mount 440f. The linkage links distalward movement of mount 440f with lateral translation and/or deflection of the mount, in a manner that guides the mount toward its primed state, e.g., into a predetermined position suitable for anchoring of the patch of the implant. For example, the distance by which mount 440f moves laterally when transitioning from its retracted state (FIG. 20A) to its primed state (FIG. 20B) can be a product of the effective length of beam 441 (e.g., its length between its points of attachment to shaft 410f and mount 440f) and/or the angle by which the beam deflects during the transition.

FIG. 20A shows tool 400f with clasp 430 closed, as though it were already grasping leaflet 10, and with mount 440f in its retracted state. Thus, FIG. 20A can be considered to generally correspond to FIG. 2H, mutatis mutandis. FIG. 20B shows mount 440f having moved into its primed state, and can therefore be considered to generally correspond to FIG. 2J, mutatis mutandis. Arrow a1 indicates deflection of beam 441, and arrow 12 indicates movement (e.g., distal advancement and lateral translation and deflection) of mount 440f. In some implementations, and as shown, beam 441 can have a notch or aperture through which wire 130f extends, in order to allow the functionality described herein without interference between these components.

In some implementations, beam 441 is biased (e.g., spring-loaded) to move mount 440f into its primed position. For example, and as shown, beam 441 can be a cantilever spring. For such implementations, beam 441 can be initially constrained (FIG. 20A), and the constraint is released in order to allow the beam to move mount 440f into its primed position (FIG. 20B). For some such implementations, the constraint is provided by a discrete dedicated component (e.g., similar to wire 130f). However, in the example shown, the constraint is provided by one or more drivers 450f, which are also configured to drive patch anchors 240f through leaflet 10. This dual-functionality is facilitated by cooperating shapes and dimensions of drivers 450f and mount 440f (e.g., channels 448f of the mount, within which patch anchors 240f are disposed).

Each driver 450f comprises a drive head 452f and a rod (e.g., a pusher rod) 454f extending proximally from the drive head, e.g., to the proximal portion of tool 400, such as to a driver controller (e.g., analogous to driver controller 112 described hereinabove) thereby operatively coupling the driver controller to the driver. Similarly to drivers 450 of tool 400, drivers 450f are configured to anchor patch 210 to leaflet 10 (e.g., to the portion of the leaflet grasped by clasp 430f) using patch anchors 240f, e.g., by driving the patch anchors through the leaflet. Similarly to as described for implant 150 and tool 400, during delivery, anchors 240f are held within channels 448f of mount 440f, e.g., such that the cords of the patch anchors extend laterally out of the channels to secure the patch to the mount. However, as noted above, drivers 450f have dual functionality-they are also configured to provide constraint that inhibits mount 440f from moving toward its primed position and/or to pull the mount out of its primed position.

In order to drive (e.g., push) patch anchors 240f out of channels 448f, drive head 452f is shaped and dimensioned to be slidable distally through the channels. For example, and as shown, each drive head 452f can fit snugly within a respective channel 448f. However, mount 440f comprises or is shaped to define an obstruction that obstructs drive head 452 from exiting the mount proximally and/or from disengaging from the mount. For example, and as shown, this obstruction is a backplate 449. As shown, backplate 449 can, for each driver 450f, define an opening through which pusher rod 454f extends, but that is shaped and/or dimensioned to obstruct passage of drive head 452 therethrough. For example, drive head 452f can simply be wider than pusher rod 454f. Therefore, pulling or restraining driver 450 proximally pulls or constrains mount 440f in its retracted position by pulling on the obstruction, e.g., on backplate 449 (FIG. 20A). Sufficiently releasing driver 450f (e.g., tension on pusher rod 454f) allows beam 441 to pull mount 440f toward its primed state (FIG. 20B). However, this transition can be achieved without any relative movement between driver 450f and mount 440f. For example, in FIG. 20B drive head 452f remain in contact with backplate 449.

From the primed position, distal movement of drivers 450f (e.g., pushing of pusher rods 454f) moves drive heads 452f away from backplate 449 and distally through channels 448f, thereby driving patch anchors 240f out of the channels and through the leaflet held in clasp 430f (FIG. 20C). As described hereinabove, this anchors the patch to the leaflet. Subsequent retraction of drivers 450 (e.g., by pulling on pusher rods 454f) brings drive heads 452f back into contact with backplate 449 (FIG. 20D), and further retraction pulls mount 440f back toward its retracted state, e.g., by the drive heads pulling on the backplate (FIG. 20E). Subsequently, leaflet 10 can be released from clasp 430f by opening the clasp by pulling wire 130f to deflect upstream support 432f proximally. This leaflet-release step is not shown, but is analogous to that shown in FIG. 2L, mutatis mutandis.

In some implementations, the delivery tool can include a spring that biases the mount to move into its primed position, without necessarily including a beam that provides such a mechanical linkage.

It is hypothesized that, in some implementations, such separation of the roles of clasp control and mount advancement can advantageously improve predictability and/or reliability of the delivery tool. For example, because advancement of mount 440f does not require interaction between the mount and wire 130f, advancement of the mount can be less likely to inadvertently affect grasping of leaflet 10.

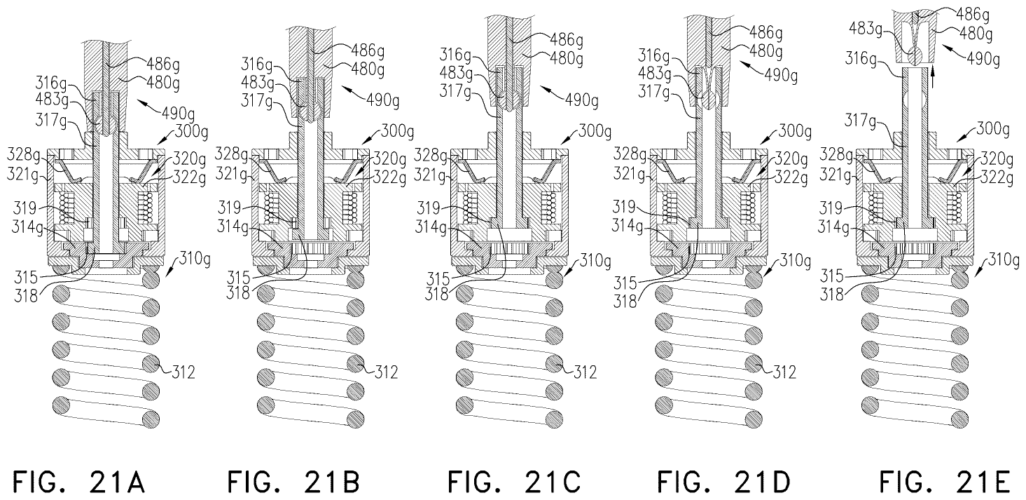

Reference is now made to FIGS. 21A-E, which are schematic illustrations of some components of a driveshaft subassembly 490g and a downstream assembly 300g, in accordance with some implementations. Driveshaft subassembly 490g can serve as the driveshaft subassembly of any of the delivery systems described herein, and downstream assembly 300g can serve as the downstream subassembly of the implants described herein.

Downstream assembly 300g comprises a winch anchor 310g that comprises a tissue-engaging element such as tissue-engaging element 312, described hereinabove. Downstream assembly 300g further comprises a winch 320g, coupled to the winch anchor, and comprising a spool 322g. Downstream assembly 300g also comprises a driver interface 316g. As shown, spool 322g can be mounted such that it and/or its axis of rotation is colinear with the anchor axis of anchor 310g. However, other spool orientations are possible.

In driveshaft subassembly 490g, the separate anchor-control driveshaft and winch-control driveshaft of driveshaft subassembly 490 have been replaced with a unitary downstream-assembly-control driveshaft 480g. It is hypothesized that, in some implementations, replacing two coaxial driveshafts with a single driveshaft can enable driveshaft subassembly 490g to be slimmer and/or more flexible than driveshaft subassembly 490, thereby advantageously being even less likely to create significant hemodynamic artifacts during adjustment of the effective length of tether 160.

Driveshaft 480g can comprise or define, at a distal end of the driveshaft, a drive head 483g that comprises one or more (e.g., two) spurs that are locked to downstream assembly 300g by a lock-rod 486g, e.g., by the lock-rod maintaining the spurs in a laterally-displaced state. That is, lock-rod 486g locks the engagement between drive head 483g and driver interface 316g. Via engagement between drive head 483g and interface 316g, driveshaft subassembly 490g (e.g., driveshaft 480g thereof) facilitates both (i) application of an anchoring force (e.g., torque) to winch anchor 310g, and (ii) actuation of winch 320, independently of each other.

Downstream assembly 300g is transitionable between an anchoring state and a winching state. In the anchoring state, force (e.g., torque) applied by driveshaft 480g to interface 316 is transferred to winch anchor 310g in a manner that facilitates driving of tissue-engaging element into the tissue. In the winching state, force (e.g., torque) applied by driveshaft 480g to interface 316 is transferred to winch 320 in a manner that actuates the winch to rotate spool 322g. Transitioning between the states can be controlled from the extracorporeal control portion of the delivery tool, e.g., a controller thereof. Downstream assembly comprises an axle 317g that typically defines and/or is fixedly attached to interface 316. Transitioning of downstream assembly 300g between its anchoring state and its winching state is achieved by shifting a position (e.g., an axial position) of axle 317g with respect to other components of the downstream assembly.

FIG. 21A shows downstream assembly 300g in its anchoring state, in which axle 317g is engaged with an anchor head 314g of anchor 310g, e.g., with axle head 318 disposed within a complementarily-shaped socket 315 defined by the anchor head (e.g., a hex drive). In this state, rotation of driveshaft 480g rotates axle 317g, which rotates tissue-engaging element 312, screwing it into the tissue. Axially moving axle 317g (e.g., proximally), via an axial (e.g., pulling) force applied by driveshaft 480g, transitions downstream assembly 300g into its winching state (FIG. 21C) in which axle 317g is engaged with spool 322g of winch 320g, e.g., with axle head 318 disposed within a complementarily-shaped socket 319 defined by spool 322g (e.g., a hex drive).

In some implementations, and as shown, downstream assembly 300g is also provided with a neutral state (FIG. 21B) via which the downstream assembly transitions from its anchoring state into its winching state. For example, in the neutral state, axle head 318 can be disposed neither in socket 315 nor in socket 319, e.g., the axle head can be disposed in a space between these sockets, in which the axle head can rotate freely without engaging the anchor head or the spool. In some implementations this can facilitate the transition from the anchoring state to the winching state. For example, if, upon completion of anchoring, socket 315 (and therefore axle head 318) happen not to be rotationally aligned with socket 319, the neutral state (e.g., the space between the sockets) can allow rotation of axle 317g in order to rotationally align the axle head with socket 319 in order for the axle head to enter the socket.

Once anchoring and winching are complete, driveshaft 480g is disengaged from interface 316g, e.g., by retracting lock-rod 486g and thereby allowing the spurs of drive head 483g to responsively (e.g., automatically) deflect medially (FIG. 21D). At this point, driveshaft subassembly 490g can be completely disengaged and retracted from downstream assembly 300g (FIG. 21E). It is to be noted that axle 317g, which is a component of downstream assembly 300g, remains in place. FIG. 21E shows axle 317g remaining in the position shown in FIGS. 21C-D, but it is to be understood that following disconnection the axle can be free to alternatively return to the neutral position shown in FIG. 21B or to the position shown in FIG. 21A.

In some implementations, winch 320g comprises one or more spring-loaded detents 328g that. in at least one state of downstream assembly 300g, press against spool 322g in a manner that rotationally locks the spool with respect to housing 321g of the winch. For example, detents 328g can protrude into recesses defined in a surface of spool 322g, and/or can press teeth in a surface of the spool into engagement with complementary teeth in a surface of the housing. For such implementations, transitioning downstream assembly 300g into its winching state can include overcoming this locking, such as by axle 317g pulling spool 322g axially with respect to housing 321g. However, for the sake of clarity this is not shown.

Although not shown in FIGS. 21A-E, driveshaft subassembly can comprise a reference-force tube such as reference-force tube 492, described hereinabove, mutatis mutandis.

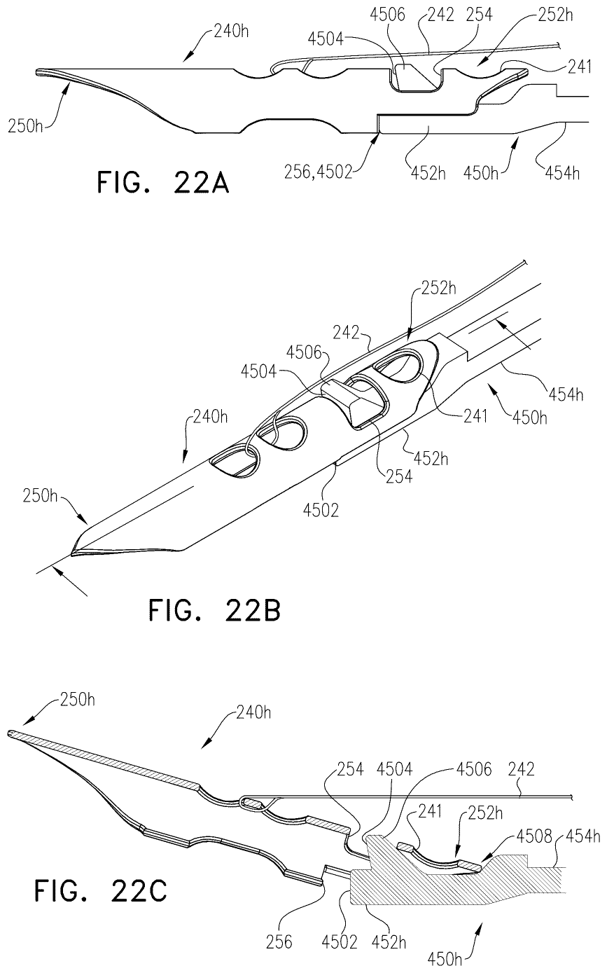

Reference is made to FIGS. 22A-C, 23A-D, 24A-C, 25A-E, 26A-E, 27A-D, 28A-B, 29A-D, 30A-B, 31A-D, 32A-B, 33A-D, 34A-B, and 35A-C, which are schematic illustrations of various patch anchors and anchor drivers, in accordance with some implementations. Each of these patch anchors can be the same as, similar to, and/or be considered to be a variant of patch anchor 240, described hereinabove. Thus, each of these patch anchors can be used to anchor a leaflet-augmentation patch (or another implantable element) to a leaflet of a valve (e.g., as described hereinabove, mutatis mutandis), can be used as a component of an implant such as implant 150 (or a variant thereof), mutatis mutandis, and/or can be used in combination with a delivery tool such as delivery tool 400 (or a variant thereof), mutatis mutandis. However, for some implementations, the patch anchors described with reference to FIGS. 22A-35B can be considered more generally to be toggle anchors, and/or can be used in other contexts.

FIGS. 22A-C, 23A-D, and 24A-C schematically illustrate patch anchors whose heel has geometry that compliments that of a corresponding driver such that the connection between the heel and the driver (e.g., a drive head thereof) (i) preferentially allows deflection rather than lateral translation of the toggle anchor with respect to the driver, and (ii) allows the heel to disconnect from the driver upon the toggle anchor reaching a predetermined angle with respect to the driver. FIGS. 22A-C show a patch anchor 240h and its corresponding driver 450h, FIGS. 23A-D show a patch anchor 240i and its corresponding driver 450i, and FIGS. 24A-C show a patch anchor 240j and its corresponding driver 450j.

Each of drivers 450h and 450i is a component of a respective delivery tool (e.g., delivery tool 400 or a variant thereof), and can be used to push the patch anchor out of a channel defined by the delivery tool (e.g., tip-first, distally out of and away from the channel), e.g., as described elsewhere hereinabove.

Patch anchor 240h has a tip 250h (which can have a sharp point, e.g., as shown) and a heel 252h, and defines an anchor axis between the tip and the heel. Driver 450h has a drive head 452h, and a rod 454h extending proximally from the drive head. The connection between heel 252h and drive head 452h inhibits lateral translation (e.g., slippage) of patch anchor 240h with respect to driver 450h, such as (and in some implementations, especially) while the driver is pushing the patch anchor into/through tissue (e.g., tissue of leaflet 10). This may advantageously reduce a likelihood of premature disengagement of the patch anchor from the driver (e.g., slipping of the drive head off of the heel of the anchor), e.g., prior to the entire patch anchor having been successfully advanced through the leaflet and out of the opposite side of the tissue. FIGS. 22A and 22B are a side view and a perspective view, respectively, of patch anchor 240h engaged substantially colinearly with driver 450h in this manner.

However, the engagement between the heel and the drive head does allow deflection of the anchor with respect to the driver. That is, the engagement between heel 252h and drive head 452h may preferentially allow deflection rather than lateral translation of the toggle anchor with respect to the driver. Such deflection may, for example, be caused by tension on cord 242 (e.g., described hereinabove). Thus, deflection may occur as toggle anchor 240h reaches a given distance from patch 210, and/or as the spring of the patch pulls on the cord. FIG. 22C shows such deflection. Typically, the engagement between the heel and the drive head furthermore allows (or even causes) patch anchor 240h to disconnect from driver 450h upon reaching a predetermined angle with respect to the driver. In the example shown, it may be understood from FIG. 22C that any further deflection of anchor 240h with respect to driver 450h can completely disconnect the anchor from the driver.

For some implementations, and as shown, the geometry that facilitates the above-described behavior includes two distally-facing faces 4502 and 4504 of drive head 452h, and a lateral opening 254 in patch anchor 240h at heel 252h. As shown, face 4504 can be defined by a shoulder 4506 of drive head 452h. The geometry can also include heel 252h having a substantially open side opposite lateral opening 254. Although face 4504 faces in substantially the same direction as face 4502, face 4504 is disposed proximally from face 4502. For some implementations, and as shown, face 4502 can be the most distal part of drive head 450h. For some implementations, although faces 4502 and 4504 face distally, they may not be exactly parallel. For example, and as shown, face 4502 can be substantially orthogonal to the axis of driver 450h while face 4504 can be slightly oblique to the axis, e.g., facing slightly toward the axis (e.g., lying at 75-85 degrees to the axis). This oblique angle may facilitate stabilization (e.g., “caging”) of anchor 240h during while it is being pushed by driver 450h.

Shoulder 4506 typically protrudes through lateral opening 254. Although opening 254 is shown adjacent to an eyelet of retrieval feature 241, for some implementations, a single eyelet serves both as both the retrieval feature and lateral opening 254.

Driver 450h is configured to push toggle anchor 240h tip-first through the leaflet by (i) face 4504 pushing distally on the toggle anchor at lateral opening 254 (e.g., against a distal rim of the opening), and/or (ii) face 4502 pushing distally on the toggle anchor substantially opposite the lateral opening, e.g., at a distal limit 256 of the substantially open side of heel 252h. Thus, although for some implementations anchor 240h and driver 450h can be shaped such that in a delivery state (e.g., while within channel 448 and/or while driver 450h and anchor 240h are held colinearly) face 4504 is slightly spaced away from contact with the anchor, e.g., as shown, for other implementations the anchor and the driver can be shaped such that the face is in contact with the anchor while in such a delivery state.

The deflection-mediated disconnection of toggle anchor 240h from drive head 452h occurs by the toggle anchor deflecting about a point 4508 on driver 450h that is proximal from face 4504 (e.g., proximal from shoulder 4506) such that lateral opening 254 moves laterally away from shoulder 4506.

Patch anchor 240i and its driver 450i have similar anti-slipping and deflection-based disconnection behaviors as patch anchor 240h and driver 450h. However, in the case of patch anchor 240i and driver 450i, the geometry is provided at least in part by (i) a knob 4510 defined by drive head 452i of driver 450i, and (ii) appendages 254i defined by heel 252i of patch anchor 240i. As shown, knob 4510 is connected via a neck 4512 to rod 454i of driver 450i, e.g., the neck being narrower than both the knob and the rod.

Patch anchor 240i has a tip 250i that is shown as having a sharp point. However, patch anchor 240i, or a variant thereof, can optionally have a blunt point, e.g., can be delivered through a needle.

During delivery, appendages 254i extend proximally beyond knob 4510 and, proximally from the knob, medially toward each other and toward the neck (FIG. 23B). Thus, for some implementations, appendages 254i can be considered to define (e.g., to delimit) a socket within which knob 4510 is disposed. For some implementations, the neck can be squeezed between the ends of appendages 254i. In this arrangement, appendages 254i inhibit proximal retraction of driver 450i from patch anchor 240i, and also inhibit lateral translation of the driver with respect to the patch anchor—at least on one lateral vector.

Deflection of patch anchor 240i with respect to driver 450i (e.g., due to tension on cord 242) urges knob 4510 between the appendages such that the appendages deflect (e.g., transiently) laterally away from each other and from the neck, allowing the knob to slip out and the patch anchor to disconnect (FIGS. 23C-D).

Although patch anchor 240i is shown as being generally similar to patch anchor 240a (described with reference to FIGS. 10A-B), such as having a cellular structure that can automatically expand, the anti-slipping and deflection-based disconnection features of patch anchor 240i and driver 450i can be applied, mutatis mutandis, to other patch anchors and/or other toggle anchors including those without a cellular structure or those that do not automatically expand.

Patch anchor 240j and its driver 450j also have similar anti-slipping and deflection-based disconnection behaviors. However, in the case of patch anchor 240j and driver 450j, the geometry is provided at least in part by (i) a socket 4514 (e.g., a recess) defined by drive head 452j of driver 450j, and (ii) a knob 258 (e.g., an appendage) defined by heel 252j of patch anchor 240j.

During delivery, knob 258 is disposed within socket 4514 in a manner that inhibits lateral translation (e.g., slipping) of patch anchor 240j from driver 450j (FIG. 24A). During anchoring (e.g., after a sufficient degree of driving of the anchor), deflection of patch anchor 240j with respect to driver 450j (e.g., due to tension on cord 242) presses a rim 4516 of socket 4514 against patch anchor 240j (e.g., against a neck or shoulder just distally from the knob) in a manner that levers knob 258 out of socket 4514, allowing the patch anchor to disconnect (FIGS. 24B-C). For some implementations, knob 258 and socket 4514 are substantially spherical and/or allow deflection in multiple planes. For some implementations, knob 258 and socket 4514 are substantially cylindrical and/or allow deflection in a limited number of planes, e.g., in only one plane.

It is to be noted that for anchors 240h, 240i, and 240j, disconnection from the anchor driver can include a lever-like movement.

Reference is now made to FIGS. 25A-E, which are schematic illustrations of a pressure-responsive needle 456 and application thereof, in accordance with some implementations. As noted hereinabove, although patch anchor 240 is shown as having a sharp point, for some implementations patch anchor 240 (or a variant thereof) is disposed within, and advanced out of, a needle that punctures the leaflet, e.g., tip 250 may not have a sharp point. Needle 456 is an example of such a needle.

FIGS. 25A-E show a delivery tool 400k that comprises needle 456, and that is for use with a patch anchor 240k that does not necessarily have a sharp point, e.g., is for use with an implant (e.g., an upstream subassembly of an implant) that comprises patch anchor 240k. Needle 456 is slidably coupled to a mount 440k of delivery tool 400k, and can be biased toward a retracted state in which a sharp tip of the needle is functionally obscured by the mount, e.g., is ensheathed inside a channel 448k of the mount. For example, delivery tool 400k can comprise a spring 441k (e.g., a compression spring, such as a helical compression spring, as shown), configured to provide this bias. Channel 448k can be as described hereinabove for channel 448, but shaped to house and retain needle 456 while allowing the movement of the needle described hereinbelow. Like channel 448, channel 448k can be laterally open in order to accommodate passage of cord 242 from patch anchor 240k to patch 210. Needle 456 can have a lateral slit for the same purpose.

For applications in which delivery tool 400k comprises spring 441k, channel 448k can also house the spring, e.g., the spring can circumscribe at least part of needle 456 within the channel, such as shown.

The distal portion of delivery tool 400k is transluminally advanceable to the heart while mount 440k is in its retracted position, with patch 210 mounted on the mount and patch anchor 240k disposed within channel 448k. This is at least in part illustrated by the main image of FIG. 25A, although in FIG. 25A shows a later state (e.g., corresponding to that shown in FIG. 2I, mutatis mutandis) in which clasp 430 of tool 400k has been transitioned into its grasping state and patch 210 has been released (e.g., unwrapped) from the shaft of the delivery tool. FIGS. 25B-E show subsequent steps in the use of delivery tool 400k, which can be considered to generally correspond to those described with reference to FIGS. 2J-K, mutatis mutandis. Although FIGS. 25B-E are cross-sections in which the upstream support and/or the downstream support of clasp 430 would be visible, for the sake of simplicity these components are not shown in these figures.

FIG. 25B shows mount 440k having been moved into its primed position, e.g., from its retracted position. However, whereas for tool 400 this is described as being achieved using a mount-control rod that can be fixed to the mount, for tool 400k this movement can be achieved by a mount-control rod 136k that has a dual function. Mount-control rod 136k is operatively coupled to mount 440k via needle 456, e.g., by being coupled to the needle. For example, from the retracted position, pushing of needle 456 by mount-control rod 136k pushes, via spring 441k, mount 440k toward the primed position. For example, the spring constant of spring 441k can be sufficiently large that the spring transfers the pushing force, applied by mount-control rod 136k on needle 456, to mount 440k while still retaining the needle in its retracted state. It will be understood that this is likewise facilitated by the moveability of mount 440k toward its primed position (e.g., its lack of resistance). Once mount 440k is in its primed position, resistance to further movement of the mount can occur, e.g., because the mount is abutting leaflet 10 and/or the upstream support of clasp 430. Thus, once in the primed position, further pushing, by mount-control rod 136k, of needle 456 overcomes the spring constant of spring 441k (e.g., strains the spring) and advances the needle out of channel 448k and through leaflet 10, e.g., piercing the leaflet (FIG. 25C).

Although the transition from FIG. 25B to FIG. 25C shows needle 456 moving distally with respect to patch anchor 240k (e.g., with the patch anchor and anchor driver 450k remaining stationary with respect to mount 440k) it is to be understood that, for some implementations, the needle and/or the anchor driver can move with respect to the mount during this transition, e.g., can remain stationary with respect to the needle.

Delivery tool 400k (e.g., anchor driver 450k thereof) then anchors patch 210 to leaflet 10 by advancing patch anchor 240k out of needle 456, on the opposite side of the leaflet from the patch (FIG. 25D). Subsequently, needle 456 is withdrawn from leaflet 10, e.g., by being retracted into channel 448k (FIG. 25E). For example, spring 441k can cause such retraction to occur automatically upon removal of the pushing force of mount-control rod 136k on needle 456. Driver 450k can be retracted before, during, or after the retraction of needle 456. For some implementations, subsequent retraction of mount 440k toward its retracted position is achieved by pulling on mount-control rod 136.

For simplicity, cord 242 has been omitted from FIGS. 25B-E.

Reference is now made to FIGS. 26A-E, 27A-D, 28A-B, 29A-D, 30A-B, and 31A-D, which are schematic illustrations of extendable members for use with patch anchor 240 (or a variant thereof), in accordance with some implementations. Each of these extendable members can be used, mutatis mutandis, as a component in system 100 (or a variant thereof), e.g., as a component of delivery tool 400 (or a variant thereof) or as a component of implant 150 (or a variant thereof). In each case, the extendable member, and/or the system of which it is a component, is configured such that, upon the driver of the delivery tool of the system pushing the tip of the patch anchor of the system against the tissue, the extendable member responsively slides axially with respect to the patch anchor (e.g., with respect to the body of the patch anchor). That is, in each case, the system is configured such that, upon the driver pushing the tip of the toggle anchor against the tissue, the extendable member automatically slides axially with respect to the patch anchor (e.g., with respect to the body of the patch anchor).

For some implementations, the responsive axial sliding is such that the extendable member extends from the body of the anchor. For some implementations, the responsive axial sliding is such that the extendable member retracts into the body of the anchor. For some implementations, the responsive axial sliding is such that the extendable member slides distally with respect to the body of the anchor. For some implementations, the responsive axial sliding is such that the extendable member slides proximally with respect to the body of the anchor. For some implementations, the extendable member comprises, or is in the form of, a needle (or awl). For some implementations, the extendable member comprises, or is in the form of, a post. For some implementations, the extendable member stabilizes the anchor with respect to the anchor driver. For some implementations, the extendable member facilitates piercing of the tissue (e.g., the leaflet) into (e.g., through) which the anchor is being driven.

Although the extendable members are described for use with patch anchors of a leaflet-augmentation patch of an implant, they can be used, mutatis mutandis, with other toggle anchors, e.g., with other implants that comprise a toggle anchor.

In each case, the delivery tool has an anchor driver that comprises a drive head (e.g., a variant of drive head 452) and a rod (e.g., a variant of rod 454) extending proximally from the drive head. For some implementations, the drive head is visibly distinct from the rod, such as by being a discrete component attached to the rod. For some implementations, the drive head is merely a surface (e.g., the distal end) of the drive rod. Irrespective of whether the drive head is visibly distinct from the rod, the drive head can be considered to be the part of the anchor driver via which the anchor drive applies the anchoring force to the anchor, e.g., the distal pushing force that pushes the anchor through the tissue.

FIGS. 26A-E show an extendable member in the form of a needle (or awl) 4520 that is a component of an anchor driver 450l, in accordance with some implementations. Anchor driver 450l is for use with a patch anchor 240l, and also comprises a spring 4522. Anchor driver comprises a rod 4541, and a drive head 4521 that can be coupled to the rod via spring 4522. Patch anchor 240l is tubular, and its tip may not have a sharp point. Rather, and similarly to needle 456, needle 4520 has a sharp point and provides the required tissue-piercing (e.g., leaflet-piercing) functionality. At rest, the sharp point of needle 4520 is functionally obscured by being disposed proximally from the tip of patch-anchor 240l (FIG. 26A). For example, the sharp point can be disposed within the lumen of patch anchor 240l, or needle 4520 can be disposed entirely proximally from the patch anchor. Spring 4522 (e.g., a helical compression spring, as shown) allows driver 450l to advance patch anchor 240l distally by transmitting the pushing force from rod 4541 to drive head 4521, e.g., the spring inhibits needle 4520 from advancing distally with respect to patch anchor.

FIG. 26A shows patch anchor 240l having been advanced into contact with leaflet 10, but not yet having been pressed against the tissue.

Upon driver 450l (e.g., drive head 452l thereof) pushing the tip of patch anchor 240l against leaflet 10, spring 4522 strains (e.g., compresses) in response to resistance from the tissue of the leaflet, allowing needle 4520 to advance distally such that its sharp point becomes exposed out of the tip of the patch anchor and pierces the leaflet (FIG. 26B). Further pushing by (e.g., advancement of) driver 450l advances patch anchor 240l into the hole pierced by needle 4520 (FIG. 26C). Once patch anchor 240l has advanced sufficiently far through the hole, resistance by leaflet 10 can reduce to the point at which patch anchor 240l can slip through the hole- and spring 4522 can responsively (e.g., abruptly) eject the patch anchor off of needle 4520 (FIG. 26D). At this point driver 450l can be withdrawn, leaving patch anchor 240l in place (FIG. 26E). Thus, needle 4520 has similar functionality to needle 456, but is disposed within/through the patch anchor rather than the patch anchor being disposed within/through the needle.

For some implementations, the presence of needle 4520 through patch anchor 240l during advancement of the patch anchor through the tissue may also advantageously stabilize the patch anchor on anchor driver 450l, e.g., preventing premature lateral slipping and/or deflection of the patch anchor with respect to the anchor driver. Thus, needle 4520 can be considered to be a stabilizer, and can be considered to be in a stabilizing position while disposed within the patch anchor. Furthermore, the ejection of the patch anchor from the needle (and thereby the withdrawal of the needle from within the patch anchor) may advantageously prevent the patch anchor from being undesirably and/or inadvertently drawn back through the leaflet by the needle upon withdrawal of the delivery tool (e.g., due to friction between the needle and the patch anchor). Such temporary stabilization is similarly provided in the system shown in FIGS. 27A-D, but with the sharp tip being provided by the patch anchor itself, rather than by the stabilizer.

FIGS. 27A-D show an extendable member in the form of a post 4530 that is a component of an anchor driver 450m, in accordance with some implementations. FIG. 27A shows patch anchor 240m having been advanced into contact with leaflet 10, but not yet having been pressed against the tissue. Anchor driver 450m, as for anchor driver 450l, includes an extendable member that provides temporary stabilization of the patch anchor on the anchor driver during pushing through the tissue. However, whereas the extendable member of anchor driver 450l (e.g., needle 4520) also provides the sharp point that pierces the tissue, the extendable member of anchor driver 450m (e.g., post 4530) can be blunt, and the sharp point may instead be provided by the tip of the patch anchor that is driven by driver 450m (e.g., patch anchor 240m).

Anchor driver comprises a rod 454m, and a drive head 452m that can be coupled to the rod via a spring 4532. Similarly to driver 450l, upon driver 450m (e.g., drive head 452m thereof) pushing the tip of patch anchor 240m against the leaflet, strains (e.g., compresses) in response to resistance from the tissue of the leaflet (FIG. 27B). This allows post 4530 to advance distally into the lumen of the patch anchor, thereby stabilizing the patch anchor on the anchor driver. Thus, post 4530 can be considered to be a stabilizer, and can be considered to be in a stabilizing position while disposed within the patch anchor. Further pushing by (e.g., advancement of) driver 450m advances patch anchor 240m through leaflet 10 (FIG. 27C). Once patch anchor 240m has advanced sufficiently far through the hole, resistance by leaflet 10 can reduce to the point at which the patch anchor can slip through the hole- and spring 4532 can responsively (e.g., abruptly) push drive head 452m to eject the patch anchor off of post 4530 (FIG. 27D). At this point driver 450m can be withdrawn, leaving patch anchor 240m in place.

FIGS. 28A-B show an extendable member in the form of a post 4540 that is a component of an anchor driver 450n, in accordance with some implementations. Anchor driver 450n can comprise a rod 454n, a drive head 452n, and/or a spring 4542, e.g., similarly to anchor driver 450m. Anchor driver 450n is shown being used with a patch anchor 240n, which can define a lateral opening 254n. Lateral opening 254n can serve a similar function to lateral opening 254, such as cooperating with a shoulder 4506n of anchor driver 450n (e.g., with a distally-facing face 4504h defined by the shoulder) to (i) preferentially allow deflection rather than lateral translation of the anchor with respect to the driver, and (ii) allow the toggle anchor to disconnect from the anchor upon reaching a predetermined angle with respect to the driver, e.g., as described with reference to FIGS. 22A-B, mutatis mutandis. However, for driver 450n and anchor 240n, this functionality is combined with that described for driver 450m and anchor 240m. This is achieved by shoulder 4056n being defined (or attached to) post 4540, and thereby being slidable with respect to the body of anchor 240n. To facilitate this, lateral opening 254n can be elongate (e.g., can be a slit), running parallel with the anchor axis of anchor 240n. FIG. 28A shows anchor driver 450n engaged with anchor 240n, in a state analogous to that of FIG. 27A, e.g., prior to the driver pushing the anchor against the leaflet. FIG. 28B shows a state analogous to that of FIG. 27B, e.g., with spring 4542 becoming contracted (e.g., compressed) and post 4540 sliding distally through the lumen of the anchor in response to driver 450n (e.g., drive head 452n thereof) pushing the anchor against the leaflet.

It is to be noted that, for driver 450n and anchor 240n, the above-described features of preferentially allowing deflection and deflection-dependent disconnection may be present only while post 4540 is retracted proximally (e.g., in the state shown in FIG. 28A), whereas while the post is extended (e.g., in the state shown in FIG. 28B) the post inhibits deflection of the anchor with respect to the driver.

It is to be noted that, for each of anchor drivers 450l, 450m, and 450n, the force by which the anchor driver drives the patch anchor through the tissue is applied via the spring of the anchor driver.

It is to be noted that, for each of anchor drivers 450l, 450m, and 450n, the needle or post of the anchor driver can be fixedly attached to the rod of the anchor driver, e.g., proximally from the spring of the anchor driver.

FIGS. 29A-D, and 30A-31D show respective extendable members that are components of respective patch anchors. In each case the distal tip of the patch anchor has a sharp point that is functionally exposed only during pushing of the patch anchor through the leaflet, e.g., the sharp point is initially functionally obscured, becomes functionally exposed responsively to pushing of the patch anchor against the leaflet, and automatically returns to being functionally exposed upon cessation of the pushing (e.g., upon arriving on the opposite side of the leaflet).

FIGS. 29A-D show, in cross-section, an extendable member in the form of a needle (or awl) 2410 that is a component of a patch anchor (or a more general toggle anchor) 2400, in accordance with some implementations. At tip 2500 of patch anchor 2400 needle 2410 defines a sharp point 2411 of the patch anchor. However, at rest, sharp point 2411 is functionally obscured by being ensheathed within the patch anchor (e.g., within a body of the patch anchor) (FIG. 29A), and only becomes exposed (e.g., functionalized) in response to the tip of the patch anchor being pressed against tissue (e.g., against leaflet 10), e.g., needle 2410 (FIGS. 29B-C). This occurs by compression of a spring 2412 of patch anchor 2400 responsively to resistance of the leaflet to the pushing. Once patch anchor 2400 has advanced sufficiently far through the leaflet, resistance by leaflet 10 can reduce to the point at which the patch anchor can slip through the hole, and spring 2412 can responsively re-extend, thereby resheathing the part of needle 2410 that defines sharp point 2411, e.g., functionally obscuring the sharp point (FIG. 29D).

As shown, spring 2412 can be a coil spring disposed around at least part of needle 2410. A proximal part of spring 2412 can be fixed to a proximal part of needle 2410.

Anchor 2400 and driver 4500 (e.g., the system of which they are components) can also include a temporary stabilization feature. However, whereas temporary stabilization features described hereinabove involve a component of the anchor driver (e.g., a rod or a needle) extending into the anchor, the temporary stabilization feature of anchor 2400 and driver 4500 involves the inverse. Driver 4500 comprises a receptacle (e.g., a cup) 4550 configured such that pushing, by the driver, of tip 2500 of the anchor against the tissue slides heel 2520 of the anchor proximally into the receptacle. In this stabilizing position, receptacle 4550 stabilizes anchor 2400 with respect to driver 4500, e.g., inhibiting deflection and/or lateral slipping of the anchor with respect to the driver.

For some implementations, and as shown, heel 2520 (or at least the part of the heel that enters receptacle 4550) is defined by needle 2410, e.g., by the proximal end of the needle, e.g., the end opposite the end that defines sharp point 2411. Heel 2520 can be dimensioned to fit snugly within receptacle 4550. Receptacle 4550 can be tubular.

For some implementations, and as shown, the temporary stabilization feature is facilitated by driver 4500 comprising a compression spring 4552 via which a drive head 4520 of the anchor driver is coupled to a rod 4540 of the anchor driver. Spring 4552 compresses as driver 4500 (e.g., drive head 4520 thereof) pushes anchor 2400 against the leaflet, thereby allowing heel 2520 to enter receptacle 4550 (FIG. 29B). Spring 4552 can eject heel 2520 from the receptacle upon the anchor having been pushed sufficiently far through the leaflet (FIG. 29D). For some implementations, spring 4552 has a spring constant that is sufficiently small to allow heel 2520 to enter receptacle 4550 in response to a pushing force that is less than that required to compress spring 2412 and expose sharp point 2411. This may therefore advantageously result in anchor 2400 becoming stabilized (FIG. 29B) prior to the anchor being driven into the tissue (FIG. 29C). As shown, spring 4552 can extend through at least part of receptacle 4550.

Driver 4500 can comprise a drive head 4520, and a rod 4540 that extends proximally from the drive head (e.g., to driver controller 112, or a variant thereof). As shown, driver 4500 can be configured such that the proximal sliding of heel 2520 into receptacle 4550 is accompanied by sliding of drive head 4520 proximally into the receptacle and/or proximally toward rod 4540.

FIGS. 30A-B and 31A-D show an extendable member in the form of a post 2420 that is a component of a patch anchor (or a more general toggle anchor) 240p, in accordance with some implementations. FIGS. 30A-B show patch anchor 24op alone, and FIGS. 31A-D show the patch anchor, with cord 242 and retrieval line 502 attached, being driven through tissue by a driver 450p. FIGS. 31A-C show patch anchor 240p in cross-section. Similarly to patch anchor 2400, patch anchor 240p temporarily becomes sharp during (e.g., due to) pushing of the tip of the patch anchor against and/or through the tissue. However, whereas the sharp point of patch anchor 2400 is functionally obscured by being ensheathed, patch anchor 240p has a sharp point 2421 that is disposed outside of the component that functionally obscures it. For example, and as shown, in FIGS. 30A and 31A, sharp point 2421 of patch anchor 240p can be functionally obscured by post 2420 extending distally beyond, but laterally close to, the sharp point. Thus, although sharp point 2421 can be visible it is not functional. Upon (e.g., due to) tip 250p of patch anchor 240p being pushed against tissue such as leaflet 10, post 2420 is obstructed by the tissue while the sharp point advances through the tissue (e.g., post 2420 becomes retracted into the body of the patch anchor), thereby functionally exposing (or functionalizing) sharp point 2421 (FIGS. 30B and 31B). This occurs by compression of a spring 2422 of patch anchor 240p responsively to resistance of the leaflet to the pushing. Once patch anchor 240p has advanced sufficiently far through the leaflet, resistance by leaflet 10 can reduce to the point at which the patch anchor (e.g., the body of the patch anchor) can begin to slip through the hole, and spring 2422 can responsively re-extend post 2420 to again functionally obscure sharp point 2421 (FIG. 30C).

As shown, spring 2422 can be a coil spring disposed within the body of the patch anchor, e.g., coaxially with post 2420. A proximal part of spring 2422 can be fixed to a proximal part of the body of the patch anchor, e.g., at heel 252p of the anchor. For some implementations spring 2422 and post 2420 can be cut from a unitary piece of stock material (e.g., stock tubing).

For some implementations, and as shown, post 2420 can be hollow. For some implementations, and as shown, post 2420 can have a lateral slit 2424 therein, rotationally aligned with eyelet(s) 244p of anchor 240p, and parallel with the axis of the anchor and/or the axis along which post 2420 slides. Slit 2424 accommodates the presence of cord 242 through eyelet(s) 244p irrespective of whether the post is extended, retracted, or partway therebetween.

32A-B, 33A-D, 34A-B, and 35A-C, which are schematic illustrations of patch anchors whose heel slides responsively to pulling (e.g., tensioning) of a longitudinal member attached to the anchor, in accordance with some implementations. The longitudinal member can be, for example, a cord connecting the anchor to another component of an implant (e.g., cord 242) or to a tissue, or a retrieval line such as retrieval line 502. This can be achieved by the anchor having a first segment, and a second segment that defines the heel and that slides axially with respect to the first segment upon pulling (e.g., tensioning) of the longitudinal member. The first segment can define the tip of the anchor.

FIGS. 32A-B and 33A-D show an extendable member in the form of a needle (or awl) 2430 that is a component of a patch anchor (or a more general toggle anchor) 240q, in accordance with some implementations. FIGS. 32A-B show patch anchor 240q alone, and FIGS. 33A-D show the patch anchor, with cord 242 and retrieval line 502 attached, being driven through tissue by a driver 450q. FIGS. 33A-D show patch anchor 240q in cross-section. Needle 2430 defines a sharp point 2431 of patch anchor 240q. Similarly to patch anchor 2400, sharp point 2431 of patch anchor 240q becomes functionally obscured following its use to penetrate tissue, e.g., by becoming ensheathed, such as by the body of the patch anchor. However, rather than this behavior being provided by a spring of the patch anchor, it occurs due to tension on cord 242.

Anchor 240q can comprise a tubular body 2436 and a stock 2434 (e.g., an elongate structure), slidable within the tubular body. Stock 2434 can thereby serve as an extendable member of anchor 240q (e.g., defining needle 2430). For some implementations, tubular body 2436 can be considered to be a first segment of anchor 240q, and/or stock 2434 can be considered to be a second segment of the anchor. For some implementations, similarly to patch anchors 2400 and 240p, and as shown, patch anchor 240q can be delivered while blunt (e.g., as shown in FIG. 33A) and can become sharp only temporarily while being driven through the tissue (e.g., as shown in FIGS. 33B-C). However, for some implementations, patch anchor 240q can be delivered while already sharp.

Anchor 240q is driven by driver 450q through leaflet 10 while sharp point 2431 is exposed (e.g., from tubular body 2436) and thereby able to facilitate piercing of the leaflet (FIG. 33B). FIG. 33C shows anchor 240q having been driven entirely through the leaflet but still in its sharp state, with sharp point 2431 exposed. FIG. 33D shows tension on cord 242 causing retraction of needle 2430 (e.g., proximal sliding of stock 2434 with respect to tubular body 2436) such that sharp point 2431 becomes functionally obscured. This tension on cord 242 can be applied in various ways, including those described elsewhere herein and/or those described in International Patent Application Publication No. WO 2022/101817 to Tennenbaum et al., which is incorporated herein by reference in its entirety. For example, the tension can be applied by an implant of which anchor 240q is a component, e.g., the tension can be applied by patch 210 (or a variant thereof), such as by spring 234 (or a variant thereof) of the patch.

Anchor 240q can be configured to respond in this way to tension on cord 242 by the manner in which the cord is threaded through and/or attached to the anchor. For example, and as shown, while needle 2430 is extended cord 242 follows a tortuous path through (e.g., laterally through) anchor 240q, whereas tension on the cord favors a less tortuous (e.g., straighter) path that corresponds to the needle being retracted. In the particular example shown, this is achieved by cord 242 entering a lateral eyelet 244q defined in the lateral wall of tubular body 2436, passing through a transverse channel 2438 defined in stock 2434, and being secured to another eyelet (or pair of eyelets) 245 defined in the lateral wall of the tubular body, opposite from eyelet 244q, e.g., see FIG. 33B. However, other arrangements can be used to similar effect, such as by cord entering lateral eyelet 244q and being secured to stock 2434. In any case, the tortuous path typically includes at least one turn between eyelet 244q, and the attachment point of the cord to the anchor. Increasing tension on cord 242 causes the cord to slide stock 2434 proximally to allow the cord to assume a less tortuous path, e.g., by bringing channel 2438 into (or at least closer to) alignment with eyelet 244q and/or with eyelet 245.

For some implementations, and as shown, stock 2434 may further define heel 252q of anchor 240q. For some such implementations, and as shown, the proximal sliding of stock 2434 can thus additionally extend heel 252q proximally, e.g., from the body of the anchor. Thus, while anchor 240q is in its sharp state and/or is being driven through leaflet 10, eyelet 244q can be disposed substantially proximally from the midpoint of the anchor (e.g., see FIG. 33B), and/or the transitioning of the anchor into its blunt state can both shorten the distal part of the anchor and lengthen the proximal part of the anchor-thus, in effect, bring eyelet 244q closer to the midpoint of the anchor.

FIG. 33C is intended to emphasize that anchor 240q can, itself, be stable in its sharp state. That is, unlike, for example anchor 240p, anchor 240q may not itself be biased toward its blunt state. Rather, as noted above, anchor 240q transitions toward its blunt state responsively to tension on cord 242. Thus, FIG. 33C shows some slack remaining in cord 242. However, it is to be noted that, for some implementations, an implant and/or a system of which anchor 240q is a component can in fact apply/maintain tension on cord 242 during the driving of the anchor through the tissue, and that for such implementations the state shown in FIG. 33C can be transient. For example, for such implementations, needle 2430 can retract proximally and/or heel 252q can extend proximally (e.g., stock 2434 can slide proximally with respect to tubular body 2436) instantaneously upon driver 450q ceasing to push stock 2434 (e.g., heel 252q defined thereby) and/or disengaging anchor 240q.

For applications in which anchor 240q includes a retrieval feature (e.g., retrieval eyelet) 241q, the retrieval feature can be defined by stock 2434, e.g., at heel 252q. Thus, for such implementations, anchor 240q can advantageously facilitate de-anchoring prior to tensioning of cord 242 due to the proximity of eyelet 244q to retrieval feature 241q and/or heel 252q at that point in the procedure (e.g., see FIG. 33C). That is, at that point in the procedure, pulling on retrieval line 502 can more effectively draw heel 252q into alignment with, and axially through, the hole in leaflet 10, e.g., compared to after the heel has been extended and/or compared to a similar anchor in which the length of the heel is fixed.

FIGS. 34A-B and 35A-C show a patch anchor 240r that has a similar de-anchoring advantage, although the advantage of patch anchor 240r persists even after tensioning of cord 242. FIG. 24AB show patch anchor 240r alone, and FIGS. 35A-C show the patch anchor being retrieved following its anchoring to leaflet 10, with cord 242 and retrieval line 502 attached. Patch anchor 240r comprises a retractable heel 252r, which is retractable (e.g., distally into a body 2446 of the anchor, which can be tubular) responsively to tensioning of (e.g., pulling on) retrieval line 502. Patch anchor 240r can comprise a stock 2444 that defines heel 252r, that is slidable within body 2446, and that can itself be tubular. For some implementations, body 2446 can be considered to be a first segment of anchor 240r, and/or stock 2444 can be considered to be a second segment of the anchor.

The mechanism for this retraction can be similar, mutatis mutandis, to that described for the retraction of needle 2430 of anchor 240q, but in the opposite orientation and involving retrieval line 502 rather than cord 242. In the example shown, while heel 252r is extended (FIGS. 34A and 35A) retrieval line 502 follows a tortuous path through (e.g., laterally through) anchor 240r, whereas tension on the retrieval line favors a less tortuous (e.g., straighter) path that corresponds to heel 252r being retracted (FIGS. 34B and 35B). For example, and as shown, this can be achieved by retrieval line 502 entering a first body-located retrieval eyelet 241r′ defined in the lateral wall of body 2446, passing transversely through heel 251r (e.g., through a transverse channel 2448 defined in the heel, the transverse channel optionally being defined by a pair of eyelets, as shown), and being secured to a second body-located retrieval eyelet (or pair of eyelets) 241r″ defined in the lateral wall of the body of the anchor, opposite from eyelet 241r′. However, other arrangements can be used to similar effect, such as by retrieval line 502 entering eyelet 241r′ and being secured to heel 252r. In any case, the tortuous path typically includes at least one turn between eyelet 244r′ and the attachment point of the retrieval line to the anchor. Pulling on retrieval line 502 causes the retrieval line to slide heel 252r distally to allow the retrieval line to assume a less tortuous path, e.g., by bringing eyelets 241r″ into (or at least closer to) alignment with eyelet 241r1 and/or with eyelet 241r″.

By being configured such that heel 252r automatically retracts in response to pulling on retrieval line 502, anchor 240r can be more effectively de-anchored, e.g., compared to a similar anchor in which the length of the heel is fixed. For example, the retraction advantageously draws heel 252r toward alignment with the hole in leaflet 10. The retrieval eyelet(s) (e.g., retrieval eyelet 241r′) being closer to eyelet 244r (compared to a similar anchor in which the length of the heel is fixed) can also contribute to this advantage.

For some implementations, and as shown, anchor 240r comprises a spring 2442 that biases heel 252r toward its extended state, e.g., such that pulling on retrieval line 502 (and thereby retracting the heel) strains the spring. For example, and as shown, spring 2442 can be a coil spring and/or a compression spring. As shown, spring 2442 can be disposed within (e.g., entirely within) body 2446. For some implementations, a distal end of spring 2442 can be fixed to body 2446, e.g., at a distal tip 250r of anchor 240r.

For some implementations, and as shown, stock 2444 defines at least one lateral slit 2445 therethrough, rotationally aligned with eyelet 244r and/or eyelet 245r, and parallel with the axis of the anchor and/or the axis along which the stock slides. Slit(s) 2445 advantageously allow(s) accommodates the presence of cord 242 through eyelet 244r, the stock, and eyelet 245r, irrespective of whether heel 252r is extended, retracted, or partway therebetween. That is, slit(s) 2445 advantageously facilitate(s) the attachment of cord 242 to anchor 240r without interfering with the heel-retraction functionality described hereinabove.

For some implementations, spring 2442 and heel 252r can be cut from a unitary piece of stock material (e.g., stock tubing), e.g., stock 2444 is cut from a unitary piece of stock material in a manner that defines the spring and the heel.

It is to be noted that, although distal tip 250r is shown as open (e.g., body 2446 has an open distal end), for some implementations it can in fact be closed.

It is to be noted that the features of anchors 240r and 240q can be combined, e.g., to provide spring-loaded retraction of a sharp pointed tip concurrently with extension of a heel.

It is to be noted that, whereas the retraction of the heel of anchor 240r reduces the axial length of the anchor, the extension of the heel of anchor 240q may not change the axial length of the anchor, e.g., due to the concurrent retraction of the needle. Moreover, for some implementations anchor 240q may be configured such that the extension of its heel can increase the axial length of the anchor.

Although examples described herein relate primarily to (A) implants that comprise an upstream assembly that includes a patch and a patch anchor, a downstream assembly that comprises a winch and a winch anchor, and a tether that tethers the upstream assembly to the downstream assembly, and (B) delivery tools for implanting such implants, it is to be noted that the scope of the present disclosure includes:
(i) for each of the upstream assemblies and patches described herein, its use as a leaflet-augmentation patch independent from a downstream assembly, e.g., with a tether anchored in the ventricle without a winch, or without a tether at all;
(ii) for each of the patch anchors described herein, its use as a tissue anchor other than for anchoring a leaflet patch to a leaflet, e.g., for anchoring a different implant to a leaflet (such as for anchoring a tether directly to a leaflet, independently from a leaflet patch), or for anchoring to a different tissue;
(iii) for each of the downstream assemblies described herein, its use independently of a leaflet-augmentation patch, e.g., with a tether anchored to a leaflet independently from a leaflet patch, or with a tether anchored to a tissue other than a leaflet; and
(iv) delivery tools that comprise only certain components or mechanisms of tool 400 (or its variants), such as (a) only those for interacting with a heart valve leaflet (e.g., for anchoring, to the leaflet, a leaflet-augmentation patch independent from a downstream assembly), (b) only those for interacting with a patch anchor described herein, (c) only those for anchoring and/or actuating a downstream assembly independent from a leaflet patch, or (d) a combination of any two of a, b, and c.

Reference is now made to FIGS. 36A-C, which illustrate a patch anchor 240s (e.g. a toggle anchor) that is in the form of a helical coil, in accordance with some implementations. In some implementations, the coil is a helical spring. In some implementations, and as illustrated by FIG. 36B, when the anchor 240s is deployed within the heart, the helical coil lies against (e.g. parallel with) the leaflet, within the ventricle. In this deployed state, the helical coil is typically relaxed—e.g. in a non-compressed state in which turns of the coil can move with respect to each other. This may advantageously allow the anchor to lie conformally against the leaflet and/or to move conformally with the leaflet responsively to the cardiac cycle of the heart, e.g. thereby preventing ends of the patch anchor from digging into the leaflet tissue.

In some implementations, and similarly to that described with respect to other toggle anchors described hereinabove, when anchor 240s is deployed within the heart, cords 242 can connect the anchor to a leaflet patch (e.g. to patch 210), by the cords extending, from patch anchor 240s (where the cords can be connected to a midsection of the anchor by looping around a turn of the helical coil), away from the anchor (e.g. orthogonally away from the anchor, e.g. as shown in FIG. 36B), and through tissue of the leaflet to the patch.

In some implementations, at least one end 244s of the coil forms a closed end, in order to prevent emboli from leaving a lumen defined by the helical coil and entering the bloodstream once the anchor is deployed within the heart. For example, and as shown in the inset of FIG. 36B, at least one end of the coil can be in the form of a flat spiral end 244s.

In some implementations, during driving of anchor 240s through the leaflet tissue, a driver 450s (e.g. a needle) extends through the lumen, in order to puncture the tissue and deliver the anchor therethrough (FIG. 36A). In some implementations, and as illustrated in FIG. 36A, during delivery of the anchor through the leaflet, driver 450s forces spiral end 244s of the anchor into a conical form, e.g. in order to assist in the puncturing of the leaflet. For example, the tip of driver 450s may itself be conical.

In some implementations, anchor 240s is delivered to the heart while a retrieval line 502s is coupled to the anchor. Similarly to as described hereinabove for retrieval line 502 hereinabove, pulling on the retrieval line causes patch anchor 240s to pivot (e.g. to reorient towards a more vertical orientation), and draws the patch anchor back through the hole in the tissue through which it was delivered (FIG. 36C). In some implementations, and as shown, retrieval line 502 can be threaded through turns of the coil, such that pulling the retrieval line causes anchor 240s to become stiffened, by the turns of the coil becoming compressed against each other. This may advantageously allow for easier passage of the anchor back through the tissue.

Reference is now made to FIGS. 37A-C, which illustrates a technique for coupling a tether to a component of an implant, in accordance with some implementations. In the example shown, this technique is used to couple tether 160 to leaflet patch 210 (or a variant thereof). However, it is to be understood that the scope of the present disclosure include other implementations of this technique—e.g. for coupling other tethers to components of other implants, mutatis mutandis.

In order to couple tether 160 to leaflet patch 210, an end portion 163 of the tether is looped around and/or through part of the leaflet patch—e.g. around part of lip brace 231 and/or through an eyelet defined by the lip brace (FIG. 37A). As shown in FIG. 37B, the resulting loop 167 is closed by burrowing the end portion of the tether through a stretch 165 of the tether. This may be achieved using a needle through which the end portion is threaded. In this context, the term “burrowing” does not mean merely passing the end portion transversely through the other stretch of the tether, but instead means passing the end portion coaxially within the tether along the stretch e.g. such that the stretch squeezes on the end portion therewithin.

In some implementations, tether 160 is braided and/or woven, in order to allow for the burrowing of the end portion through stretch 165, e.g. strands of the braid or weave are pushed apart by the burrowing. For some implementations stretch 165 may simply appear wider than other parts of tether 160. FIG. 37C shows end portion 163 having been trimmed away, and the other end of tether 160 coupled to downstream assembly 300.

Rather than necessitating knots and/or coupling elements for coupling tether 160 to leaflet patch 210 (e.g. that may rub against the opposing leaflet during ventricular systole thereby causing irritation, and/or that may increase the risk of a potential embolus forming thereon), the above described coupling of the tether to the leaflet patch (e.g. via burrowing the tether through itself) may advantageously provide a smooth and non-abrasive coupling for the tether to the leaflet patch.

In addition, the technique shown in FIGS. 37A-C may be particularly suited for implants that are required to withstand the forces induced by the cardiac cycle of the heart over a relatively long period of time. For example, forces acting on tether 160 to pull end portion 163 out of stretch 165 may cause stretch 165 to squeeze end portion 163 tighter, such that the stretch acts in a similar fashion to a Chinese finger trap, thereby preventing loop 167 from opening, and thus maintaining the coupling between tether 160 and leaflet patch 210.

For some implementations, stretch 165 has a length of greater than 3 mm and/or no more than 6 mm (e.g. 3-6 mm).

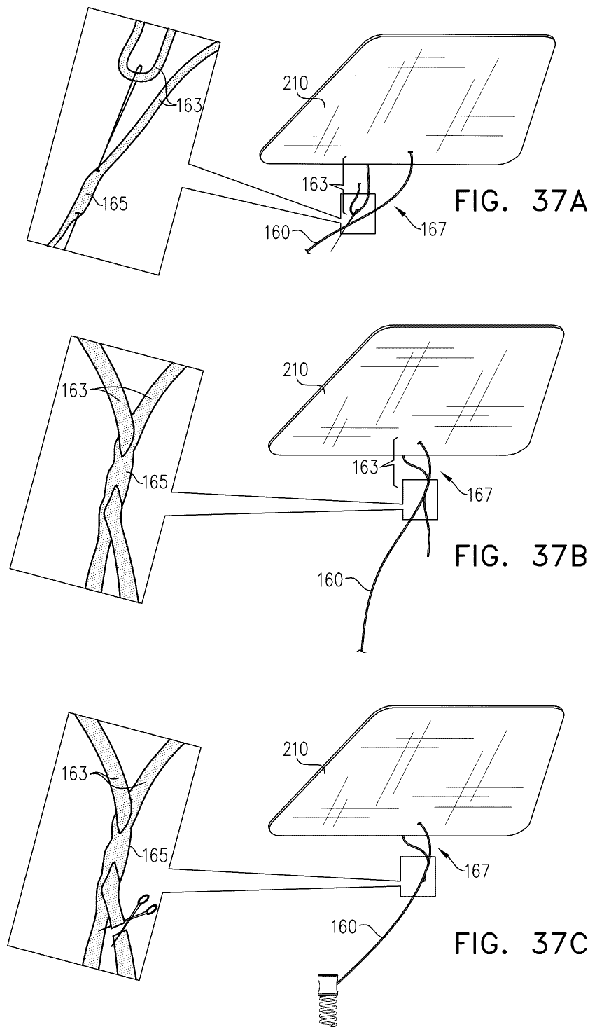

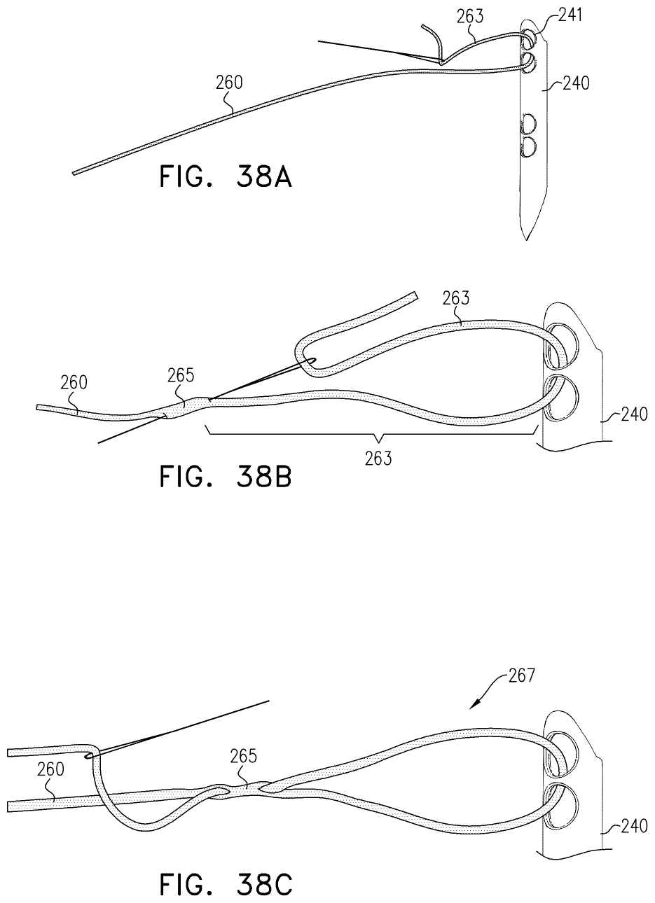

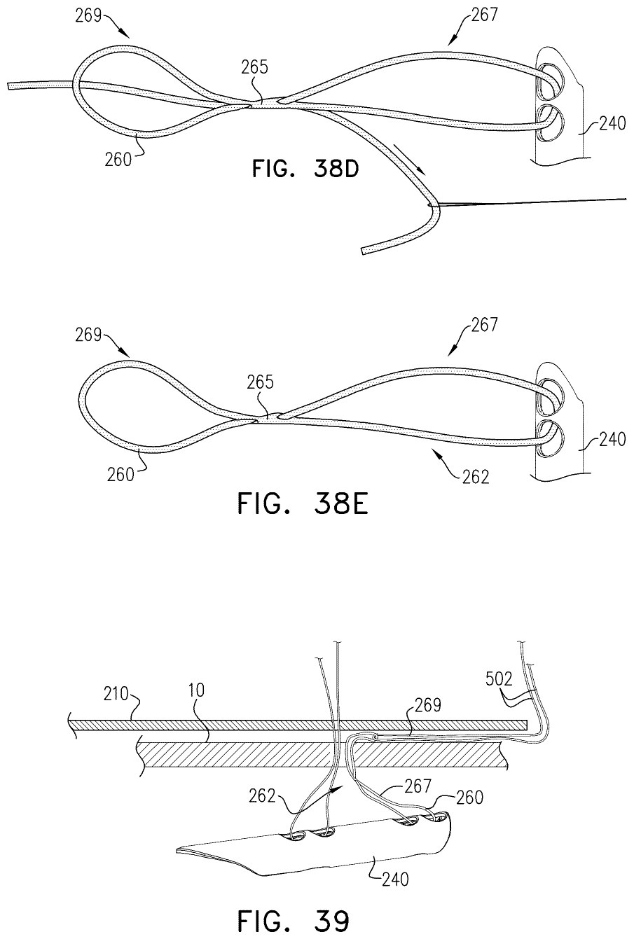

Reference is now made to FIGS. 38A-E and 39, which show a technique for coupling, via a tether 260 (e.g. a wire, line, or adapter), two components of a system thereto, in accordance with some implementations. In order to couple the two components to each other, the tether is arranged into two loops, each of the components being connected to a corresponding loop of the tether. In the example shown, this technique is used to connect a patch anchor 240 to a retrieval line 502 (e.g. to allow for retrieval of patch anchor 240, e.g. as described above with reference to FIGS. 11A-B), the tether thereby serving as a retrieval adapter 262 for the implant. However, it is to be understood that the scope of the present disclosure include other implementations of this technique—e.g. for coupling other components of other implants and/or delivery tools to each other, mutatis mutandis.

A first step of the technique involves coupling tether 260 to patch anchor 240, using a technique similar to the technique described with reference to FIGS. 37A-C above. An end portion 263 of the tether is looped around and/or through part of the patch anchor to form a first loop 267—e.g. the end portion is looped through retrieval eyelet 241 (FIG. 38A). As shown in FIG. 38B, the resulting loop 267 is closed by burrowing the end portion of the tether through a stretch 265 of the tether. This may be achieved using a needle coupled to the end portion (e.g. by threading of the end portion therethrough). Similarly to as described with reference to FIGS. 37A-C hereinabove, in this context, the term “burrowing” does not mean merely passing the end portion transversely through the other stretch of the tether, but instead means passing the end portion coaxially within the tether along the stretch e.g. such that the stretch squeezes on the end portion therewithin.

Once the first loop 267 is formed (FIG. 38C), a second loop 269 can then be formed by looping end portion 263 back towards the first loop and the stretch, and subsequently burrowing the end portion again through the tether (FIG. 38D). In some implementations, this is achieved by burrowing the end portion a second time through stretch 265, e.g. such that two lengths of the end portion extend, side-by-side (e.g. colinearly), within the stretch. As shown in FIG. 38E, such an approach may result in retrieval adapter 262 having a “figure-of-eight” arrangement. Alternatively, in some implementations, the second loop may be formed by burrowing the end portion through the through a different stretch of the tether, e.g. such that the retrieval adapter has a “handcuff” type arrangement, with its loops at opposite ends of a length of the tether.

In some implementations, and as shown in FIG. 39, retrieval adapter 262 facilitates an implementation of retrieval line 502 without the retrieval line passing through the hole in leaflet 10 through which toggle anchor 240 passed (and through which cord 242 extends). Inter alia, this advantageously reduces a likelihood of retrieval line 502 injuring the leaflet (e.g. widening the hole in the leaflet) upon its decoupling from the toggle anchor and withdrawal from the body. Instead, retrieval adapter 262 passes through the hole in leaflet 10, and retrieval line 502 is looped through second loop 269 on the side of the leaflet at which leaflet patch 210 is disposed. Retrieval adapter 262 (e.g. second loop 269 thereof) may be sandwiched between leaflet patch 210 and leaflet 10. For some implementations, retrieval line 502 is looped through second loop 269 between leaflet patch 210 and leaflet 10. Alternatively, retrieval line 502 may be looped through second loop 269 adjacent to leaflet patch 210.

Similarly to as described hereinabove for retrieval line 502 and various retrieval eyelets, pulling on the retrieval line causes patch anchor 240 to pivot (e.g. to reorient towards a more vertical orientation), and draws the patch anchor back through the hole in the tissue through which it was delivered.

In some implementations, once it has been determined that patch anchor 240 has been satisfactorily anchored within the heart, retrieval line 502 can be unlooped from retrieval adapter 262 (e.g. from second loop 269 thereof), leaving second loop 269 sandwiched between leaflet 10 and patch 210. In such implementations, this sandwiching of the second loop can advantageously conceal the retrieval adapter 262 that is left in place once the retrieval line has been withdrawn.

In some implementations, a single retrieval line can be connected to two patch anchors 240 by the retrieval line looping through the respective retrieval adapter of each of the anchors (e.g. by looping through a second loop 269 of each of the retrieval adapters of the anchors). In such implementations, once the anchors 240 are anchored in the heart, should it be desired that the anchors 240 be retrieved, both anchors may be retrieved by pulling on the single retrieval line.

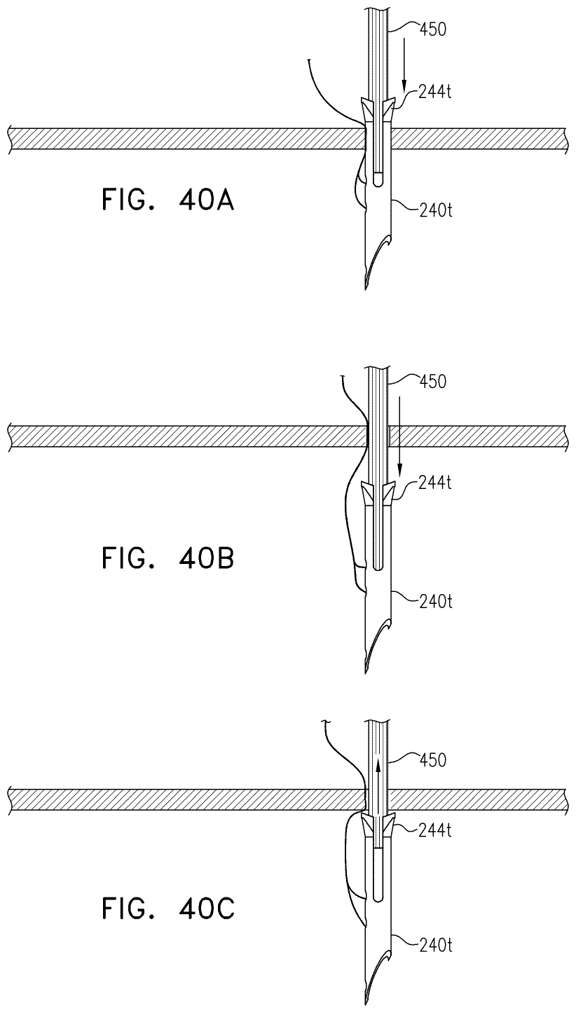

Reference is now made to FIGS. 40A-C, 41A-C, and 42, which illustrate various systems and techniques for preventing a patch anchor (e.g. a variant of patch anchor 240) from being inadvertently withdrawn back through tissue of the heart, e.g. by ensuring that the anchor has been disengaged from an anchor driver that was used to deliver the anchor. As described above, during implantation of an anchor at the heart a driver 450 may extend through the anchor in order to push the anchor through the tissue, and thus it is important that the anchor is disengaged from the driver prior to the withdrawal of the driver back through the tissue and out of the heart. For example, in some implementations, the systems described hereinbelow are advantageously adapted to facilitate passage of the anchor through the leaflet during implantation of the anchor, but to inhibit passage of the anchor back through the leaflet in a reverse direction.

FIGS. 40A-C show a patch anchor 240t having a heel 244t that is flared, e.g. such that the heel inhibits withdrawal of the patch anchor back through the tissue. For example, should the heel be pulled in a reverse direction against the tissue, heel 244t may advantageously prevent the anchor from being pulled through the tissue by the heel abutting the tissue (FIG. 40C). In such implementations, the patch anchor may be particularly suitable for anchoring through the leaflet (FIG. 40A), for example, the anchor may define a streamlined (e.g. arrow-shaped) form.

In some implementations, heel 244t may define wings (e.g. a pair of wings, or three wings) that are adapted to transiently flex medially toward each other during passage of the heel through the leaflet, and to flex laterally away from each other upon the heel being pushed against the leaflet in a reverse direction. This may advantageously assist in the disengagement of the anchor from the driver, e.g. by causing the anchor to release its grip on the driver, thereby allowing the driver to be withdrawn proximally through the tissue and/or increasing the area of heel 244t thus further preventing the withdrawal of the anchor back through the tissue.

Reference is now made to FIGS. 41A-C, which illustrate a clasp 430g for which each slot 437g has an associated tooth 439g that is configured to facilitate patch anchor 240 being driven through the slot, and to inhibit the patch anchor from being withdrawn, in a reverse direction, through the slot. For example, each tooth 439g may be biased to obstruct its respective slot (e.g. by being positioned directly downstream of the slot, e.g. as shown in FIG. 41C), but may be configured to be transiently be pushed aside as patch anchor 240 is driven by the driver through the slot (FIG. 41A), e.g. by the tooth being resilient and/or elastic, and return to its obstructing state as soon as the anchor has passed through the slot (FIG. 41B).

Reference is now made to FIG. 42, which illustrate a delivery tool 4001 (e.g. a variant of delivery tool 400) that is configured to orient driver 450 in a manner that prevents patch anchor 240 from being accidentally withdrawn back through the tissue, along with the driver. For example, and as shown, delivery tool 400l can be adapted to orient driver with respect to clasp 430 (e.g. with respect to slot 437 thereof) such that as the driver drives patch anchor 240 through the slot, the angle of attack of the anchor is such that the anchor rubs (e.g. scrapes) along a rim 439 of the slot, and retrieval of the driver from the slot causes the patch anchor to abut, and therefore be obstructed by, the rim of the slot.

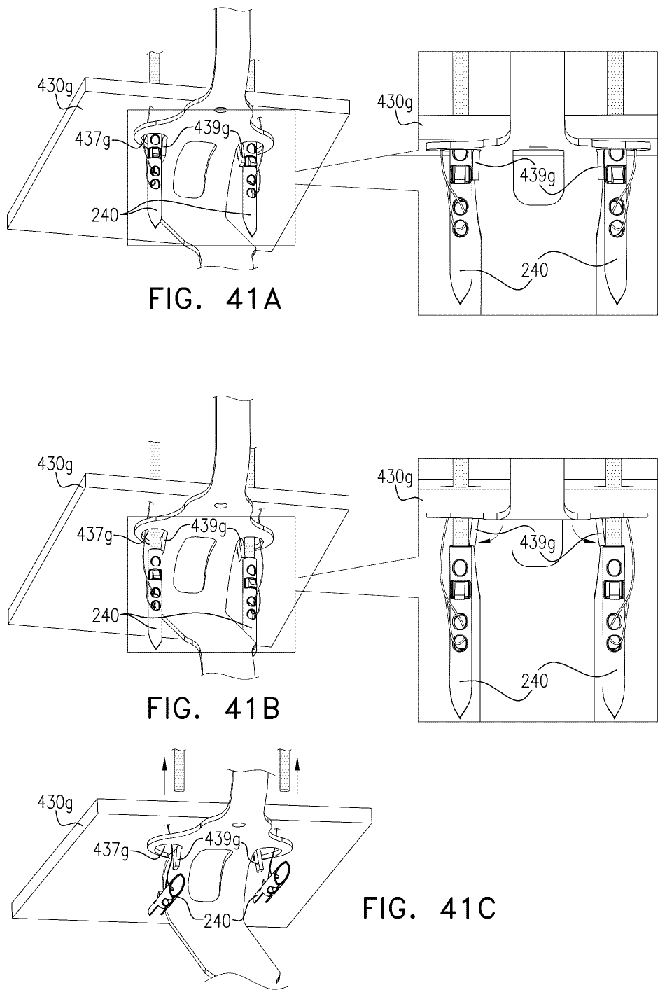

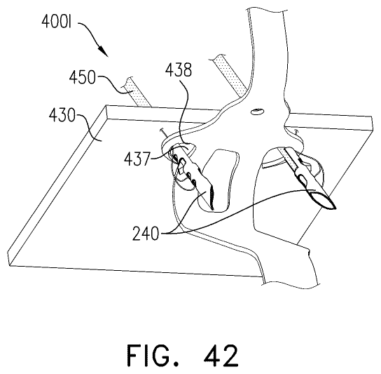

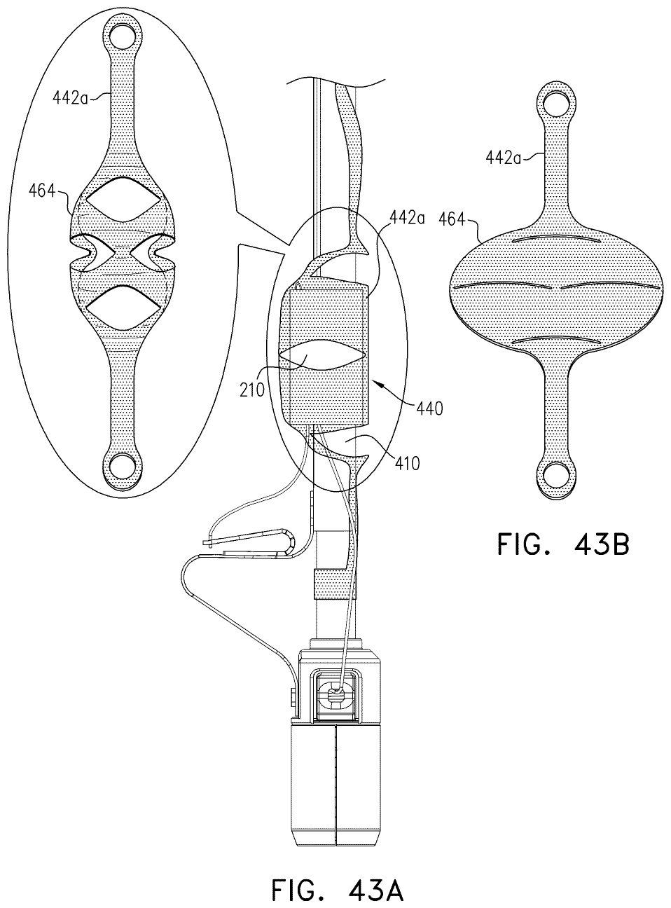

Reference is now made to FIGS. 43A-B, which show a Kirigami wrap 442a, for holding a component of an implant against a delivery tool, in accordance with some implementations. In the example shown, this technique is used to hold patch 210 against mount 440, e.g. wrap 442a can be considered to achieve the same function as that of wraps 442. However, it is to be understood that the scope of the present disclosure includes other implementations of this technique—e.g. for holding components of other implants and/or delivery tools to each other, mutatis mutandis.

In some implementations, wrap 442a can be formed using a Kirigami-style technique, by cutting a flexible material (e.g. a fabric, a polymer (such as polymer laminate or nitinol), or any other suitable materials) to form the wrap. In some implementations, the wrap forms a grasping structure that, when under tension, can hold the patch against the mount (and/or against shaft 410). In some implementations, this is achieved by wrap 442a defining at least two arms 464, that, upon tensioning of the wrap, curve and/or deflect towards each other. In some implementations, the wrap holds patch 210 against mount 440 by extending (e.g. curving) around the body of the mount on the opposite side of the mount to the patch, and protruding over (e.g. overlapping against) at least the edges of the patch, e.g. by arms 464 of the wrap 442a holding (e.g. hugging) the patch against the mount.

As described in more detail hereinabove, mount 440 is configured to carry patch 210 toward clasp 430, e.g. while wrap 442a is maintained under tension, thereby holding the patch against the mount. In order to release wrap 442a (e.g., to release patch 210 from being held against mount 440 by the wrap), the tension is released. For example, a release mechanism (not shown) that extends, from wrap 442a, transluminally along shaft 410 (e.g. alongside, or through the shaft), may be actuated to release the wrap. Responsively to the release of the tension, the wrap assumes a flatter form in which arms 464 move away from each other (e.g. illustrated in FIG. 43B), thereby releasing its hold on the patch. The release mechanism can be in the form of a rod that is attached to a proximal end of the patch, such that actuating the release mechanism is achieved by pulling or pushing on the rod.

It is to be noted that, for some implementations, and as shown, the tension that transforms wrap 442a into its curved holding state is applied along an axis of the wrap that is transverse to an axis of the wrap on which arms 464 lie.

Techniques for mitigating forces on a tether of an implant during a cardiac cycle of the heart are now described. In some implementations, a shock absorber (e.g. a spring) is mounted on a component of the implant to which a tether is connected, in order to mitigate forces acting on the implant due to the cardiac cycle, e.g. to prevent the tether from pulling on or tugging at various components of the implant. In some such implementations, the spring is a spring that defines a helix, and the spring grips the tether in-between turns of the helix, such that when tension is induced in the tether, the turns of the spring, along with the tether, move rhythmically with the heart, thereby advantageously acting as a shock absorber for the implant.

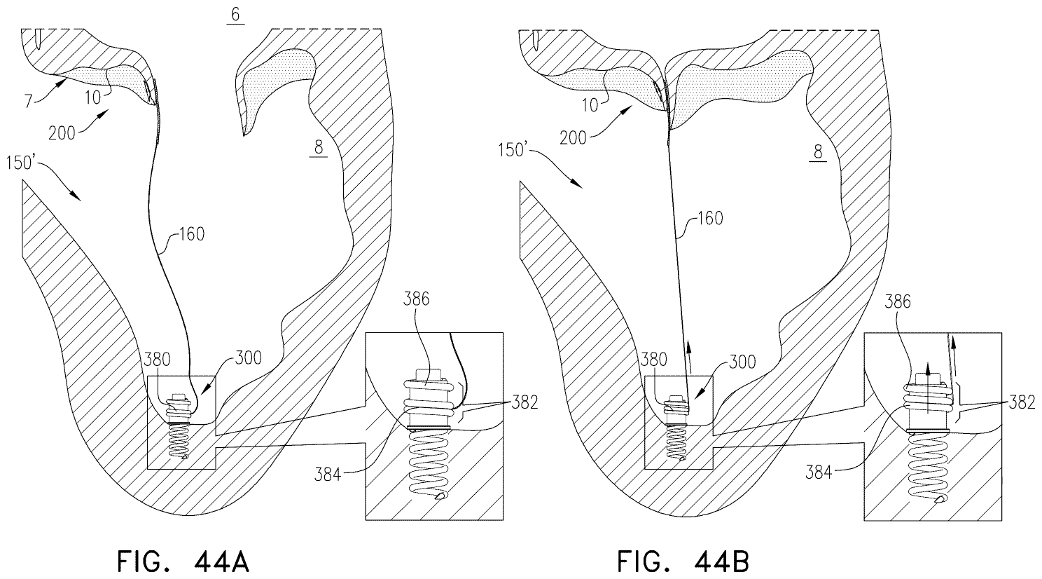

Reference is now made to FIGS. 44A-B, which illustrate an implant 150′, which shows an implementation of the shock-absorption described above, in accordance with some implementations. Implant 150′ may be substantively identical to implant 150 described hereinabove (e.g. comprising winch 320, winch anchor 310, and tether 160), with the addition of a shock absorber (e.g. a shock-absorbing spring) 380, mounted on the winch (e.g. on housing 321 thereof). In the example shown, shock absorber 380 comprises a helical spring. However, in some implementations, shock absorber 380 comprises a volute spring, a cantilever spring, and/or a wave spring.

As described hereinabove with respect to implant 150, tether 160 can extend, from winch 320, out of an aperture 326 of housing 321, and upstream through the ventricle towards upstream assembly 200. In some implementations, shock absorber 380 is coupled to the housing (e.g. to an exterior of the housing) in a manner that urges the tether away from contact with a rim of the aperture, e.g. to prevent the tether from rubbing against the rim of the aperture (e.g. the rim that is furthest away from the winch anchor, such as the upper rim of the aperture) during the cardiac cycle. As shown in the transition from FIG. 44A to 44B, the spring moves with respect to the housing responsively to the cardiac cycle, thus serving as a shock absorber for the tether by mitigating the forces acting on the implant (e.g. on the upstream and/or downstream assembly), and maintaining the tether urged away from contact with the rim.

In some implementations, shock absorber 380 comprises (e.g. defines) a gripping region adapted to grip the tether (e.g. in a manner that maintains the tether urged away from contact with the rim), and a spring element that moves, responsively to the cardiac cycle, with respect to the housing. For example, and as shown in FIGS. 44A and 44B, the spring can define a helix 382 having a series of turns that extend circumferentially around the housing, the spring gripping (e.g. trapping) the tether in between turns of a first portion 384 of the helix (e.g. such that the turns of the first portion are fixed with respect to each other), and the spring having a second portion 386 of the helix that serves as a spring that relaxes and contracts responsively to the cardiac cycle (e.g. by the distance between the turns of the second portion increasing and decreasing responsively to the transition between ventricular systole and ventricular diastole). That is, a single coil defines both the helical spring and the gripping region.

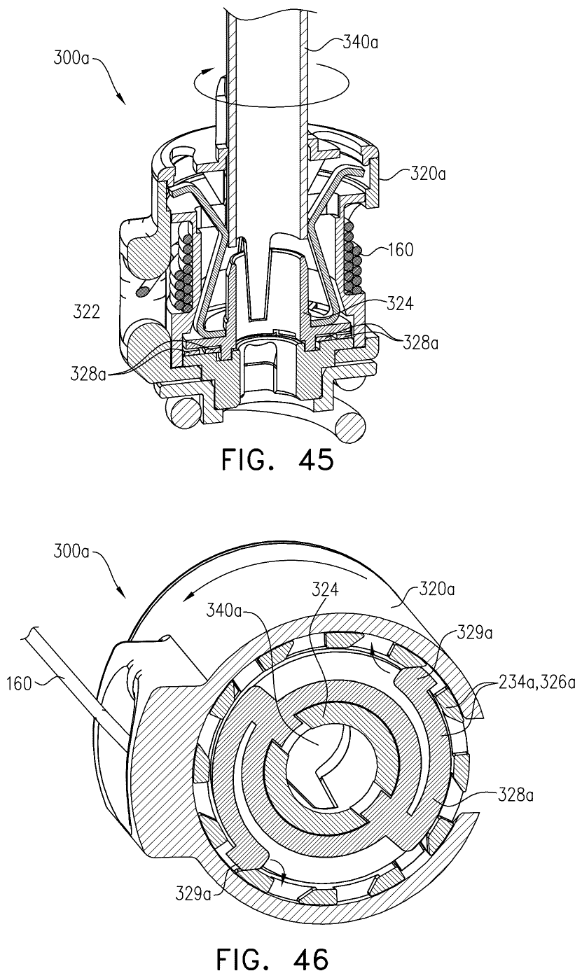

Reference is now made to FIGS. 45 and 46, which show a torque limiter 328a, for preventing over-tensioning of tether 160, in accordance with some implementations. As described hereinabove, tether 160 can be operatively coupled to downstream assembly 300a by being coupled to a winch 320a (e.g. a variant of winch 320) of the downstream assembly, such that actuation of the winch (e.g. spool 322 thereof) can adjust (e.g. shorten) an effective length of the tether between the winch and patch 210, e.g. via a driveshaft 480 (which may be a variant of driveshaft 480) applying torque to the spool (e.g. as shown by the arrow in FIG. 45). In some implementations, driveshaft 480a is coupled to spool 322 via a driveshaft interface 324a, such that application of torque to the driveshaft interface in a first direction, by the driveshaft, winds the tether further around the spool (and optionally, application of torque in a second, opposite direction unwinds the tether from around the spool).

In some implementations, in order to prevent the tether from become overly tensioned, e.g. and therefore undesirably tugging or pulling on patch 210 and/or on winch anchor 310, downstream assembly 300a can comprise a torque limiter 328a that is adapted to prevent winch 320a from being overly tensioned by the driveshaft. For example, torque limiter 328a can be in the form of a slip clutch, such that driveshaft 480a can be coupled to driveshaft interface 324a in a manner in which over-application of torque to the winch causes the slip clutch to slip without rotating the spool. This may be achieved by one or more spring-loaded detents 329a of the slip clutch being disposed within corresponding slots 326a of driveshaft interface 324a, such that application of torque below a predetermined threshold rotates the spool by the detents remaining engaged within the slots, and application of torque above the predetermined threshold forces each arm to slip to the next slot (e.g. as shown by the arrow), thereby transiently disengaging the driveshaft from the winch.

For some implementations, torque limiter 328a is configured to function only during actuation of winch 320a—e.g. to prevent over-tensioning of tether 160 during the procedure. For such implementations, torque limiter 328a may become locked at the end of the procedure—e.g. the locking of the winch may functionally disable the torque limiter, such that it cannot slip subsequently to the procedure (e.g. as a result of transient hemodynamic changes).

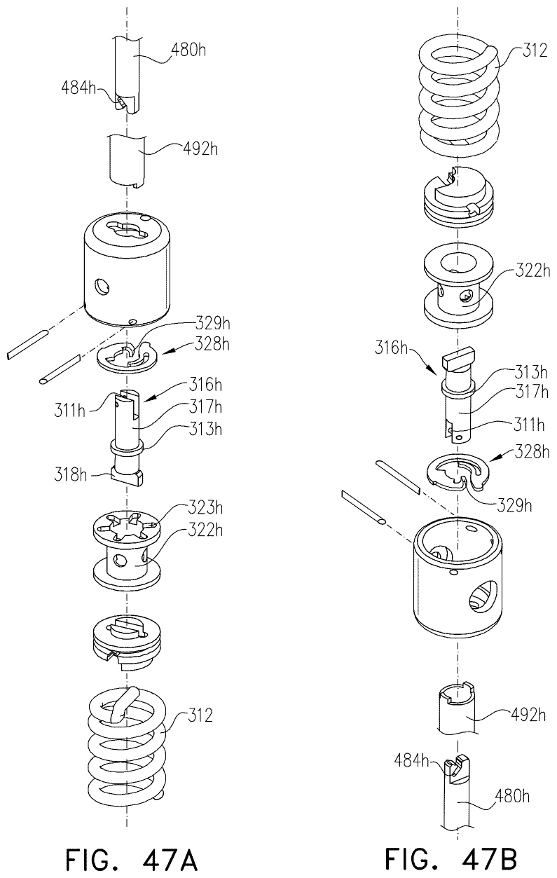

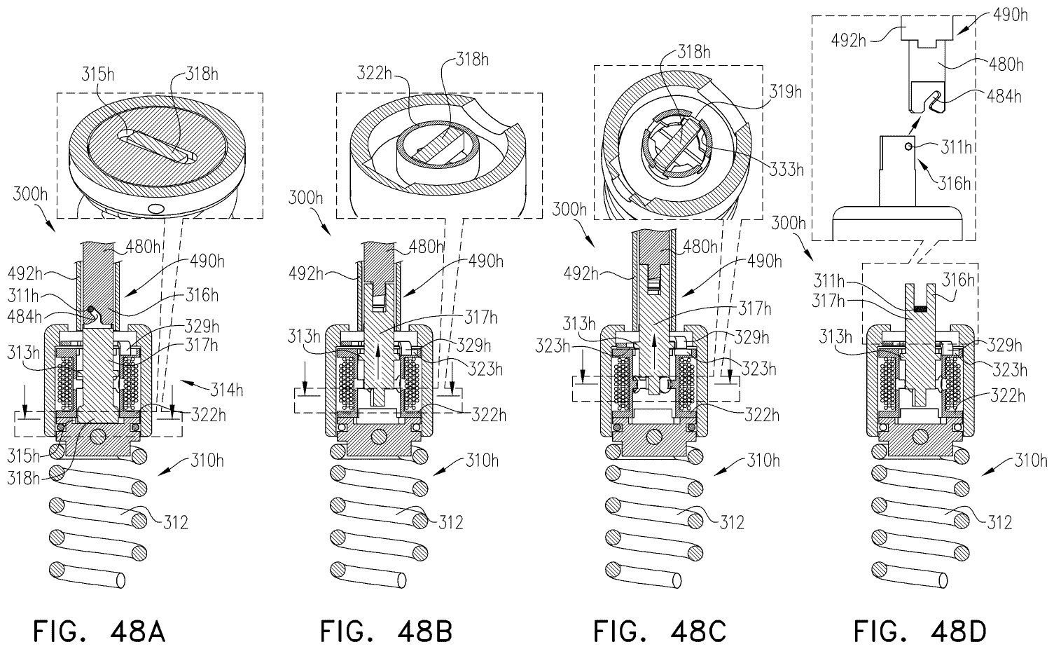

Reference is now made to FIGS. 47A-B, which illustrate an exemplary downstream assembly 300h, and FIGS. 48A-D, which illustrate an exemplary coupling of a driveshaft subassembly 490h to a driver interface 316h of downstream assembly 300h, in accordance with some implementations. As described hereinabove with reference to FIGS. 21A-E, inter alia, in some implementations, a unitary downstream-assembly-control driveshaft (e.g. a variant of driveshaft 480g) can be used to both apply an anchoring force to the winch anchor, and to actuate winch 320 to rotate spool 322. FIGS. 48A-D illustrate driver interface 316h, which may be considered to be a variant of interface 316g, being used to achieved both such functions.

Also similarly to FIGS. 21A-E, downstream assembly comprises an axle 317h that typically defines and/or is fixedly attached to interface 316h. Transitioning of downstream assembly 300h between its anchoring state and its winching state is achieved by shifting a position (e.g., an axial position) of axle 317h with respect to other components of the downstream assembly.

FIG. 48A shows downstream assembly 300h in its anchoring state, in which axle 317h is engaged with an anchor head 314h of an anchor 310h, e.g., with an axle head 318h disposed within a complementarily-shaped socket 315h defined by the anchor head (e.g., a hex drive). In this state, rotation of driveshaft 480h rotates axle 317h, which rotates tissue-engaging element 312, screwing it into the tissue. In the example shown, axle head 318h resembles a flat-bladed screwdriver head, and socket 315 resembles a complementary slot, but it is to be noted that other drive configurations are possible.

Following this anchoring to the tissue, axle 317h may be moved (e.g., proximally), via an axial (e.g., pulling) force applied by driveshaft 480h, transitions downstream assembly 300h into its winching state (FIG. 48C) in which axle 317h is engaged with spool 322h of winch 320h, e.g., with axle head 318h disposed within a complementarily-shaped socket 319h defined by spool 322h. In the example shown, socket 319h is defined by multiple protrusions 333h that protrude inward from an inner surface of spool 322h. Also in the example shown, socket 319h is shaped to receive axle head 318h in more than one rotational orientation—e.g. effectively defining multiple intersecting slots.

In some implementations, in order to prevent unintended rotation of spool 322h (e.g. in order to prevent unintended winding/unwinding of the tether from around the spool), downstream assembly 300h comprises a lock 328h that comprises a spring-loaded detent 329h that can protrude into recesses 323h defined in a surface of spool 322h, thereby retaining the spool in a locked state in which it cannot rotate. In some implementations, axle 317h defines a protruding rim 313h, that, upon the axle being pulled proximally (e.g. in order to engage axle head 318h within socket 319h), pushes detent 329h out of recess 323h, thereby temporarily unlocking the spool and allowing for subsequent actuation of the spool.

In some implementations, and as shown, downstream assembly 300h is also provided with a neutral state (FIG. 48B) via which the downstream assembly transitions from its anchoring state into its winching state. For example, in the neutral state, axle head 318h can be disengaged from socket 315h and socket 319h, e.g., the axle head can be disposed in a space between these sockets, in which the axle head can rotate freely without rotating the anchor head or the spool. In some implementations this can facilitate the transition from the anchoring state to the winching state—e.g. to allow rotational alignment of the axle head with socket 319h. In the neutral state, spool 322h is typically locked by lock 328h.

Once anchoring and winching are complete, driveshaft 480h is disengaged from interface 316h, in order to decouple the driveshaft from the anchor, and leave the anchor implanted within the heart. In some implementations, the coupling between driveshaft 480h and interface 316h is provided by a slot- and—pin mechanism. For example, and as shown, driveshaft 480h can define an oblique slot 484h through which a transverse pin 311h of interface 316h is disposed. In some implementations, the driveshaft subassembly comprises a reference-force tube 492h through which driveshaft 480h extends. Due to the oblique orientation of slot 484h, disengagement of driveshaft 480h by pin 311h exiting the slot requires the driveshaft to move laterally with respect to interface 316. While reference-force tube 492h is disposed over interface 316h, the reference-force tube prevents such lateral movement of driveshaft 480h with respect to the interface, and thereby maintains the engagement between driveshaft 480h and the interface. Once it has been determined that anchoring and winching are complete, reference-force tube 492h can be retracted from interface 316, thereby allowing disengagement of driveshaft 480h from the interface, e.g. by slot 484h sliding obliquely off of pin 311h responsively to proximal pulling of the driveshaft. FIG. 48D shows axle 317h having returned to the neutral position shown in FIG. 48B, but it is to be understood that following disconnection the axle can be free to alternatively return to other positions.

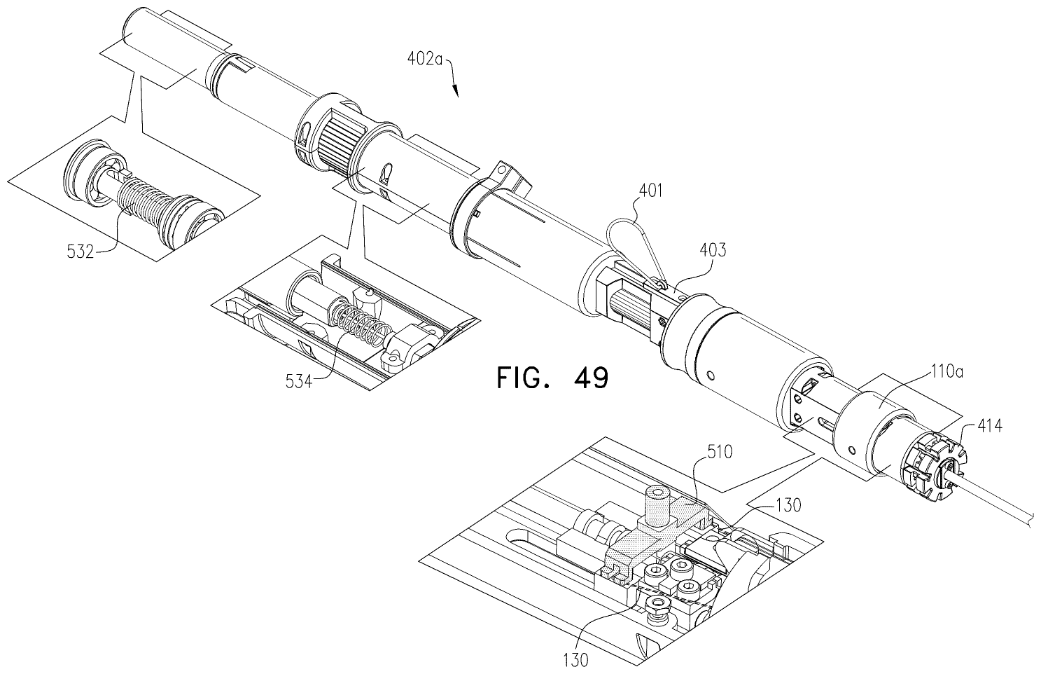

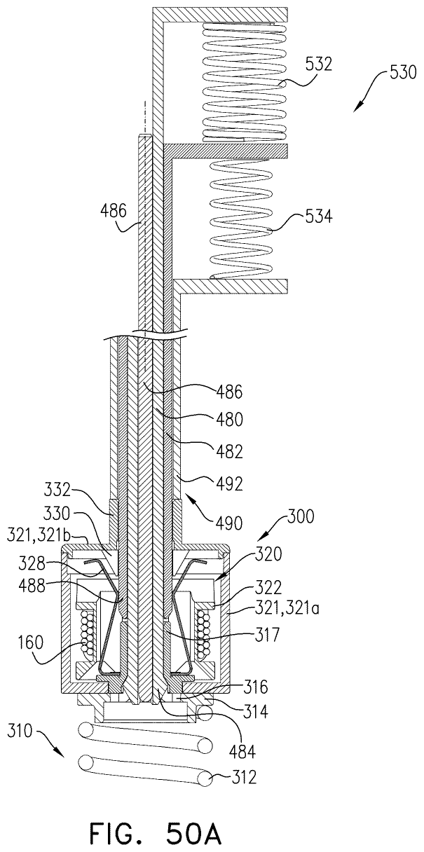

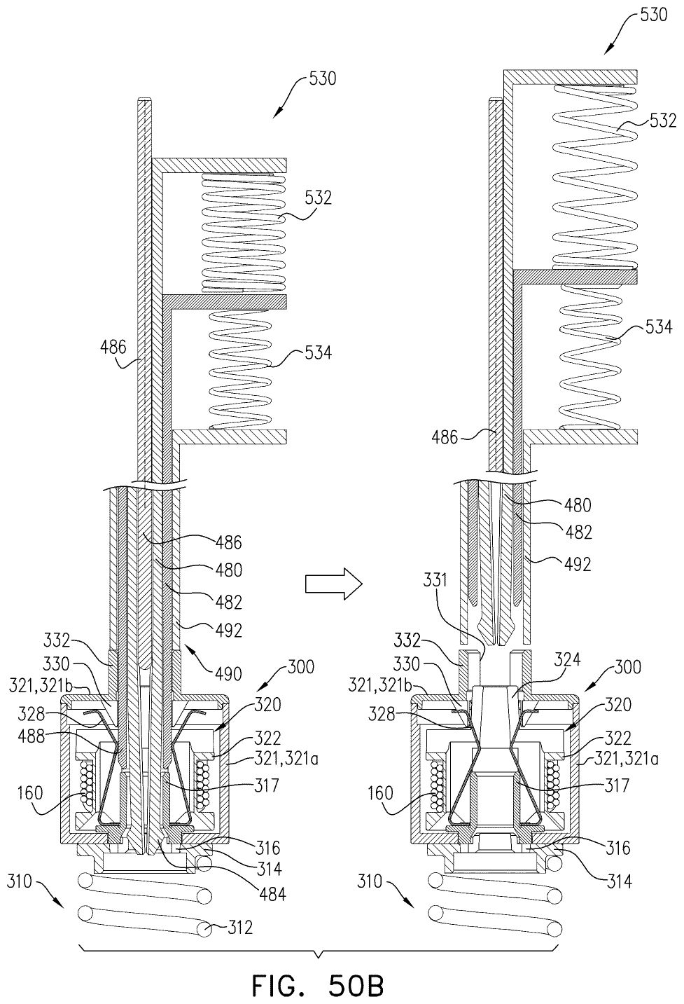

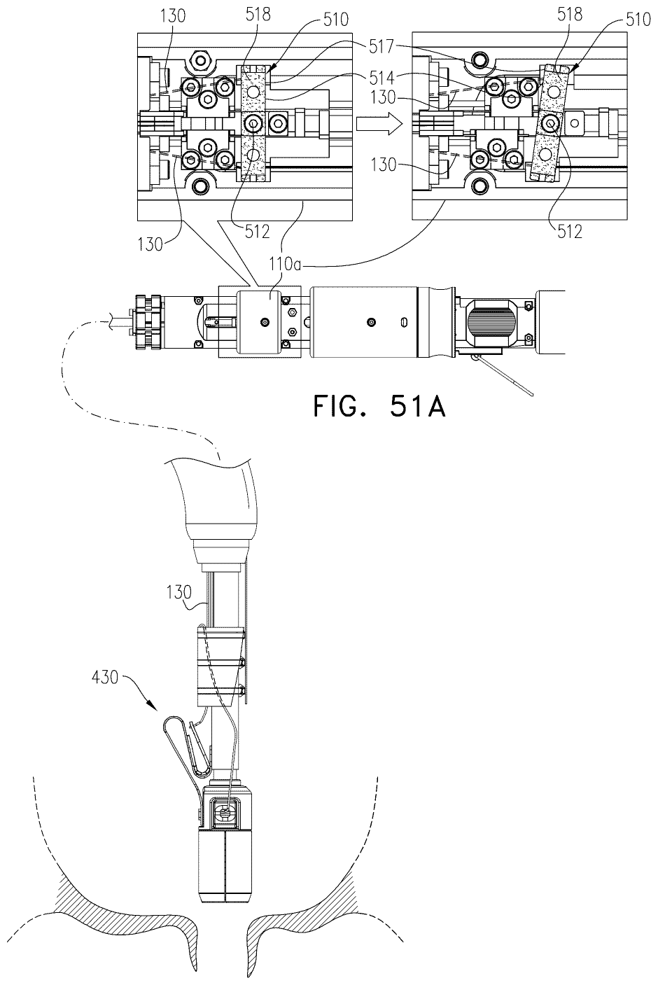

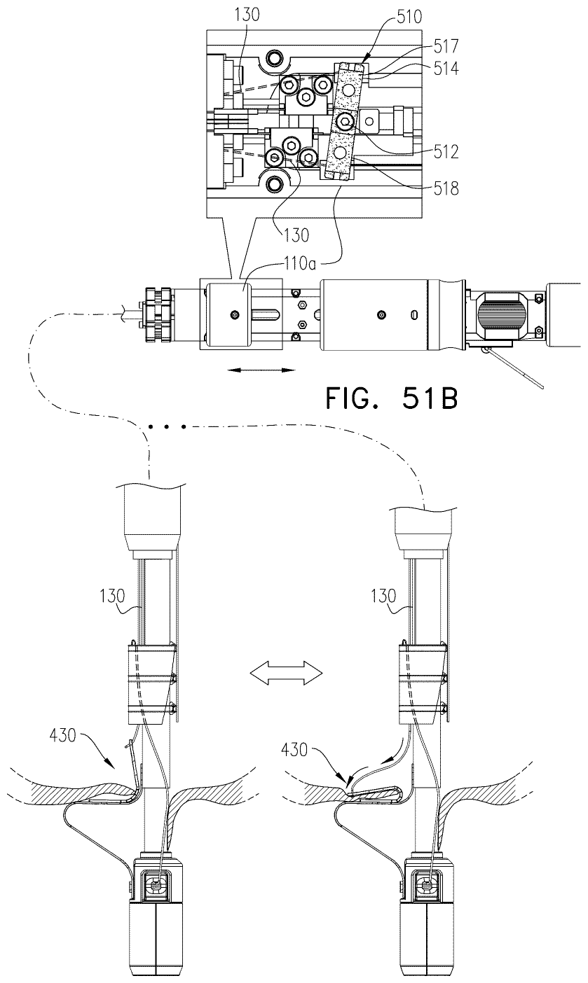

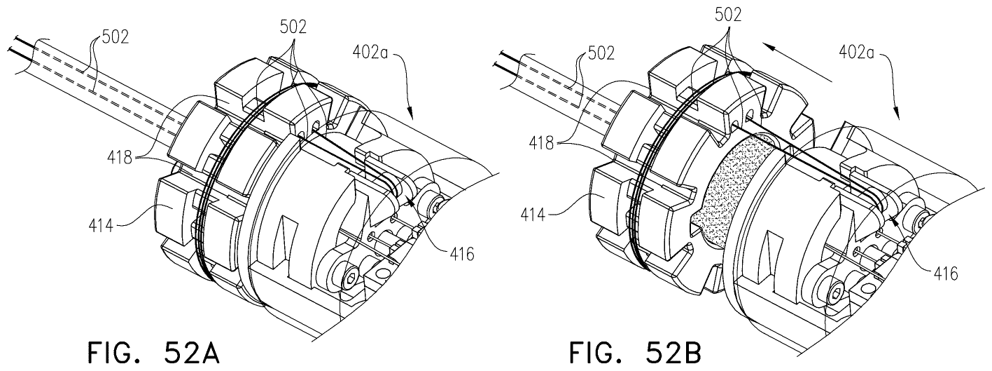

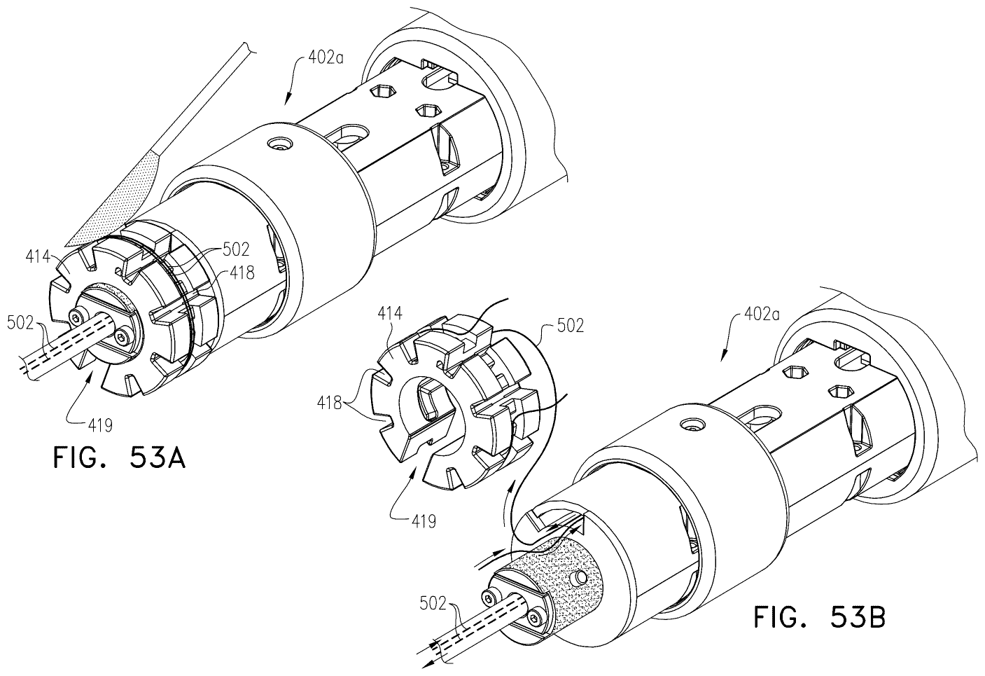

Reference is now made to FIGS. 49, 50A-B, 51A-B, 52A-B, and 53A-B, which are schematic illustrations of, inter alia, a proximal portion 402a (e.g. a handle) of a delivery tool, in accordance with some implementations. Proximal portion 402a is a variant of, and may be interchanged with, proximal portion 402 (described hereinabove), in a variant of delivery tool 400—e.g., in order to deliver and implant implant 150.

As described hereinabove, in some implementations, wraps 442 can extend around shaft 410, and are releasable by retraction of rod 446, so as to facilitate subsequent steps of the procedure e.g. the step in which mount 440 is moved into its primed position, carrying patch 210 away from shaft 410 and towards leaflet 10. In some implementations, the retraction of rod 446 is facilitated by the rod being coupled to a proximal grip 401 (e.g. loop, pin and/or handle) that is accessible at proximal portion 402a, such that pulling grip 401 proximally and/or away from the proximal portion retracts the rod from wraps 442. In some implementations, proximal portion 402a advantageously includes (e.g. defines) an interlock that prevents the mount controller 116 from being operated until rod 446 has been retracted, in order to ensure that patch 210 has been unwrapped and is therefore no longer fastened to shaft 410 by wraps 442. For example, interlock may comprise a detent 403 that is removed by pulling of grip 401 (FIG. 49).

In some implementations, proximal portion 402a comprises various interlocks to enforce the order of any or some of the parts of the sequence of implanting implant 150 as described hereinabove (e.g. as described with reference to FIGS. 2A-R).

FIGS. 50A-B illustrate a schematic illustration of a release mechanism 530 for release of the delivery tool from downstream assembly 300, in accordance with some implementations. In some implementations, in order to ensure that, once anchoring and winching of downstream assembly 300 is complete, driveshaft subassembly 490 disengages completely from the downstream assembly (e.g. and does not remain stuck and or wedged within the downstream assembly), a release mechanism 530 comprising at least one spring is provided. The release mechanism (e.g. the spring(s) thereof) is biased to pull at least one component of the driveshaft subassembly out of, and away from, downstream assembly 300.

In the example shown, release mechanism 530 comprises a first release spring 532, which may be connected to a proximal part of driveshaft 480. Spring 532 is biased to pull anchor-control driveshaft 480 proximally out of and away from (e.g. out of engagement with) anchor 310. However, during implantation and adjustment of downstream assembly 300, spring 532 is prevented from doing so by engagement (e.g. locking) between driveshaft 480 and anchor 310—e.g. this engagement resists the bias of the spring. That is, during implantation and adjustment of downstream assembly 300, spring 532 is constrained (e.g. compressed). For example, and as explained hereinabove with reference to FIGS. 4A-C, lock-rod 486 can extend through driveshaft 480, in a manner that maintains spurs 484 locked to anchor 310, thereby preventing the driveshaft from being pulled out of anchor 310 (FIG. 50A). In such implementations, disengagement of driveshaft 480 from anchor 310 (e.g., by retraction of lock-rod 486 from the distal end of driveshaft 480, as shown in FIG. 50B) triggers release spring 532 to pull the anchor-control driveshaft proximally out of away from anchor 310, e.g. by instantaneously allowing the spring to relax (e.g. expand) and pull the driveshaft in a proximal direction, thereby pulling the distal end of the driveshaft away from the anchor.

In some implementations, release mechanism 530 comprises a second release spring 534, which may be connected to a proximal part of winch-control driveshaft 482. Spring 534 is biased to pull driveshaft 482 proximally out of and away from (e.g. out of engagement with) winch 320. However, during implantation and adjustment of downstream assembly 300, spring 534 is prevented from doing so. Spring 532 may contribute to this prevention. For example, and as shown, spring 532 may be disposed operatively between driveshaft 480 and driveshaft 482 such that the same biasing of the spring that pulls driveshaft 480 proximally also pushes driveshaft 482 distally—e.g. spring 532 applies equal but opposite forces to driveshafts 480 and 482. That is, spring 532 opposes spring 534. Thus, the bias of spring 532 pushes driveshaft 482 distally in a manner that maintains the driveshaft engaged with winch 320. Spring 532 may be stronger (e.g. may have a greater spring force) than spring 534—e.g. to ensure that spring 532 sufficiently opposes spring 534.

In some implementations, due to the relationship between driveshafts 480 and 482, and springs 532 and 534 described in the preceding paragraphs, engagement between anchor-control driveshaft 480 and lock-rod 486 may, in addition to preventing spring 532 from pulling driveshaft 480 out of and away from anchor 310, also prevent spring 534 from pulling driveshaft 482 proximally out of and away from winch 320. In such implementations, retraction of lock-rod 486 from the distal end of anchor-control driveshaft 480 therefore triggers release spring 534 to pull driveshaft 482 proximally out of and away from winch 320: Responsively to the retraction of the lock-rod, spring 534 relaxes (e.g. expands), pulling driveshaft 482 proximally out of and away from the winch at the same time that spring 532 also relaxes (e.g. expands). That is, and as is shown in the transition between the two steps illustrated in FIG. 50B, in some implementations, pulling lock-rod 486 out of driveshaft subassembly 490 triggers the automatic release (e.g. ejection) of both driveshafts 480 and 482 from downstream assembly 300 by allowing the previously-constrained springs 532 and 534 to relax.

In some implementations, retraction of lock-rod 486 triggers separation of the entire driveshaft subassembly from downstream assembly 300. For example, the momentum of driveshafts 480 and 482 moving proximally may pull reference-force tube 492 away from downstream assembly 300.

Reference is now made to FIGS. 51A-B, which illustrate a feature for dynamic force-balancing between a pair of wires or rods that extend transluminally through a delivery tool, in accordance with some implementations. In the example shown, this feature is used with a pair of wires 130 that, as described hereinabove (e.g. with reference to FIGS. 2A-N), are used to transition clasp 430 between its open and closed states. However, it is to be understood that the scope of the present disclosure includes other implementations of this technique—e.g. for balancing forces between wires of other transluminally extended wires, mutatis mutandis.

In some implementations, wires 130 extend, from a clasp controller 110a (e.g. a variant of clasp controller 110), at proximal portion 402a of the delivery tool, transluminally through the delivery tool, to a distal region of the delivery tool. As shown in FIG. 51B, clasp controller 110a can be actuated by an operator sliding the clasp controller axially along proximal portion 402a, e.g. sliding clasp controller 110a in a proximal direction transitions the clasp to an open state, and sliding the clasp controller in a distal direction along the proximal portion transitions the clasp towards a closed (e.g. grasping) state.

In some implementations, the tortuous path through the vasculature along which the delivery tool is advanced results in an imbalance in tension between the two wires 130 that extend along the tool. For example, at any given curve in the delivery tool (e.g. in its shaft), should a first wire 130 of the pair be situated closer than a second wire 130 of the pair to the outside of the curve, the first wire may become tensioned more than the second wire due to its path around the curve being longer. Thus, at the distal end of the tool, the first wire may pull harder than the second wire on clasp 430, resulting in the clasp being slanted—e.g. with respect to leaflet 10 and/or downstream support 434. In order to facilitate the grasping of a sufficient extent or area of leaflet 10, it may be advantageous to ensure that the wires maintain clasp 430 level—e.g. by ensuring that actuating clasp controller 110a applies uniform force to both wires 130.

A balancing mechanism 510 that is disposed at clasp controller 110a is therefore described, that is adapted to accommodate, and therefore effectively cancel out, these imbalances. In some implementations, and as shown, balancing mechanism 510 is in the form of a lever 514 that pivots responsively to imbalances in tension between the wires. For example, the lever can have a fulcrum 512 at which clasp controller 110a is pivotably attached, and each wire 130 can be coupled to the lever at respective opposite sides 517, 518 of the fulcrum. Responsively to differences in tension between wires 130 occur (e.g. as the delivery tool is advanced transluminally towards the heart), lever 514 automatically pivots to adjust (e.g. to accommodate) these differences (e.g. as shown in the transition between the insets of FIG. 51A). Once the distal end of the delivery tool has become disposed in the heart (e.g. and while lever 514 remains pivoted to accommodate for the differences in tension between the wires), the user can simply pull and push clasp controller 110a axially along proximal portion 402a to open and close clasp 430 (FIG. 51B). In this manner, balancing mechanism 510 dynamically and automatically maintains clasp 430 in a level state with respect to the leaflet while the user actuates clasp controller 110a. It is to be noted that balancing mechanism 510 is entirely passive, and is typically not operated by the user of the delivery tool.

Similar balancing mechanisms can be additionally or alternatively used, mutatis mutandis, to balance a pair of mount-control rods 136 and/or a pair of drivers 450, e.g. by placing such a balancing mechanism (or a variant thereof) at mount controller 116 and/or driver controller 112. This may, respectively, ensure that mount 440 is advanced toward clasp 430 while level, and that both patch anchors 240 are driven through the leaflet simultaneously and to the same depth.

Reference is now made to FIGS. 52A-B, and 53A-B, which illustrate an exemplary coupling of retrieval line 502 to proximal portion 402a, in accordance with some implementations. As described hereinabove, retrieval line 502 can be coupled to one or both patch anchor 240 (or to a retrieval adapter) in a manner in which pulling on the retrieval line draws the patch anchor(s) back out of the tissue.

In some implementations, retrieval line 502 extends distally from proximal portion 402a through the delivery tool to patch anchor(s) 240, looping through the patch anchor(s) (or retrieval adapter), back to the proximal portion, such that both ends of the retrieval line are at proximal portion 402a, and a bight of the retrieval line is at the patch anchor(s). Thus, pulling on both ends of the retrieval line retrieves the patch anchor(s), while pulling on only one end of the retrieval line decouples (e.g. unloops) the retrieval line from the patch anchor(s).

FIGS. 52A-B illustrate an implementation in which both ends of retrieval line 502 are attached to a bobbin 414 that is mounted at (e.g. on) proximal portion 402a, such that the user can pull on both ends of retrieval line 502 (and thereby retrieve the patch anchor(s)) by simply moving the bobbin. In some implementations, each end of retrieval line 502 extends, from the bobbin, proximally along proximal portion 402a towards a bearing 416 on the proximal portion, and, at the bearing surface, turns distally back on itself, where it extends distally through the delivery tool, past the bobbin and towards the implant (FIG. 52A). This arrangement is such that sliding the bobbin distally along proximal portion 402a causes the loop at the distal end of the retrieval line to be pulled proximally (FIG. 52B), thereby retrieving the patch anchor(s). However, it is to be understood that although FIG. 52B shows bearing 416 being disposed on proximal portion 402a more proximally than bobbin 414 (e.g. such that the bobbin can be slid distally along proximal portion 402a to induce tension in the retrieval line), it is to be understood that a bearing could be disposed more distally to the bobbin, mutatis mutandis.

FIGS. 53A-B illustrate the withdrawal of retrieval line 502 from the patch anchor(s), e.g. once it has been determined that implantation of patch anchor(s) is complete, and that retrieval of the anchor(s) is not necessary. As described hereinabove, this can be achieved by pulling a single end of retrieval line 502, to unloop the retrieval line from around the patch anchor(s) and pull the retrieval line out of the heart. For some implementations, bobbin 414 may be configured to facilitate this, by facilitating detachment of one end (a first end 502′) of the retrieval line from the bobbin while the other end (a second end 502″) remains attached to the bobbin. First end 502′ of the retrieval line can be wrapped circumferentially around bobbin 414. In order to facilitate the pulling of a single end of the retrieval line, the bobbin can define one or more troughs 418 that are spaced radially around a circumference of the bobbin, such that first end 502′ of the retrieval line is accessible (e.g. presented) for cutting via one of troughs 418 (e.g. using a scalpel as illustrated in FIG. 53A), thereby allowing detachment of the first end from the bobbin and providing a free end 513 of the retrieval line.

In order to subsequently withdraw the retrieval line from the anchor(s), as shown in FIG. 53B, bobbin 414 can then be dismounted from proximal portion 402a, e.g. by pulling the bobbin laterally off the proximal portion (e.g. via a lateral slit 419 of the bobbin), and away from the proximal portion, the bobbin pulling second end 502″ with it. As shown in FIG. 53B, this causes second end 502″ to be pulled out of the delivery tool while free end 513 at first end 502′ is concurrently pulled into the delivery tool as retrieval line 502 progressively slides through and out of the anchor(s) and/or retrieval adapter at downstream assembly 300. Once free end 513 reaches and becomes unlooped from the downstream assembly, retrieval line 502 is effectively disabled, and may be removed from the subject along with, or independently from, the delivery tool.

For some implementations, and as shown, bobbin 414 defines a series of troughs, arranged circumferentially around the bobbin, thereby advantageously providing access to the retrieval line irrespective of a rotational orientation of proximal portion 402a with respect to the user.

Reference is again made to FIGS. 1A-53B. Although the different terms “tether,” “cord,” and “line,” are used to distinguish one element from another within the specification and the claims, it is to be understood that each of these components can be considered to be a longitudinal member. The structural, mechanical, and/or chemical properties of these elements can be different to that of the others, or many be the same as that of the others. For example, each of these elements can comprise a natural or synthetic suture, can comprise a metal and/or a polymer, or can be braided or a monofilament.

Reference is again made to FIGS. 1A-53B. It is to be understood that the particular positions in which downstream assemblies are shown to be anchored are by way of illustration and not limitation. Similarly, the particular leaflet, and part of the leaflet, to which the upstream assemblies are shown to be anchored are by way of illustration and not limitation.

Reference is yet again made to FIGS. 1A-53B. Although the techniques described herein are generally shown as being performed on the left ventricle and/or the mitral valve, the techniques can also be performed, mutatis mutandis, on the right ventricle and/or tricuspid valve. Additionally, techniques described herein can be used during open-heart, minimally-invasive, and transcatheter procedures, mutatis mutandis.

The systems, methods, etc. herein can include adjusting a length of the artificial chords (tethers) following initial implantation (e.g., once the delivery tools have been extracted from within the body) in response to the application of energy (e.g., radiofrequency or ultrasound) toward the heart from a source of energy disposed externally to the body of the patient.

Any of the various systems, devices, apparatuses, components, etc. in this disclosure can be sterilized (e.g., with heat, radiation, ethylene oxide, hydrogen peroxide, etc.) to ensure they are safe for use with patients, and any of the methods herein can include sterilization of the associated system, device, apparatus, component, etc. (e.g., with heat, radiation, ethylene oxide, hydrogen peroxide, etc.) as one of the steps of the method.

As appropriate, techniques described herein can be practiced in conjunction with methods, systems, and apparatuses described in one or more of the following patent applications, each of which is assigned to the assignee of the present application and is incorporated herein by reference for all purposes:
U.S. patent application Ser. No. 12/435,291 to Maisano et al., entitled “Adjustable repair chords and spool mechanism therefor,” filed on May 4, 2009, which issued as U.S. Pat. No. 8,147,542;
U.S. patent application Ser. No. 12/437,103 to Zipory et al., entitled “Annuloplasty ring with intra-ring anchoring,” filed on May 7, 2009, which issued as U.S. Pat. No. 8,715,342;
U.S. patent application Ser. No. 12/548,991 to Maisano et al., entitled “Implantation of repair chords in the heart,” filed on Aug. 27, 2009, which issued as U.S. Pat. No. 8,808,368;
International Patent Application No. PCT/IL2009/001209 to Cabiri et al., entitled “Adjustable annuloplasty devices and mechanisms therefor,” filed on Dec. 22, 2009, which published as PCT Publication No. WO 2010/073246;
International Patent Application No. PCT/IL2010/000357 to Maisano et al., entitled “Implantation of repair chords in the heart,” filed on May 4, 2010, which published as WO 2010/128502; and/or
International Patent Application No. PCT/IL2010/000358 to Zipory et al., entitled “Deployment techniques for annuloplasty ring and over-wire rotation tool,” filed on May 4, 2010, which published as WO 2010/128503.
International Patent Application No. PCT/IB2021/060436 to Tennenbaum et al., entitled “Valve leaflet treatment systems and methods,” filed on Nov. 11, 2021, which published as WO 2022/101817.

Example Applications (some non-limiting examples of the concepts herein are recited below):

Example 1. A system for use with a valve disposed between an atrium and a ventricle of a heart of a subject, the system comprising:
an implant, comprising:
a patch, comprising a flexible sheet,
a patch anchor,
a downstream assembly, comprising a ventricular anchor, and
a tether, tethering the downstream assembly to the patch; and
a delivery tool, having a distal portion transluminally advanceable to the heart while the implant is mounted on the delivery tool, the delivery tool comprising:
a shaft, defining a longitudinal axis of the delivery tool;
a clasp, comprising an upstream support and a downstream support, the clasp being transitionable between:
an open state in which the upstream support and the downstream support are positioned away from each other, and in which the clasp is configured to receive a portion of a leaflet of the valve between the upstream support and the downstream support, and
a grasping state, the clasp being configured to grasp the portion of the leaflet received between the upstream support and the downstream support by being transitioned from the open state toward the grasping state while the portion of the leaflet remains disposed between the upstream support and the downstream support; and
a driver, configured to anchor the patch to the portion of the leaflet using the patch anchor while the portion of the leaflet remains grasped by the clasp.

Example 2. The system according to example 1, wherein the clasp is transitionable toward the open state subsequently to anchoring of the patch to the leaflet in order to release, from the clasp, the portion of the leaflet with the patch anchored thereto.

Example 3. The system according to any one of examples 1-2, wherein in the grasping state, the upstream support and the downstream support are closer to each other than in the open state.

Example 4. The system according to any one of examples 1-3, wherein:
the ventricular anchor is a first ventricular anchor,
the downstream assembly further comprises a second ventricular anchor, and the tether tethers the patch to both the first ventricular anchor and the second ventricular anchor.

Example 5. The system according to any one of examples 1-4, wherein the patch comprises a first part of the sheet, and a second part of the sheet is shaped to extend away from the patch in a manner that defines the tether.

Example 6. The system according to any one of examples 1-5, wherein the clasp comprises a grasping indicator, flexibly coupled to the upstream support in a manner in which, upon grasping of the portion of the leaflet between the upstream support and the downstream support, the portion of the leaflet moves the grasping indicator with respect to the upstream support in a manner that is detectable fluoroscopically.

Example 7. The system according to any one of examples 1-6, wherein the patch anchor is coupled to the patch in a manner that facilitates the anchoring of the patch to the portion of the leaflet by:
allowing the driver to temporarily move the patch anchor away from the patch while the patch anchor remains coupled to the patch, and
biasing the patch anchor to return toward the patch.

Example 8. The system according to any one of examples 1-7, wherein the delivery tool is configured such that a steerable part of the shaft, distal from the clasp, is steerable via operation of an extracorporeal proximal portion of the delivery tool.

Example 9. The system according to any one of examples 1-8, wherein the implant is mounted or mountable on the delivery tool such that the tether extends from the downstream assembly, alongside the shaft, past the clasp, and to the patch.

Example 10. The system according to any one of examples 1-9, wherein the clasp, in both the open state and the grasping state, is disposed entirely laterally from the shaft.

Example 11. The system according to any one of examples 1-10, wherein the ventricular anchor comprises a helical tissue-engaging element.

Example 12. The system according to any one of examples 1-11, wherein the tether extends from the downstream assembly to the patch, and back to the downstream assembly.

Example 13. The system according to example 12, wherein:
the ventricular anchor is a first ventricular anchor,
the downstream assembly further comprises a second ventricular anchor, and
the tether extends from the first ventricular anchor to the patch, and back to the second ventricular anchor.

Example 14. The system according to example 12, wherein:
the implant comprises an upstream assembly comprising the patch and the patch anchor, and
the tether is slidably coupled to the upstream assembly.

Example 15. The system according to example 14, wherein the upstream assembly defines an eyelet, and wherein the tether is slidably coupled to the upstream assembly by being threaded through the eyelet.

Example 16. The system according to example 14, wherein:
the downstream assembly comprises a winch coupled to the ventricular anchor, and the tether is arranged in a pulley arrangement in which:
a first end of the tether is operatively coupled to the winch,
a bight of the tether is slidably coupled to the upstream assembly, and
a second end of the tether is fixed to the downstream assembly.

Example 17. The system according to example 16, wherein the winch has a housing, fixedly attached to the ventricular anchor, and wherein the second end of the tether is fixed to the housing.

Example 18. The system according to any one of examples 1-17, wherein the patch has a lip region, and wherein the tether is attached to the patch via two lateral lines that diverge away from the tether and from each other, and that are attached to opposing lateral sites in the lip region.

Example 19. The system according to example 18, wherein the attachment of the tether to the patch via the two lateral lines is such that tension applied to the tether flexes the patch medially, the patch being configured to elastically flex medially.

Example 20. The system according to example 19, further comprising a medial line connecting the tether to a medial site in the lip region in a manner that limits an extent to which tension applied to the tether flexes the patch medially.

Example 21. The system according to any one of examples 1-20, wherein:
the downstream assembly further comprises a winch, the ventricular anchor being a winch anchor that is coupled to the winch, and
the tether tethers the winch to the patch.

Example 22. The system according to example 21, wherein:
the winch comprises a housing and a spool disposed therein, the spool operatively coupled to the tether such that actuation of the winch tensions the tether,
the tether extends, from the spool and out of an aperture of the housing, the aperture having a rim, and
the downstream assembly further comprises a spring, coupled to the housing in a manner that urges the tether away from contact with the rim.

Example 23. The system according to example 22, wherein the spring is a volute spring.

Example 24. The system according to example 22, wherein the spring is a cantilever spring.

Example 25. The system according to example 22, wherein the spring is a wave spring.

Example 26. The system according to example 22, wherein the spring is coupled to the housing in a manner that urges the tether away from contact with a side of the rim that is furthest away from the winch anchor.

Example 27. The system according to example 22, wherein the downstream assembly comprises a helix that defines:
the spring, and
a gripping region adapted to grip the tether.

Example 28. The system according to example 22, wherein the spring defines a helix having a series of turns.

Example 29. The system according to example 28, wherein the helix extends circumferentially around an exterior of the winch housing.

Example 30. The system according to example 28, wherein the spring is adapted to grip the tether in between the turns of the helix.

Example 31. The system according to example 21, wherein the delivery tool further comprises a driveshaft subassembly, the driveshaft subassembly comprising one or more driveshafts, extending through the shaft, and operatively coupled to the downstream assembly in a manner that configures the driveshaft subassembly:
to anchor the winch anchor to ventricular tissue of the heart by applying an anchoring force to the winch anchor, and
to actuate the winch independently of applying the anchoring force.

Example 32. The system according to example 31, wherein:
the delivery tool is configured to actuate the winch by applying torque to the winch via the driveshaft subassembly, and
the downstream assembly comprises a slip clutch that operatively couples the driveshaft subassembly to the winch in a manner that limits a magnitude of torque that the delivery tool may apply to the winch.

Example 33. The system according to example 31, wherein:
the driveshaft subassembly comprises a reference-force tube that extends through the shaft and is engaged with the downstream assembly, and
the one or more driveshafts extend through the reference-force tube to the downstream assembly, and
the driveshaft subassembly is configured to actuate the winch by applying torque to the winch while the reference-force tube provides a reference force to the downstream assembly.

Example 34. The system according to example 21, wherein the downstream assembly and the delivery tool are configured to facilitate the delivery tool rotating the winch anchor with respect to the shaft without actuating the winch.

Example 35. The system according to any one of examples 1-34, wherein the driver is configured to anchor the patch to the portion of the leaflet by driving the patch anchor through the portion of the leaflet grasped by the clasp.

Example 36. The system according to example 35, wherein the patch anchor is a toggle that is biased to automatically widen upon deployment.

Example 37. The system according to example 36, wherein the toggle has a cellular structure that is biased to automatically widen by foreshortening.

Example 38. The system according to any one of examples 1-37, wherein the delivery tool is configured to anchor the downstream assembly to ventricular tissue of the ventricle by anchoring the ventricular anchor to the ventricular tissue.

Example 39. The system according to example 38, wherein the ventricular anchor comprises a tissue-engaging element, and the delivery tool is configured to anchor the downstream assembly to the ventricular tissue by driving the tissue-engaging element into the ventricular tissue.

Example 40. The system according to example 39, wherein the implant is mounted or mountable on the delivery tool such that the ventricular anchor is disposed at a distal end of the shaft.

Example 41. The system according to example 40, wherein the delivery tool further comprises a driveshaft subassembly, the driveshaft subassembly comprising one or more driveshafts extending through the shaft and operatively coupled to the downstream assembly in a manner that configures the driveshaft subassembly to anchor the ventricular anchor to the ventricular tissue by applying an anchoring force to the ventricular anchor.

Example 42. The system according to example 40, wherein the delivery tool comprises a capsule coupled to a distal end of the shaft, the distal portion of the delivery tool being transluminally advanceable to the heart while the downstream assembly is housed within the capsule.

Example 43. The system according to example 42, wherein the capsule comprises a shroud formed from a resilient polymer.

Example 44. The system according to example 43, wherein the capsule further comprises a housing having multiple fingers that are flexible, distributed circumferentially to approximate a tubular shape, and embedded within the shroud.

Example 45. The system according to example 40, wherein:
the downstream assembly further comprises a winch, the ventricular anchor being a winch anchor that is coupled to the winch, and
the delivery tool further comprises a driveshaft subassembly, the driveshaft subassembly comprising one or more driveshafts, extending through the shaft, and operatively coupled to the downstream assembly in a manner that configures the driveshaft subassembly to screw the tissue-engaging element into the ventricular tissue by applying torque to the winch anchor without rotating the winch with respect to the ventricular tissue.

Example 46. The system according to example 45, wherein the distal portion of the delivery tool is coupled to the implant in a manner that configures the driveshaft subassembly to screw the tissue-engaging element into the ventricular tissue by applying the torque to the winch anchor without rotating the winch with respect to the shaft.

Example 47. The system according to example 46, wherein:
the delivery tool comprises a capsule coupled to a distal end of the shaft, the distal portion of the delivery tool being transluminally advanceable to the heart while the downstream assembly is housed within the capsule, and
the capsule and the winch are shaped to inhibit rotation of the winch with respect to the shaft while the driveshaft subassembly screws the tissue-engaging element into the ventricular tissue.

Example 48. The system according to example 47, wherein:
the capsule defines a track with which the winch is engaged while housed by the capsule, and
the capsule and the downstream assembly configure the driveshaft subassembly to screw the tissue-engaging element into the ventricular tissue in a manner in which the downstream assembly advances distally out of the capsule, with the winch sliding linearly along the track.

Example 49. The system according to example 48, wherein:
the track is a lateral opening in the capsule, the winch defines an aperture through which the tether extends out of the winch to the patch, and
protrusion of the aperture into the lateral opening configures the driveshaft subassembly to screw the tissue-engaging element into the ventricular tissue in a manner in which the downstream assembly advances distally out of the capsule, with the aperture of the winch sliding linearly along the lateral opening.

Example 50. The system according to example 49, wherein the capsule comprises:
a housing that houses the winch and that defines the lateral opening; and
a resilient shroud that shrouds the housing.

Example 51. The system according to example 50, wherein:
the shroud shrouds a distal region of the lateral opening, leaving a proximal region of the lateral opening exposed to define a window of the capsule, and
the distal portion of the delivery tool is transluminally advanceable to the heart while the downstream assembly is housed within the capsule with the aperture exposed at the window.

Example 52. The system according to example 51, wherein the shroud defines a slit that extends distally from the window, aligned with the lateral opening.

Example 53. The system according to example 52, wherein protrusion of the aperture into the lateral opening configures the driveshaft subassembly to screw the tissue-engaging element into the ventricular tissue in a manner in which the downstream assembly advances distally out of the capsule, with the aperture of the winch transiently separating the shroud at the slit as the aperture slides linearly along the lateral opening.

Example 54. The system according to any one of examples 1-53, wherein the implant comprises an upstream assembly comprising the patch anchor coupled to the patch.

Example 55. The system according to example 54, wherein the upstream assembly further comprises a cord via which the patch anchor is coupled to the patch.

Example 56. The system according to example 55, wherein the patch anchor is a toggle anchor.

Example 57. The system according to example 56, wherein the toggle anchor is a helical coil that defines a lumen therethrough.

Example 58. The system according to example 57, wherein the driver is configured to drive the anchor through the leaflet while the driver extends through the lumen.

Example 59. The system according to example 57, further comprising a retrieval line that extends away from the toggle anchor, the retrieval line being threaded through turns of the coil in a manner in which tensioning the retrieval line stiffens the anchor by compressing the turns against each other.

Example 60. The system according to example 57, wherein the helical coil extends helically around and along a toggle axis, and the system further comprises a retrieval line that extends along the toggle axis and away from the toggle anchor in a manner in which tensioning the retrieval line stiffens the anchor by compressing the turns against each other.

Example 61. The system according to example 60, wherein the retrieval line is fixed to a first end of the toggle anchor and extends along the toggle axis to a second end of the toggle anchor and, from the second end of the toggle anchor, away from the toggle anchor.

Example 62. The system according to example 57, wherein the cord is connected to a midportion of the coil.

Example 63. The system according to example 62, wherein the cord is connected to the midportion by looping around a turn of the coil.

Example 64. The system according to example 56, wherein:

the toggle anchor has a tip, a heel, and an eyelet partway between the tip and the heel, and
the heel is flared in a manner that:
facilitates passage of the heel through the leaflet in a first direction, and inhibits passage of the heel through the leaflet in a second direction that is opposite to the first direction.

Example 65. The system according to example 64, wherein the heel defines wings adapted to transiently flex medially toward each other during passage of the heel through the leaflet in the first direction.

Example 66. The system according to example 64, wherein the heel defines wings adapted to flex laterally away from each other upon the heel being pushed against the leaflet in the second direction.

Example 67. The system according to example 56, wherein:
the toggle anchor has a tip, a heel, and a lateral eyelet partway between the tip and the heel, and
the cord is connected to the toggle anchor via the lateral eyelet in a manner in which tensioning the cord extends the heel away from the lateral eyelet.

Example 68. The system according to example 67, wherein:
the toggle anchor has a sharp point, and
the cord is connected to the toggle anchor via the lateral eyelet in a manner in which tensioning the cord concurrently (i) extends the heel away from the lateral eyelet and (ii) retracts the point toward the lateral eyelet.

Example 69. The system according to example 67, wherein the system further comprises a retrieval line, threaded through the toggle anchor in a manner in which tensioning the retrieval line retracts the heel toward the lateral eyelet.

Example 70. The system according to example 69, wherein the toggle anchor further comprises a spring, configured to bias the heel to extend away from the lateral eyelet.

Example 71. The system according to example 70, wherein the toggle anchor has a sharp point, and the spring is configured to bias the point to retract toward the lateral eyelet.

Example 72. The system according to example 56, wherein:
the toggle anchor has a tip, a heel, and a lateral eyelet partway between the tip and the heel,
a first segment of the toggle anchor defines the tip,
a second segment of the toggle anchor is slidably coupled to the first segment, and the system further comprises a longitudinal member, extending through the lateral eyelet, and connected to the toggle anchor in a manner in which pulling of the longitudinal member slides the second segment axially with respect to the first segment.

Example 73. The system according to example 72, wherein the second segment of the toggle anchor defines the heel.

Example 74. The system according to example 72, wherein the driver is configured to push the toggle anchor tip-first through the portion of the leaflet, the driver having a drive head, and a rod extending proximally from the drive head, the drive head being connected to the heel via complimentary geometry in a manner that (i) preferentially allows deflection rather than lateral translation of the toggle anchor with respect to the driver, and (ii) allows the heel to disconnect from the driver upon the toggle anchor reaching a predetermined angle with respect to the driver.

Example 75. The system according to example 74, wherein:
the drive head has a first distally-facing face, and defines a shoulder that defines a second distally-facing face proximal from the first face,
at the heel, the toggle anchor defines a lateral opening through which the shoulder protrudes, such that:

the driver is configured to push the toggle anchor tip-first through the portion of the leaflet by (i) the second distally-facing face pushing distally on the toggle anchor at the lateral opening, and (ii) the first distally-facing face pushing distally on the toggle anchor substantially opposite the lateral opening, and the toggle anchor is allowed to disconnect from the driver by deflecting about a point on the driver proximal from the second distally-facing face such that the lateral opening moves laterally away from the shoulder.

Example 76. The system according to example 74, wherein:
the drive head defines a knob, and a neck that connects the knob to the rod, at the heel, the toggle anchor defines appendages that extend proximally beyond the knob and, proximally from the knob, medially toward each other and toward the neck, such that:
the appendages inhibit proximal retraction and lateral translation of the driver from the toggle anchor, and
deflection of the toggle anchor with respect to the driver urges the knob between the appendages such that the appendages deflect laterally away from each other and from the neck.

Example 77. The system according to example 74, wherein:
the drive head defines a socket that has a rim,
at the heel, the toggle anchor defines a knob,
the knob is disposed in the socket in a manner that inhibits lateral translation of the toggle anchor from the driver, and
deflection of the toggle anchor with respect to the driver presses the rim against the toggle anchor in a manner that levers the knob distally out of the socket.

Example 78. The system according to example 55, wherein coupling of the tether to the upstream assembly is such that pulling on the tether pulls on the cord in a manner that draws the patch anchor toward the patch.

Example 79. The system according to example 55, wherein the upstream assembly comprises a one-way mechanism through which the cord extends, the one-way mechanism being:
mounted on the patch,
configured to facilitate passage of the cord through the one-way mechanism in a first direction that draws the patch anchor toward the patch, and
configured to inhibit passage of the cord through the one-way mechanism in a second direction that is opposite to the first direction.

Example 80. The system according to example 79, wherein the upstream assembly is configured such that pulling on the tether pulls the cord through the one-way mechanism in the first direction.

Example 81. The system according to example 80, wherein the delivery tool is configured to pull on the tether such that the tether pulls the cord through the one-way mechanism in the first direction.

Example 82. The system according to example 81, wherein the delivery tool is configured to pull on the tether by moving the downstream assembly away from the upstream assembly subsequently to anchoring the patch to the portion of the leaflet.

Example 83. The system according to example 55, wherein the patch anchor has a sharpened tip, and is configured to be driven by the driver through the leaflet with the sharpened tip penetrating the leaflet.

Example 84. The system according to example 55, wherein the delivery tool further comprises a hollow needle, and wherein the patch anchor is configured to be driven by the driver through the leaflet while disposed within the hollow needle.

Example 85. The system according to example 55, wherein the delivery tool further comprises a hollow needle configured to pierce the leaflet, and wherein the driver is configured to drive the patch anchor out of the hollow needle while the hollow needle extends through the leaflet.

Example 86. The system according to example 55, wherein the patch anchor comprises a toggle that defines an eyelet partway along the toggle, the cord being attached to the patch anchor at the eyelet.

Example 87. The system according to example 86, wherein the eyelet extends transversely entirely through the toggle.

Example 88. The system according to example 86, wherein the toggle is substantially tubular, having a lateral wall that defines a lumen.

Example 89. The system according to example 55, wherein the upstream assembly further comprises a spring configured to tension the cord.

Example 90. The system according to example 89, wherein the spring is a compression spring.

Example 91. The system according to example 89, wherein the spring lies substantially flat with respect to the patch.

Example 92. The system according to example 89, wherein the spring is configured to facilitate the driver driving the patch anchor through the leaflet by transiently straining in response to tension applied to the cord by the driver pushing the patch anchor away from the patch and through the leaflet.

Example 93. The system according to example 92, wherein the spring is coupled to the sheet in a manner in which the patch transiently linearly contracts as the spring transiently strains.

Example 94. The system according to example 92, wherein the spring is coupled to the sheet in a manner in which the spring slides across the sheet as the spring transiently strains.

Example 95. The system according to example 92, wherein:
the patch has a lip and a root,
the driver is configured to anchor the root of the patch to the leaflet such that the lip of the patch extends toward an opposing leaflet of the valve, and
the patch comprises at least one frame that defines:

a lip brace at the lip of the patch, and a root brace at the root of the patch.

Example 96. The system according to example 95, wherein the spring is configured such that the transient straining consists substantially of transient compression of the spring between the lip brace and the root brace.

Example 97. The system according to example 95, wherein the at least one frame defines a patch-anchor support coupled to the root brace, the cord extending from the spring, through the patch-anchor support, to the patch anchor.

Example 98. The system according to example 95, wherein the tether is connected to the lip brace.

Example 99. The system according to example 95, wherein the spring is attached to the root brace.

Example 100. The system according to example 99, wherein the spring extends from the root brace to the lip brace.

Example 101. The system according to example 100, wherein the spring extends from the root brace to the lip brace along a midline of the patch.

Example 102. The system according to example 99, wherein the spring does not extend to the lip brace.

Example 103. The system according to any one of examples 1-102, wherein the clasp defines slot, and the driver is configured to anchor the patch to the leaflet by driving the patch anchor through the leaflet and the slot.

Example 104. The system according to example 103, wherein the clasp defines a resilient tooth configured to facilitate the patch anchor being driven by the driver through the slot, and to inhibit the patch anchor from being withdrawn, in a reverse direction, through the slot.

Example 105. The system according to example 104, wherein the tooth is configured to be transiently pushed aside by the patch anchor being driven by the driver through the slot.

Example 106. The system according to example 103, wherein the delivery tool is configured to orient the driver with respect to the slot, such that, as the driver drives the patch anchor through the slot, the patch anchor rubs along a rim of the slot.

Example 107. The system according to example 103, wherein the slot is defined by the downstream support of the clasp.

Example 108. The system according to example 103, wherein the clasp defines a slot guard, configured to obstruct tissue of the heart from entering the slot.

Example 109. The system according to example 108, wherein the patch is coupled to the patch anchor via a cord, and the slot guard:
is resilient,
has a resting position in which it covers an entrance to the slot, thereby obstructing tissue of the heart from entering the slot, and
is transiently deflectable away from the slot by the cord, thereby facilitating exiting of the cord from the slot.

Example 110. The system according to example 109, wherein a free end of the slot guard is tucked underneath the downstream support.

Example 111. The system according to any one of examples 1-110, wherein the delivery tool further comprises a capsule at a distal end of the shaft, the capsule configured to house the downstream assembly.

Example 112. The system according to example 111, wherein the capsule comprises a shroud formed from a resilient polymer.

Example 113. The system according to example 112, wherein the capsule further comprises a housing having multiple fingers that are flexible, distributed circumferentially to approximate a tubular shape, and embedded within the shroud.

Example 114. The system according to example 111, wherein the capsule is shaped to define a lateral window therein.

Example 115. The system according to example 114, wherein the capsule is shaped to define a narrow slit that extends between the lateral window and an open distal end of the capsule.

Example 116. The system according to any one of examples 1-115, wherein the delivery tool has an extracorporeal proximal portion that comprises a clasp controller operatively coupled to the clasp such that operation of the clasp controller transitions the clasp between the open state and the grasping state.

Example 117. The system according to example 116, wherein the clasp controller is operatively coupled to the upstream support of the clasp such that operation of the clasp controller transitions the clasp between the open state and the grasping state via movement of the upstream support with respect to the shaft.

Example 118. The system according to example 117, wherein:
the delivery tool further comprises a pair of clasp-control wires via which the clasp controller is operatively coupled to the clasp, and
the extracorporeal portion comprises a lever:

via which the clasp controller is operatively coupled to both wires of the pair, and adapted to pivot in a manner that balances the wires of the pair with respect to each other.

Example 119. The system according to example 118, wherein the lever has a fulcrum at which the clasp controller is pivotably attached to the lever, and wherein each wire of the pair is coupled to the lever at respective opposite sides of the fulcrum.

Example 120. The system according to example 116, wherein the extracorporeal proximal portion further comprises a driver controller operatively coupled to the driver such that operation of the driver controller induces the driver to anchor the patch anchor to the leaflet.

Example 121. The system according to example 120, wherein:
the patch anchor is a first patch anchor,
the implant further comprises a second patch anchor,
the driver is a first driver, configured to anchor the patch to the portion of the leaflet by pushing the first patch anchor through the portion of the leaflet,
the delivery tool further comprises a second driver, configured to anchor the patch to the portion of the leaflet by pushing the second patch anchor through the portion of the leaflet, and
the extracorporeal portion comprises a lever:

via which the driver controller is operatively coupled to the first and second drivers, and adapted to pivot in a manner that balances the first driver with the second driver.

Example 122. The system according to example 121, wherein the lever has a fulcrum at which the driver controller is pivotably attached to the lever, and wherein the first and second drivers are coupled to the lever at respective opposite sides of the fulcrum.

Example 123. The system according to example 116, wherein:
within the distal portion of the delivery tool the shaft has a proximal part and a distal part, and
the extracorporeal proximal portion of the delivery tool further comprises a shaft extender, operatively coupled to the shaft such that operation of the shaft extender reversibly extends the distal part of the shaft distally from the proximal part of the shaft.

Example 124. The system according to example 123, wherein the clasp is coupled to the shaft such that extension of the distal part of the shaft distally from the proximal part of the shaft deflects the downstream support with respect to the shaft.

Example 125. The system according to example 124, wherein:
the delivery tool comprises a frame that defines the downstream support, and
a first part of the frame is attached to the proximal part of the shaft, and a second part of the frame is attached to the distal part of the shaft, such that adjusting a degree of extension of the distal part of the shaft from the proximal part of the shaft deflects the downstream support with respect to the shaft.

Example 126. The system according to example 125, wherein the distal part of the shaft includes a steerable part, and wherein the attachment of the first part of the frame and the second part of the frame to the proximal part of the shaft and the second part of the shaft, respectively, is such that extension of the distal part of the shaft distally from the proximal part of the shaft beyond a threshold extent causes the frame to pull the distal part of the shaft to deflect.

Example 127. The system according to example 124, wherein the clasp is coupled to the shaft such that extension of the distal part of the shaft distally from the proximal part of the shaft deflects both the downstream support and the upstream support with respect to the shaft.

Example 128. The system according to example 127, wherein the clasp is coupled to the shaft such that extension of the distal part of the shaft distally from the proximal part of the shaft deflects both the downstream support and the upstream support with respect to the shaft without changing a disposition between the downstream support and the upstream support.

Example 129. The system according to example 127, wherein the clasp is coupled to the shaft such that extension of the distal part of the shaft distally from the proximal part of the shaft deflects both the downstream support and the upstream support with respect to the shaft while the clasp remains in the grasping state.

Example 130. The system according to example 116, wherein:
the extracorporeal proximal portion further comprises an anchor controller, and
the delivery tool further comprises a driveshaft subassembly that comprises one or more driveshafts extending through the shaft, the driveshaft subassembly configured such that, in at least one state of the delivery tool, the driveshaft subassembly operatively couples the anchor controller to the ventricular anchor such that operation of the anchor controller applies an anchoring force to the ventricular anchor.

Example 131. The system according to example 130, wherein:
the downstream assembly further comprises a winch, the ventricular anchor being a winch anchor that is coupled to the winch,
the tether tethers the winch to the patch, and
the extracorporeal proximal portion further comprises a winch controller, the driveshaft subassembly configured such that, in at least one state of the delivery tool, the driveshaft subassembly operatively couples the winch controller to the winch such that operation of the winch controller actuates the winch.

Example 132. The system according to example 131, wherein:
operation of the winch controller actuates the winch by applying torque to the winch via the driveshaft subassembly, and
the downstream assembly comprises a slip clutch that operatively couples the driveshaft subassembly to the winch in a manner that limits a magnitude of torque that the driveshaft assembly may apply to the winch.

Example 133. The system according to example 131, wherein the driveshaft subassembly comprises:
a winch-control driveshaft via which the winch controller is operatively coupled to the winch, and
an anchor-control driveshaft disposed through the winch-control driveshaft, and via which the anchor controller is operatively coupled to the anchor.

Example 134. The system according to example 133, wherein:
the anchor-control driveshaft operatively couples the anchor controller to the anchor via engagement of the anchor by a distal-end portion of the anchor-control driveshaft,
the delivery tool further comprises a release spring that is biased to pull the anchor-control driveshaft proximally away from the anchor, the engagement of the anchor by the distal-end portion of the anchor-control driveshaft resisting the pulling of the anchor-control driveshaft by the release spring, and
the driveshaft subassembly further comprises, at the distal-end portion of the anchor-control driveshaft, a lock-rod that maintains the engagement of the anchor by the distal-end portion of the anchor-control driveshaft, such that retraction of the lock-rod from the distal-end portion of the anchor-control driveshaft triggers the release spring to pull the anchor-control driveshaft proximally away from the anchor.

Example 135. The system according to example 134, wherein:
the release spring is a first release spring, and
the delivery tool further comprises a second release spring that is biased to pull the winch-control driveshaft proximally away from the winch, the engagement of the anchor by the distal-end portion of the anchor-control driveshaft resisting the pulling of the winch-control driveshaft by the second release spring, such that the retraction of the lock-rod from the distal-end portion of the anchor-control driveshaft also triggers the second release spring to pull the winch-control driveshaft proximally away from the winch.

Example 136. The system according to example 135, wherein the bias of the first release spring also maintains the winch-control driveshaft in engagement with the winch by exerting a distally-directed force on the winch-control driveshaft.

Example 137. The system according to example 135, wherein the system is configured such that the triggering of the first release spring and the second release spring by the retraction of the lock-rod from the distal-end portion of the anchor-control driveshaft separates the downstream assembly from the delivery tool.

Example 138. The system according to example 130, wherein:
the downstream assembly further comprises a winch, the ventricular anchor being a winch anchor that is coupled to the winch,
the driveshaft subassembly comprises a downstream-assembly-control driveshaft, the system has an anchoring state in which the anchor controller is operatively coupled to the winch anchor via the downstream-assembly-control driveshaft such that operation of the anchor controller applies the anchoring force to the winch anchor, and
the system has a winching state in which the downstream-assembly-control driveshaft is:

operatively uncoupled from the winch anchor such that operation of the anchor controller does not apply the anchoring force to the winch anchor, and operatively coupled to the winch such that rotation of the downstream-assembly-control driveshaft actuates the winch.

Example 139. The system according to example 138, wherein the downstream assembly includes an axle that is axially movable within the downstream assembly such that:
positioning the axle in a first axial position within the downstream assembly places
the system in the anchoring state, and
positioning the axle in a second axial position within the downstream assembly places the system in the winching state.

Example 140. The system according to example 139, wherein:
the winch comprises a spool disposed therein, the spool operatively coupled to the tether such that rotation of the spool tensions the tether,
the axle defines a protruding rim therearound,
the downstream assembly comprises a spring-loaded detent that is biased to protrude into a recess defined by a surface of the spool, thereby maintaining the spool in a locked state in which the spool cannot rotate, and
transitioning the axle to the second axle position automatically unlocks the winch by the rim pushing the detent out of the recess, thereby allowing rotation of the spool.

Example 141. The system according to example 139, wherein:
the downstream-assembly-control driveshaft defines an oblique slot, the axle defines a transverse pin,
in an engaged state of the downstream-assembly-control driveshaft in which the downstream-assembly-control driveshaft is locked to the axle:
the transverse pin is disposed transversely within the slot of the downstream-assembly-control driveshaft, and
a reference-force tube of the driveshaft subassembly is disposed over the oblique slot and the axle in a manner that cooperates with the transverse pin within the slot to prevent proximal movement of the downstream-assembly-control driveshaft away from the axle by obstructing lateral movement of the downstream-assembly-control driveshaft with respect to the axle, and
retracting the reference-force tube from over the oblique slot and the axle allows the downstream-assembly-control driveshaft to move proximally away from the axle by allowing the slot to slide obliquely off the pin.

Example 142. The system according to example 139, wherein the first axial position is distal to the second axial position.

Example 143. The system according to example 138, wherein the system further has a neutral state in which the downstream-assembly-control driveshaft is coupled to the downstream assembly but is operatively uncoupled from both the winch anchor and the winch.

Example 144. The system according to example 138, wherein:
in the anchoring state, the downstream-assembly-control driveshaft is disposed in a first axial position with respect to the downstream assembly,
in the winching state, the downstream-assembly-control driveshaft is disposed in a second, different, axial position with respect to the downstream assembly, and the delivery tool is transitionable between the anchoring state and the winching state via axial movement of the downstream-assembly-control driveshaft with respect to the downstream assembly.

Example 145. The system according to example 144, wherein:
the first axial position is distal to the second axial position, and
the delivery tool is transitionable from the anchoring state to the winching state via proximal movement of the downstream-assembly-control driveshaft with respect to the downstream assembly.

Example 146. The system according to any one of examples 1-145, wherein the delivery tool further comprises a mount, configured to support the patch mounted thereon, and configured to carry the patch toward the clasp while the clasp is in the grasping state.

Example 147. The system according to example 146, wherein the mount is configured to carry the patch toward the upstream support of the clasp by moving, with the patch mounted thereon, distally toward the clasp while the clasp is in the grasping state.

Example 148. The system according to example 147, wherein the mount is configured to carry the patch toward the upstream support of the clasp by moving, with the patch mounted thereon, distally and laterally toward the clasp while the clasp is in the grasping state.

Example 149. The system according to example 148, wherein the delivery tool comprises a beam that provides a mechanical linkage between the shaft and the mount, the mechanical linkage linking distalward movement of the mount with lateral movement of the mount.

Example 150. The system according to example 146, wherein:
the mount has a retracted position, the distal portion of the delivery tool being transluminally advanceable to the heart while the mount is in the retracted position with the patch mounted on the mount,
the mount has a primed position in which the mount is disposed closer to the clasp than in the retracted position, and
the driver is configured to anchor the patch to the leaflet by, while the mount is in the primed position with the patch mounted on the mount, driving the patch anchor through the leaflet.

Example 151. The system according to example 150, wherein:
the mount defines a channel therein,
the distal portion of the delivery tool is transluminally advanceable to the heart while the mount is in the retracted position with the patch mounted on the mount and the patch anchor disposed within the channel, and
the driver is configured to anchor the patch to the portion of the leaflet by driving the patch anchor out of the channel and through the portion of the leaflet.

Example 152. The system according to example 151, wherein:
the delivery tool comprises a needle disposed within the channel, the distal portion of the delivery tool is transluminally advanceable to the heart while the mount is in the retracted position with the patch mounted on the mount and the patch anchor disposed within the needle within the channel, and
the delivery tool is configured to facilitate the driver driving the patch anchor through the portion of the leaflet by advancing the needle out of the channel.

Example 153. The system according to example 152, wherein the delivery tool further comprises a spring that biases the needle to retract into the channel.

Example 154. The system according to example 153, wherein the delivery tool further comprises a mount-control rod, operatively coupled to the mount in a manner that configures the mount-control rod to transition the mount between the retracted position and the primed position.

Example 155. The system according to example 154, wherein the mount-control rod is operatively coupled to the mount by being coupled to the needle.

Example 156. The system according to example 155, wherein the operative coupling of the mount-control rod to the mount is such that:
from the retracted position, pushing of the needle by the mount-control rod pushes, via the spring, the mount toward the primed position, and
while the mount is in the primed position, pushing of the needle by the mount-control rod strains the spring and advances the needle out of the channel.

Example 157. The system according to example 150, wherein the delivery tool comprises a spring configured to bias the mount toward assuming the primed position.

Example 158. The system according to example 157, wherein the spring is a spring-loaded beam that provides a mechanical linkage between the shaft and the mount, and that biases the mount toward assuming the primed position by biasing the mount to move distalward and laterally.

Example 159. The system according to example 157, wherein the driver comprises a rod and a drive head, the drive head being coupled to the mount such that tension on the rod constrains the mount in the retracted position.

Example 160. The system according to example 159, wherein:
the patch anchor and the drive head are disposed within a channel defined in the mount,
relieving the tension on the rod allows the spring to move the mount into the primed position, and
while the mount is in the primed position, pushing on the rod moves the drive head through the channel and deploys the patch anchor out of the channel.

Example 161. The system according to example 150, wherein the clasp is transitionable between the open state and the grasping state while the mount remains in the retracted position.

Example 162. The system according to example 150, wherein the delivery tool has an extracorporeal proximal portion that comprises a mount controller operatively coupled to the mount such that operation of the mount controller moves the mount between the retracted position and the primed position.

Example 163. The system according to example 162, wherein the delivery tool further comprises a mount-control rod via which the mount controller is operatively coupled to the mount.

Example 164. The system according to example 163, wherein:
the mount-control rod is a first mount-control rod,
the delivery tool further comprises a second mount-control rod via which the mount controller is operatively coupled to the mount, and
the extracorporeal portion comprises a lever:

via which the mount controller is operatively coupled to the first and second mount-control rods, and adapted to pivot in a manner that balances the first mount-control rod with the second mount-control rod.

Example 165. The system according to example 164, wherein the lever has a fulcrum at which the mount controller is pivotably attached to the lever, and wherein the first and second mount-control rods are coupled to the lever at respective opposite sides of the fulcrum.

Example 166. The system according to example 163, wherein the extracorporeal proximal portion further comprises a driver controller operatively coupled to the driver such that operation of the driver controller induces the driver to drive the patch anchor through the leaflet.

Example 167. The system according to example 166, wherein the mount-control rod is tubular, and the driver extends from the driver controller, through the mount-control rod.

Example 168. The system according to example 163, wherein the extracorporeal proximal portion of the delivery tool further comprises a clasp controller operatively coupled to the clasp such that operation of the clasp controller transitions the clasp between the open state and the grasping state.

Example 169. The system according to example 168, wherein the delivery tool further comprises a clasp-control wire via which the clasp controller is operatively coupled to the mount.

Example 170. The system according to example 169, wherein:
the clasp-control wire is a first clasp-control wire,
the delivery tool further comprises a second clasp-control wire via which the clasp controller is operatively coupled to the mount, and
the extracorporeal portion comprises a lever:

via which the clasp controller is operatively coupled to the first and second clasp-control wires, and adapted to pivot in a manner that balances the first clasp-control wire with the second clasp-control wire.

Example 171. The system according to example 170, wherein the lever has a fulcrum at which the clasp controller is pivotably attached to the lever, and wherein the first and second clasp-control wires are coupled to the lever at respective opposite sides of the fulcrum.

Example 172. The system according to example 169, wherein the mount controller is configured to, while the clasp is in the grasping state, move the mount between the retracted position and the primed position by sliding the mount over and along the clasp-control wire toward the clasp.

Example 173. The system according to example 172, wherein the clasp controller is configured to, while the mount is in the retracted position, transition the clasp from the grasping state to the open state by retracting the clasp-control wire through the mount.

Example 174. The system according to example 150, wherein the delivery tool further comprises one or more wraps, the distal portion of the delivery tool being transluminally advanceable to the heart while the mount is in the retracted position with the patch held against the mount by the one or more wraps wrapped around the patch and the mount.

Example 175. The system according to example 174, wherein the one or more wraps are one or more kirigami wraps.

Example 176. The system according to example 175, wherein the delivery tool further comprises a release mechanism, adapted to release the patch from against the mount by applying tension to the one or more kirigami wraps.

Example 177. The system according to example 175, wherein the delivery tool further comprises a release mechanism, adapted to release the patch from against the mount by releasing tension in the one or more kirigami wraps.

Example 178. The system according to example 174, wherein the distal portion of the delivery tool is transluminally advanceable to the heart while the mount is in the retracted position with the patch held against the mount by the one or more wraps wrapped around the patch, the mount, and the shaft.

Example 179. The system according to example 174, wherein the delivery tool further comprises one or more spring-loaded brackets configured to hold the wraps taut.

Example 180. The system according to example 179, wherein the delivery tool further comprises a rod that cooperates with the spring-loaded brackets to hold the wraps taut, and that is retractable to release the one or more wraps.

Example 181. The system according to example 150, wherein, in the retracted position, the mount curves in an arc partway around the shaft.

Example 182. The system according to example 150, wherein the mount has a convex outer surface, and the patch is mounted on the mount in a manner in which the patch lies in a curve against the convex outer surface of the mount.

Example 183. The system according to example 150, wherein the mount is shaped to house the patch anchor while the patch is mounted on the mount.

Example 184. The system according to example 183, wherein the patch anchor is coupled to the patch, and the system is configured such that housing of the patch anchor by the mount secures the patch to the mount.

Example 185. The system according to example 184, wherein the patch is coupled to the patch anchor via a cord, and is secured to a surface of the mount by the patch anchor being disposed in a channel defined in the surface of the mount, the channel being shaped to:
facilitate sliding of the patch anchor along the channel, and
obstruct the patch anchor from exiting the channel laterally.

Example 186. The system according to example 185, wherein the driver is configured to anchor the patch to the leaflet by, while the mount is in the primed position with the patch mounted on the mount, driving the patch anchor along the channel, out of an end of the channel, and through the leaflet.

Example 187. The system according to example 185, wherein the cord extends from the patch anchor, laterally out of the channel to the patch.

Example 188. The system according to any one of examples 1-187, wherein:
within the distal portion of the delivery tool the shaft is telescopic,
the delivery tool has a delivery state in which:
the shaft is telescopically extended,
the clasp faces distally, and
the distal portion of the delivery tool is transluminally advanceable to the heart.

Example 189. The system according to example 188, wherein, in the delivery state, the downstream support is deflected distally compared to in the open state.

Example 190. The system according to example 188, wherein, in the delivery state, the downstream support is disposed adjacent to, and substantially parallel with, the shaft.

Example 191. The system according to example 188, wherein, in the delivery state, the clasp is closed.

Example 192. The system according to example 188, wherein the delivery tool has a contracted state in which:
the shaft is telescopically contracted, and
the clasp faces proximally.

Example 193. The system according to example 192, wherein the distal portion of the delivery tool is configured to be advanced downstream through the valve while in the contracted state.

Example 194. The system according to example 192, wherein, in the contracted state, the clasp is closed.

Example 195. The system according to example 192, wherein, in the contracted state, the downstream support is deflected proximally compared to in the open state.

Example 196. The system according to example 192, wherein in the contracted state, the clasp extends further laterally from the shaft than in the open state.

Example 197. The system according to any one of examples 1-196, wherein the delivery tool has an extracorporeal proximal portion that comprises a shaft extender, operatively coupled to the shaft such that operation of the shaft extender reversibly extends a distal part of the shaft distally from a proximal part of the shaft.

Example 198. The system according to example 197, wherein the clasp is coupled to the shaft such that extension of the distal part of the shaft distally from the proximal part of the shaft deflects the downstream support with respect to the shaft.

Example 199. The system according to any one of examples 1-198, wherein the patch is substantially trapezoid.

Example 200. The system according to example 199, wherein:
the patch has a lip and a root,
the driver is configured to anchor the root of the patch to the leaflet such that the lip of the patch extends toward an opposing leaflet of the valve, and
the lip is longer than the root.

Example 201. The system according to any one of examples 1-200, wherein the delivery tool further comprises a retrieval line, releasably coupled to the anchor such that tensioning the retrieval line de-anchors the patch anchor from the leaflet.

Example 202. The system according to example 201, wherein the delivery tool has an extracorporeal portion, and wherein, the retrieval line extends:
from a first end portion of the retrieval line at the extracorporeal portion, transluminally through the delivery tool to the anchor,
at the anchor, the retrieval line loops through the anchor, and from the anchor, transluminally back through the delivery tool to the extracorporeal portion where the retrieval line defines a second end portion.

Example 203. The system according to example 202, wherein both the first end portion and the second end portion are coupled to a bobbin that is mounted on the extracorporeal portion.

Example 204. The system according to example 203, wherein each of the first end portion and the second end portion extends, from the bobbin, proximally along the extracorporeal portion, towards a bearing, and, at the bearing, turns back on itself to extend distally through the delivery tool to the anchor such that sliding the bobbin distally along the extracorporeal portion tensions the retrieval line.

Example 205. The system according to example 203, wherein:
the first end portion is wrapped circumferentially around the bobbin,
the bobbin defines a trough that provides accessibility to the first end portion, and the retrieval line is releasable from the anchor by cutting the first end portion via the trough, and subsequently dismounting the bobbin from the extracorporeal portion and moving the bobbin away from the extracorporeal portion.

Example 206. The system according to example 205, wherein the bobbin defines a lateral slit, and wherein the bobbin is dismountable from the extracorporeal portion by moving the bobbin laterally off the extracorporeal portion via the lateral slit.

Example 207. The system according to example 205, wherein the trough is a trough of a series of troughs that are distributed circumferentially around the bobbin.

Example 208. The system according to example 201, wherein:
the patch anchor comprises a tubular toggle, and includes a retrieval feature comprising a notch at a heel of the toggle and a retrieval eyelet, and
the retrieval line:

extends, colinearly with the toggle, into a lumen of the toggle at the heel of the toggle, exits a lateral wall of the toggle via the retrieval eyelet, and loops back to itself via the notch to connect to itself.

Example 209. The system according to example 201, wherein the retrieval line is releasably coupled to the anchor such that tensioning the retrieval line facilitates de-anchoring of the patch anchor from the leaflet by reorienting the patch anchor.

Example 210. An apparatus for use with a valve disposed between an atrium and a ventricle of a heart of a subject, the valve having at least a first leaflet and a second leaflet, and the apparatus comprising an implant that comprises:
a leaflet-augmentation patch comprising:
a flexible sheet, and
a frame that supports the flexible sheet; and
a patch anchor, coupled to the patch in a manner that facilitates anchoring of the patch to the first leaflet by:
allowing the patch anchor to be temporarily moved away from the patch while the patch anchor remains coupled to the patch, and
biasing the patch anchor to return toward the patch.

Example 211. The apparatus according to example 210, wherein the patch anchor has a sharpened tip, and is configured to be driven through the first leaflet with the sharpened tip penetrating the first leaflet.

Example 212. The apparatus according to any one of examples 210-211, wherein the patch anchor is configured to be driven through the first leaflet while disposed within a hollow needle.

Example 213. The apparatus according to any one of examples 210-212, wherein the patch anchor comprises a tubular toggle and includes a retrieval feature comprising a notch at a heel of the toggle, and a retrieval eyelet, the apparatus further comprising a retrieval line that:

extends, colinearly with the toggle, into a lumen of the toggle at the heel of the toggle, exits a lateral wall of the toggle via the retrieval eyelet, and loops back to itself via the notch to connect to itself.

Example 214. The apparatus according to any one of examples 210-213, wherein the implant comprises:
an upstream assembly comprising the patch and the patch anchor,
a downstream assembly comprising a ventricular anchor, and
a tether, tethering the patch to the ventricular anchor.

Example 215. The apparatus according to example 214, wherein:
the ventricular anchor is a first ventricular anchor,
the downstream assembly further comprises a second ventricular anchor, and
the tether tethers the patch to both the first ventricular anchor and the second ventricular anchor.

Example 216. The apparatus according to example 214, wherein the tether extends from the downstream assembly to the patch, and back to the downstream assembly.

Example 217. The apparatus according to example 216, wherein:
the ventricular anchor is a first ventricular anchor,
the downstream assembly further comprises a second ventricular anchor, and
the tether extends from the first ventricular anchor to the patch, and back to the second ventricular anchor.

Example 218. The apparatus according to example 216, wherein the tether is slidably coupled to the upstream assembly.

Example 219. The apparatus according to example 218, wherein the upstream assembly defines an eyelet, and wherein the tether is slidably coupled to the upstream assembly by being threaded through the eyelet.

Example 220. The apparatus according to example 218, wherein:
the downstream assembly comprises a winch coupled to the ventricular anchor, and
the tether is arranged in a pulley arrangement in which:
a first end of the tether is operatively coupled to the winch,
a bight of the tether is slidably coupled to the upstream assembly, and
a second end of the tether is fixed to the downstream assembly.

Example 221. The apparatus according to example 220, wherein the winch has a housing, fixedly attached to the ventricular anchor, and wherein the second end of the tether is fixed to the housing.

Example 222. The apparatus according to any one of examples 210-221, wherein the patch anchor is a toggle that is biased to automatically widen upon deployment.

Example 223. The apparatus according to example 222, wherein the toggle has a cellular structure that is biased to automatically widen by foreshortening.

Example 224. The apparatus according to any one of examples 210-223, further comprising a delivery tool, configured to deliver the implant to the heart, and to anchor the patch to the first leaflet by:
anchoring the patch anchor to the first leaflet by temporarily moving the patch anchor away from the patch while the patch anchor remains coupled to the patch, and
subsequently, releasing the patch anchor such that the implant responsively returns the patch anchor toward the patch.

Example 225. The apparatus according to example 224, wherein the delivery tool is configured to move the patch anchor away from the patch by driving the patch anchor through the first leaflet.

Example 226. The apparatus according to example 224, wherein the delivery tool is configured to deliver the implant to the heart with the patch mounted laterally on the delivery tool.

Example 227. The apparatus according to any one of examples 210-226, wherein:

the implant comprises an upstream assembly that comprises the patch and the patch anchor, and
the implant further comprises:
a downstream assembly comprising a winch coupled to a winch anchor that is configured to anchor the downstream assembly to tissue of the ventricle; and
a tether, tethering the winch to the patch.

Example 228. The apparatus according to example 227, wherein the patch comprises a first part of the sheet, and a second part of the sheet is shaped to extend away from the patch in a manner that defines the tether.

Example 229. The apparatus according to example 227, wherein the implant further comprises a cord via which the patch anchor is coupled to the patch.

Example 230. The apparatus according to example 227, wherein the patch includes a spring, and wherein the patch anchor is coupled to the spring in the manner that biases the patch anchor to return toward the patch.

Example 231. The apparatus according to example 230, wherein the frame defines the spring.

Example 232. The apparatus according to example 230, wherein the spring is a compression spring.

Example 233. The apparatus according to example 230, wherein the spring lies substantially flat with respect to the patch.

Example 234. The apparatus according to example 230, wherein the implant further comprises a cord via which the patch anchor is coupled to the spring.

Example 235. The apparatus according to example 234, wherein the spring is configured to facilitate driving of the patch anchor through the first leaflet by transiently straining in response to tension applied to the cord by pushing the patch anchor away from the patch and through the first leaflet.

Example 236. The apparatus according to example 235, wherein the spring is coupled to the sheet in a manner in which the patch transiently linearly contracts as the spring transiently strains.

Example 237. The apparatus according to example 235, wherein the spring is coupled to the sheet in a manner in which the spring slides across the sheet as the spring transiently strains.

Example 238. The apparatus according to example 235, wherein:
the patch has a lip and a root,
the cord couples the patch anchor to the patch in a manner in which anchoring the patch anchor to the first leaflet positions the patch such that the lip of the patch
extends toward the second leaflet, and
the frame defines:
a lip brace at the lip of the patch, and
a root brace at the root of the patch.

Example 239. The apparatus according to example 238, wherein the spring is configured such that the transient straining consists substantially of transient compression of the spring between the lip brace and the root brace.

Example 240. The apparatus according to example 238, wherein the patch defines, along a midline of the patch, a root-to-lip axis between the lip and the root, and wherein the spring is configured such that the transient straining consists substantially of deflection of the spring with respect to the root-to-lip axis.

Example 241. The apparatus according to example 240, wherein the spring is configured such that the transient straining consists substantially of deflection of the spring toward the root-to-lip axis.

Example 242. The apparatus according to example 240, wherein the spring is a first spring, and wherein the frame further comprises a second spring, the first spring and the second spring configured such that the transient straining consists substantially of deflection of the first spring and the second spring toward each other.

Example 243. The apparatus according to example 242, wherein the cord extends back and forth between the first spring and the second spring.

Example 244. The apparatus according to example 238, wherein the frame defines a patch-anchor support coupled to the root brace, the cord extending from the spring, through the patch-anchor support, to the patch anchor.

Example 245. The apparatus according to example 238, wherein the spring is attached to the root brace.

Example 246. The apparatus according to example 245, wherein the spring is configured such that the transient straining consists substantially of transient deflection of the spring with respect to the root brace.

Example 247. The apparatus according to example 245, wherein the spring does not extend to the lip brace.

Example 248. The apparatus according to example 245, wherein the spring extends from the root brace to the lip brace.

Example 249. The apparatus according to example 248, wherein the spring extends from the root brace to the lip brace along a midline of the patch.

Example 250. The apparatus according to example 248, wherein:
the spring is a first spring, extending from the root brace to the lip brace along a first lateral edge of the patch, and
the frame defines a second spring, extending from the root brace to the lip brace along a second lateral edge of the patch.

Example 251. The apparatus according to example 229, wherein the patch anchor comprises a toggle that defines an eyelet substantially midway along the toggle, the cord being attached to the patch anchor at the eyelet.

Example 252. The apparatus according to example 251, further comprising a retrieval line, extending from an end of the toggle, and configured to de-anchor the patch anchor from the first leaflet upon tensioning of the retrieval line.

Example 253. The apparatus according to example 251, wherein the eyelet extends transversely entirely through the toggle.

Example 254. The apparatus according to example 251, wherein the toggle is substantially tubular, having a lateral wall that defines a lumen.

Example 255. The apparatus according to example 254, wherein the lateral wall defines two lateral holes adjacent each other, the eyelet being defined by a part of the lateral wall disposed between the two lateral holes.

Example 256. A system for use with a valve disposed between an atrium and a ventricle of a heart of a subject, the system comprising:
an implant, comprising:
a tether; and
an assembly, comprising:
a winch, comprising a housing and a spool disposed therein, the tether extending from the winch, and the spool operatively coupled to the tether such that actuation of
the winch tensions the tether; and
a winch anchor, coupled to the winch; and
a delivery tool, having a distal portion transluminally advanceable to the heart while coupled to the implant, the delivery tool comprising a driveshaft subassembly, comprising:
a reference-force tube, coupled to the housing; and
a driveshaft, extending through the reference-force tube, wherein the system:
has an anchoring state in which the driveshaft is:
operatively coupled to the winch anchor such that rotation of the driveshaft applies an anchoring force to the winch anchor, and
operatively uncoupled from the winch such that rotation of the driveshaft does not actuate the winch, and
has a winching state in which the driveshaft is:
operatively uncoupled from the winch anchor such that rotation of the driveshaft does not apply the anchoring force to the winch anchor, and
operatively coupled to the winch such that rotation of the driveshaft actuates the winch.

Example 257. The system according to example 256, wherein the system further has a neutral state in which the driveshaft is coupled to the implant but is operatively uncoupled from both the winch anchor and the winch.

Example 258. The system according to any one of examples 256-257, wherein the assembly includes an axle that is axially movable within the assembly such that:
positioning the axle in a first axial position within the assembly places the system in the anchoring state, and
positioning the axle in a second axial position within the assembly places the system in the winching state.

Example 259. The system according to example 258, wherein:
the winch comprises a spool disposed therein, the spool operatively coupled to the tether such that rotation of the spool tensions the tether,
the axle defines a protruding rim therearound,
the assembly comprises a spring-loaded detent that is biased to protrude into a recess defined by a surface of the spool, thereby maintaining the spool in a locked state in which the spool cannot rotate, and
transitioning the axle to the second axle position automatically unlocks the winch by the rim pushing the detent out of the recess, thereby allowing rotation of the spool.

Example 260. The system according to example 258, wherein:
the driveshaft defines an oblique slot,
the axle defines a transverse pin,
in an engaged state of the driveshaft in which the driveshaft is locked to the axle:
the transverse pin is disposed transversely within the slot of the downstream-assembly-control driveshaft, and
the reference-force tube is disposed over the oblique slot and the axle in a manner that cooperates with the transverse pin within the slot to prevent proximal movement of the driveshaft away from the axle by obstructing lateral movement of the driveshaft with respect to the axle, and
retracting the reference-force tube from over the oblique slot and the axle allows the driveshaft to move proximally away from the axle by allowing the slot to slide obliquely off the pin.

Example 261. The system according to example 258, wherein the first axial position is distal to the second axial position.

Example 262. The system according to any one of examples 256-261, wherein:
in the anchoring state, the driveshaft is disposed in a first axial position with respect to the winch,
in the winching state, the driveshaft is disposed in a second, different, axial position with respect to the winch, and
the delivery tool is transitionable between the anchoring state and the winching state via axial movement of the driveshaft with respect to the winch.

Example 263. The system according to example 262, wherein:
the first axial position is distal to the second axial position, and
the delivery tool is transitionable from the anchoring state to the winching state via proximal movement of the driveshaft with respect to the winch.

Example 264. A system, for use with a tissue of a subject, the system comprising:
a toggle anchor, having a tip and a heel, and defining an anchor axis therebetween; and
a delivery tool:
defining a channel in which the toggle anchor is disposed; and
comprising a driver configured to push the toggle anchor, tip-first, distally out of and away from the channel, the driver having a drive head, and a rod extending proximally from the drive head, the drive head being connected to the heel via complimentary geometry in a manner that (i) preferentially allows deflection rather than lateral translation of the toggle anchor with respect to the driver, and (ii) allows the heel to disconnect from the driver upon the anchor reaching a predetermined angle with respect to the driver.

Example 265. The system according to example 264, wherein:
the drive head defines a knob, and a neck that connects the knob to the rod, and
at the heel, the toggle anchor defines appendages that extend proximally beyond the knob and, proximally from the knob, medially toward each other and toward the neck, such that:
the appendages inhibit proximal retraction and lateral translation of the driver from the toggle anchor, and
deflection of the toggle anchor with respect to the driver urges the knob between the appendages such that the appendages deflect laterally away from each other and from the neck.

Example 266. The system according to any one of examples 264-265, wherein:
the drive head defines a socket that has a rim,
at the heel, the toggle anchor defines a knob,
the knob is disposed in the socket in a manner that inhibits lateral translation of the toggle anchor from the driver, and
deflection of the toggle anchor with respect to the driver presses the rim against the toggle anchor in a manner that levers the knob distally out of the socket.

Example 267. The system according to any one of examples 264-266, wherein the delivery tool is transluminally advanceable to the tissue.

Example 268. The system according to any one of examples 264-267, wherein the tip of the anchor has a sharp point.

Example 269. The system according to any one of examples 264-268, wherein:
the drive head has a first distally-facing face, and defines a shoulder that defines a second distally-facing face proximal from the first face, and
at the heel, the toggle anchor defines a lateral opening through which the shoulder protrudes, such that:
the driver is configured to push the toggle anchor tip-first through the tissue by (i) the second distally-facing face pushing distally on the toggle anchor at the lateral opening, and (ii) the first distally-facing face pushing distally on the toggle anchor substantially opposite the lateral opening, and
the toggle anchor is allowed to disconnect from the drive head by deflecting about a point on the driver proximal from the second distally-facing face such that the lateral opening moves laterally away from the shoulder.

Example 270. The system according to example 269, wherein the driver further comprises a stabilizer, configured such that pushing, by the driver, of the tip of the toggle anchor against the tissue moves the stabilizer into a stabilizing position with respect to the toggle anchor via axial sliding of the stabilizer relative to the toggle anchor, the stabilizer, in the stabilizing position, inhibiting deflection of the toggle anchor with respect to the driver.

Example 271. The system according to any one of examples 264-270, further comprising a cord attached to the toggle anchor.

Example 272. The system according to example 271, wherein the system comprises an implant comprising the toggle anchor, the cord, and another component, the cord connecting the other component to the toggle anchor such that the toggle anchor is configured to anchor the other component to the tissue.

Example 273. A system, comprising:
an implant, comprising a toggle anchor, having a body, a tip, and a heel, the toggle anchor defining an anchor axis between the tip and the heel; and
a delivery tool:
configured to transluminally advance the implant to a tissue of a subject while the implant is coupled to a distal portion of the tool, and
comprising a driver that comprises a drive head and a rod extending proximally from the drive head, the driver configured to push the toggle anchor tip-first through the tissue,
wherein the system comprises an extendable member, and is configured such that, upon the driver pushing the tip of the toggle anchor against the tissue, the extendable member responsively slides axially with respect to the body.

Example 274. The system according to example 273, wherein at least the tip of the toggle anchor is hollow.

Example 275. The system according to any one of examples 273-274, wherein the extendable member is a component of the delivery tool.

Example 276. The system according to any one of examples 273-275, wherein the extendable member is a component of the toggle anchor.

Example 277. The system according to any one of examples 273-276, wherein the extendable member is a post.

Example 278. The system according to any one of examples 273-277, wherein:
the system defines a sharp point, configured to pierce the tissue in a manner that facilitates the driver pushing the toggle anchor tip-first through the tissue,
the system has a resting state in which the sharp point is functionally obscured, and
the system is configured such that, upon the driver pushing the tip of the toggle anchor against the tissue, the extendable member automatically functionally exposes the sharp point by sliding axially with respect to the body.

Example 279. The system according to example 278, wherein:
the sharp point is defined by the tip of the toggle anchor, in the resting state, the extendable member functionally obscures the sharp point, and
the system is configured such that, upon the driver pushing the tip of the toggle anchor against the tissue, the extendable member automatically functionally exposes
the sharp point by sliding proximally away from the sharp point.

Example 280. The system according to example 279, wherein the extendable member is a component of the toggle anchor.

Example 281. The system according to example 279, wherein the system is configured such that, upon the driver pushing the tip of the toggle anchor against the tissue, the extendable member slides proximally away from the sharp point by sliding into an interior of the toggle anchor.

Example 282. The system according to example 279, wherein the system is configured such that, upon the driver pushing the tip of the toggle anchor against the tissue, the extendable member slides proximally away from the sharp point by sliding over an exterior of the toggle anchor.

Example 283. The system according to example 278, wherein:
the sharp point is defined by the extendable member,
in the resting state, the toggle anchor functionally obscures the sharp point, and the system is configured such that, upon the driver pushing the tip of the toggle anchor against the tissue, the extendable member automatically slides distally beyond the tip, thereby functionally exposing the sharp point.

Example 284. The system according to example 283, wherein the extendable member is a component of the delivery tool, and comprises a needle that defines the sharp point.

Example 285. The system according to example 283, wherein, in the resting state, the sharp point is functionally obscured by the toggle anchor.

Example 286. The system according to example 285, wherein, in the resting state, the sharp point is functionally obscured by being disposed within the body of the toggle anchor.

Example 287. A system, comprising:
an implant, comprising a toggle anchor, having a body, a tip, and a heel, the toggle anchor defining an anchor axis between the tip and the heel; and
a delivery tool:
configured to transluminally advance the implant to a tissue of a subject while the implant is coupled to a distal portion of the tool, and comprising:
a driver that comprises a drive head and a rod extending proximally from the drive head, the driver configured to push the toggle anchor tip-first through the tissue, and
a stabilizer, configured such that pushing, by the driver, of the tip of the toggle anchor against the tissue moves the stabilizer into a stabilizing position with respect to the toggle anchor via axial sliding of the stabilizer relative to the toggle anchor, the stabilizer, in the stabilizing position, inhibiting deflection of the toggle anchor with respect to the driver.

Example 288. The system according to example 287, wherein the delivery tool comprises a spring that biases the stabilizer away from the stabilizing position.

Example 289. The system according to any one of examples 287-288, wherein the delivery tool is configured such that the axial sliding of the stabilizer relative to the toggle anchor is accompanied by movement of the drive head proximally toward the rod.

Example 290. The system according to any one of examples 287-289, wherein the drive head is coupled to the rod via a compression spring that compresses upon the driver pushing the tip of the toggle anchor against the tissue, the compression of the spring facilitating the axial sliding of the stabilizer relative to the toggle anchor.

Example 291. The system according to example 290, wherein the compression spring is configured to facilitate disengagement of the toggle anchor from the driver upon cessation of the pushing by the driver.

Example 292. The system according to any one of examples 287-291, wherein the stabilizer comprises a post configured such that pushing, by the driver, of the tip of the toggle anchor against the tissue moves the stabilizer into the stabilizing position via sliding of the post distally into the toggle anchor.

Example 293. The system according to example 292, wherein at least the heel of the toggle anchor is tubular, and wherein the post is configured such that pushing, by the driver, of the tip of the toggle anchor against the tissue moves the stabilizer into the stabilizing position via axial sliding of the post into a tubular lumen defined by the toggle anchor.

Example 294. The system according to example 292, wherein the stabilizer is disposed inside the driver.

Example 295. The system according to example 292, wherein the drive head is coupled to the rod via a compression spring that extends over at least part of the post.

Example 296. The system according to example 292, wherein the delivery tool is configured such that the sliding of the post distally into the toggle anchor is accompanied by movement of the drive head proximally toward the rod.

Example 297. The system according to any one of examples 287-296, wherein the stabilizer comprises a receptacle configured such that pushing, by the driver, of the tip of the toggle anchor against the tissue moves the stabilizer into the stabilizing position via sliding of the heel proximally into the receptacle.

Example 298. The system according to example 297, wherein the heel is dimensioned to fit snugly within the receptacle.

Example 299. The system according to example 297, wherein the receptacle is tubular.

Example 300. The system according to example 297, wherein the receptacle is a cup.

Example 301. The system according to example 297, wherein the drive head is coupled to the rod via a compression spring that extends through at least part of the receptacle.

Example 302. The system according to example 297, wherein the delivery tool is configured such that the axial sliding of the heel into the receptacle is accompanied by sliding of the drive head proximally into the receptacle.

Example 303. The system according to example 297, wherein the delivery tool is configured such that the axial sliding of the heel into the receptacle is accompanied by movement of the drive head proximally toward the rod.

Example 304. Apparatus for use with a tissue, the apparatus comprising an implant that comprises:
a toggle anchor having a tip, a heel, and an anchor axis between the tip and the heel, and defining a lateral eyelet partway between the tip and the heel, the toggle anchor comprising:
a first segment, defining the tip, and
a second segment, slidably coupled to the first segment, and defining the heel; and
a longitudinal member, extending through the lateral eyelet, and connected to the toggle anchor in a manner in which pulling of the longitudinal member slides the second segment axially with respect to the first segment.

Example 305. The apparatus according to example 304, wherein the longitudinal member is connected to the toggle anchor in a manner in which pulling of the longitudinal member slides the heel toward the lateral eyelet.

Example 306. The apparatus according to any one of examples 304-305, wherein the longitudinal member is connected to the toggle anchor in a manner in which pulling of the longitudinal member slides the heel away from the lateral eyelet.

Example 307. The apparatus according to any one of examples 304-306, wherein the longitudinal member is connected to the toggle anchor in a manner in which pulling of the longitudinal member extends the heel away from the first segment such that the lateral eyelet becomes disposed substantially midway between the tip and the heel of the toggle anchor.

Example 308. The apparatus according to any one of examples 304-307, wherein:
the longitudinal member is attached to an attachment point of the toggle anchor, and
prior to the longitudinal member being pulled, the longitudinal member defines a path that includes at least one turn between the lateral eyelet and the attachment point.

Example 309. The apparatus according to any one of examples 304-308, wherein the longitudinal member is connected to the toggle anchor in a manner in which the sliding of the second segment axially with respect to the first segment is accompanied by sliding of the longitudinal member out of the lateral eyelet.

Example 310. The apparatus according to any one of examples 304-309, wherein at least part of the second segment is coaxial with at least part of the first segment.

Example 311. The apparatus according to any one of examples 304-310, wherein the second segment is telescopically coupled to the first segment, and wherein the longitudinal member is connected to the toggle anchor in a manner in which pulling of the longitudinal member slides the second segment telescopically with respect to the first segment.

Example 312. The apparatus according to any one of examples 304-311, wherein the second segment is coupled to the first segment such that the second segment is axially slidable within the first segment.

Example 313. The apparatus according to any one of examples 304-312, wherein the toggle anchor comprises a spring that biases the second segment toward a predetermined axial position with respect to the first segment.

Example 314. The apparatus according to example 313, wherein the longitudinal member is connected to the toggle anchor in a manner in which pulling of the longitudinal member slides the second segment axially away from the predetermined axial position with respect to the first segment.

Example 315. The apparatus according to example 313, wherein the longitudinal member is connected to the toggle anchor in a manner in which pulling of the longitudinal member strains the spring.

Example 316. The apparatus according to any one of examples 304-315, wherein the longitudinal member is connected to the toggle anchor in a manner in which pulling of the longitudinal member changes an axial length of the toggle anchor by sliding the second segment with respect to the first segment.

Example 317. The apparatus according to example 316, wherein the longitudinal member is connected to the toggle anchor in a manner in which pulling of the longitudinal member increases the axial length of the toggle anchor by sliding the second segment with respect to the first segment.

Example 318. The apparatus according to example 316, wherein the longitudinal member is connected to the toggle anchor in a manner in which pulling of the longitudinal member reduces the axial length of the toggle anchor by sliding the second segment with respect to the first segment.

Example 319. The apparatus according to any one of examples 304-318, wherein the second segment defines a sharp point at an opposite end of the second segment from the heel, and wherein the apparatus is configured such that pulling of the longitudinal member slides the second segment with respect to the first segment in a manner that draws the sharp point into the first segment.

Example 320. The apparatus according to example 319, wherein the apparatus is configured such that pulling of the longitudinal member slides the second segment with respect to the first segment in a manner that draws the sharp point into the first segment and extends the heel away from the first segment.

Example 321. The apparatus according to any one of examples 304-320, wherein the implant further comprises a frame, and the longitudinal member is a cord that connects the toggle anchor to the frame.

Example 322. The apparatus according to example 321, wherein the frame comprises a spring that pulls on the cord.

Example 323. The apparatus according to any one of examples 304-322, wherein the longitudinal member is a retrieval line, configured to pull the toggle anchor out of the tissue.

Example 324. The apparatus according to example 323, wherein the lateral eyelet is disposed at an end of the first segment that is closest to the heel.

Example 325. Apparatus for use with a tissue, the apparatus comprising an implant that comprises:
a toggle anchor having:
a tip,
a heel, and
a lateral eyelet partway between the tip and the heel;
a cord, connected to the toggle anchor via the lateral eyelet; and
a retrieval line, threaded through the toggle anchor in a manner in which tensioning the retrieval line retracts the heel toward the lateral eyelet.

Example 326. The apparatus according to example 325, wherein the toggle anchor is a helical coil that defines a lumen therethrough.

Example 327. The apparatus according to example 326, wherein the retrieval line is threaded through turns of the helical coil in a manner in which tensioning the retrieval line stiffens the anchor by compressing the turns against each other.

Example 328. The apparatus according to any one of examples 325-327, wherein the toggle anchor further comprises a spring, configured to bias the heel to extend away from the lateral eyelet.

Example 329. The apparatus according to example 328, wherein:
the toggle anchor has a sharp point, and
the spring is configured to bias the point toward the lateral eyelet.

Example 330. The apparatus according to example 328, wherein:
the toggle anchor comprises:
a body that defines a lumen and a retrieval eyelet, the retrieval eyelet opening into the lumen; and
a stock, at least part of which is disposed within the lumen, and
the cord extends through the retrieval eyelet to the stock such that tensioning the retrieval line retracts the heel by sliding the stock with respect to the body.

Example 331. The apparatus according to example 330, wherein the cord extends through the retrieval eyelet and a transverse channel in the stock, and is attached to a side of the body opposite the retrieval eyelet.

Example 332. The apparatus according to example 330, wherein the stock is shaped to define the heel.

Example 333. The apparatus according to example 330, wherein the body defines the lateral eyelet.

Example 334. Apparatus for use with a tissue, the apparatus comprising an implant that comprises:
a toggle anchor having:
a tip,
a heel, and
a lateral eyelet partway between the tip and the heel; and
a cord, connected to the toggle anchor via the lateral eyelet in a manner in which tensioning the cord extends the heel away from the lateral eyelet.

Example 335. The apparatus according to example 334, wherein:
the toggle anchor has a sharp point, and
the cord is connected to the toggle anchor via the lateral eyelet in a manner in which tensioning the cord concurrently (i) extends the heel away from the lateral eyelet and (ii) retracts the point toward the lateral eyelet.

Example 336. The apparatus according to example 335, wherein:
the toggle anchor comprises:
a body that defines a lumen and the lateral eyelet, the lateral eyelet opening into the lumen; and
a stock, at least part of which is disposed within the lumen, and the cord extends through the lateral eyelet to the stock such that tensioning the cord concurrently extends the heel and retracts the point by sliding the stock with respect to the body.

Example 337. The apparatus according to example 336, wherein the stock is shaped to define the heel and the point.

Example 338. The apparatus according to example 336, wherein the cord extends through the lateral eyelet and a transverse channel in the stock, and is attached to a side of the body opposite the lateral eyelet.

Example 339. A system for use with a heart of a subject, the system comprising an implant comprising:
a tether; and
an assembly, comprising:
a winch, comprising a housing and a spool disposed therein, the spool operatively coupled to the tether such that actuation of the winch tensions the tether, the tether extending, from the spool and out of an aperture of the housing, the aperture having a rim;
a winch anchor, coupled to the winch; and
a spring, coupled to the housing in a manner that urges the tether away from contact with the rim.

Example 340. The system according to example 339, wherein the spring is a volute spring.

Example 341. The system according to any one of examples 339-340, wherein the spring is a cantilever spring.

Example 342. The system according to any one of examples 339-341, wherein the spring is a wave spring.

Example 343. The system according to any one of examples 339-342, wherein the spring is coupled to the housing in a manner that urges the tether away from contact with a side of the rim that is furthest away from the winch anchor.

Example 344. The system according to any one of examples 339-343, wherein the assembly is a first assembly of the implant, and wherein the implant further comprises a second assembly comprising an anchor, the first assembly and the second assembly being connected via the tether.

Example 345. The system according to example 344, wherein:
the heart has an atrium, a ventricle, and a valve therebetween,
the system further comprises a delivery tool, having a distal portion transluminally advanceable to the heart while coupled to the implant, and adapted to:
anchor the anchor into a leaflet of the valve, and anchor the winch anchor into tissue of the ventricle, such that the tether extends from the anchor at the leaflet, to the winch anchor within the ventricle.

Example 346. The system according to example 345, wherein the first assembly comprises a helix that is shaped to define:
the spring; and
a gripping region adapted to grip the tether between turns of the helix.

Example 347. The system according to example 345, wherein the spring defines a helix having a series of turns that extend circumferentially around the housing, and wherein during ventricular systole of the heart, a pitch between turns of a first portion of the helix is reduced.

Example 348. The system according to example 347, wherein the spring is adapted to grip the tether in between turns of a second portion of the helix.

Example 349. The system according to any one of examples 339-348, wherein the spring defines a helix having a series of turns.

Example 350. The system according to example 349, wherein the helix extends circumferentially around an exterior of the winch housing.

Example 351. The system according to example 349, wherein the spring is adapted to grip the tether in between the turns of the helix.

Example 352. A method of connecting a tether to a component of an implant, the method comprising:
forming a bight in the tether by looping an end portion of the tether around a part of the component, and
closing the bight into a loop by burrowing the end portion coaxially through a stretch of the tether, such that the stretch squeezes on the end portion therewithin.

Example 353. The method according to example 352, wherein:
the bight is a first bight,
the loop is a first loop, burrowing the end portion coaxially through the stretch comprises burrowing a first part of the end portion coaxially through a stretch, and
the method further comprises:

forming a second bight in the end portion, and closing the second bight into a second loop by burrowing a second part of the end portion coaxially through the stretch, such that the first part and the second part extend alongside each other within the stretch.

Example 354. The method according to any one of examples 352-353, wherein:
the bight is a first bight,
the loop is a first loop,
the stretch is a first stretch, and
the method further comprises:
forming a second bight in the end portion, and closing the second bight into a second loop by burrowing the end portion coaxially through a second stretch of the tether, such that the second stretch squeezes on the end portion therewithin.

Example 355. The method according to any one of examples 352-354, wherein:
the end portion is a first end portion of the tether,
a second end portion of the tether extends, from the stretch, away from the first end portion, an end of the second end portion being coupled to a downstream assembly, and
looping the end portion of the tether around the part of the component comprises looping the first end portion of the tether around a part of a leaflet patch of an upstream assembly.

Example 356. The method according to any one of examples 352-355, wherein the stretch is a braid, and wherein burrowing the end portion coaxially through the stretch comprises burrowing the end portion coaxially through the stretch such that strands of the braid are pushed apart.

Example 357. The method according to any one of examples 352-356, wherein the stretch comprises strands of a weave, and wherein burrowing the end portion coaxially through the stretch comprises burrowing the end portion coaxially through the stretch such that the strands of the weave are pushed apart.

Example 358. The method according to any one of examples 352-357, the method further comprising, subsequently to burrowing the end portion coaxially through the stretch, trimming an end part of the end portion that extends, from out of the stretch to an end of the tether.

Example 359. Apparatus for use with a tissue, the apparatus comprising an implant that comprises:
a toggle anchor having a heel defining a retrieval eyelet, and
a retrieval adapter, having a first loop at a first end and a second loop at a second end, the first loop extending through the retrieval eyelet, and
a retrieval line, looped through the second loop in a manner in which pulling on the retrieval line reorients the toggle anchor for retrieval.

Example 360. The apparatus according to example 359, wherein:
the toggle anchor is a first toggle anchor,
the retrieval adapter is a first retrieval adapter,
the apparatus further comprises a second toggle anchor, and a second retrieval adapter, and
the retrieval line is looped through both the second loop of the first retrieval adapter, and through a second loop of the second retrieval adapter, such that pulling on the retrieval line reorients both the first toggle anchor and the second toggle anchor for retrieval.

Example 361. A system for use with a subject, the system comprising:
a toggle anchor that is in the form of a helical coil, the coil having a longitudinal axis that extends from a first end portion of the coil to a second end portion of the coil,
a cord connected to the coil at a site between the first end portion and the second end portion, and extending, from the site, orthogonally away from the longitudinal axis, and
a retrieval line that extends from the first end portion to the second end portion, and away from the toggle anchor, the retrieval line being fixed to the toggle anchor in a manner in which tensioning the retrieval line stiffens the anchor by compressing turns of the coil against each other.

Example 362. The system according to example 361, the retrieval line is fixed to the end portion toggle anchor.

Example 363. The system according to any one of examples 361-362, wherein the system further comprises a driver, adapted to drive the toggle anchor from a first side of a cardiovascular tissue of the subject, through the tissue to an opposite side of the tissue, such that, at the opposite side:

the longitudinal axis of the helical coil lies parallel with the tissue, and the coil is in a non-compressed state in which turns of the coil can move with respect to each other.

Example 364. The system according to example 363, wherein the driver is adapted to deliver the toggle anchor through the tissue while the driver extends through a lumen defined by the coil.

Example 365. Apparatus for use with a subject, the apparatus comprising a medical tool that comprises:
an extracorporeal part at a proximal end of the tool;
a shaft that extends distally from the extracorporeal part, and that is configured to be transluminally advanced into the subject; and
a pair of wires that extend, from the extracorporeal part, along the shaft, to a distal part of the tool,
wherein the extracorporeal part comprises:
a controller, and
a lever that operatively couples the controller to the distal part via the pair of wires by:
the controller being pivotably attached to the lever at a fulcrum of the lever, and each wire of the pair being coupled to the lever at a respective opposite side of the fulcrum,
such that actuation of the controller manipulates the distal part of the tool while the lever balances the wires with respect to each other by pivoting dynamically.

Example 366. The apparatus according to example 365, wherein the controller is slidable axially along the extracorporeal part such that, while the lever continues to balance the wires with respect to each other, sliding the controller in a first axial direction moves the distal part of the tool in the first axial direction.

Example 367. Apparatus for use with a subject, the apparatus comprising:
an implant; and
a delivery tool that comprises:
a kirigami wrap adapted to hold the implant, the delivery tool being configured to transluminally advance the implant into the subject while the implant is held by the kirigami wrap, and
a release mechanism, operatively coupled to the kirigami wrap in a manner in which actuating the release mechanism releases the hold of the kirigami wrap on the implant.

Example 368. A system for use with a heart of a subject, the system comprising an implant comprising:
a tether; and
an assembly, comprising:
a winch, comprising a housing and a spool disposed therein, the spool operatively coupled to the tether such that actuation of the winch tensions the tether, the tether extending, from the spool and out of an aperture of the housing;
a winch anchor, coupled to the winch; and
a shock absorber, coupled to the housing in a manner that mitigates forces acting on the winch anchor.

Example 369. The system or apparatus according to any one of the above examples, wherein the delivery tool is sterilized.

Example 370. The system or apparatus according to any one of the above examples, wherein the implant is sterilized.

Example 371. A method comprising sterilizing the delivery tool of any one of the above examples.

Example 372. A method comprising sterilizing the implant of any one of the above examples.

Example 373. The method according to any one of the above examples, further comprising sterilizing the implant.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the various techniques, methods, operations, steps, etc. described or suggested anywhere herein (including in documents incorporated by reference herein) can be performed on a living animal or on a non-living simulation, such as on a cadaver, cadaver heart, simulator (e.g., with the body parts, tissue, etc. being simulated), etc.

What is claimed is:

1. A system for use with a valve disposed between an atrium and a ventricle of a heart of a subject, the system comprising:
an implant, comprising:
a patch, comprising a flexible sheet,
a patch anchor,
a downstream assembly, comprising a ventricular anchor, and
a tether, tethering the downstream assembly to the patch; and
a delivery tool, having a distal portion transluminally advanceable to the heart while the implant is mounted on the delivery tool, the delivery tool comprising:
a shaft, defining a longitudinal axis of the delivery tool;
a clasp, comprising an upstream support and a downstream support, the clasp being transitionable between:
an open state in which the upstream support and the downstream support are positioned away from each other, and in which the clasp is configured to receive a portion of a leaflet of the valve between the upstream support and the downstream support, and
a grasping state, the clasp being configured to grasp the portion of the leaflet received between the upstream support and the downstream support by being transitioned from the open state toward the grasping state while the portion of the leaflet remains disposed between the upstream support and the downstream support; and
a driver, configured to anchor the patch to the portion of the leaflet using the patch anchor while the portion of the leaflet remains grasped by the clasp.

2. The system according to claim 1, wherein the delivery tool has an extracorporeal proximal portion that comprises a clasp controller operatively coupled to the clasp such that operation of the clasp controller transitions the clasp between the open state and the grasping state.

3. The system according to claim 2, wherein the clasp controller is operatively coupled to the upstream support of the clasp such that operation of the clasp controller transitions the clasp between the open state and the grasping state via movement of the upstream support with respect to the shaft.

4. The system according to claim 3, wherein:
the delivery tool further comprises a pair of clasp-control wires via which the clasp controller is operatively coupled to the clasp, and
the extracorporeal portion comprises a lever:
via which the clasp controller is operatively coupled to both wires of the pair, and
adapted to pivot in a manner that balances the wires of the pair with respect to each other.

5. The system according to claim 2, wherein:
the extracorporeal proximal portion further comprises an anchor controller, and
the delivery tool further comprises a driveshaft subassembly that comprises one or more driveshafts extending through the shaft, the driveshaft subassembly configured such that, in at least one state of the delivery tool, the driveshaft subassembly operatively couples the anchor controller to the ventricular anchor such that operation of the anchor controller applies an anchoring force to the ventricular anchor.

6. The system according to claim 5, wherein:
the downstream assembly further comprises a winch, the ventricular anchor being a winch anchor that is coupled to the winch,
the tether tethers the winch to the patch, and
the extracorporeal proximal portion further comprises a winch controller, the driveshaft subassembly configured such that, in at least one state of the delivery tool, the driveshaft subassembly operatively couples the winch controller to the winch such that operation of the winch controller actuates the winch.

7. The system according to claim 6, wherein the driveshaft subassembly comprises:
a winch-control driveshaft via which the winch controller is operatively coupled to the winch, and
an anchor-control driveshaft disposed through the winch-control driveshaft, and via which the anchor controller is operatively coupled to the anchor.

8. The system according to claim 1, wherein:
the ventricular anchor is a first ventricular anchor,
the downstream assembly further comprises a second ventricular anchor, and
the tether tethers the patch to both the first ventricular anchor and the second ventricular anchor.

9. The system according to claim 1, wherein:
the downstream assembly further comprises a winch, the ventricular anchor being a winch anchor that is coupled to the winch, and
the tether tethers the winch to the patch.

10. The system according to claim 9, wherein the delivery tool further comprises a driveshaft subassembly, the driveshaft subassembly comprising one or more driveshafts, extending through the shaft, and operatively coupled to the downstream assembly in a manner that configures the driveshaft subassembly:
to anchor the winch anchor to ventricular tissue of the heart by applying an anchoring force to the winch anchor, and
to actuate the winch independently of applying the anchoring force.

11. The system according to claim 1, wherein the driver is configured to anchor the patch to the portion of the leaflet by driving the patch anchor through the portion of the leaflet grasped by the clasp.

12. The system according to claim 11, wherein the patch anchor is a toggle that is biased to automatically widen upon deployment.

13. The system according to claim 1, wherein:
the delivery tool is configured to anchor the downstream assembly to ventricular tissue of the ventricle by anchoring the ventricular anchor to the ventricular tissue,
the ventricular anchor comprises a tissue-engaging element, and
the delivery tool is configured to anchor the downstream assembly to the ventricular tissue by driving the tissue-engaging element into the ventricular tissue.

14. The system according to claim 1, wherein:
the implant comprises an upstream assembly comprising the patch anchor coupled to the patch, and
the upstream assembly further comprises a cord via which the patch anchor is coupled to the patch.

15. The system according to claim 14, wherein the patch anchor is a toggle anchor.

16. The system according to claim 15, wherein:
the toggle anchor has a tip, a heel, and an eyelet partway between the tip and the heel, and
the heel is flared in a manner that:
facilitates passage of the heel through the leaflet in a first direction, and
inhibits passage of the heel through the leaflet in a second direction that is opposite to the first direction.

17. The system according to claim 1, wherein the clasp defines slot, and the driver is configured to anchor the patch to the leaflet by driving the patch anchor through the leaflet and the slot.

18. The system according to claim 17, wherein the clasp defines a resilient tooth configured to facilitate the patch anchor being driven by the driver through the slot, and to inhibit the patch anchor from being withdrawn, in a reverse direction, through the slot.

19. The system according to claim 17, wherein the clasp defines a resilient slot guard, configured to obstruct tissue of the heart from entering the slot.

20. The system according to claim 1, wherein the delivery tool further comprises a retrieval line, releasably coupled to the anchor such that tensioning the retrieval line de-anchors the patch anchor from the leaflet.
